(19) United States
(12) Patent Application Publication
(10) Pub. No.: US 2024/0335476 A1
(43) Pub. Date: Oct. 10, 2024

(54) SYSTEMS AND METHODS FOR THE TREATMENT OF HEMOGLOBINOPATHIES

(71) Applicant: EDITAS MEDICINE, INC., Cambridge, MA (US)

(72) Inventor: KaiHsin CHANG, Medfield, MA (US)

(73) Assignee: EDITAS MEDICINE, INC., Cambridge, MA (US)

(21) Appl. No.: 18/294,995

(22) PCT Filed: Aug. 2, 2022
(86) PCT No.: PCT/US22/39192

§ 371 Date: Feb. 2, 2024

Related U.S. Application Data
(60) Provisional application No. 63/228,509, filed on Aug. 2, 2021.
(60) Provisional application No. 63/278,899, filed on Nov. 12, 2021.

(51) Int. Cl. A61K 35/28; A61P 7/06; C12N 9/22; C12N 15/11; C12N 15/90

(52) U.S. Cl. CPC A61K 35/28; A61P 7/06; C12N 9/22; C12N 15/11; C12N 15/907; C12N 2310/20

(57) ABSTRACT

Genome editing systems, guide RNAs, and CRISPR-mediated methods are provided for altering portions of the HBG1 and HBG2 loci in cells and increasing expression of fetal hemoglobin.

### PRIORITY CLAIM

This application claims the benefit of U.S. Provisional Application No. 63/228,509, filed Aug. 2, 2021, and U.S. Provisional Application No. 63/278,899, filed Nov. 12, 2021, both of which are incorporated herein by reference in their entirety.

### SEQUENCE LISTING

This application contains a Sequence Listing, which was submitted in ASCII format via EFS-Web, and is hereby incorporated by reference in its entirety. The ASCII copy, created on Nov. 12, 2021, is named SequenceListing.txt and is 699 KB in size.

### FIELD

This disclosure relates to genome editing systems and methods for altering a target nucleic acid sequence, or modulating expression of a target nucleic acid sequence, and applications thereof in connection with the alteration of genes encoding hemoglobin subunits and/or treatment of hemoglobinopathies.

### BACKGROUND

Hemoglobin (Hb) carries oxygen in erythrocytes or red blood cells (RBCs) from the lungs to tissues. During prenatal development and until shortly after birth, hemoglobin is present in the form of fetal hemoglobin (HbF), a tetrameric protein composed of two alpha (α)-globin chains and two gamma (γ)-globin chains. HbF is largely replaced by adult hemoglobin (HbA), a tetrameric protein in which the γ-globin chains of HbF are replaced with beta (β)-globin chains, through a process known as globin switching. The average adult makes less than 1% HbF out of total hemoglobin (Thein 2009). The α-hemoglobin gene is located on chromosome 16, while the β-hemoglobin gene (HBB), A gamma (Aγ)-globin chain (HBG1, also known as gamma globin A), and G gamma (Gγ)-globin chain (HBG2, also known as gamma globin G) are located on chromosome 11 within the globin gene cluster (also referred to as the globin locus).

Mutations in HBB can cause hemoglobin disorders (i.e., hemoglobinopathies) including sickle cell disease (SCD) and beta-thalassemia (β-Thal). Approximately 93,000 people in the United States are diagnosed with a hemoglobinopathy. Worldwide, 300,000 children are born with hemoglobinopathies every year (Angastiniotis 1998). Because these conditions are associated with HBB mutations, their symptoms typically do not manifest until after globin switching from HbF to HbA.

SCD is the most common inherited hematologic disease in the United States, affecting approximately 80,000 people (Brousseau 2010). SCD is most common in people of African ancestry, for whom the prevalence of SCD is 1 in 500. In Africa, the prevalence of SCD is 15 million (Aliyu 2008). SCD is also more common in people of Indian, Saudi Arabian and Mediterranean descent. In those of Hispanic-American descent, the prevalence of sickle cell disease is 1 in 1,000 (Lewis 2014).

SCD is caused by a single homozygous mutation in the HBB gene, c.17A>T (HbS mutation). The sickle mutation is a point mutation (GAG>GTG) on HBB that results in substitution of valine for glutamic acid at amino acid position 6 in exon 1. The valine at position 6 of the β-hemoglobin chain is hydrophobic and causes a change in conformation of the β-globin protein when it is not bound to oxygen. This change of conformation causes HbS proteins to polymerize in the absence of oxygen, leading to deformation (i.e., sickling) of RBCs. SCD is inherited in an autosomal recessive manner, so that only patients with two HbS alleles have the disease. Heterozygous subjects have sickle cell trait, and may suffer from anemia and/or painful crises if they are severely dehydrated or oxygen deprived.

Sickle shaped RBCs cause multiple symptoms, including anemia, sickle cell crises, vaso-occlusive crises, aplastic crises, and acute chest syndrome. Sickle shaped RBCs are less elastic than wild-type RBCs and therefore cannot pass as easily through capillary beds and cause occlusion and ischemia (i.e., vaso-occlusion). Vaso-occlusive crisis occurs when sickle cells obstruct blood flow in the capillary bed of an organ leading to pain, ischemia, and necrosis. These episodes typically last 5-7 days. The spleen plays a role in clearing dysfunctional RBCs, and is therefore typically enlarged during early childhood and subject to frequent vaso-occlusive crises. By the end of childhood, the spleen in SCD patients is often infarcted, which leads to autosplenectomy. Hemolysis is a constant feature of SCD and causes anemia. Sickle cells survive for 10-20 days in circulation, while healthy RBCs survive for 90-120 days. SCD subjects are transfused as necessary to maintain adequate hemoglobin levels. Frequent transfusions place subjects at risk for infection with HIV, Hepatitis B, and Hepatitis C. Subjects may also suffer from acute chest crises and infarcts of extremities, end organs, and the central nervous system.

Subjects with SCD have decreased life expectancies. The prognosis for patients with SCD is steadily improving with careful, life-long management of crises and anemia. As of 2001, the average life expectancy of subjects with sickle cell disease was the mid-to-late 50's. Current treatments for SCD involve hydration and pain management during crises, and transfusions as needed to correct anemia.

Thalassemias (e.g., β-Thal, δ-Thal, and β/δ-Thal) cause chronic anemia. β-Thal is estimated to affect approximately 1 in 100,000 people worldwide. Its prevalence is higher in certain populations, including those of European descent, where its prevalence is approximately 1 in 10,000. β-Thal major, the more severe form of the disease, is life-threatening unless treated with lifelong blood transfusions and chelation therapy. In the United States, there are approximately 3,000 subjects with β-Thal major. β-Thal intermedia does not require blood transfusions, but it may cause growth delay and significant systemic abnormalities, and it frequently requires lifelong chelation therapy. Although HbA makes up the majority of hemoglobin in adult RBCs, approximately 3% of adult hemoglobin is in the form of HbA2, an HbA variant in which the two γ-globin chains are replaced with two delta (Δ)-globin chains. δ-Thal is associated with mutations in the A hemoglobin gene (HBD) that cause a loss of HBD expression. Co-inheritance of the HBD mutation can mask a diagnosis of β-Thal (i.e., β/δ-Thal) by decreasing the level of HbA2 to the normal range (Bouva 2006). β/δ-Thal is usually caused by deletion of the HBB and HBD sequences in both alleles. In homozygous (δo/δo βo/ρo) patients, HBG is expressed, leading to production of HbF alone.

Like SCD, β-Thal is caused by mutations in the HBB gene. The most common HBB mutations leading to β-Thal are: c.-136C>G, c.92+1G>A, c.92+6T>C, c.93-21G>A, c.118C>T, c.316-106C>G, c.25_26delAA, c.27_28insG, c.92+5G>C, c.118C>T, c.135delC, c.315+1G>A, c.-78A>G, c.52A>T, c.59A>G, c.92+5G>C, c.124_127delTTCT, c.316-197C>T, c.-78A>G, c.52A>T, c.124_127delTTCT, c.316-197C>T, c.-138C>T, c.-79A>G, c.92+5G>C, c.75T>A, c.316-2A>G, and c.316-2A>C. These and other mutations associated with β-Thal cause mutated or absent β-globin chains, which causes a disruption of the normal Hb α-hemoglobin to β-hemoglobin ratio. Excess α-globin chains precipitate in erythroid precursors in the bone marrow.

In β-Thal major, both alleles of HBB contain nonsense, frameshift, or splicing mutations that leads to complete absence of β-globin production (denoted βo/βo). β-Thal major results in severe reduction in β-globin chains, leading to significant precipitation of α-globin chains in RBCs and more severe anemia.

β-Thal intermedia results from mutations in the 5′ or 3′ untranslated region of HBB, mutations in the promoter region or polyadenylation signal of HBB, or splicing mutations within the HBB gene. Patient genotypes are denoted βo/β+ or β+/β+. βo represents absent expression of a β-globin chain; β+ represents a dysfunctional but present β-globin chain. Phenotypic expression varies among patients. Since there is some production of β-globin, β-Thal intermedia results in less precipitation of α-globin chains in the erythroid precursors and less severe anemia than β-Thal major. However, there are more significant consequences of erythroid lineage expansion secondary to chronic anemia.

Subjects with β-Thal major present between the ages of 6 months and 2 years, and suffer from failure to thrive, fevers, hepatosplenomegaly, and diarrhea. Adequate treatment includes regular transfusions. Therapy for β-Thal major also includes splenectomy and treatment with hydroxyurea. If patients are regularly transfused, they will develop normally until the beginning of the second decade. At that time, they require chelation therapy (in addition to continued transfusions) to prevent complications of iron overload. Iron overload may manifest as growth delay or delay of sexual maturation. In adulthood, inadequate chelation therapy may lead to cardiomyopathy, cardiac arrhythmias, hepatic fibrosis and/or cirrhosis, diabetes, thyroid and parathyroid abnormalities, thrombosis, and osteoporosis. Frequent transfusions also put subjects at risk for infection with HIV, hepatitis B and hepatitis C.

β-Thal intermedia subjects generally present between the ages of 2-6 years. They do not generally require blood transfusions. However, bone abnormalities occur due to chronic hypertrophy of the erythroid lineage to compensate for chronic anemia. Subjects may have fractures of the long bones due to osteoporosis. Extramedullary erythropoiesis is common and leads to enlargement of the spleen, liver, and lymph nodes. It may also cause spinal cord compression and neurologic problems. Subjects also suffer from lower extremity ulcers and are at increased risk for thrombotic events, including stroke, pulmonary embolism, and deep vein thrombosis. Treatment of β-Thal intermedia includes splenectomy, folic acid supplementation, hydroxyurea therapy, and radiotherapy for extramedullary masses. Chelation therapy is used in subjects who develop iron overload.

Life expectancy is often diminished in β-Thal patients. Subjects with β-Thal major who do not receive transfusion therapy generally die in their second or third decade. Subjects with β-Thal major who receive regular transfusions and adequate chelation therapy can live into their fifth decade and beyond. Cardiac failure secondary to iron toxicity is the leading cause of death in β-Thal major subjects due to iron toxicity.

A variety of new treatments are currently in development for SCD and β-Thal. Delivery of an anti-sickling HBB gene via gene therapy is currently being investigated in clinical trials. However, the long-term efficacy and safety of this approach is unknown. Transplantation with hematopoietic stem cells (HSCs) from an HLA-matched allogeneic stem cell donor has been demonstrated to cure SCD and β-Thal, but this procedure involves risks including those associated with ablation therapy, which is required to prepare the subject for transplant, increases risk of life-threatening opportunistic infections, and risk of graft vs. host disease after transplantation. In addition, matched allogeneic donors often cannot be identified. Thus, there is a need for improved methods of managing these and other hemoglobinopathies.

### SUMMARY

In certain aspects, a method of alleviating one or more symptoms of beta-thalassemia (β-Thal) in a subject in need thereof is provided. In certain embodiments, the method comprises a) isolating a population of CD34+ or hematopoietic stem cells from the subject; b) modifying the population of isolated cells ex vivo by delivering an RNP complex to the population of isolated cells, thereby altering a promoter of an HBG gene in one or more isolated cells in the population, the RNP complex comprising: a Cpf1 and a gRNA comprising: a 5′ end and a 3′ end, an RNA or DNA extension at the 5′ end, a modification, e.g., a phosphorothioate linkage and/or a 2′-O-methyl modification at the 5′ and/or 3′ end, and a targeting domain that is complementary to a target site in the promoter of the HBG gene, and c) administering the modified population of isolated cells to the subject, thereby alleviating one or more symptoms of β-Thal in the subject. In certain embodiments, the modification may be a 2′-O-methyl modification (e.g., a 2′-O-methyladenosine) at the 3′ end, at the 5′ end, or at the 3′ and 5′ ends. In certain embodiments, the modification may be a phosphorothioate linkage followed by a 2′-O-methyladenosine at the 3′ end. In certain embodiments, the DNA extension comprises a sequence selected from the group consisting of SEQ ID NOs:1235-1250. In certain embodiments, the targeting domain comprises, or consists of, a sequence set forth in Tables 7, 8, 11, or 12. In certain embodiments, the target site comprises nucleotides located between Chr 11 (NC_000011.10) 5,249,904-5,249,927 (Table 6, Region 6); Chr 11 (NC_000011.10) 5,254,879-5,254,909 (Table 6, Region 16); or a combination thereof. In certain embodiments, the Cpf1 comprises one or more modifications selected from the group consisting of one or more mutations in a wild-type Cpf1 amino acid sequence, one or more mutations in a wild-type Cpf1 nucleic acid sequence, one or more nuclear localization signals (NLS), one or more purification tags, and a combination thereof. In certain embodiments, the Cpf1 comprises or consists of a sequence selected from the group consisting of SEQ ID NOs: 1000, 1001, 1008-1018, 1032, 1035-39, 1094-1097, and 1107-09. In certain embodiments, the Cpf1 comprises or consists of a sequence selected from the group consisting of SEQ ID NOs:1019-1021 and 1110-17. In certain embodiments, the RNP complex is delivered to the cell using electroporation.

In certain aspects, a method of inducing expression of hemoglobin (Hb) in a population of CD34+ or hematopoietic stem cells from a subject with beta-thalassemia (3-Thal) is provided. In certain embodiments, the method comprises delivering an RNP complex comprising a guide RNA (gRNA) and a Cpf1 to a population of unmodified CD34+ or hematopoietic stem cells from a subject with β-Thal to generate a population of modified CD34+ or hematopoietic stem cells comprising indels, the gRNA comprising a gRNA targeting domain, in which each modified CD34+ or hematopoietic stem cell comprises an indel in an HBG gene promoter, and in which the population of modified CD34+ or hematopoietic stem cells comprises higher Hb levels than the population of unmodified CD34+ or hematopoietic stem cells. In certain embodiments, the gRNA comprises a DNA extension comprising a sequence selected from the group consisting of SEQ ID NOs:1235-1250. In certain embodiments, the gRNA targeting domain comprises, or consists of, a sequence set forth in Tables 7, 8, 11, or 12. In certain embodiments, the gRNA comprises a targeting domain that is complementary to a target site in the promoter of an HBG gene, wherein the target site comprises nucleotides located between Chr 11 (NC_000011.10) 5,249,904-5,249,927 (Table 6, Region 6); Chr 11 (NC_000011.10) 5,254,879-5,254,909 (Table 6, Region 16); or a combination thereof. In certain embodiments, the RNP complex comprises a Cpf1 comprising one or more modifications selected from the group consisting of one or more mutations in a wild-type Cpf1 amino acid sequence, one or more mutations in a wild-type Cpf1 nucleic acid sequence, one or more nuclear localization signals (NLS), one or more purification tags, and a combination thereof. In certain embodiments, the Cpf1 comprises or consists of a sequence selected from the group consisting of SEQ ID NOs: 1000, 1001, 1008-1018, 1032, 1035-39, 1094-1097, and 1107-09. In certain embodiments, the Cpf1 comprises or consists of a sequence selected from the group consisting of SEQ ID NOs:1019-1021 and 1110-17. In certain embodiments, the RNP complex is delivered to the cell using electroporation.

Provided herein are genome editing systems, ribonucleoprotein (RNP) complexes, guide RNAs, Cpf1 proteins, including modified Cpf1 proteins (Cpf1 variants), and CRISPR-mediated methods for altering the promoter region of one or more γ-globin genes (e.g., HBG1, HBG2, or HBG1 and HBG2) and increasing expression of fetal hemoglobin (HbF). In certain embodiments, an RNP complex may include a guide RNA (gRNA) complexed to a wild-type Cpf1 or modified Cpf1 RNA-guided nuclease (modified Cpf1 protein).

In certain embodiments, a gRNA may comprise a sequence set forth in Tables 7, 8, 11, or 12. In certain embodiments, the RNP complex may comprise an RNP complex set forth in Table 10. For example, an RNP complex may include a gRNA comprising the sequence set forth in SEQ ID NO:1051 and a modified Cpf1 protein encoded by the sequence set forth in SEQ ID NO: 1097 (RNP32, Table 10).

In certain embodiments, the modified Cpf1 protein may contain one or more modifications. In certain embodiments, the one or more modifications may include, without limitation, one or more mutations in a wild-type Cpf1 amino acid sequence, one or more mutations in a wild-type Cpf1 nucleic acid sequence, one or more nuclear localization signals (NLS), one or more purification tags (e.g., His tag), or a combination thereof. In certain embodiments, a modified Cpf1 may be encoded by a sequence set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences).

In certain embodiments, an RNP complex comprising a modified Cpf1 protein may increase editing of a target nucleic acid. In certain embodiments, an RNP complex comprising a modified Cpf1 protein may increase editing resulting in an increase of productive indels. In various embodiments, an increase in editing of the target nucleic acid may be assessed by any means known to skilled artisans, such as, but not limited to, PCR amplification of the target nucleic acid and subsequent sequencing analysis (e.g., Sanger sequencing, next generation sequencing).

In certain embodiments, the gRNA may comprise one or more modifications including a phosphorothioate linkage modification, a phosphorodithioate (PS2) linkage modification, a 2′-O-methyl modification, one or more or a stretch of deoxyribonucleic acid (DNA) bases (also referred to herein as a “DNA extension”), one or more or a stretch of ribonucleic acid (RNA) bases (also referred to herein as a “RNA extension”), or combinations thereof. In certain embodiments, the DNA extension may comprise a sequence set forth in Table 13. For example, in certain embodiments, the DNA extension may comprise a sequence set forth in SEQ ID NOs:1235-1250. In certain embodiments, the RNA extension may comprise a sequence set forth in Table 13. For example, in certain embodiments, the RNA extension may comprise a sequence set forth in SEQ ID NOs:1231-1234, 1251-1253. In certain embodiments, an RNP complex comprising a modified gRNA may increase editing of a target nucleic acid. In certain embodiments, an RNP complex comprising a modified gRNA may increase editing resulting in an increase of productive indels.

In one aspect, the disclosure relates to an RNP complex comprising a CRISPR from Prevotella and Franciscella 1 (Cpf1) RNA-guided nuclease or a variant thereof and a gRNA, wherein the gRNA is capable of binding to a target site in a promoter of an HBG gene in a cell. In certain embodiments, the gRNA may be modified or unmodified. In certain embodiments, the gRNA may comprise one or more modifications including a phosphorothioate linkage modification, a phosphorodithioate (PS2) linkage modification, a 2′-O-methyl modification, a DNA extension, an RNA extension, or combinations thereof. In certain embodiments, the DNA extension may comprise a sequence set forth in Table 13. In certain embodiments, the RNA extension may comprise a sequence set forth in Table 13. In certain embodiments, the gRNA may comprise a sequence set forth in Tables 7, 8, 11, or 12. In certain embodiments, the RNP complex may comprise an RNP complex set forth in Table 10. For example, the RNP complex may include a gRNA comprising the sequence set forth in SEQ ID NO:1051 and a Cpf1 variant protein encoded by the sequence set forth in SEQ ID NO:1097 (RNP32, Table 10). In certain embodiments, the Cpf1 variant protein may contain one or more modifications. In certain embodiments, the one or more modifications may include, without limitation, one or more mutations in a wild-type Cpf1 amino acid sequence, one or more mutations in a wild-type Cpf1 nucleic acid sequence, one or more nuclear localization signals (NLS), one or more purification tags (e.g., His tag), or a combination thereof. In certain embodiments, a Cpf1 variant protein may be encoded by a sequence set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences).

In one aspect, the disclosure relates to a method of altering a promoter of an HBG gene in a cell comprising contacting the cell with an RNP complex disclosed herein. In certain embodiments, the alteration may comprise an indel within one or more regions set forth in Table 6. In certain embodiments, the alteration may comprise an indel within a CCAAT box target region of the promoter of an HBG gene. For example, in certain embodiments, the alteration may comprise an indel within Chr 11 (NC_000011.10): 5,249,955-5,249,987 (Table 6, Region 6), Chr 11 (NC_000011.10): 5,254,879-5,254,909 (Table 6, Region 16), or a combination thereof. In certain embodiments, the RNP complex may comprise a gRNA and a Cpf1 protein. In certain embodiments, the gRNA may comprise an RNA targeting domain set forth in Table 8. In certain embodiments, the gRNA targeting domain may comprise a sequence selected from the group consisting of SEQ ID NOs:1002, 1254, 1258, 1260, 1262, and 1264. In certain embodiments, the gRNA may comprise a gRNA sequence set forth in Table 8. In certain embodiments, the gRNA may comprise a sequence selected from the group consisting of SEQ ID NOs:1022, 1023, 1041-1105. In certain embodiments, a gRNA may be configured to provide an editing event at Chr11:5249973, Chr11:5249977 (HBG1); Chr11:5250042, Chr11:5250046 (HBG1); Chr11:5250055, Chr11:5250059 (HBG1); Chr11:5250179, Chr11:5250183 (HBG1); Chr11:5254897, Chr11:5254901 (HBG2); Chr11:5254897, Chr11:5254901 (HBG2); Chr11:5254966, 5254970 (HBG2); Chr11:5254979, 5254983 (HBG2) (Table 6, Table 7).

In one aspect, the disclosure relates to an isolated cell comprising an alteration in a promoter of HBG gene generated by the delivery of an RNP complex to the cell. In certain embodiments, the RNP complex may comprise a gRNA and a Cpf1 protein. In certain embodiments, the gRNA may be modified or unmodified. In certain embodiments, the gRNA may comprise one or more modifications including a phosphorothioate linkage modification, a phosphorodithioate (PS2) linkage modification, a 2′-O-methyl modification, a DNA extension, an RNA extension, or combinations thereof. In certain embodiments, the DNA extension may comprise a sequence set forth in Table 13. In certain embodiments, the RNA extension may comprise a sequence set forth in Table 13. In certain embodiments, the gRNA may comprise a sequence set forth in Tables 7, 8, 11, or 12. In certain embodiments, the RNP complex may comprise an RNP complex set forth in Table 10. For example, the RNP complex may include a gRNA comprising the sequence set forth in SEQ ID NO:1051 and a Cpf1 variant protein encoded by the sequence set forth in SEQ ID NO:1097 (RNP32, Table 10). In certain embodiments, the Cpf1 variant protein may contain one or more modifications. In certain embodiments, the one or more modifications may include, without limitation, one or more mutations in a wild-type Cpf1 amino acid sequence, one or more mutations in a wild-type Cpf1 nucleic acid sequence, one or more nuclear localization signals (NLS), one or more purification tags (e.g., His tag), or a combination thereof. In certain embodiments, a Cpf1 variant protein may be encoded by a sequence set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences).

In one aspect, the disclosure relates to an ex vivo method of increasing the level of fetal hemoglobin (HbF) in a human cell by genome editing using an RNP complex comprising a gRNA and a Cpf1 RNA-guided nuclease or a variant thereof to affect an alteration in a promoter of an HBG gene, thereby to increase expression of HbF. In certain embodiments, the gRNA may be modified or unmodified. In certain embodiments, the gRNA may comprise one or more modifications including a phosphorothioate linkage modification, a phosphorodithioate (PS2) linkage modification, a 2′-O-methyl modification, a DNA extension, an RNA extension, or combinations thereof. In certain embodiments, the DNA extension may comprise a sequence set forth in Table 13. In certain embodiments, the RNA extension may comprise a sequence set forth in Table 13. In certain embodiments, the gRNA may comprise a sequence set forth in Tables 7, 8, 11, or 12. In certain embodiments, the RNP complex may comprise an RNP complex set forth in Table 10. For example, the RNP complex may include a gRNA comprising the sequence set forth in SEQ ID NO:1051 and a Cpf1 variant protein encoded by the sequence set forth in SEQ ID NO:1097 (RNP32, Table 10). In certain embodiments, the Cpf1 variant protein may contain one or more modifications. In certain embodiments, the one or more modifications may include, without limitation, one or more mutations in a wild-type Cpf1 amino acid sequence, one or more mutations in a wild-type Cpf1 nucleic acid sequence, one or more nuclear localization signals (NLS), one or more purification tags (e.g., His tag), or a combination thereof. In certain embodiments, a Cpf1 variant protein may be encoded by a sequence set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences).

In one aspect, the disclosure relates to a population of CD34+ or hematopoietic stem cells, wherein one or more cells in the population comprises an alteration in a promoter of an HBG gene, which alteration is generated by delivering an RNP complex comprising a gRNA and a Cpf1 RNA-guided nuclease or a variant thereof to the population of CD34+ or hematopoietic stem cells. In certain embodiments, the gRNA may be modified or unmodified. In certain embodiments, the gRNA may comprise one or more modifications including a phosphorothioate linkage modification, a phosphorodithioate (PS2) linkage modification, a 2′-O-methyl modification, a DNA extension, an RNA extension, or combinations thereof. In certain embodiments, the DNA extension may comprise a sequence set forth in Table 13. In certain embodiments, the RNA extension may comprise a sequence set forth in Table 13. In certain embodiments, the gRNA may comprise a sequence set forth in Tables 7, 8, 11, or 12. In certain embodiments, the RNP complex may comprise an RNP complex set forth in Table 10. For example, the RNP complex may include a gRNA comprising the sequence set forth in SEQ ID NO:1051 and a Cpf1 variant protein encoded by the sequence set forth in SEQ ID NO:1097 (RNP32, Table 10). In certain embodiments, the Cpf1 variant protein may contain one or more modifications. In certain embodiments, the one or more modifications may include, without limitation, one or more mutations in a wild-type Cpf1 amino acid sequence, one or more mutations in a wild-type Cpf1 nucleic acid sequence, one or more nuclear localization signals (NLS), one or more purification tags (e.g., His tag), or a combination thereof. In certain embodiments, a Cpf1 variant protein may be encoded by a sequence set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences).

In one aspect, the disclosure relates to a method of alleviating one or more symptoms of beta thalassemia in a subject in need thereof, the method comprising: a) isolating a population of CD34+ or hematopoietic stem cells from the subject; b) modifying the population of isolated cells ex vivo by delivering an RNP complex comprising a gRNA and a Cpf1 RNA-guided nuclease or a variant thereof to the population of isolated cells, thereby to affect an alteration in a promoter of an HBG gene in one or more cells in the population; and c) administering the modified population of cells to the subject, thereby to alleviate one or more symptoms of beta thalassemia in the subject. In certain embodiments, the method may further comprise detecting progeny/daughter cells of the administered modified cells in the subject, e.g., in the form of BM-engrafted CD34+ hematopoietic stem cells or blood cells derived from those (e.g., myeloid progenitor or differentiated myeloid cells (e.g., erythrocyte, mast cells, myoblast); or lymphoid progenitors or differentiated lymphoid cells (e.g., T- or B-lymphocyte, or NK cell), e.g., at least [1, 2, 3, 4, 5, 6, 7, 8, 12, 16, or 20] weeks, or at least [1, 2, 3, 4, 5, or 6] months, or at least [1, 2, 3, 4, or 5] years after administration. In certain embodiments, the method may result in a reconstitution of all hematopoietic cell lineages, e.g., without any differentiation bias, e.g., without an erythroid lineage differentiation bias. In certain embodiments, the method may comprise administering a plurality of edited cells, and the method may result in long-term engraftment [e.g., at least [1, 2, 3, 4, 5, 6, 7, 8, 12, 16, or 20] weeks, or at least [1, 2, 3, 4, 5, or 6] months, or at least [1, 2, 3, 4, or 5] years after administration] of a plurality of [at least 5, 10, 15, 20, 25, . . . 100] different HSC clones in the BM. In certain embodiments, the method may further comprise detecting the level of total hemoglobin expression in the subject, at least [1, 2, 3, 4, 5, 6, 7, 8, 12, 16, or 20] weeks, or at least [1, 2, 3, 4, 5, or 6] months, or at least [1, 2, 3, 4, or 5] years after administration. In certain embodiments, the method may result in long-term expression [e.g., at least [1, 2, 3, 4, 5, 6, 7, 8, 12, 16, or 20] weeks, or at least [1, 2, 3, 4, 5, or 6] months, or at least [1, 2, 3, 4, or 5] years after administration] of [at least 50%, at least 60% . . . at least 99%] of total hemoglobin as compared to a healthy subject (e.g., as total Hb (e.g., HbA and HbF (if any) combined)). In certain embodiments, the alteration may comprise an indel within a CCAAT box target region of the promoter of the HBG gene. In certain embodiments, the RNP complex may be delivered using electroporation. In certain embodiments, at least about 5%, at least about 10%, at least about 20%, at least about 30%, at least about 40%, at least about 50%, at least about 60%, at least about 70%, at least about 80% or at least about 90% of the cells in the population of cells comprise a productive indel.

In one aspect, the disclosure relates to a method of alleviating one or more symptoms of beta-thalassemia (3-Thal) in a subject in need thereof, the method including: a) isolating a population of CD34+ or hematopoietic stem cells from the subject; b) modifying the population of isolated cells ex vivo by delivering an RNP complex to the population of isolated cells, thereby altering a promoter of an HBG gene in one or more isolated cells in the population, the RNP complex comprising: a Cpf1 and a gRNA comprising: a 5′ end and a 3′ end, a DNA extension at the 5′ end, a 2′-O-methyl-3′-phosphorothioate modification at the 3′ end, and a targeting domain that is complementary to a target site in the promoter of the HBG gene, and c) administering the modified population of isolated cells to the subject, thereby alleviating one or more symptoms of β-Thal in the subject. In certain embodiments, the DNA extension may include a sequence selected from the group consisting of SEQ ID NOs:1235-1250. In certain embodiments, the targeting domain may include a sequence selected from the group consisting of a set forth in Tables 7, 8, 11, and 12. In certain embodiments, the target site may include nucleotides located between Chr 11 (NC_000011.10) 5,249,904-5,249,927 (Table 6, Region 6); Chr 11 (NC_000011.10) 5,254,879-5,254,909 (Table 6, Region 16); or a combination thereof. In certain embodiments, the Cpf1 may include one or more modifications selected from the group consisting of one or more mutations in a wild-type Cpf1 amino acid sequence, one or more mutations in a wild-type Cpf1 nucleic acid sequence, one or more nuclear localization signals (NLS), one or more purification tags, and a combination thereof. In certain embodiments, the Cpf1 may be a Cpf1 variant and may comprise or consist of a sequence selected from the group consisting of SEQ ID NOs: 1000, 1001, 1008-1018, 1032, 1035-39, 1094-1097, and 1107-09. In certain embodiments, the Cpf1 may be a Cpf1 variant and may comprise or consist of a sequence selected from the group consisting of SEQ ID NOs:1019-1021 and 1110-17. In certain embodiments, the RNP complex may be delivered to the cell using electroporation.

In one aspect, the disclosure relates to a method of inducing expression of hemoglobin (Hb) in a first population of modified cells from a subject with beta-thalassemia (β-Thal) comprising a plurality of modified CD34+ or hematopoietic stem cells, the method including delivering a first RNP complex including a first guide RNA (gRNA) and a Cpf1 to a first population of unmodified cells from a subject with β-Thal comprising a plurality of unmodified CD34+ or hematopoietic stem cells to generate indels, the first gRNA including a first gRNA targeting domain, in which each modified CD34+ or hematopoietic stem cell comprises an indel in an HBG gene promoter, and in which the first population of modified cells comprises higher Hb levels than the first population of unmodified cells. In certain embodiments, the first gRNA may include a DNA extension comprising a sequence selected from the group consisting of SEQ ID NOs:1235-1250. In certain embodiments, the first gRNA targeting domain may include a sequence selected from the group consisting of a set forth in Tables 7, 8, 11, and 12. In certain embodiments, the first gRNA may include a targeting domain that is complementary to a target site in the promoter of an HBG gene, wherein the target site comprises nucleotides located between Chr 11 (NC_000011.10) 5,249,904-5,249,927 (Table 6, Region 6); Chr 11 (NC_000011.10) 5,254,879-5,254,909 (Table 6, Region 16); or a combination thereof. In certain embodiments, the first RNP complex may include a Cpf1 variant comprising one or more modifications selected from the group consisting of one or more mutations in a wild-type Cpf1 amino acid sequence, one or more mutations in a wild-type Cpf1 nucleic acid sequence, one or more nuclear localization signals (NLS), one or more purification tags, and a combination thereof. In certain embodiments, the Cpf1 variant may comprise or consist of a sequence selected from the group consisting of SEQ ID NOs: 1000, 1001, 1008-1018, 1032, 1035-39, 1094-1097, and 1107-09. In certain embodiments, the Cpf1 variant may comprise or consist of a sequence selected from the group consisting of SEQ ID NOs:1019-1021 and 1110-17. In certain embodiments, the first RNP complex may be delivered to the cell using electroporation.

In certain embodiments, the modified CD34+ or hematopoietic stem cells may be erythroblasts differentiated from the modified CD34+ or hematopoietic stem cells. In certain embodiments, the unmodified CD34+ or hematopoietic stem cells may be erythroblasts differentiated from the unmodified CD34+ or hematopoietic stem cells. In certain embodiments, erythroblasts may comprise one or more selected from a live cell, a nucleated cell, a cell that fluoresces using an anti-human CD235a antibody via fluorescence activated cell sorting (FACS), or a combination thereof.

In certain embodiments, 10%, 15%, 20%, 25%, 30%, 35%, 40%, 45%, or 50% or more of erythroblasts differentiated from modified CD34+ or hematopoietic stem cells may be late erythroblasts relative to erythroblasts differentiated from unmodified CD34+ or hematopoietic stem cells. In certain embodiments, late erythroblasts may comprise cells that comprise low or negative CD71 expression.

In certain embodiments, 10%, 15%, 20%, 25%, 30%, 35%, 40%, 45%, or 50% or more of erythroblasts differentiated from modified CD34+ or hematopoietic stem cells may be enucleated erythroid cells relative to erythroblasts differentiated from unmodified CD34+ or hematopoietic stem cells. In certain embodiments, enucleated erythroid cells may be erythroid cells that do not contain a nucleus. In certain embodiments, enucleated erythroid cells may include erythroid cells that do not fluoresce (stain) when using a reagent to detect a cell nucleus (e.g., NucRed reagent).

In certain embodiments, 5%, 10%, 15%, 20%, 25%, 30%, 35%, 40%, 45%, or 50% or more of erythroblasts differentiated from unmodified CD34+ or hematopoietic stem cells may be nonviable erythroblasts relative to erythroblasts differentiated from modified CD34+ or hematopoietic stem cells. In certain embodiments, nonviable erythroblasts comprise cells that fluoresce (stain) with 4′,6-diamidino-2-phenylindole (DAPI).

In certain embodiments, erythroblasts differentiated from modified CD34+ or hematopoietic stem cells may have 5%, 10%, 15%, 20%, 25%, 30%, 35%, 40%, 45%, or 50% higher total hemoglobin content relative to erythroblasts differentiated from unmodified CD34+ or hematopoietic stem cells. In certain embodiments, total hemoglobin content may be measured using reverse phase ultra-performance liquid chromatography (RP-UPLC).

In one aspect, the disclosure relates to a gRNA comprising a 5′ end and a 3′ end, and comprising a DNA extension at the 5′ end and a 2′-O-methyl-3′-phosphorothioate modification at the 3′ end, wherein the gRNA includes an RNA segment capable of hybridizing to a target site and an RNA segment capable of associating with a Cpf1 RNA-guided nuclease. In certain embodiments, the DNA extension may comprise a sequence set forth in SEQ ID NOs:1235-1250. In certain embodiments, the gRNA may be modified or unmodified. In certain embodiments, the gRNA may comprise one or more modifications including a phosphorothioate linkage modification, a phosphorodithioate (PS2) linkage modification, a 2′-O-methyl modification, a DNA extension, an RNA extension, or combinations thereof. In certain embodiments, the DNA extension may comprise a sequence set forth in Table 13. In certain embodiments, the RNA extension may comprise a sequence set forth in Table 13. In certain embodiments, the gRNA may comprise a sequence set forth in Tables 7, 8, 11, or 12.

In one aspect, the disclosure relates to an RNP complex comprising a Cpf1 RNA-guided nuclease as disclosed herein and a gRNA as disclosed herein.

Also provided herein are genome editing systems, guide RNAs, and CRISPR-mediated methods for altering one or more γ-globin genes (e.g., HBG1, HBG2, or HBG1 and HBG2) and increasing expression of fetal hemoglobin (HbF). In certain embodiments, one or more gRNAs comprising a sequence set forth in Tables 7, 8, 11, or 12 may be used to introduce alterations in the promoter region of the HBG gene. In certain embodiments, genome editing systems, guide RNAs, and CRISPR-mediated methods may alter a 13 nucleotide (nt) target region that is 5′ of the transcription site of the HBG1, HBG2, or HBG1 and HBG2 gene (“13 nt target region”). In certain embodiments, genome editing systems, guide RNAs, and CRISPR-mediated methods may alter a CCAAT box target region that is 5′ of the transcription site of the HBG1, HBG2, or HBG1 and HBG2 gene (“CCAAT box target region”). In certain embodiments, the CCAAT box target region may be the region that is at or near the distal CCAAT box and includes the nucleotides of the distal CCAAT box and 25 nucleotides upstream (5′) and 25 nucleotides downstream (3′) of the distal CCAAT box (i.e., HBG1/2 c.-86 to -140). In certain embodiments, the CCAAT box target region may be the region that is at or near the distal CCAAT box and includes the nucleotides of the distal CCAAT box and 5 nucleotides upstream (5′) and 5 nucleotides downstream (3′) of the distal CCAAT box (i.e., HBG1/2 c.-106 to -120). In certain embodiments, the CCAAT box target region may comprise a 18 nt target region, a 13 nt target region, a 11 nt target region, a 4 nt target region, a 1 nt target region, a -117G>A target region, or a combination thereof as disclosed herein. In certain embodiments, the alteration may be a 18 nt deletion, 13 nt deletion, 11 nt deletion, 4 nt deletion, 1 nt deletion, a substitution from G to A at c.-117 of the HBG1, HBG2, or HBG1 and HBG2 gene, or a combination thereof. In certain embodiments, the alteration may be a non-naturally occurring alteration or a naturally occurring alteration.

In certain embodiments, the genome editing systems, guide RNAs, and CRISPR-mediated methods for altering one or more γ-globin genes (e.g., HBG1, HBG2, or HBG1 and HBG2), may include an RNA-guided nuclease. In certain embodiments, the RNA-guided nuclease may a Cpf1 or modified Cpf1 as disclosed herein.

In one aspect, the disclosure relates to compositions including a plurality of cells generated by the methods disclosed above, in which at least 20%, 30%, 40%, 50%, 60%, 70%, 80% or 90% of the cells include an alteration of a sequence of a 13 nt target region of the human HBG1 or HBG2 gene or a plurality of cells generated by the methods disclosed above, wherein at least 20%, 30%, 40%, 50%, 60%, 70%, 80% or 90% of the cells include an alteration of a sequence of a 13 nt target region of the human HBG1 or HBG2 gene. In certain embodiments, at least a portion of the plurality of cells may be within an erythroid lineage. In certain embodiments, the plurality of cells may be characterized by an increased level of fetal hemoglobin expression relative to an unmodified plurality of cells. In certain embodiments, the level of fetal hemoglobin may be increased by at least 20%, 30%, 40%, 50%, 60%, 70%, 80% or 90%. In certain embodiments, the compositions may further include a pharmaceutically acceptable carrier.

The disclosure herein also relates to methods of altering a cells, including contacting a cell with any of the genome editing systems disclosed herein. In certain embodiments, the step of contacting the cell may comprise contacting the cell with a solution comprising first and second ribonucleoprotein complexes. In certain embodiments, the step of contacting the cell with the solution further comprises electroporating the cells, thereby introducing the first and second ribonucleoprotein complexes into the cell.

A genome editing system or method including any of all of the features described above may include a target nucleic acid comprising a human HBG1, HBG2 gene, or a combination thereof. In certain embodiments, the target region may be a CCAAT box target region of the human HBG1, HBG2 gene, or a combination thereof. In certain embodiments, the first targeting domain sequence may be complementary to a first sequence on a side of a CCAAT box target region of the human HBG1, HBG2 gene, or a combination thereof, in which the first sequence optionally overlaps the CCAAT box target region of the human HBG1, HBG2 gene, or a combination thereof. In certain embodiments, the second targeting domain sequence may be complementary to a second sequence on a side of a CCAAT box target region of the human HBG1, HBG2 gene, or a combination thereof, in which the second sequence optionally overlaps the CCAAT box target region of the human HBG1, HBG2 gene, or a combination thereof.

In certain embodiments, a cell may include at least one modified allele of the HBG locus generated by any of the methods for altering a cell disclosed herein, in which the modified allele of the HBG locus comprises an alteration of the human HBG1 gene, HBG2, gene, or a combination thereof.

In certain embodiments, an isolated population of cells may be modified by any of the methods for altering a cells disclosed herein, wherein the population of cells may include a distribution of indels that may be different from an isolated population of cells or their progenies of the same cell type that have not been modified by the method.

In certain embodiments, a plurality of cells may be generated by any of the methods for altering a cells disclosed herein, in which at least 20%, 30%, 40%, 50%, 60%, 70%, 80%, or 90% of the cells may include an alteration of a sequence in the CCAAT box target region of the human HBG1 gene, HBG2 gene or a combination thereof.

In certain embodiments, the cells disclosed herein may be used for a medicament. In certain embodiments, the cells may be for use in the treatment of β-hemoglobinopathy. In certain embodiments, β-hemoglobinopathy may be selected from the group consisting of sickle cell disease and beta-thalassemia. In certain embodiments, the beta-thalassemia may be transfusion-dependent beta thalassemia (TDT).

In one aspect, the disclosure relates to compositions including a plurality of cells generated by a method disclosed above, in which at least 20%, 30%, 40%, 50%, 60%, 70%, 80% or 90% of the cells include an alteration of a sequence of a CCAAT box target region of the human HBG1 or HBG2 gene or a plurality of cells generated by the method disclosed above, wherein at least 20%, 30%, 40%, 50%, 60%, 70%, 80% or 90% of the cells include an alteration of a sequence of a CCAAT box target region of the human HBG1 or HBG2. In certain embodiments, at least a portion of the plurality of cells may be within an erythroid lineage. In certain embodiments, the plurality of cells may be characterized by an increased level of fetal hemoglobin expression relative to an unmodified plurality of cells. In certain embodiments, the level of fetal hemoglobin may be increased by at least 20%, 30%, 40%, 50%, 60%, 70%, 80% or 90%. In certain embodiments, the compositions may further include a pharmaceutically acceptable carrier.

In one aspect, the disclosure relates to a population of cells modified by a genome editing system described above, wherein the population of cells comprise a higher percentage of a productive indel relative to a population of cells not modified by the genome editing system. The disclosure also relates to a population of cells modified by the genome editing system, wherein a higher percentage of the population of cells are capable of differentiating into a population of cells of an erythroid lineage that express HbF relative to a population of cells not modified by the genome editing system. In certain embodiments, the higher percentage may be at least about 15%, at least about 20%, at least about 25%, at least about 30%, or at least about 40% higher. In certain embodiments, the cells may be hematopoietic stem cells. In certain embodiments, the cells may be capable of differentiating into an erythroblast, erythrocyte, or a precursor of an erythrocyte or erythroblast. In certain embodiments, the indel may be created by a repair mechanism other than microhomology-mediated end joining (MMEJ) repair.

The disclosure also relates to the use of any of the cells disclosed herein in the manufacture of a medicament for treating β-hemoglobinopathy in a subject.

In one aspect, the disclosure relates to a method of treating a β-hemoglobinopathy in a subject in need thereof, comprising administering to the subject the cells disclosed herein. In certain embodiments, a method of treating a β-hemoglobinopathy in a subject in need thereof, may include administering a population of modified hematopoietic cells to the subject, wherein one or more cells have been altered according to the methods of altering a cell disclosed herein. In certain embodiments, the method may further comprise detecting progeny/daughter cells of the administered modified cells in the subject, e.g., in the form of BM-engrafted CD34+ hematopoietic stem cells or blood cells derived from those (e.g., myeloid progenitor or differentiated myeloid cells (e.g., erythrocyte, mast cells, myoblast); or lymphoid progenitors or differentiated lymphoid cells (e.g., T- or B-lymphocyte, or NK cell), e.g., at least [1, 2, 3, 4, 5, 6, 7, 8, 12, 16, or 20] weeks, or at least [1, 2, 3, 4, 5, or 6] months, or at least [1, 2, 3, 4, or 5] years after administration. In certain embodiments, the method may result in a reconstitution of all hematopoietic cell lineages, e.g., without any differentiation bias, e.g., without an erythroid lineage differentiation bias. In certain embodiments, the method may comprise administering a plurality of edited cells, and the method may result in long-term engraftment [e.g., at least [1, 2, 3, 4, 5, 6, 7, 8, 12, 16, or 20] weeks, or at least [1, 2, 3, 4, 5, or 6] months, or at least [1, 2, 3, 4, or 5] years after administration] of a plurality of [at least 5, 10, 15, 20, 25, . . . 100] different HSC clones in the BM. In certain embodiments, the method may further comprise detecting the level of total hemoglobin expression in the subject, at least [1, 2, 3, 4, 5, 6, 7, 8, 12, 16, or 20] weeks, or at least [1, 2, 3, 4, 5, or 6] months, or at least [1, 2, 3, 4, or 5] years after administration. In certain embodiments, the method may result in long-term expression [e.g., at least [1, 2, 3, 4, 5, 6, 7, 8, 12, 16, or 20] weeks, or at least [1, 2, 3, 4, 5, or 6] months, or at least [1, 2, 3, 4, or 5] years after administration] of [at least 50%, at least 60% . . . at least 99%] of total hemoglobin as compared to a healthy subject (e.g., as total Hb (e.g., HbA and HbF (if any) combined)). In certain embodiments, the alteration may comprise an indel within a CCAAT box target region of the promoter of the HBG gene.

In one aspect, the disclosure relates to a method of altering a cell comprising contacting a cell with a genome editing system. In certain embodiments, the step of contacting the cell with the genome editing system may comprise contacting the cell with a solution comprising first and second ribonucleoprotein complexes. In certain embodiments, the step of contacting the cell with the solution may further comprise electroporating the cells, thereby introducing the first and second ribonucleoprotein complexes into the cell. In certain embodiments, the method of altering a cell may further comprise contacting the cell with a genome editing system, wherein the step of contacting the cell with the genome editing system may comprise contacting the cell with a solution comprising first, second, third, and optionally, fourth ribonucleoprotein complexes. In certain embodiments, the step of contacting the cell with the solution may further comprise electroporating the cells, thereby introducing the first, second, third, and optionally, fourth ribonucleoprotein complexes into the cell. In certain embodiments, the cell may be capable of differentiating into an erythroblast, erythrocyte, or a precursor of an erythrocyte or erythroblast. In certain embodiments, the cell may be a CD34+ cell.

In one aspect, the disclosure relates to a composition that may comprise a plurality of cells generated by a method of altering a cell disclosed herein, wherein at least 20%, 30%, 40%, 50%, 60%, 70%, 80% or 90% of the cells may comprise an alteration of a sequence of a CCAAT box target region of the human HBG1 gene, HBG2 gene, or a combination thereof. In certain embodiments, at least a portion of the plurality of cells may be within an erythroid lineage. In certain embodiments, the plurality of cells may be characterized by an increased level of fetal hemoglobin expression relative to an unmodified plurality of cells. In certain embodiments, the level of fetal hemoglobin may be increased by at least 20%, 30%, 40%, 50%, 60%, 70%, 80% or 90%. In certain embodiments, the composition may further comprise a pharmaceutically acceptable carrier.

In one aspect, the disclosure relates to a cell comprising a synthetic genotype generated by a method of altering a cell disclosed herein, wherein the cell may comprise a 18 nt deletion, a 11 nt deletion, a 4 nt deletion, a 1 nt deletion, a 13 nt deletion, a substitution from G to A at the -117, of the human HBG1 gene, HBG2 gene, or a combination thereof.

In one aspect, the disclosure relates to a cell comprising at least one allele of the HBG locus generated by a method of altering a cell disclosed herein, wherein the cell may encode a 18 nt deletion, a 11 nt deletion, a 4 nt deletion, a 1 nt deletion, a 13 nt deletion, a substitution from G to A at the -117, of the human HBG1 gene, HBG2 gene, or a combination thereof.

In one aspect, the disclosure relates to a composition, comprising a population of cells generated by a method of altering a cell disclosed herein, wherein the cells comprise a higher frequency of an alteration of a sequence of a CCAAT box target region of the human HBG1 gene, HBG2 gene, or a combination thereof relative to an unmodified population of cells. In certain embodiments, the higher frequency is at least about 10%, 20%, 30%, 40%, 50%, 60%, 70%, 80% or 90% higher. In certain embodiments, at least a portion of the population of cells are within an erythroid lineage.

This listing is intended to be exemplary and illustrative rather than comprehensive and limiting. Additional aspects and embodiments may be set out in, or apparent from, the remainder of this disclosure and the claims.

### BRIEF DESCRIPTION OF THE DRAWINGS

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic two- or three-dimensional structures; these depictions are intended to be illustrative rather than limiting or binding to any particular model or theory regarding their structure.

FIG. 1 depicts, in schematic form, HBG1 and HBG2 gene(s) in the context of the β-globin gene cluster on human chromosome 11. FIG. 1. Each gene in the β-globin gene cluster is transcriptionally regulated by a proximal promoter. While not wishing to be bound by any particular theory, it is generally thought that A_{γ} and/or G_{γ} expression is activated by engagement between the proximal promoter with the distal strong erythroid-specific enhancer, the locus control region (LCR). Long-range transactivation by the LCR is thought to be mediated by alteration of chromatin configuration/confirmation. The LCR is marked by 4 erythroid specific DNase I hypersensitive sites (HS1-4) and 2 distal enhancer elements (5′ HS and 3′ HS1). β-like gene globin gene expression is regulated in a developmental stage-specific manner, and expression of globin genes changes coincide with changes in the main site of blood production.

FIGS. 2A-2B depict HBG1 and HBG2 genes, coding sequences (CDS) and small deletions and point mutations in and upstream of the HBG1 and HBG2 proximal promoters that have been identified in patients and associated with elevation of fetal hemoglobin (HbF). Core elements within the proximal promoters (CAAT box, 13 nt sequence) that have been deleted in some patients with hereditary persistence of fetal hemoglobin (HPFH). The ‘target sequence’ region of each locus, which has been screened for gRNA binding target sites, is also identified.

FIG. 3A shows the percentage of indels in CD34+ cells from three donors with TDT (“B-thal”) and three normal healthy donors (“HD”) at Days 1 to 3 post electroporation with RNP32 (“treated”). “Mock” represents cells electroporated without RNP (unedited cells). Indel=insertions and/or deletions. FIG. 3B shows the percentage of indels in CD34+ cells from one donor with TDT (“Treated”) at Days 1 to 3 post electroporation with RNP32. “Mock” represents cells electroporated without RNP (unedited cells). Indel=insertions and/or deletions. FIG. 3C shows the percentage of viable cells in CD34+ cells from three donors with TDT at Days 1 to 3 post electroporation. “Treated” (dashed line) represents cells electroporated with RNP32. “Mock” (solid line) represents cells electroporated without RNP (unedited cells).

FIG. 4A shows the percentage of CD235a⁺ cells from three donors with TDT at Day 18 in erythroid culture. “RNP32” represents erythroblasts differentiated from RNP32 edited CD34+ cells from donors with TDT. “Mock” represents erythroblasts differentiated from cells electroporated without RNP (unedited cells). N=3 independent donors with triplicate cultures. *p<0.05. FIG. 4B shows the percentage of CD235a⁺ cells from donor 2 at Days 7, 11, 14, and 18 in erythroid culture. “RNP32” (dashed line) represents erythroblasts differentiated from RNP32 edited CD34+ cells from donors with TDT. “Mock” (solid line) represents erythroblasts differentiated from cells electroporated without RNP (unedited cells). N=3 independent donors with triplicate cultures. FIG. 4C shows the percentage of erythroblasts that reached late erythroblast stage. “RNP32” represents erythroblasts differentiated from RNP32 edited CD34+ cells from donors with TDT. “Mock” represents erythroblasts differentiated from cells electroporated without RNP (unedited cells). N=3 independent donors with triplicate cultures. *p<0.05; **p<0.01; ***p<0.001; ****p<0.0001. FIG. 4D shows the percentage of erythroid cells that underwent terminal maturation and enucleated. “RNP32” represents erythroblasts differentiated from RNP32 edited CD34+ cells from donors with TDT. “Mock” represents erythroblasts differentiated from cells electroporated without RNP (unedited cells). N=3 independent donors with triplicate cultures. *p<0.05; **p<0.01; ***p<0.001; ****p<0.0001. FIG. 4E shows cell death frequency of erythroblasts (i.e., percentage of non-viable erythroblasts). “RNP32” represents erythroblasts differentiated from RNP32 edited CD34+ cells from donors with TDT. “Mock” represents erythroblasts differentiated from cells electroporated without RNP (unedited cells). N=3 independent donors with triplicate cultures. *p<0.05; **p<0.01; ***p<0.001; ****p<0.0001. FIG. 4F shows the percentage of erythroblasts from donor 2 at Days 7, 11, 14, and 18 in erythroid culture that reached late erythroblast stage. The dashed line represents erythroblasts differentiated from RNP32 edited CD34+ cells from donor 2 with TDT. The solid line represents erythroblasts differentiated from cells electroporated without RNP (unedited cells). N=1 independent donor with triplicate cultures. *p<0.05; **p<0.01; ***p<0.001; ****p<0.0001. FIG. 4G shows the percentage of erythroid cells from donor 2 at Days 7, 11, 14, and 18 in erythroid culture that underwent terminal maturation and enucleated. The dashed line represents erythroblasts differentiated from RNP32 edited CD34+ cells from donor 2 with TDT. The solid line represents erythroblasts differentiated from cells electroporated without RNP (unedited cells). N=1 independent donor with triplicate cultures. *p<0.05; **p<0.01; ***p<0.001; ****p<0.0001. FIG. 4H shows cell death frequency of erythroblasts (i.e., percentage of non-viable erythroblasts) from donor 2 at Days 7, 11, 14, and 18 in erythroid culture. The dashed line represents erythroblasts differentiated from RNP32 edited CD34+ cells from donor 2 with TDT. The solid line represents erythroblasts differentiated from cells electroporated without RNP (unedited cells). N=1 independent donor with triplicate cultures. *p<0.05; **p<0.01; ***p<0.001; ****p<0.0001.

FIG. 5A shows HBG/GAPDH mRNA content for erythroblasts differentiated from RNP32 edited and unedited CD34+ cells from three donors with TDT (“Donor 1,” “Donor 2,” “Donor 3”). Data from erythroblasts differentiated from RNP32 edited CD34+ cells are shown on the right side for each donor and data from erythroblasts differentiated from cells electroporated without RNP (unedited cells) are shown on the left side for each donor. N=3 independent donors with three technical replicate cultures. *p<0.05; **p<0.01; ***p<0.001. GAPDH: Glyceraldehyde-3-phosphate dehydrogenase; HBG: γ-globin. FIG. 5B shows γ-globin protein content (picograms (pg) per cell) for erythroblasts differentiated from RNP32 edited and unedited CD34+ cells from three donors with TDT (“Donor 1,” “Donor 2,” “Donor 3”). Data from erythroblasts differentiated from RNP32 edited CD34+ cells are shown on the right side for each donor and data from erythroblasts differentiated from cells electroporated without RNP (unedited cells) are shown on the left side for each donor. N=3 independent donors with six technical replicate cultures. *p<0.05; **p<0.01; ***p<0.001. FIG. 5C shows total globin/GAPDH mRNA content for erythroblasts differentiated from RNP32 edited and unedited CD34+ cells from three donors with TDT (“Donor 1,” “Donor 2,” “Donor 3”). Data from erythroblasts differentiated from RNP32 edited CD34+ cells are shown on the right side for each donor and data from erythroblasts differentiated from cells electroporated without RNP (unedited cells) are shown on the left side for each donor. N=3 independent donors with three technical replicate cultures. *p<0.05; **p<0.01; ***p<0.001. FIG. 5D shows total hemoglobin protein content per cell for erythroblasts differentiated from RNP32 edited and unedited CD34+ cells from three donors with TDT (“Donor 1,” “Donor 2,” “Donor 3”). Data from erythroblasts differentiated from RNP32 edited CD34+ cells are shown on the right side for each donor and data from erythroblasts differentiated from cells electroporated without RNP (unedited cells) are shown on the left side for each donor. N=3 independent donors with six technical replicate cultures. *p<0.05; **p<0.01; ***p<0.001. FIG. 5E shows total hemoglobin protein content per cell for erythroblasts differentiated from RNP32 edited and unedited CD34+ cells from three donors with TDT (“Thal donor 1,” “Thal donor 2,” “Thal donor 3”). “RNP32” represents erythroblasts differentiated from RNP32 edited CD34+ cells from donors with TDT. “Mock” represents erythroblasts differentiated from cells electroporated without RNP (unedited cells). Total hemoglobin production was measured evaluated using reverse phase ultra-performance liquid chromatography (RP-UPLC).

FIG. 6 depicts the sequences of Cpf1 protein variants set forth in Table 9. Nuclear localization sequences are shown as bolded letters, six-histidine sequences are shown as underlined letters. Additional permutations of the identity and N-terminal/C-terminal positions of NLS sequences, e.g., appending two or more nNLS sequences or combinations of nNLS and sNLS sequences (or other NLS sequences) to either the N-terminal/C-terminal positions, as well as sequences with and without purification sequences, e.g., six-histidine sequences, are within the scope of the instantly disclosed subject matter.

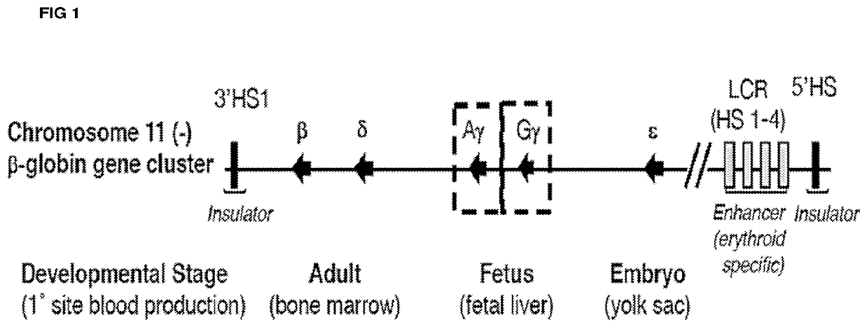

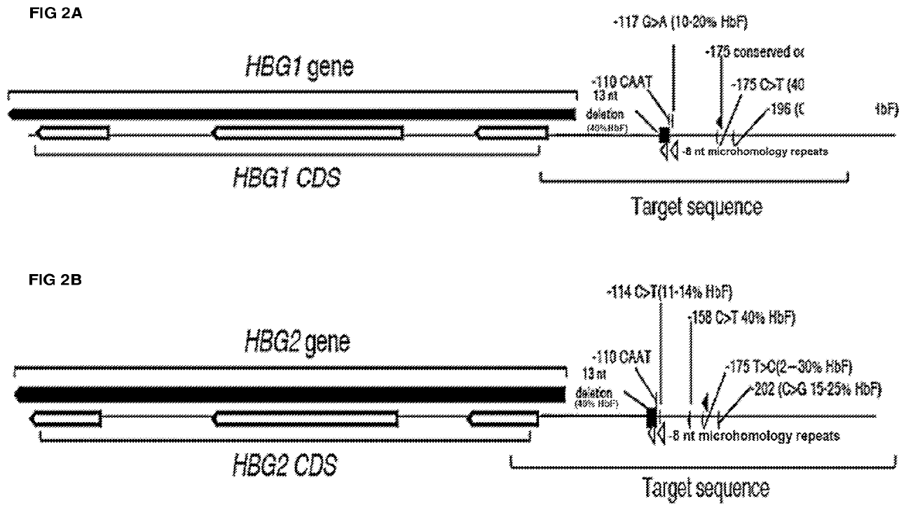

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

“Domain” is used to describe a segment of a protein or nucleic acid. Unless otherwise indicated, a domain is not required to have any specific functional property.

“Productive indel” refers to an indel (deletion and/or insertion) that results in HbF expression. In certain embodiments, a productive indel may induce HbF expression. In certain embodiments, a productive indel may result in an increased level of HbF expression.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below.

“Gene conversion” refers to the alteration of a DNA sequence by incorporation of an endogenous homologous sequence (e.g. a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing may still be used) and are quantified by the relative frequency of numerical changes (e.g., ±1, 2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai 2016 (incorporated by reference herein) or by other means well known in the art. Genome editing outcomes may also be assessed by in situ hybridization methods such as the FiberComb™ System Commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g. a nucleic acid or protein), does not require a process limitation but merely indicates that the replacement entity is present.

“Subject” means a human, mouse, or non-human primate. A human subject can be any age (e.g., an infant, child, young adult, or adult), and may suffer from a disease, or may be in need of alteration of a gene.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a subject, including (a) avoiding or precluding the disease; (b) affecting the predisposition toward the disease; or (c) preventing or delaying the onset of at least one symptom of the disease.

A “kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g. suspended in, or suspendable in) a pharmaceutically acceptable carrier. In certain embodiments, the kit may include a booster element. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example in gRNA targeting domains.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

The notation “CCAAT box target region” and the like refer to a sequence that is 5′ of the transcription start site (TSS) of the HBG1 and/or HBG2 gene. CCAAT boxes are highly conserved motifs within the promoter region of α-like and β-like globin genes. The regions within or near the CCAAT box play important roles in globin gene regulation. For example, the γ-globin distal CCAAT box is associated with hereditary persistence of fetal hemoglobin. A number of transcription factors have been reported to bind to the duplicated CCAAT box region of the γ-globin promoter, e.g., NF-Y, COUP-TFII (NF-E3), CDP, GATA1/NF-E1 and DRED (Martyn 2017). While not wishing to be bound by theory, it is believed that the binding sites of the transcriptional activator NF-Y overlaps with transcriptional repressors at the γ-globin promoter. HPFH mutations present within the distal γ-globin promoter region, e.g., within or near the CCAAT box, may alter the competitive binding of those factors and thus contribute to the increased γ-globin expression and elevated levels of HbF. Genomic locations provided herein for HBG1 and HBG2 are based on the coordinates provided in NCBI Reference Sequence NC_000011, “Homo sapiens chromosome 11, GRCh38.p12 Primary Assembly,” (Version NC_000011.10). The distal CCAAT box of HBG1 and HBG2 is positioned at HBG1 and HBG2 c.-111 to -115 (Genomic location is Hg38 Chr11:5,249,968 to Chr11:5,249,972 and Hg38 Chr11:5,254,892 to Chr11:5,254,896, respectively). The HBG1 c.-111 to -115 region is exemplified in SEQ ID NO:902 (HBG1) at positions 2823-2827, and the HBG2 c.-111 to -115 region is exemplified in SEQ ID NO:903 (HBG2) at positions 2747-2751. In certain embodiments, the “CCAAT box target region” denotes the region that is at or near the distal CCAAT box and includes the nucleotides of the distal CCAAT box and 25 nucleotides upstream (5′) and 25 nucleotides downstream (3′) of the distal CCAAT box (i.e., HBG1/2 c.-86 to -140) (Genomic location is Hg38 Chr11:5249943 to Hg38 Chr11:5249997 and Hg38 Chr11:5254867 to Hg38 Chr11:5254921, respectively). The HBG1 c.-86 to -140 region is exemplified in SEQ ID NO:902 (HBG1) at positions 2798-2852, and the HBG2 c.-86 to -140 region is exemplified in SEQ ID NO:903 (HBG2) at positions 2723-2776. In other embodiments, the “CCAAT box target region” denotes the region that is at or near the distal CCAAT box and includes the nucleotides of the distal CCAAT box and 5 nucleotides upstream (5′) and 5 nucleotides downstream (3′) of the distal CCAAT box (i.e., HBG1/2 c.-106 to -120 (Genomic location is Hg38 Chr11:5249963 to Hg38 Chr11:5249977 (HGB1 and Hg38 Chr11:5254887 to Hg38 Chr11:5254901, respectively)). The HBG1 c.-106 to -120 region is exemplified in SEQ ID NO:902 (HBG1) at positions 2818-2832, and the HBG2 c.-106 to -120 region is exemplified in SEQ ID NO:903 (HBG2) at positions 2742-2756. The term “CCAAT box target site alteration” and the like refer to alterations (e.g., deletions, insertions, mutations) of one or more nucleotides of the CCAAT box target region. Examples of exemplary CCAAT box target region alterations include, without limitation, the 1 nt deletion, 4 nt deletion, 11 nt deletion, 13 nt deletion, and 18 nt deletion, and -117 G>A alteration. As used herein, the terms “CCAAT box” and “CAAT box” can be used interchangeably.

The notations “c.-114 to -102 region,” “c.-102 to -114 region,” “−102:-114,” “13 nt target region” and the like refer to a sequence that is 5′ of the transcription start site (TSS) of the HBG1 and/or HBG2 gene at the genomic location Hg38 Chr11:5,249,959 to Hg38 Chr11:5,249,971 and Hg38 Chr11:5,254,883 to Hg38 Chr11:5,254,895, respectively. The HBG1 c.-102 to -114 region is exemplified in SEQ ID NO:902 (HBG1) at positions 2824-2836 and the HBG2 c.-102 to -114 region is exemplified in SEQ ID NO:903 (HBG2) at positions 2748-2760. The term “13 nt deletion” and the like refer to deletions of the 13 nt target region.

The notations “c.-121 to -104 region,” “c.-104 to -121 region,” “−104:-121,” “18 nt target region,” and the like refer to a sequence that is 5′ of the transcription start site (TSS) of the HBG1 and/or HBG2 gene at the genomic location Hg38 Chr11:5,249,961 to Hg38 Chr11:5,249,978 and Hg38 Chr11:5,254,885 to Hg38 Chr1l: 5,254,902, respectively. The HBG1 c.-104 to -121 region is exemplified in SEQ ID NO:902 (HBG1) at positions 2817-2834, and the HBG2 c.-104 to -121 region is exemplified in SEQ ID NO:903 (HBG2) at positions 2741-2758. The term “18 nt deletion” and the like refer to deletions of the 18 nt target region.

The notations “c.-105 to -115 region,” “c.-115 to -105 region,” “−105:-115,” “11 nt target region,” and the like refer to a sequence that is 5′ of the transcription start site (TSS) of the HBG1 and/or HBG2 gene at the genomic location Hg38 Chr11:5,249,962 to Hg38 Chr11:5,249,972 and Hg38 Chr11:5,254,886 to Hg38 Chr11:5,254,896, respectively. The HBG1 c.-105 to -115 region is exemplified in SEQ ID NO:902 (HBG1) at positions 2823-2833, and the HBG2 c.-105 to -115 region is exemplified in SEQ ID NO:903 (HBG2) at positions 2747-2757. The term “11 nt deletion” and the like refer to deletions of the 11 nt target region.

The notations “c.-115 to -112 region,” “c.-112 to -115 region,” “−112:-115,” “4 nt target region,” and the like refer to a sequence that is 5′ of the transcription start site (TSS) of the HBG1 and/or HBG2 gene at the genomic location Hg38 Chr11:5,249,969 to Hg38 Chr11:5,249,972 and Hg38 Chr11:5,254,893 to Hg38 Chr11:5,254,896, respectively. The HBG1 c.-112 to -115 region is exemplified in SEQ ID NO:902 at positions 2823-2826, and the HBG2 c.-112 to -115 region is exemplified in SEQ ID NO:903 (HBG2) at positions 2747-2750. The term “4 nt deletion” and the like refer to deletions of the 4 nt target region.

The notations “c.-116 region,” “HBG-116,” “1 nt target region,” and the like refer to a sequence that is 5′ of the transcription start site (TSS) of the HBG1 and/or HBG2 gene at the genomic location Hg38 Chr11:5,249,973 and Hg38 Chr11:5,254,897, respectively. The HBG1 c.-116 region is exemplified in SEQ ID NO:902 at position 2822, and the HBG2 c.-116 region is exemplified in SEQ ID NO:903 (HBG2) at position 2746. The term “1 nt deletion” and the like refer to deletions of the 1 nt target region.

The notations “c.-117 G>A region,” “HBG-117 G>A,” “−117 G>A target region” and the like refer to a sequence that is 5′ of the transcription start site (TSS) of the HBG1 and/or HBG2 gene at the genomic location Hg38 Chr11:5,249,974 to Hg38 Chr11:5,249,974 and Hg38 Chr11:5,254,898 to Hg38 Chr11:5,254,898, respectively. The HBG1 c.-117 G>A region is exemplified by a substitution from guanine (G) to adenine (A) in SEQ ID NO:902 at position 2821, and the HBG2 c.-117 G>A region is exemplified by a substitution from G to A in SEQ ID NO:903 (HBG2) at position 2745. The term “−117 G>A alteration” and the like refer to a substitution from G to A at the -117G>A target region.

The term “proximal HBG1/2 promoter target sequence” denotes the region within 50, 100, 200, 300, 400, or 500 bp of a proximal HBG1/2 promoter sequence including the 13 nt target region. Alterations by genome editing systems according to this disclosure facilitate (e.g. cause, promote or tend to increase the likelihood of) upregulation of HbF production in erythroid progeny.

Where ranges are provided herein, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

### Overview

The various embodiments of this disclosure generally relate to genome editing systems configured to introduce alterations (e.g., a deletion or insertion, or other mutation) into chromosomal DNA that enhance transcription of the HBG1 and/or HBG2 genes, which encode the Aγ and Gγ subunits of hemoglobin, respectively. In certain embodiments, increased expression of one or more γ-globin genes (e.g., HBG1, HBG2) using the methods provided herein results in preferential formation of HbF over HbA and/or increased HbF levels as a percentage of total hemoglobin. In certain embodiments, the disclosure generally relates to the use of RNP complexes comprising a gRNA complexed to a Cpf1 molecule. In certain embodiments, the gRNA may be unmodified or modified, the Cpf1 molecule may be a wild-type Cpf1 protein or a modified Cpf1 protein. In certain embodiments, the gRNA may comprise a sequence set forth in Tables 12, 13, 16, or 17. In certain embodiments, a modified Cpf1 may be encoded by a sequence set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences). In certain embodiments, the RNP complex may comprise an RNP complex set forth in Table 10. For example, the RNP complex may include a gRNA comprising the sequence set forth in SEQ ID NO: 1051 and a modified Cpf1 protein encoded by the sequence set forth in SEQ ID NO: 1097 (RNP32, Table 10).

It has previously been shown that patients with the condition Hereditary Persistence of Fetal Hemoglobin (HPFH) contain mutations in an γ-globin regulatory element that results in fetal γ-globin expression throughout life, rather than being repressed around the time of birth (Martyn 2017). This results in elevated fetal hemoglobin (HbF) expression. HPFH mutations may be deletional or non-deletional (e.g., point mutations). Subjects with HPFH exhibit lifelong expression of HbF, i.e., they do not undergo or undergo only partial globin switching, with no symptoms of anemia.

HbF expression can be induced through point mutations in an γ-globin regulatory element that is associated with a naturally occurring HPFH variant, including, for example, HBG1 c.-114 C>T; c.-117 G>A; c.-158 C>T; c.-167 C>T; c.-170 G>A; c.-175 T>G; c.-175 T>C; c.-195 C>G; c.-196 C>T; c.-197 C>T; c.-198 T>C; c.-201 C>T; c.-202 C>T; c.-211 C>T, c.-251 T>C; or c.-499 T>A; or HBG2 c.-109 G>T; c.-110 A>C; c.-114 C>A; c.-114 C>T; c.-114 C>G; c.-157 C>T; c.-158 C>T; c.-167 C>T; c.-167 C>A; c.-175 T>C; c.-197 C>T; c.-200+C; c.-202 C>G; c.-211 C>T; c.-228 T>C; c.-255 C>G; c.-309 A>G; c.-369 C>G; or c.-567 T>G.

Naturally occurring mutations at the distal CCAAT box motif found within the promoter of the HBG1 and/or HBG2 genes (i.e., HBG1/2 c.-111 to -115) have also been shown to result in continued γ-globin expression and the HPFH condition. It is thought that alteration (mutation or deletion) of the CCAAT box may disrupt the binding of one or more transcriptional repressors, resulting in continued expression of the γ-globin gene and elevated HbF expression (Martyn 2017). For example, a naturally occurring 13 base pair del c.-114 to -102 (“13 nt deletion”) has been shown to be associated with elevated levels of HbF (Martyn 2017). The distal CCAAT box likely overlaps with the binding motifs within and surrounding the CCAAT box of negative regulatory transcription factors that are expressed in adulthood and repress HBG (Martyn 2017).

A gene editing strategy disclosed herein is to increase HbF expression by disrupting one or more nucleotides in the distal CCAAT box and/or surrounding the distal CCAAT box. In certain embodiments, the “CCAAT box target region” may be the region that is at or near the distal CCAAT box and includes the nucleotides of the distal CCAAT box and 25 nucleotides upstream (5′) and 25 nucleotides downstream (3′) of the distal CCAAT box (i.e., HBG1/2 c.-86 to -140). In other embodiments, the “CCAAT box target region” may be the region that is at or near the distal CCAAT box and includes the nucleotides of the distal CCAAT box and 5 nucleotides upstream (5′) and 5 nucleotides downstream (3′) of the distal CCAAT box (i.e., HBG1/2 c.-106 to -120).

Unique, non-naturally occurring alterations of the CCAAT box target region are disclosed herein that induce HBG expression including, without limitation, HBG del c. -104 to -121 (“18 nt deletion”), HBG del c.-105 to -115 (“11 nt deletion”), HBG del c.-112 to -115 (“4 nt deletion”), and HBG del c.-116 (“1 nt deletion”). In certain embodiments, genome editing systems disclosed herein may be used to introduce alterations into the CCAAT box target region of HBG1 and/or HBG2. In certain embodiments, the genome editing systems may include an RNA guided nuclease including a Cas9, modified Cas 9, a Cpf1, or modified Cpf1. In certain embodiments, the genome editing systems may include an RNP comprising a gRNA and a Cpf1 molecule. In certain embodiments, a gRNA may be unmodified or modified, the Cpf1 molecule may be a wild-type Cpf1 protein or a modified Cpf1 protein, or a combination thereof. In certain embodiments, the gRNA may comprise a sequence set forth in Tables 7, 8, 11, or 12. In certain embodiments, a modified Cpf1 may be encoded by a sequence set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences). In certain embodiments, the RNP complex may comprise an RNP complex set forth in Table 10. For example, the RNP complex may include a gRNA comprising the sequence set forth in SEQ ID NO:1051 and a modified Cpf1 protein encoded by the sequence set forth in SEQ ID NO: 1097 (RNP32, Table 10).

The genome editing systems of this disclosure can include an RNA-guided nuclease such as Cpf1 and one or more gRNAs having a targeting domain that is complementary to a sequence in or near the target region, and optionally one or more of a DNA donor template that encodes a specific mutation (such as a deletion or insertion) in or near the target region, and/or an agent that enhances the efficiency with which such mutations are generated including, without limitation, a random oligonucleotide, a small molecule agonist or antagonist of a gene product involved in DNA repair or a DNA damage response, or a peptide agent.

A variety of approaches to the introduction of mutations into the CCAAT box target region, 13 nt target region, and/or proximal HBG1/2 promoter target sequence may be employed in the embodiments of the present disclosure. In one approach, a single alteration, such as a double-strand break, is made within the CCAAT box target region, 13 nt target region, and/or proximal HBG1/2 promoter target sequence, and is repaired in a way that disrupts the function of the region, for example by the formation of an indel or by the incorporation of a donor template sequence that encodes the deletion of the region. In a second approach, two or more alterations are made on either side of the region, resulting in the deletion of the intervening sequence, including the CCAAT box target region and/or 13 nt target region.

The treatment of hemoglobinopathies by gene therapy and/or genome editing is complicated by the fact that the cells that are phenotypically affected by the disease, erythrocytes or RBCs, are enucleated, and do not contain genetic material encoding either the aberrant hemoglobin protein (Hb) subunits nor the Aγ or Gγ subunits targeted in the exemplary genome editing approaches described above. This complication is addressed, in certain embodiments of this disclosure, by the alteration of cells that are competent to differentiate into, or otherwise give rise to, erythrocytes. Cells within the erythroid lineage that are altered according to various embodiments of this disclosure include, without limitation, hematopoietic stem and progenitor cells (HSCs), erythroblasts (including basophilic, polychromatic and/or orthochromatic erythroblasts), proerythroblasts, polychromatic erythrocytes or reticulocytes, embryonic stem (ES) cells, and/or induced pluripotent stem (iPSC) cells. These cells may be altered in situ (e.g. within a tissue of a subject) or ex vivo. Implementations of genome editing systems for in situ and ex vivo alteration of cells is described under the heading “Implementation of genome editing systems: delivery, formulations, and routes of administration” below.

In certain embodiments, alterations that result in induction of Aγ and/or Gγ expression are obtained through the use of a genome editing system comprising an RNA-guided nuclease and at least one gRNA having a targeting domain complementary to a sequence within the CCAAT box target region of HBG1 and/or HBG2 or proximate thereto (e.g., within 10, 20, 30, 40, or 50, 100, 200, 300, 400 or 500 bases of the CCAAT box target region). As is discussed in greater detail below, the RNA-guided nuclease and gRNA form a complex that is capable of associating with and altering the CCAAT box target region or a region proximate thereto. Examples of suitable gRNAs and gRNA targeting domains directed to the CCAAT box target region of HBG1 and/or HBG2 or proximate thereto for use in the embodiments disclosed herein include those set forth herein.

In certain embodiments, alterations that result in induction of Aγ and/or Gγ expression are obtained through the use of a genome editing system comprising an RNA-guided nuclease and at least one gRNA having a targeting domain complementary to a sequence within the 13 nt target region of HBG1 and/or HBG2 or proximate thereto (e.g., within 10, 20, 30, 40, or 50, 100, 200, 300, 400 or 500 bases of the 13 nt target region). As is discussed in greater detail below, the RNA-guided nuclease and gRNA form a complex that is capable of associating with and altering the 13 nt target region or a region proximate thereto. Examples of suitable gRNAs and gRNA targeting domains directed to the 13 nt target region of HBG1 and/or HBG2 or proximate thereto for use in the embodiments disclosed herein include those set forth herein.

The genome editing system can be implemented in a variety of ways, as is discussed below in detail. As an example, a genome editing system of this disclosure can be implemented as a ribonucleoprotein complex or a plurality of complexes in which multiple gRNAs are used. This ribonucleoprotein complex can be introduced into a target cell using art-known methods, including electroporation, as described in commonly-assigned International Patent Publication No. WO 2016/182959 by Jennifer Gori (“Gori”), published Nov. 17, 2016, which is incorporated by reference in its entirety herein.

The ribonucleoprotein complexes within these compositions are introduced into target cells by art-known methods, including without limitation electroporation (e.g. using the Nucleofection™ technology commercialized by Lonza, Basel, Switzerland or similar technologies commercialized by, for example, Maxcyte Inc. Gaithersburg, Maryland) and lipofection (e.g. using Lipofectamine™ reagent commercialized by Thermo Fisher Scientific, Waltham Massachusetts). Alternatively, or additionally, ribonucleoprotein complexes are formed within the target cells themselves following introduction of nucleic acids encoding the RNA-guided nuclease and/or gRNA. These and other delivery modalities are described in general terms below and in Gori.

Cells that have been altered ex vivo according to this disclosure can be manipulated (e.g. expanded, passaged, frozen, differentiated, de-differentiated, transduced with a transgene, etc.) prior to their delivery to a subject. The cells are, variously, delivered to a subject from which they are obtained (in an “autologous” transplant), or to a recipient who is immunologically distinct from a donor of the cells (in an “allogeneic” transplant).

In some cases, an autologous transplant includes the steps of obtaining, from the subject, a plurality of cells, either circulating in peripheral blood, or within the marrow or other tissue (e.g. spleen, skin, etc.), and manipulating those cells to enrich for cells in the erythroid lineage (e.g. by induction to generate iPSCs, purification of cells expressing certain cell surface markers such as CD34, CD90, CD49f and/or not expressing surface markers characteristic of non-erythroid lineages such as CD10, CD14, CD38, etc.). The cells are, optionally or additionally, expanded, transduced with a transgene, exposed to a cytokine or other peptide or small molecule agent, and/or frozen/thawed prior to transduction with a genome editing system targeting the CCAAT box target region, the 13 nt target region, and/or proximal HBG1/2 promoter target sequence. The genome editing system can be implemented or delivered to the cells in any suitable format, including as a ribonucleoprotein complex, as separated protein and nucleic acid components, and/or as nucleic acids encoding the components of the genome editing system.

In certain embodiments, CD34+ hematopoietic stem and progenitor cells (HSPCs) that have been edited using the genome editing methods disclosed herein may be used for the treatment of a hemoglobinopathy in a subject in need thereof. In certain embodiments, the hemoglobinopathy may be severe sickle cell disease (SCD) or thalassemia, such as β-thalassemia, δ-thalassemia, or β/δ-thalassemia. In certain embodiments, an exemplary protocol for treatment of a hemoglobinopathy may include harvesting CD34+ HSPCs from a subject in need thereof, ex vivo editing of the autologous CD34+ HSPCs using the genome editing methods disclosed herein, followed by reinfusion of the edited autologous CD34+ HSPCs into the subject. In certain embodiments, treatment with edited autologous CD34+ HSPCs may result in increased HbF induction.

Prior to harvesting CD34+ HSPCs, in certain embodiments, a subject may discontinue treatment with hydroxyurea, if applicable, and receive blood transfusions to maintain sufficient hemoglobin (Hb) levels. In certain embodiments, a subject may be administered intravenous plerixafor (e.g., 0.24 mg/kg) to mobilize CD34+ HSPCs from bone marrow into peripheral blood. In certain embodiments, a subject may undergo one or more leukapheresis cycles (e.g., approximately one month between cycles, with one cycle defined as two plerixafor-mobilized leukapheresis collections performed on consecutive days). In certain embodiments, the number of leukapheresis cycles performed for a subject may be the number required to achieve a dose of edited autologous CD34+ HSPCs (e.g., ≥2×10⁶ cells/kg, ≥3×10⁶ cells/kg, ≥4×10⁶ cells/kg, ≥5×10⁶ cells/kg, 2×10⁶ cells/kg to 3×10⁶ cells/kg, 3×10⁶ cells/kg to 4×10⁶ cells/kg, 4×10⁶ cells/kg to 5×10⁶ cells/kg) to be reinfused back into the subject, along with a dose of unedited autologous CD34+ HSPCs/kg for backup storage (e.g., ≥1.5×10⁶ cells/kg). In certain embodiments, the CD34+ HSPCs harvested from the subject may be edited using any of the genome editing methods discussed herein. In certain embodiments, any one or more of the gRNAs and one or more of the RNA-guided nucleases disclosed herein may be used in the genome editing methods.

In certain embodiments, the treatment may include an autologous stem cell transplant. In certain embodiments, a subject may undergo myeloablative conditioning with busulfan conditioning (e.g., dose-adjusted based on first-dose pharmacokinetic analysis, with a test dose of 1 mg/kg). In certain embodiments, conditioning may occur for four consecutive days. In certain embodiments, following a three-day busulfan washout period, edited autologous CD34+ HSPCs (e.g., ≥2×10⁶ cells/kg, ≥3×10⁶ cells/kg, ≥4×10⁶ cells/kg, ≥5×10⁶ cells/kg, 2×10⁶ cells/kg to 3×10⁶ cells/kg, 3×10⁶ cells/kg to 4×10⁶ cells/kg, 4×10⁶ cells/kg to 5×10⁶ cells/kg) may be reinfused into the subject (e.g., into peripheral blood). In certain embodiments, the edited autologous CD34+ HSPCs may be manufactured and cryopreserved for a particular subject. In certain embodiments, a subject may attain neutrophil engraftment following a sequential myeloablative conditioning regimen and infusion of edited autologous CD34+ cells. Neutrophil engraftment may be defined as three consecutive measurements of ANC≥0.5×10⁹/L.

However it is implemented, a genome editing system may include, or may be co-delivered with, one or more factors that improve the viability of the cells during and after editing, including without limitation an aryl hydrocarbon receptor antagonist such as StemRegenin-1 (SRI), UM171, LGC0006, alpha-napthoflavone, and CH-223191, and/or an innate immune response antagonist such as cyclosporin A, dexamethasone, reservatrol, a MyD88 inhibitory peptide, an RNAi agent targeting Myd88, a B18R recombinant protein, a glucocorticoid, OxPAPC, a TLR antagonist, rapamycin, BX795, and a RLR shRNA. These and other factors that improve the viability of the cells during and after editing are described in Gori, under the heading “I. Optimization of Stem Cells” from page 36 through page 61, which is incorporated by reference herein.

The cells, following delivery of the genome editing system, are optionally manipulated e.g. to enrich for HSCs and/or cells in the erythroid lineage and/or for edited cells, to expand them, freeze/thaw, or otherwise prepare the cells for return to the subject. The edited cells are then returned to the subject, for instance in the circulatory system by means of intravenous delivery or delivery or into a solid tissue such as bone marrow.

Functionally, alteration of the CCAAT box target region, the 13 nt target region, and/or proximal HBG1/2 promoter target sequence using the compositions, methods and genome editing systems of this disclosure results in significant induction, among hemoglobin-expressing cells, of Aγ and/or Gγ subunits (referred to interchangeably as HbF expression), e.g. at least 5%, 10%, 15%, 20%, 25%, 30%, 35%, 40%, 45%, 50% or greater induction of Aγ and/or Gγ subunit expression relative to unmodified controls. This induction of protein expression is generally the result of alteration of the CCAAT box target region, 13 nt target region, and/or proximal HBG1/2 promoter target sequence (expressed, e.g. in terms of the percentage of total genomes comprising indel mutations within the plurality of cells) in some or all of the plurality of cells that are treated, e.g. at least 5%, 10%, 15%, 20%, 25%, 30%, 35%, 40%, 45%, 50% of the plurality of cells comprise at least one allele comprising a sequence alteration, including, without limitation, an indel, insertion, or deletion in or near the CCAAT box target region, 13 nt target region, and/or proximal HBG1/2 promoter target sequence.

The functional effects of alterations caused or facilitated by the genome editing systems and methods of the present disclosure can be assessed in any number of suitable ways. For example, the effects of alterations on expression of fetal hemoglobin can be assessed at the protein or mRNA level. Expression of HBG1 and HBG2 mRNA can be assessed by digital droplet PCR (ddPCR), which is performed on cDNA samples obtained by reverse transcription of mRNA harvested from treated or untreated samples. Primers for HBG1, HBG2, HBB, and/or HBA may be used individually or multiplexed using methods known in the art. For example, ddPCR analysis of samples may be conducted using the QX200™ ddPCR system commercialized by Bio Rad (Hercules, CA), and associated protocols published by BioRad. Fetal hemoglobin protein may be assessed by high pressure liquid chromatography (HPLC), for example, according to the methods discussed on pp. 143-44 in Chang 2017 (incorporated by reference herein), or fast protein liquid chromatography (FPLC), using ion-exchange and/or reverse phase columns to resolve HbF, HbB and HbA and/or Aγ and Gγ globin chains as is known in the art.

The embodiments described herein may be used in all classes of vertebrate including, but not limited to, primates, mice, rats, rabbits, pigs, dogs, and cats.

This overview has focused on a handful of exemplary embodiments that illustrate the principles of genome editing systems and CRISPR-mediated methods of altering cells. For clarity, however, this disclosure encompasses modifications and variations that have not been expressly addressed above, but will be evident to those of skill in the art. With that in mind, the following disclosure is intended to illustrate the operating principles of genome editing systems more generally. What follows should not be understood as limiting, but rather illustrative of certain principles of genome editing systems and CRISPR-mediated methods utilizing these systems, which, in combination with the instant disclosure, will inform those of skill in the art about additional implementations and modifications that are within its scope.

This overview has focused on a handful of exemplary embodiments that illustrate the principles of genome editing systems and CRISPR-mediated methods of altering cells. For clarity, however, this disclosure encompasses modifications and variations that have not been expressly addressed above, but will be evident to those of skill in the art. With that in mind, the following disclosure is intended to illustrate the operating principles of genome editing systems more generally. What follows should not be understood as limiting, but rather illustrative of certain principles of genome editing systems and CRISPR-mediated methods utilizing these systems, which, in combination with the instant disclosure, will inform those of skill in the art about additional implementations and modifications that are within its scope.

### Genome Editing Systems

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Genome editing systems can be implemented (e.g. administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as, without limitation, a lipid or polymer micro- or nano-particle, micelle, or liposome. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus (see section below under the heading “Implementation of genome editing systems: delivery, formulations, and routes of administration”); and in certain embodiments, the genome editing system is implemented as a combination of any of the foregoing. Additional or modified implementations that operate according to the principles set forth herein will be apparent to the skilled artisan and are within the scope of this disclosure. Exemplary RNPs are set forth in Table 10. See International Publication No. WO 2021/119040 (see, e.g., Table 15).

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (“Maeder”), which is incorporated by reference herein, describes a genome editing system for correcting a point mutation (C.2991+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino et al. (“Cotta-Ramusino”), which is incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

As disclosed herein, in certain embodiments, genome editing systems may comprise multiple gRNAs that may be used to introduce mutations into the 13 nt target region of HBG1 and/or HBG2. In certain embodiments, genome editing systems disclosed herein may comprise multiple gRNAs used to introduce mutations into the 13 nt target region of HBG1 and/or HBG2.

Genome editing systems can, in some instances, form double strand breaks that are repaired by cellular DNA double-strand break mechanisms such as NHEJ or HDR. These mechanisms are described throughout the literature (see, e.g., Davis & Maizels 2014 (describing Alt-HDR); Frit 2014 (describing Alt-NHEJ); Iyama & Wilson 2013 (describing canonical HDR and NHEJ pathways generally)).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

In certain embodiments, genome editing systems modify a target sequence, or modify expression of a gene in or near the target sequence, without causing single- or double-strand breaks. For example, a genome editing system may include an RNA-guided nuclease fused to a functional domain that acts on DNA, thereby modifying the target sequence or its expression. As one example, an RNA-guided nuclease can be connected to (e.g. fused to) a cytidine deaminase functional domain, and may operate by generating targeted C-to-A substitutions. Exemplary nuclease/deaminase fusions are described in Komor 2016, which is incorporated by reference herein. Alternatively, a genome editing system may utilize a cleavage-inactivated (i.e. a “dead”) nuclease, such as a dead Cas9 (dCas9), and may operate by forming stable complexes on one or more targeted regions of cellular DNA, thereby interfering with functions involving the targeted region(s) including, without limitation, mRNA transcription, chromatin remodeling, etc. In certain embodiments, a genome editing system may include an RNA-guided helicase that unwinds DNA within or proximal to the target sequence, without causing single- or double-stranded breaks. For example a genome editing system may include an RNA-guided helicase configured to associate within or near the target sequence to unwind DNA and induce accessibility to the target sequence. In certain embodiments, the RNA-guided helicase may be complexed to a dead guide RNA that is configured to lack cleavage activity allowing for unwinding of the DNA without causing breaks in the DNA.

Guide RNA (gRNA) Molecules

The terms “guide RNA” and “gRNA” refer to any nucleic acid that promotes the specific association (or “targeting”) of an RNA-guided nuclease such as a Cpf1 molecule to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature (see, e.g., Briner 2014, which is incorporated by reference; Cotta-Ramusino). Examples of modular and unimolecular gRNAs that may be used according to the embodiments herein include, without limitation, the sequences set forth in SEQ ID NOs:29-31 and 38-51. Examples of gRNA proximal and tail domains that may be used according to the embodiments herein include, without limitation, the sequences set forth in SEQ ID NOs:32-37.

In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g. GAAA) “tetraloop” or “linker” sequence bridging complementary regions of the crRNA (at its 3′ end) and the tracrRNA (at its 5′ end). (Mali 2013; Jiang 2013; Jinek 2012; all incorporated by reference herein).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu 2013, incorporated by reference herein), “complementarity regions” (Cotta-Ramusino), “spacers” (Briner 2014) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cpf1 gRNA.

In addition to the targeting domains, gRNAs typically (but not necessarily, as discussed below) include a plurality of domains that may influence the formation or activity of gRNA/Cas9 complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes (Nishimasu 2014; Nishimasu 2015; both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner 2014). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner 2014.

While the foregoing description has focused on gRNAs for use with Cas9, it should be appreciated that other RNA-guided nucleases exist which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche 2015, incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA). Exemplary targeting domains of Cpf1 gRNAs are set forth in Tables 7, 8, 11, or 12. See International Publication No. WO 2021/119040 (see, e.g., Tables 12, 13, 16, 17). gRNA sequences targeting several domains of the HBG promoter (Table 6) are provided in Table 7. See International Publication No. WO 2021/119040 (see, e.g., Tables 11 and 12).

Those of skill in the art will appreciate, however, that although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

gRNA Design

Methods for selection and validation of target sequences as well as off-target analyses have been described previously (see, e.g., Mali 2013; Hsu 2013; Fu 2014; Heigwer 2014; Bae 2014; Xiao 2014). Each of these references is incorporated by reference herein. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

Targeting domain sequences of gRNAs that were designed to target disruption of the CCAAT box target region include, but are not limited to, SEQ ID NO:1002. In certain embodiments, gRNAs comprising the sequence set forth in SEQ ID NO:1002 may be complexed with a Cpf1 protein or modified Cpf1 protein to generate alterations at the CCAAT box target region. In certain embodiments, gRNAs comprising any of the Cpf1 gRNAs set forth in Tables 7, 8, 11, and 12 may be complexed with a Cpf1 protein or modified Cpf1 protein forming an RNP (“gRNA-Cpf1-RNP”) to generate alterations at the CCAAT box target region. In certain embodiments, the modified Cpf1 protein may be His-AsCpf1-nNLS (SEQ ID NO: 1000) or His-AsCpf1-sNLS-sNLS (SEQ ID NO:1001). In certain embodiments, the Cpf1 molecule of the gRNA-Cpf1-RNP may be encoded by a sequence set forth in SEQ ID NOs:1000, 1001, 1008-1018, 1032, 1035-39 (Cpf1 polypeptide sequences) or SEQ ID NOs:1019-1021 (Cpf1 polynucleotide sequences).

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

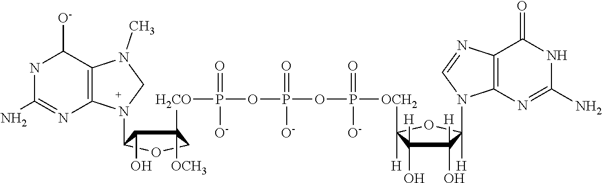

The cap or cap analog can be included during either chemical synthesis or in vitro transcription of the gRNA.

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g. a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

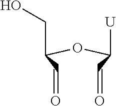

wherein “U” can be an unmodified or modified uridine.

The 3′ terminal U ribose can be modified with a 2′3′ cyclic phosphate as shown below:

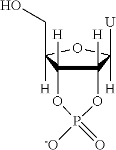

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphorothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, or 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (TI) bases. In certain embodiments, the DNA extension includes a combination of different DNA bases. In certain embodiments, a DNA extension may comprise a sequence set forth in Table 13. For example, a DNA extension may comprise a sequence set forth in SEQ ID NOs:1235-1250. In certain embodiments, a gRNA used herein includes a DNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a DNA extension may comprise a sequence set forth in Table 13 that includes a DNA extension. In a particular embodiment, a gRNA including a DNA extension may comprise the sequence set forth in SEQ ID NO:1051. In certain embodiments, a gRNA including a DNA extension may comprise a sequence selected from the group consisting of SEQ ID NOs:1046-1060, 1067, 1068, 1074, 1075, 1078, 1081-1084, 1086-1087, 1089-1090, 1092-1093, 1098-1102, and 1106. Without wishing to be bound by theory, it is contemplated that any DNA extension may be used herein, so long as it does not hybridize to the target nucleic acid being targeted by the gRNA and it also exhibits an increase in editing at the target nucleic acid site relative to a gRNA which does not include such a DNA extension. Exemplary DNA and RNA extensions are set forth in Table 13. See International Publication No. WO 2021/119040 (see, e.g., Table 18).

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA, 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, an RNA extension may comprise a sequence set forth in Table 13. For example, an RNA extension may comprise a sequence set forth in 1231-1234, 1251-1253. In certain embodiments, a gRNA used herein includes an RNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a RNA extension may comprise a sequence set forth in Table 13 that includes an RNA extension. gRNAs including an RNA extension at the 5′ end of the gRNA may comprise a sequence selected from the group consisting of SEQ ID NOs:1042-1045, 1103-1105. gRNAs including an RNA extension at the 3′ end of the gRNA may comprise a sequence selected from the group consisting of SEQ ID NOs:1070-1075, 1079, 1081, 1098-1100.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes both a phosphorothioate modification at the 3′ end as well as a DNA extension at the 5′ end is complexed with a RNA-guided nuclease, e.g., Cpf1, to form an RNP, which is then employed to edit a hematopoietic stem cell (HSC) or a CD34+ cell ex vivo (i.e., outside the body of a subject from whom such a cell is derived), at the HBG locus.

An example of a gRNA as used herein comprises the sequence set forth in SEQ ID NO:1051.

### RNA-Guided Nucleases

RNA-guided nucleases according to the present disclosure include, but are not limited to, naturally-occurring Class 2 CRISPR nucleases such as Cpf1, and Cas9, as well as other nucleases derived or obtained therefrom. It has also been shown that certain RNA-guided nucleases, such as Cas9, also have helicase activity that enables them to unwind nucleic acid. In certain embodiments, the RNA-guided helicases according to the present disclosure may be any of the RNA-nucleases described herein and supra in the section entitled “RNA-guided nucleases.” In certain embodiments, the RNA-guided nuclease is not configured to recruit an exogenous trans-acting factor to a target region. In certain embodiments, an RNA-guided helicase may be an RNA-guided nuclease configured to lack nuclease activity. For example, in certain embodiments, an RNA-guided helicase may be a catalytically inactive RNA-guided nuclease that lacks nuclease activity, but still retains its helicase activity. In certain embodiments, an RNA-guided nuclease may be mutated to abolish its nuclease activity (e.g., dead Cas9), creating a catalytically inactive RNA-guided nuclease that is unable to cleave nucleic acid, but which can still unwind DNA. In certain embodiments, an RNA-guided helicase may be complexed with any of the dead guide RNAs as described herein. For example, a catalytically active RNA-guided helicase (e.g., Cas9 or Cpf1) may form an RNP complex with a dead guide RNA, resulting in a catalytically inactive dead RNP (dRNP). In certain embodiments, a catalytically inactive RNA-guided helicase (e.g., dead Cas9) and a dead guide RNA may form a dRNP. These dRNPs, although incapable of providing a cleavage event, still retain their helicase activity that is important for unwinding nucleic acid.

In functional terms, RNA-guided nucleases are defined as those nucleases that: (a) interact with (e.g. complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g. full-length vs. truncated or split; naturally-occurring PAM specificity vs. engineered PAM specificity, etc.) of RNA-guided nuclease. For example, in certain embodiments, the RNA-guided nuclease may be Cas-qD (Pausch 2020).

Various RNA-guided nucleases may require different sequential relationships between PAMs and protospacers. In general, Cas9s recognize PAM sequences that are 3′ of the protospacer. Cpf1, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cpf1 recognizes a TTN PAM sequence. PAM sequences have been identified for a variety of RNA-guided nucleases, and a strategy for identifying novel PAM sequences has been described by Shmakov 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease). Examples of PAMs that may be used according to the embodiments herein include, without limitation, the sequences set forth in SEQ ID NOs:199-205.

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above and in Ran & Hsu 2013, incorporated by reference herein), or that do not cut at all.

### Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g. a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity. Examples of polypeptide sequences encoding Cas9 RuvC-like and Cas9 HNH-like domains that may be used according to the embodiments herein are set forth in SEQ ID NOs:15-23, 52-123 (RuvC-like domains) and SEQ ID NOs:24-28, 124-198 (HNH-like domains).

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains). Examples of polypeptide sequences encoding Cas9 molecules that may be used according to the embodiments herein are set forth in SEQ ID NOs:1-2, 4-6, 12, and 14.

### Cpf1

The crystal structure of Acidaminococcus sp. Cpf1 in complex with crRNA and a double-stranded (ds) DNA target including a TTTN PAM sequence has been solved by Yamano 2016 (incorporated by reference herein). Cpf1 and Cas12a are synonyms and can be used interchangeably herein. Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, —II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, —II and —III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

In certain embodiments, a Cpf1 protein may be a modified Cpf1 protein. In certain embodiments, a modified Cpf1 protein may include one or more modifications. In certain embodiments the modifications may be, without limitation, one or more mutations in a Cpf1 nucleotide sequence or Cpf1 amino acid sequence, one or more additional sequences such as a His tag or a nuclear localization signal (NLS), or a combination thereof. In certain embodiments, a modified Cpf1 may also be referred to herein as a Cpf1 variant.

In certain embodiments, the Cpf1 protein may be derived from a Cpf1 protein selected from the group consisting of Acidaminococcus sp. strain BV3L6 Cpf1 protein (AsCpf1), Lachnospiraceae bacterium ND2006 Cpf1 protein (LbCpf1), and Lachnospiraceae bacterium MA2020 (Lb2Cpf1). In certain embodiments, the Cpf1 protein may comprise a sequence selected from the group consisting of SEQ ID NOs:1016-1018, having the codon-optimized nucleic acid sequences of SEQ ID NOs:1019-1021, respectively.

In certain embodiments, the modified Cpf1 protein may comprise a nuclear localization signal (NLS). For example, but not by way of limitation, NLS sequences useful in connection with the methods and compositions disclosed herein will comprise an amino acid sequence capable of facilitating protein import into the cell nucleus. NLS sequences useful in connection with the methods and compositions disclosed herein are known in the art. Examples of such NLS sequences include the nucleoplasmin NLS having the amino acid sequence: KRPAATKKAGQAKKKK (SEQ ID NO:1006) and the simian virus 40 “SV40” NLS having the amino acid sequence PKKKRKV (SEQ ID NO:1007).

In certain embodiments, the NLS sequence of the modified Cpf1 protein is positioned at or near the C-terminus of the Cpf1 protein sequence. For example, but not by way of limitation, the modified Cpf1 protein can be selected from the following: His-AsCpf1-nNLS (SEQ ID NO:1000); His-AsCpf1-sNLS (SEQ ID NO:1008) and His-AsCpf1-sNLS-sNLS (SEQ ID NO: 1001), where “His” refers to a six-histidine purification sequence, “AsCpf1” refers to the Acidaminococcus sp. Cpf1 protein sequence, “nNLS” refers to the nucleoplasmin NLS, and “sNLS” refers to the SV40 NLS. Additional permutations of the identity and C-terminal positions of NLS sequences, e.g., appending two or more nNLS sequences or combinations of nNLS and sNLS sequences (or other NLS sequences), as well as sequences with and without purification sequences, e.g., six-histidine sequences, are within the scope of the instantly disclosed subject matter.

In certain embodiments, the NLS sequence of the modified Cpf1 protein may be positioned at or near the N-terminus of the Cpf1 protein sequence. For example, but not by way of limitation, the modified Cpf1 protein may be selected from the following: His-sNLS-AsCpf1 (SEQ ID NO:1009), His-sNLS-sNLS-AsCpf1 (SEQ ID NO:1010), and sNLS-sNLS-AsCpf1 (SEQ ID NO: 1011). Additional permutations of the identity and N-terminal positions of NLS sequences, e.g., appending two or more nNLS sequences or combinations of nNLS and sNLS sequences (or other NLS sequences), as well as sequences with and without purification sequences, e.g., six-histidine sequences, are within the scope of the instantly disclosed subject matter.

In certain embodiments, the modified Cpf1 protein may comprise NLS sequences positioned at or near both the N-terminus and C-terminus of the Cpf1 protein sequence. For example, but not by way of limitation, the modified Cpf1 protein may be selected from the following: His-sNLS-AsCpf1-sNLS (SEQ ID NO:1012) and His-sNLS-sNLS-AsCpf1-sNLS-sNLS (SEQ ID NO:1013). Additional permutations of the identity and N-terminal/C-terminal positions of NLS sequences, e.g., appending two or more nNLS sequences or combinations of nNLS and sNLS sequences (or other NLS sequences) to either the N-terminal/C-terminal positions, as well as sequences with and without purification sequences, e.g., six-histidine sequences, are within the scope of the instantly disclosed subject matter.

In certain embodiments, the modified Cpf1 protein may comprise an alteration (e.g., a deletion or substitution) at one or more cysteine residues of the Cpf1 protein sequence. For example, but not by way of limitation, modified Cpf1 protein may comprise an alteration at a position selected from the group consisting of: C65, C205, C334, C379, C608, C674, C1025, and C1248. In certain embodiments, the modified Cpf1 protein may comprise a substitution of one or more cysteine residues for a serine or alanine. In certain embodiments, the modified Cpf1 protein may comprise an alteration selected from the group consisting of: C65S, C205S, C334S, C379S, C608S, C674S, C1025S, and C1248S. In certain embodiments, the modified Cpf1 protein may comprise an alteration selected from the group consisting of: C65A, C205A, C334A, C379A, C608A, C674A, C1025A, and C1248A. In certain embodiments, the modified Cpf1 protein may comprise alterations at positions C334 and C674 or C334, C379, and C674. In certain embodiments, the modified Cpf1 protein may comprise the following alterations: C334S and C674S, or C334S, C379S, and C674S. In certain embodiments, the modified Cpf1 protein may comprise the following alterations: C334A and C674A, or C334A, C379A, and C674A. In certain embodiments, the modified Cpf1 protein may comprise both one or more cysteine residue alteration as well as the introduction of one or more NLS sequences, e.g., His-AsCpf1-nNLS Cys-less (SEQ ID NO:1014) or His-AsCpf1-nNLS Cys-low (SEQ ID NO:1015). In various embodiments, the Cpf1 protein comprising a deletion or substitution in one or more cysteine residues exhibits reduced aggregation.

In certain embodiments, other modified Cpf1 proteins known in the art may be used with the methods and systems described herein. For example, in certain embodiments, the modified Cpf1 may be Cpf1 containing the mutation S542R/K548V/N552R (“Cpf1 RVR”). Cpf1 RVR has been shown to cleave target sites with a TATV PAM. In certain embodiments, the modified Cpf1 may be Cpf1 containing the mutation S542R/K607R (“Cpf1 RR”). Cpf1 RR has been shown to cleave target sites with a TYCV/CCCC PAM.

In some embodiments, a Cpf1 variant is used herein, wherein the Cpf1 variant comprises mutations at one or more residues of AsCpf1 (Acidaminococcus sp. BV3L6) selected from the group consisting of 11, 12, 13, 14, 15, 16, 17, 34, 36, 39, 40, 43, 46, 47, 50, 54, 57, 58, 111, 126, 127, 128, 129, 130, 131, 132, 133, 134, 135, 136, 157, 158, 159, 160, 161, 162, 163, 164, 165, 166, 167, 168, 169, 170, 171, 172, 173, 174, 175, 176, 177, 178, 532, 533, 534, 535, 536, 537, 538, 539, 540, 541, 542, 543, 544, 545, 546, 547, 548, 549, 550, 551, 552, 553, 554, 555, 556, 565, 566, 567, 568, 569, 570, 571, 572, 573, 574, 575, 592, 593, 594, 595, 596, 597, 598, 599, 600, 601, 602, 603, 604, 605, 606, 607, 608, 609, 610, 611, 612, 613, 614, 615, 616, 617, 618, 619, 620, 626, 627, 628, 629, 630, 631, 632, 633, 634, 635, 636, 637, 638, 642, 643, 644, 645, 646, 647, 648, 649, 651, 652, 653, 654, 655, 656, 676, 679, 680, 682, 683, 684, 685, 686, 687, 688, 689, 690, 691, 692, 693, 707, 711, 714, 715, 716, 717, 718, 719, 720, 721, 722, 739, 765, 768, 769, 773, 777, 778, 779, 780, 781, 782, 783, 784, 785, 786, 870, 871, 872, 873, 874, 875, 876, 877, 878, 879, 880, 881, 882, 883, 884, or 1048 or the corresponding position of an AsCpf1 orthologue, homologue, or variant.

In certain embodiments, a Cpf1 variant as used herein may include any of the Cpf1 proteins described in International Publication Number WO 2017/184768 A1 by Zhang et al. (“'768 Publication”), which is incorporated by reference herein.

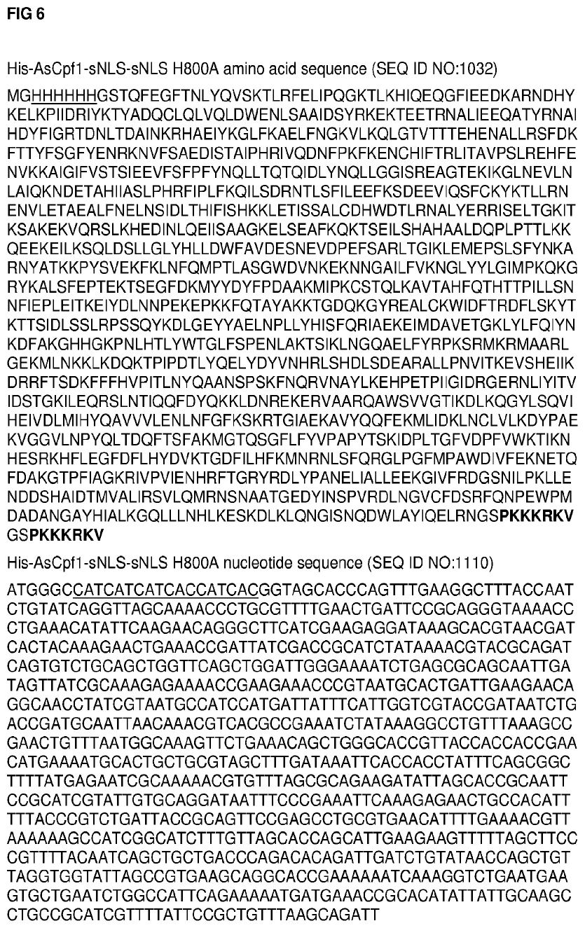

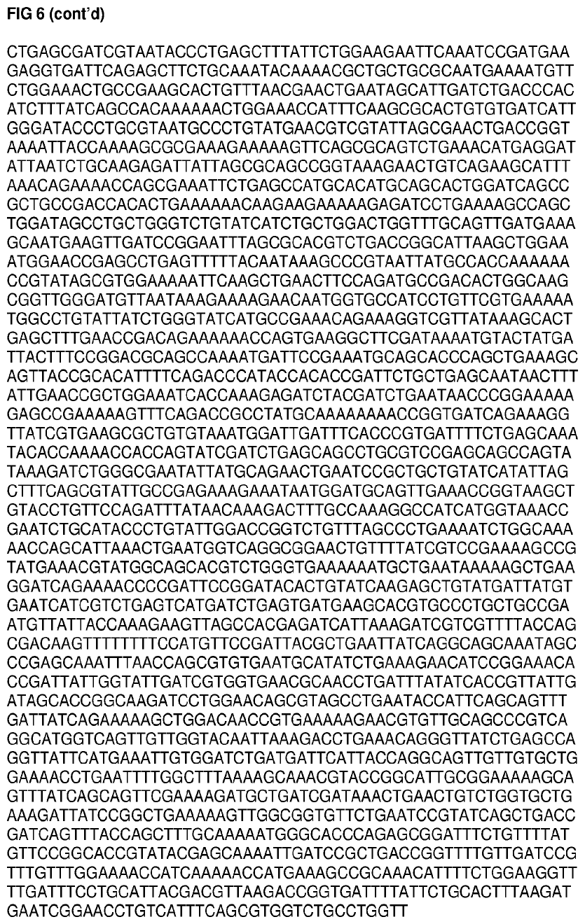

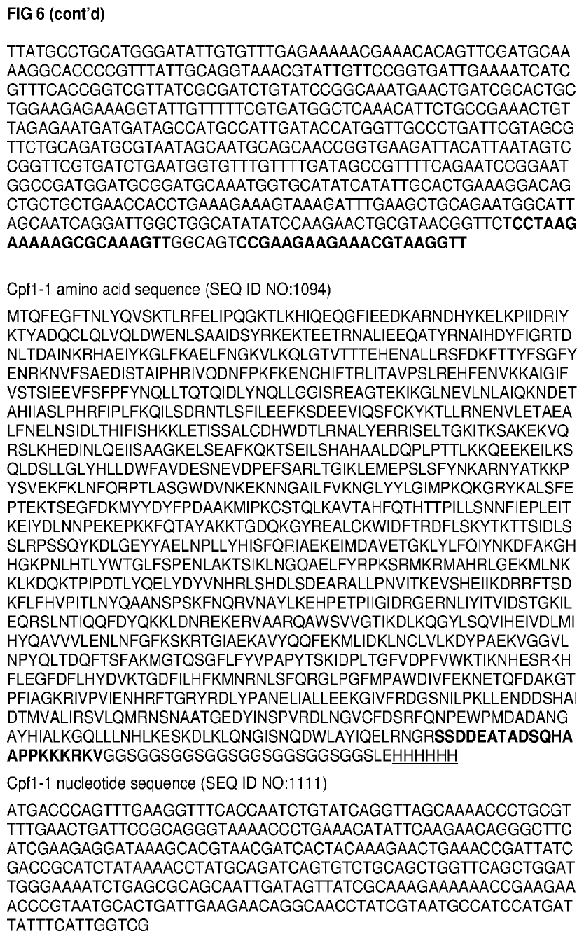

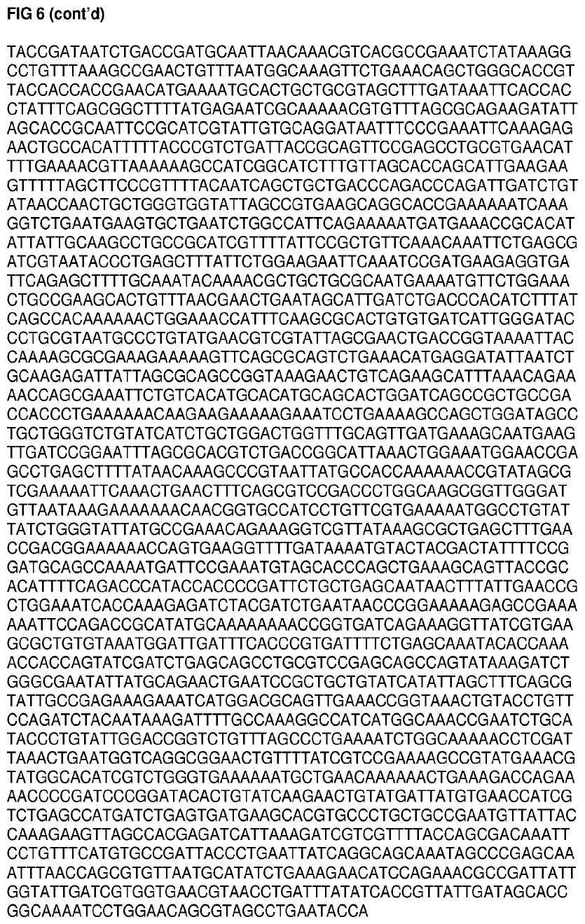

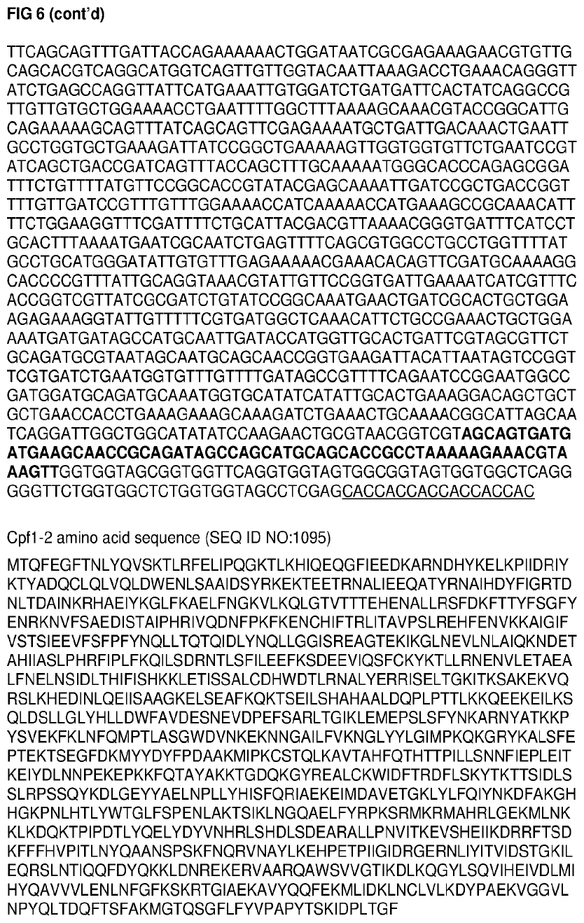

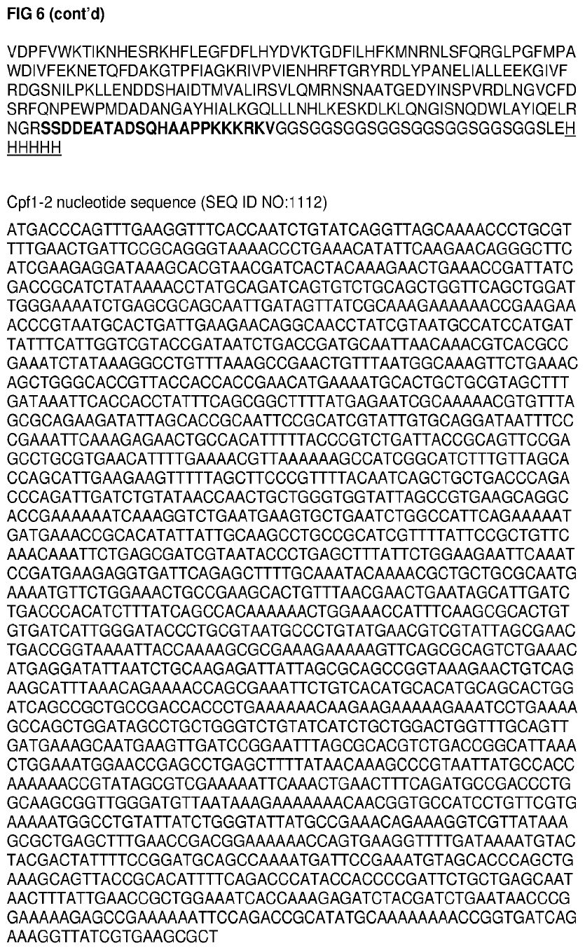

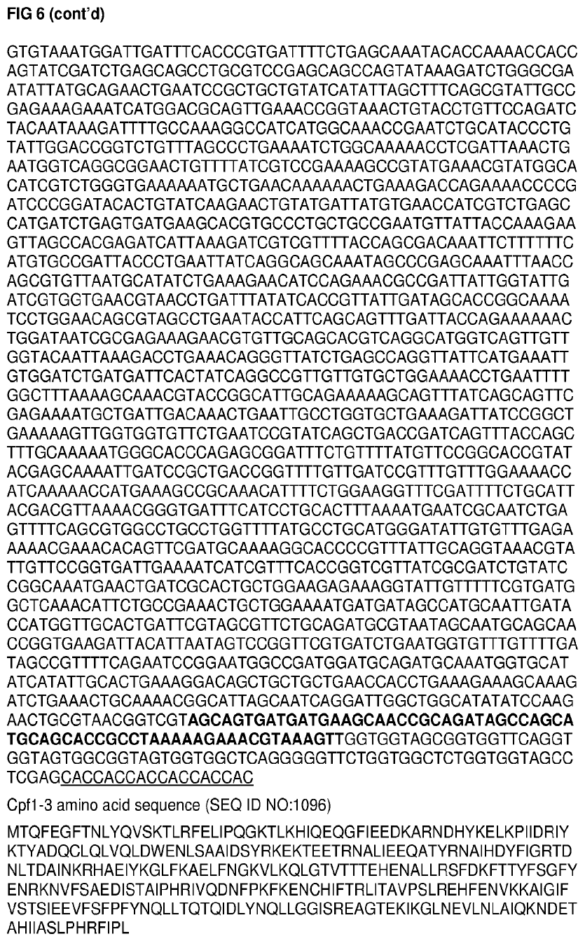

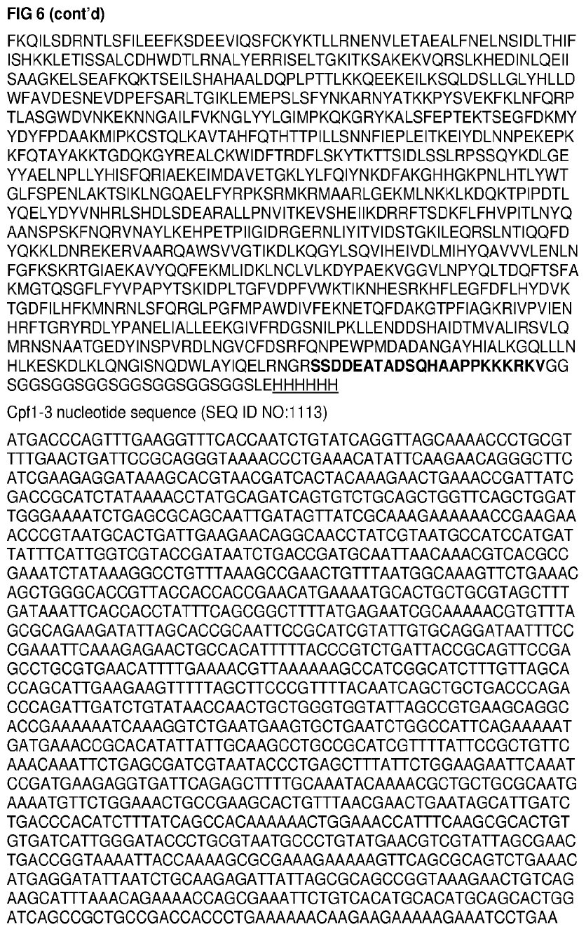

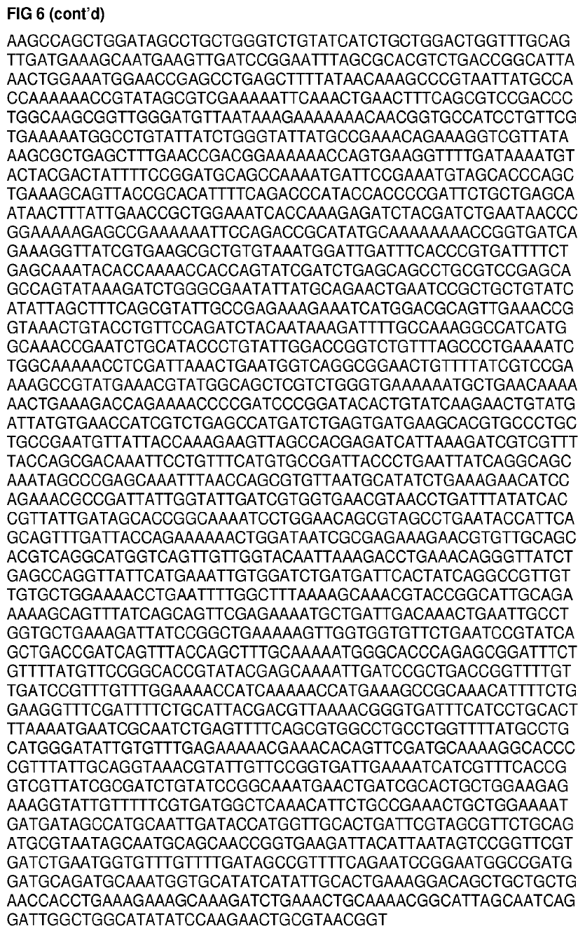

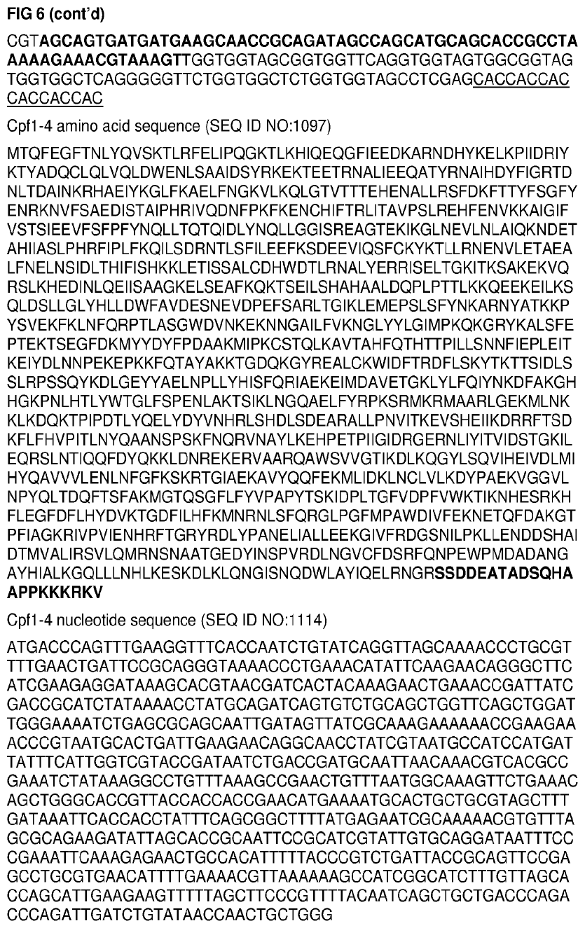

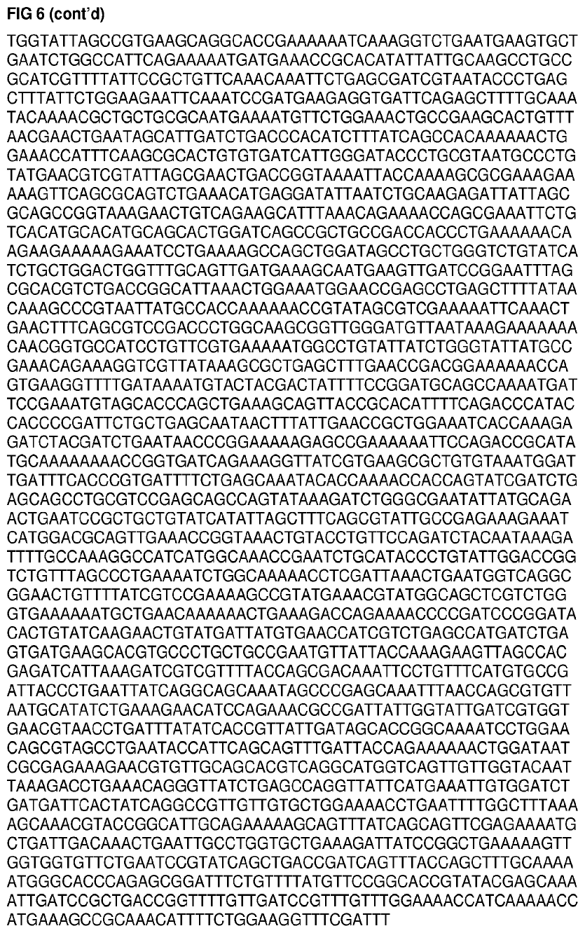

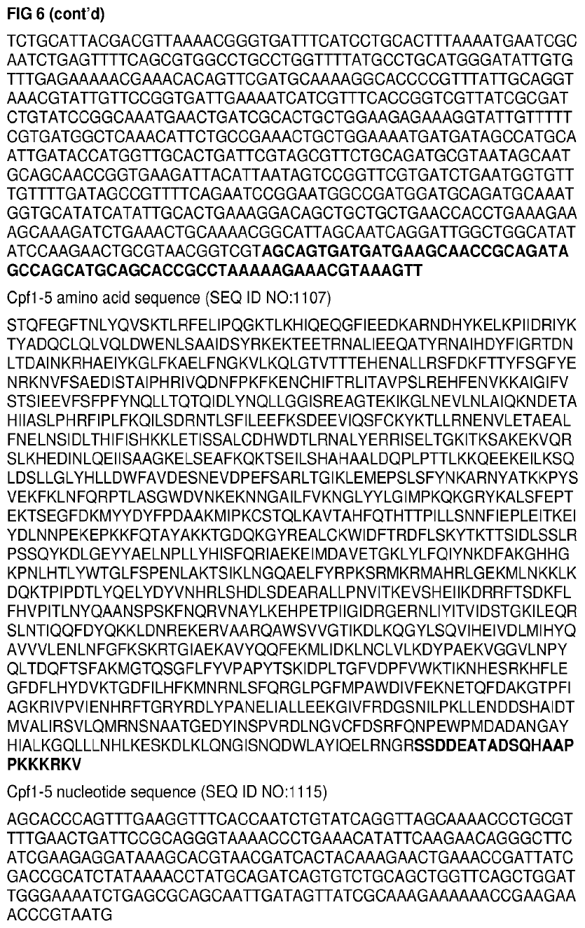

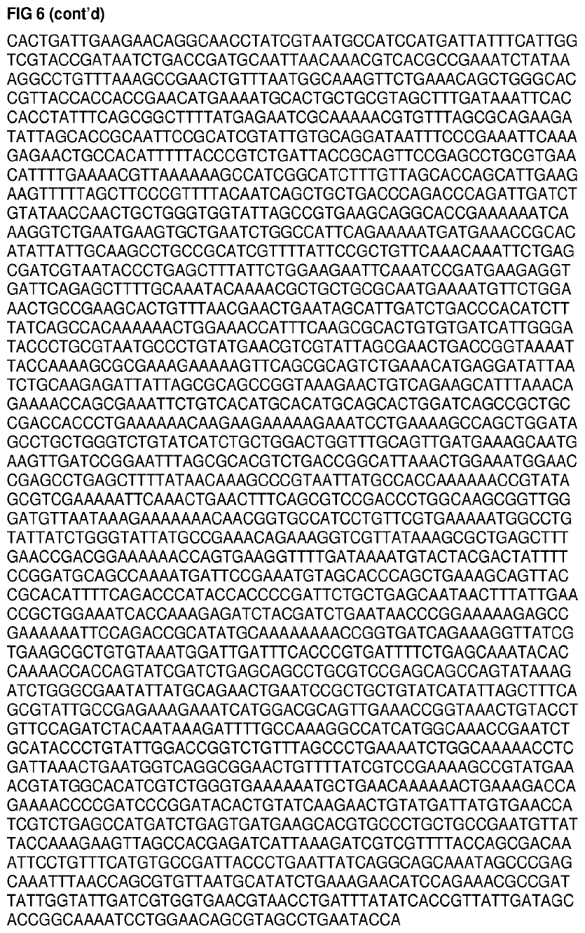

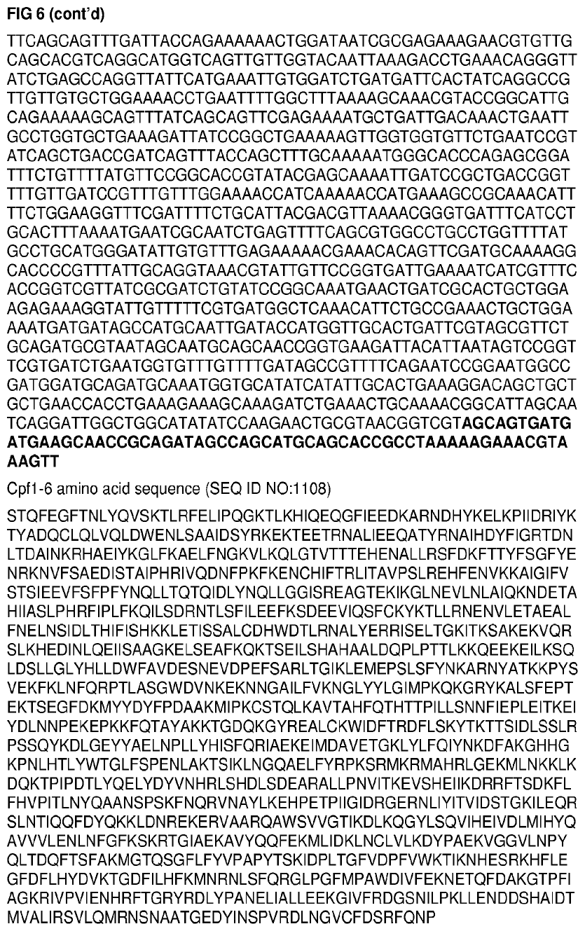

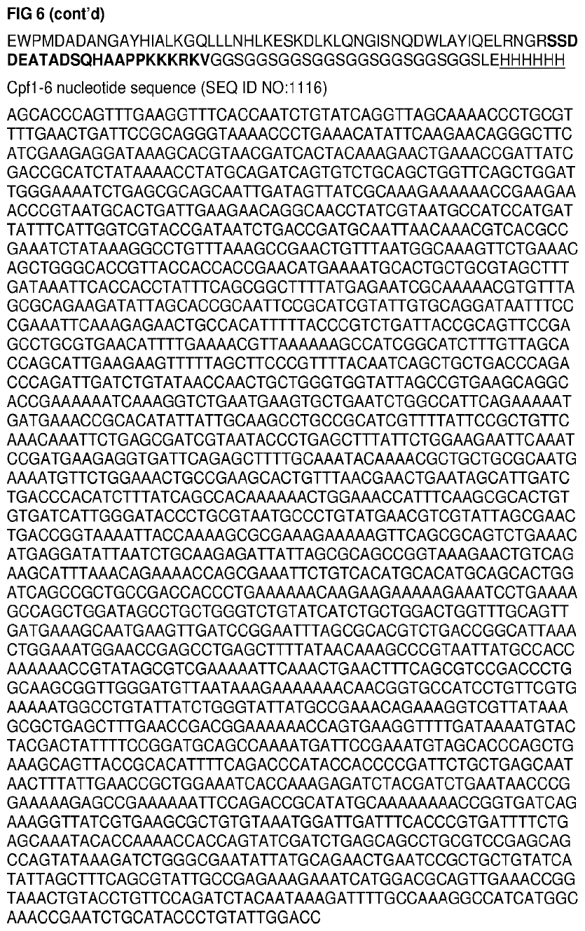

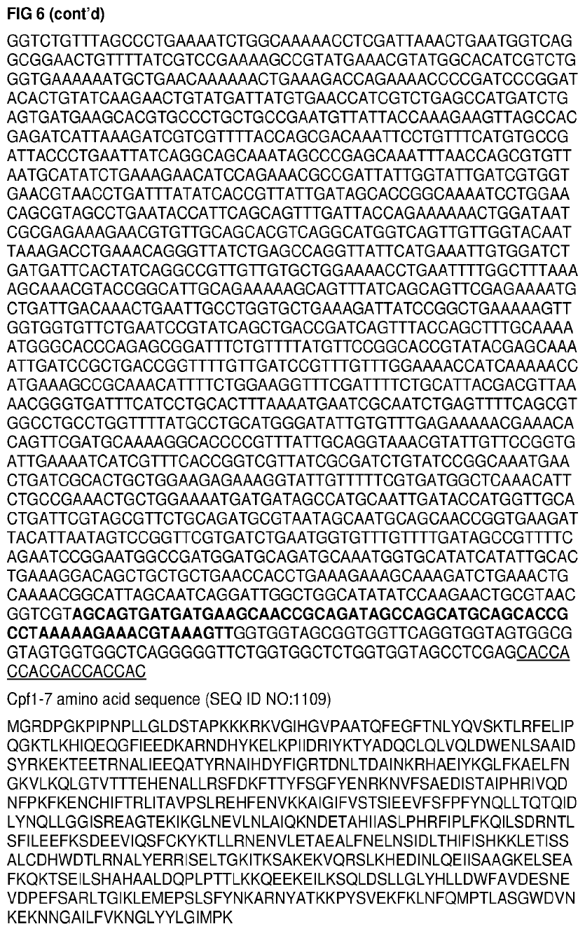

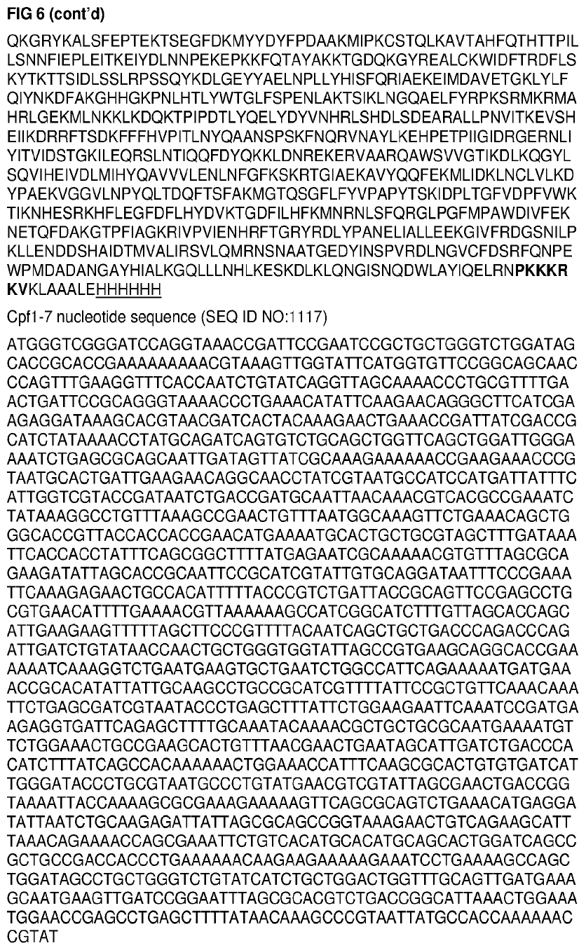

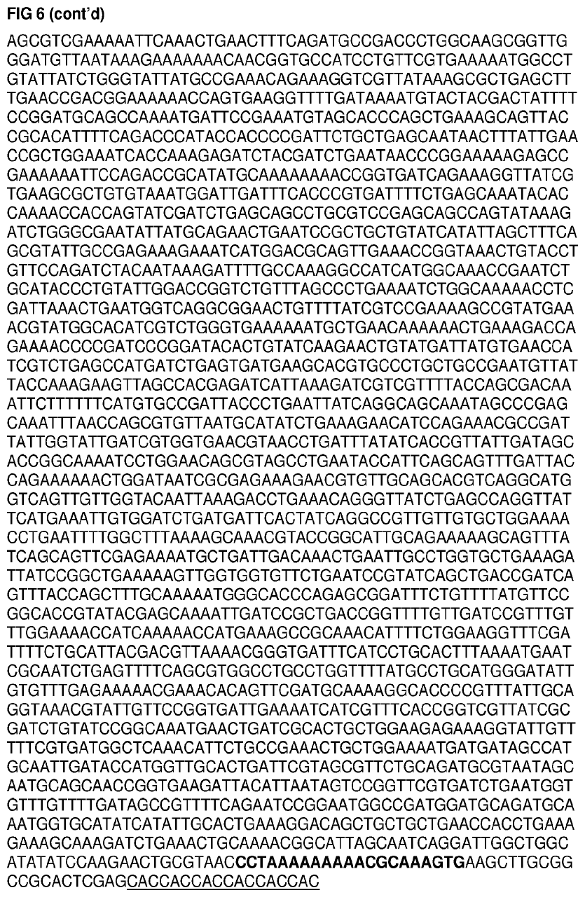

In certain embodiments, a modified Cpf1 protein (also referred to as a Cpf1 variant) used herein may be encoded by any of the sequences set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences). Table 9 sets forth exemplary Cpf1 variant amino acid and nucleotide sequences. See International Publication No. WO 2021/119040 (see, e.g., Table 14). These sequences are set forth in FIG. 6, which details the positions of six-histidine sequences (underlined letters) and NLS sequences (bolded letters). Additional permutations of the identity and N-terminal/C-terminal positions of NLS sequences, e.g., appending two or more nNLS sequences or combinations of nNLS and sNLS sequences (or other NLS sequences) to either the N-terminal/C-terminal positions, as well as sequences with and without purification sequences, e.g., six-histidine sequences, are within the scope of the instantly disclosed subject matter.

In certain embodiments, any of the Cpf1 proteins or modified Cpf1 proteins disclosed herein may be complexed with one or more gRNA comprising the targeting domain set forth in SEQ ID NOs 1002 and/or 1004 to alter a CCAAT box target region. In certain embodiments, any of the Cpf1 proteins or modified Cpf1 proteins disclosed herein may be complexed with one or more gRNA comprising a sequence set forth in Tables 7, 8, 11, or 12. In certain embodiments, the modified Cpf1 protein may be His-AsCpf1-nNLS (SEQ ID NO:1000) or His-AsCpf1-sNLS-sNLS (SEQ ID NO:1001). In certain embodiments, a modified Cpf1 protein used herein may be encoded by any of the sequences set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences). In certain embodiments, the modified Cpf1 protein may comprise the sequence set forth in SEQ ID NO:1097.

In certain embodiments, the modified Cpf1 protein may include a Cpf1 variant described in Kleinstiver 2019. For example, without limitation, in certain embodiments, the modified Cpf1 protein may be enAsCas12a, as described in Kleinstiver 2019. In certain embodiments, the modified Cpf1 protein may cleave target sites with a TTTV PAM. In certain embodiments, the modified Cpf1 protein may cleave target sites with a NWYN PAM.

### Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cpf1 Nuc domain are described in Ran & Hsu 2013 and Yamano 2016, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand as shown below (where C denotes the site of cleavage).

On the other hand, inactivation of a Cas9 HNH domain results in a nickase that cleaves the bottom or non-complementary strand.

Modifications of PAM specificity relative to naturally occurring Cas9 reference molecules has been described by Kleinstiver et al. for both S. pyogenes (Kleinstiver 2015a) and S. aureus (Kleinstiver 2015b). Kleinstiver et al. have also described modifications that improve the targeting fidelity of Cas9 (Kleinstiver 2016). Kleinstiver et al. have also described modifications of Cpf1 that provide increased activity and improved targeting ranges (Kleinstiver 2019). Each of these references is incorporated by reference herein.

RNA-guided nucleases have been split into two or more parts, as described by Zetsche 2015 and Fine 2015 (both incorporated by reference herein).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger 2014, incorporated by reference herein for all purposes.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### Nucleic Acids Encoding RNA-Guided Nucleases

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Functional Analysis of Candidate Molecules

Candidate RNA-guided nucleases, gRNAs, and complexes thereof, can be evaluated by standard methods known in the art. See, e.g. Cotta-Ramusino. The stability of RNP complexes may be evaluated by differential scanning fluorimetry, as described below.

### Differential Scanning Fluorimetry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA:RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g. 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of Cas9 in water+10×SYPRO Orange® (Life Technologies cat #S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g. 2 μM) Cas9 in optimal buffer from assay 1 above and incubating (e.g. at RT for 10′) in a 384 well plate. An equal volume of optimal buffer+10×SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson 2016 (incorporated by reference herein). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran & Hsu 2013 and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. 1, 2, 3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

### Multiplex Strategies

Genome editing systems according to this disclosure may also be employed for multiplex gene editing to generate two or more DSBs, either in the same locus or in different loci. Any of the RNA-guided nucleases and gRNAs disclosed herein may be used in genome editing systems for multiplex gene editing. Strategies for editing that involve the formation of multiple DSBs, or SSBs, are described in, for instance, Cotta-Ramusino. In certain embodiments, multiple gRNAs and an RNA-guided nuclease may be used in genome editing systems to introduce alterations (e.g., deletions, insertions) into the CCAAT box target region of HBG1 and/or HBG2. In certain embodiments, the RNA-guided nuclease may be a Cpf1 or modified Cpf1.

### Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs or to boost overall editing rate, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g. flanking or adjoining) a target sequence to be cleaved. These homologous regions are referred to here as “homology arms,” and are illustrated schematically below:

[5′homology arm]-[replacement sequence]-[3′homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson 2016, which is incorporated by reference herein, found that the relative asymmetry of 3′ and 5′ homology arms of single stranded donor templates influenced repair rates and/or outcomes.

Replacement sequences in donor templates have been described elsewhere, including in Cotta-Ramusino. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or then, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 80-200 nucleotides (e.g., 80, 90, 100, 110, 120, 130, 140, 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g. in an AAV or lentiviral genome) that includes certain genomic backbone elements (e.g. inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino, which is incorporated by reference.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

In certain embodiments, silent, non-pathogenic SNPs may be included in the ssODN donor template to allow for identification of a gene editing event.

A donor template or template nucleic acid, as that term is used herein, refers to a nucleic acid sequence which can be used in conjunction with an RNA nuclease molecule and one or more gRNA molecules to alter (e.g., delete, disrupt, or modify) a target DNA sequence. In certain embodiments, the template nucleic acid results in an alteration (e.g., deletion) at the CCAAT box target region of HBG1 and/or HBG2. In certain embodiments, the alteration is a non-naturally occurring alteration.

In certain embodiments, the ssODN comprises, consists essentially of, or consists of one or more sequences selected from the group consisting of SEQ ID NO:974-995, 1040. See International Publication No. WO 2021/119040 (see, e.g., Examples 2, 9, 10, 11, 12).

In certain embodiments, the 5′ homology arm comprises a 5′ phosphorothioate (PhTx) modification. In certain embodiments, the 3′ homology arm comprises a 3′ PhTx modification. In certain embodiments, the template nucleic acid comprises a 5′ and 3′ PhTx modification.

### Target Cells

Genome editing systems according to this disclosure can be used to manipulate or alter a cell, e.g., to edit or alter a target nucleic acid. The manipulating can occur, in various embodiments, in vivo or ex vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it may be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication.

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g. administered to a subject) immediately, or they can be maintained or stored for later use. Those of skill in the art will appreciate that cells can be maintained in culture or stored (e.g. frozen in liquid nitrogen) using any suitable method known in the art.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 2 and 3 set forth several, non-limiting examples of genome editing system implementations. Those of skill in the art will appreciate, however, that these listings are not comprehensive, and that other implementations are possible. With reference to Table 2 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 2
Genome Editing System Components

| RNA- | | | |
|---|---|---|---|
| guided | | Donor | |
| Nuclease | gRNA | Template | Comments |
| Protein | RNA | [N/A] | An RNA-guided nuclease protein |
| | | | complexed with a gRNA molecule |
| | | | (an RNP complex) |
| Protein | RNA | DNA | An RNP complex as described above |
| | | | plus a single-stranded or double |
| | | | stranded donor template. |
| Protein | DNA | [N/A] | An RNA-guided nuclease protein |
| | | | plus gRNA transcribed from DNA. |
| Protein | DNA | DNA | An RNA-guided nuclease protein |
| | | | plus gRNA-encoding DNA and a |
| | | | separate DNA donor template. |
| Protein | DNA | | An RNA-guided nuclease protein and |
| | | | a single DNA encoding both |
| | | | a gRNA and a donor template. |
| DNA | | | A DNA or DNA vector encoding an |
| | | | RNA-guided nuclease, a gRNA and a |
| | | | donor template. |
| DNA | DNA | [N/A] | Two separate DNAs, or two separate |
| | | | DNA vectors, encoding the RNA- |
| | | | guided nuclease and the gRNA, |
| | | | respectively. |
| DNA | DNA | DNA | Three separate DNAs, or three |
| | | | separate DNA vectors, encoding the |
| | | | RNA-guided nuclease, the gRNA and |
| | | | the donor template, respectively. |
| DNA | | [N/A] | A DNA or DNA vector encoding an |
| | | | RNA-guided nuclease and a gRNA |
| DNA | | DNA | A first DNA or DNA vector encoding |
| | | | an RNA-guided nuclease and a gRNA, |
| | | | and a second DNA or DNA vector |
| | | | encoding a donor template. |
| DNA | DNA | | A first DNA or DNA vector encoding |
| | | | an RNA-guided nuclease and second |
| | | | DNA or DNA vector encoding a |
| | | | gRNA and a donor template. |
| | DNA | | A first DNA or DNA vector encoding |
| | DNA | | an RNA-guided nuclease and a donor |
| | | | template, and a second DNA or DNA |
| | | | vector encoding a gRNA |
| | DNA | | A DNA or DNA vector encoding an |
| | RNA | | RNA-guided nuclease and a donor |
| | | | template, and a gRNA |

| RNA | [N/A] | An RNA or RNA vector encoding an |
|---|---|---|
| | | RNA-guided nuclease and comprising |
| | | a gRNA |
| RNA | DNA | An RNA or RNA vector encoding an |

RNA-guided nuclease and comprising
a gRNA, and a DNA or DNA vector
encoding a donor template.

Table 3 summarizes various delivery methods for the components of genome editing systems, as described herein. Again, the listing is intended to be exemplary rather than limiting.

TABLE 3

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium | | | | | and Proteins |
| Phosphate transfection, cell | | | | | |
| compression or squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

### Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 3, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 3, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g. lipid and/or polymer) nonparticles can be suitable for use as delivery vehicles in certain embodiments of this disclosure. Exemplary lipids for use in nanoparticle formulations, and/or gene transfer are shown in Table 4 and Table 5 lists exemplary polymers for use in gene transfer and/or nanoparticle formulations.

TABLE 4
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 2Oc | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]-dimethylammonium | CLIP-1 | Cationic |
| chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] imidazolinium | DOTIM | Cationic |
| chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

TABLE 5
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly(2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the Genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the Genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo. A protective, interactive, non-condensing (PINC) system may be used for delivery.

In vitro delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

### Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein. In some embodiments, genome editing systems, system components and/or nucleic acids encoding system components, are delivered with a block copolymer such as a poloxamer or a poloxamine.

### Multi-Modal or Differential Delivery of Components

Skilled artisans will appreciate, in view of the instant disclosure, that different components of genome editing systems disclosed herein can be delivered together or separately and simultaneously or non-simultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only be formed in the tissue that is targeted by both vectors.

### EXAMPLES

The principles and embodiments described above are further illustrated by the non-limiting examples that follow:

### Example 1: Use of Ribonucleoprotein for the Treatment of β-Hemoglobinopathy

Described herein is an autologous cell therapy for beta thalassemia comprising administering genetically modified CD34+ cells to subjects suffering from beta thalassemia to promote gamma globin expression. In certain embodiments, the beta thalassemia may be transfusion-dependent beta thalassemia (TDT). Beta thalassemia is one of the most common recessive hematological disorders in the world with more than 200 mutations identified to date. These mutations reduce or completely abrogate beta globin expression. As beta globin pairs with alpha globin to form adult hemoglobin (HbA, α2β2), reduced or absent beta globin results in excessive alpha globin chains, which form toxic aggregates. These aggregates cause maturation blockade and premature death of erythroid precursors, and hemolysis of red blood cells (RBC), leading to varying degrees of anemia. Patients with the most severe form of beta thalassemia, namely beta thalassemia major, are transfusion-dependent, i.e., requiring life-long RBC transfusions accompanied by the burden of iron chelation therapy.

The autologous cell therapy described herein is a therapeutic approach for treating beta thalassemia to promote the expression of fetal hemoglobin by directly targeting the promoter of the HBG1 and HBG2 genes which encode for the fetal gamma globin chains. Gamma globin decreases the alpha to beta globin chain imbalance in beta thalassemia by pairing with the over-abundant alpha globin chains to form fetal hemoglobin (HbF, α2γ2). Gamma globin induction, and consequently HbF induction, can be achieved through Cpf1 (Cas12a) ribonucleoprotein (RNP)-mediated editing of the distal CCAAT-box region of the HBG1 and HBG2 promoters, where naturally occurring hereditary persistence of fetal hemoglobin (HFPH) mutations exist.

RNP32 (Table 10), comprising a gRNA (comprising the sequence set forth in SEQ ID NO:1051) and a modified Cpf1 protein (comprising the sequence set forth in SEQ ID NO: 1097), edits the HBG1 and HBG2 promoter distal CCAAT box with high efficiency and specificity.

To test whether RNP32 may be an efficacious therapy for beta thalassemia (e.g., TDT), mPB CD34+ cells from individuals with TDT were electroporated with RNP32 targeting the HBG1 and HBG2 promoters. The efficiency of RNP32 editing for such a cell therapy was determined using mPB CD34+ cells obtained from individuals with TDT and normal donors and was compared. Briefly, CD34+ cells from normal or TDT donors were pre-stimulated in media consisting of X-Vivo 10, supplemented with 1×Glutamax, 100 ng/mL stem cell factor (SCF), 100 ng/mL thrombopoietin (TPO), and 100 ng/mL FMS-like tyrosine kinase 3 ligand (Flt3L) for 2 days in a humidified incubator at 37° C., 5% carbon dioxide (CO₂). After 2 days of culture, cells were collected and resuspended in MaxCyte electroporation buffer. RNP32 (6 μM, at a gRNA/protein molar ratio of 2) was delivered to CD34+ cells via a MaxCyte GT electroporation device. 1×10⁶ to 6.25×10⁶ cells can be used per OC-100 cartridge for electroporation. Pre-warmed complete media was then added to the cells to give a final cell density of approximately 1×10⁶ cells/mL. The electroporated cells, along with untreated control cells (cells that did not undergo electroporation), were then placed in a humidified incubator at 37° C., 5% CO₂. At Days 1, 2, and 3 post-electroporation, an aliquot of cells was harvested for further analyses. Crude genomic deoxyribonucleic acid (gDNA) extraction was conducted by subjecting the lysate to the following conditions in a thermocycler: 15 min at 65° C. followed by 10 min at 95° C. Crude gDNA was then analyzed for indels by next generation sequencing using the following primers: Forward=CATGGCGTCTGGACTAGGAG (SEQ ID NO:1266) and Reverse=AAACACATTTCACAATCCCTGAAC (SEQ ID NO: 1267).

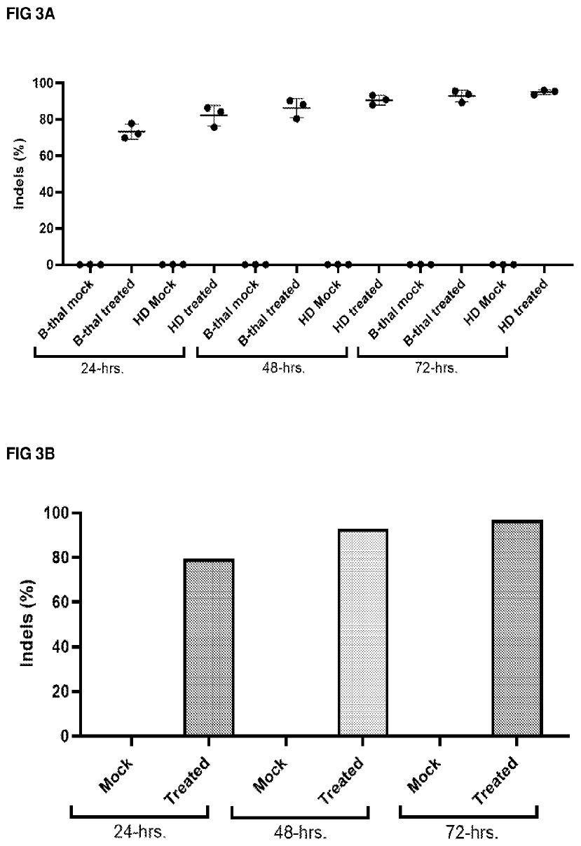

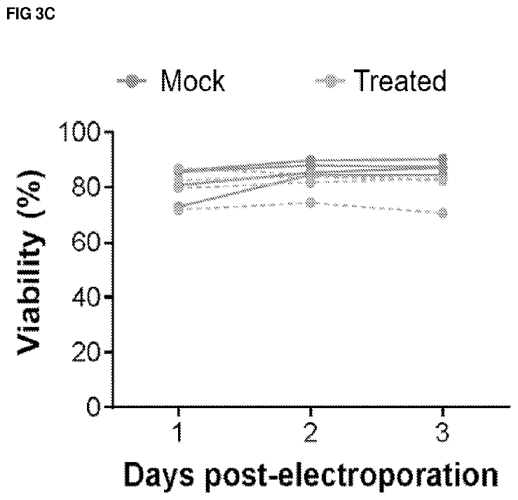

As shown in FIG. 3A, RNP32 edited mPB CD34+ cells from individuals with TDT as efficiently as CD34+ cells from normal donors. The percentage of indels increased from Day 1 post electroporation to Day 3 post-electroporation for cells from both TDT donors (FIG. 3A, 3B) and normal donors (FIG. 3A). Moreover, in addition to efficient editing (FIG. 3A), RNP32 edited mPB CD34+ cells from individuals with TDT maintained high viability from Day 1 post electroporation to Day 3 post-electroporation (FIG. 3C).

Next, the erythroid differentiation of RNP32 edited beta thalassemia CD34+ cells from three individuals with TDT (donors 1-3) was tested to assess maturation and health of RNP edited erythroid cells as maturation blockade and premature death of erythroid precursors are hallmarks of TDT.

Briefly, at Day 1 post-electroporation with RNP32, cells were cultured in erythroid-inducing media to generate erythroid cells. CD34+ cells were cultured for 7 days in Step-1 media consisting of Iscove's modified Dulbecco's medium (IMDM) supplemented with 1×GlutaMAX (Gibco), 100 U/mL penicillin, 100 mg/mL streptomycin, 5% human AB+ plasma, 330 μg/mL human holo transferrin, 20 mg/mL human insulin, 2 U/mL heparin, 3 U/mL recombinant human erythropoietin (EPO), 100 ng/mL SCF, and 5 ng/mL interleukin (IL)-3. On Day 7, cells were transferred to Step-2 media, which was identical to Step-1 media except the absence of IL-3 and cultured for 4 days. Next, cells were cultured for 7 days in Step-3 media, which was similar to Step-2 media but without the addition of SCF, and 5% human AB+ plasma was replaced with 5% KnockOut Serum Replacement (Gibco). At the end of the 18-day culture, erythroid maturation, enucleation, and cell death frequency was determined using fluorescence activated cell sorting (FACS).

Erythroid differentiation of edited beta thalassemia CD34+ cells showed significant improvement in erythroid maturation and health. Day 18 erythroid cells were stained with antibodies against CD71 and CD235a, with NucRed that stains cells that contain nucleus, and DAPI that stains dead cells. Erythroblasts were classified as live, nucleated, and CD235a high population. Late erythroblasts were classified as the erythroblasts that have low or negative CD71 expression.

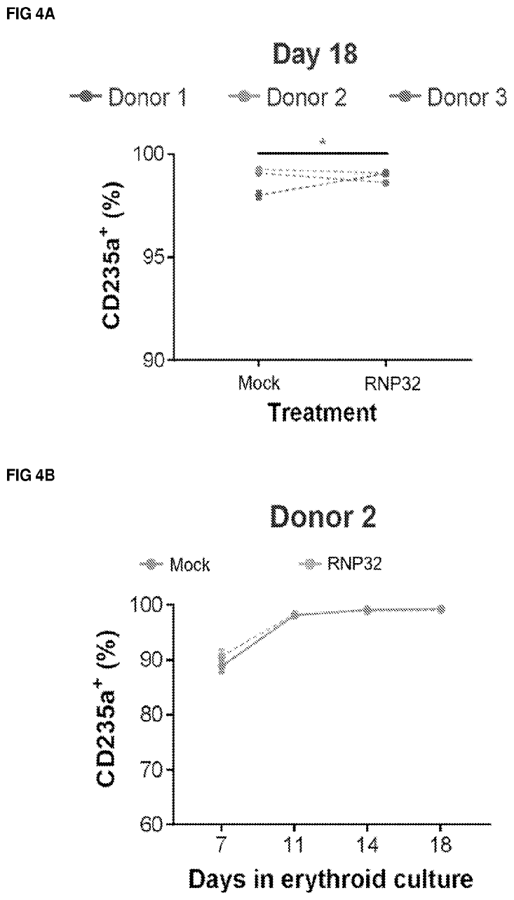

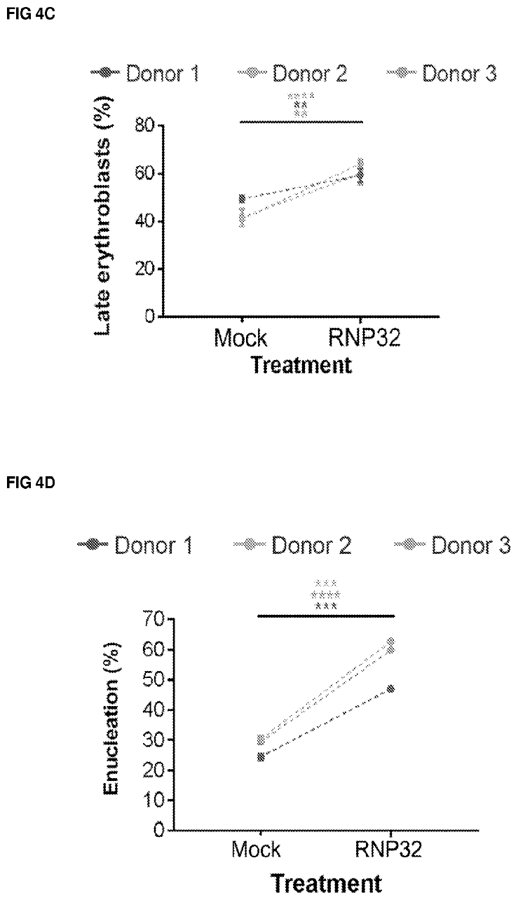

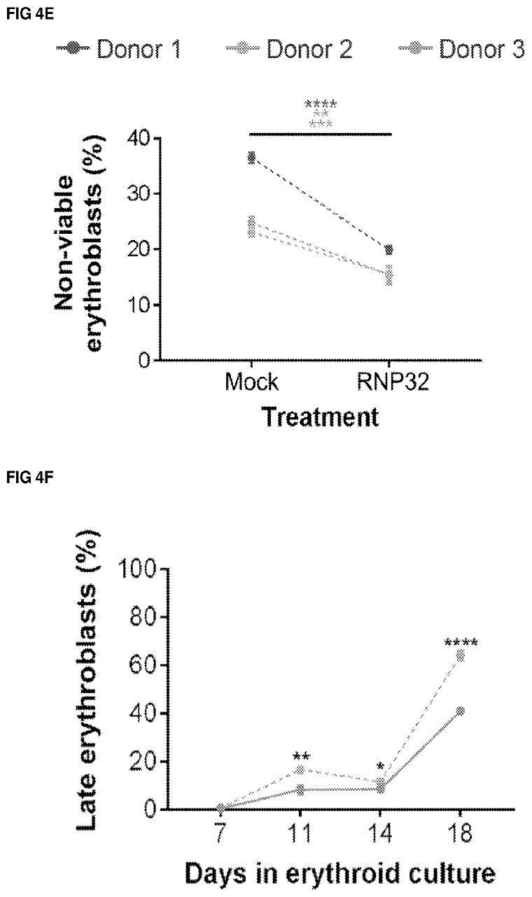

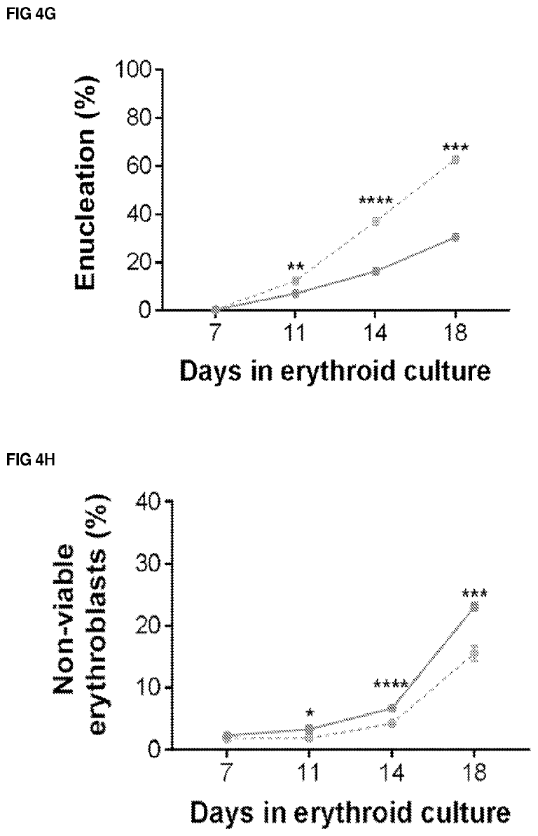

Beta thalassemia CD34+ donor cells edited with RNP32 successfully underwent erythroid differentiation at a similar rate to unedited control cells (FIGS. 4A, 4B). Approximately 70% edited erythroblasts reached late erythroblast stage compared to approximately 53% unedited erythroblasts (FIG. 4C). Enucleated erythroid cells were classified as NucRed negative cells within live and CD235a high population. Approximately 56% edited erythroid cells underwent terminal maturation and enucleated compared to approximately 28% of unedited erythroid cells (FIG. 4D). Non-viable erythroblasts were classified as those that were stained positive with DAPI within the nucleated and CD235a high population. Non-viable erythroblasts decreased from approximately 33% to approximately 22% after editing (FIG. 4E). FIGS. 4F-4H show the percentage of cells (edited and unedited) that reached late erythroblast stage, the percentage of enucleated erythroid cells, and the percentage of non-viable erythroblasts, respectively, from one donor at Days 7, 11, 14, and 18 in erythroid culture.

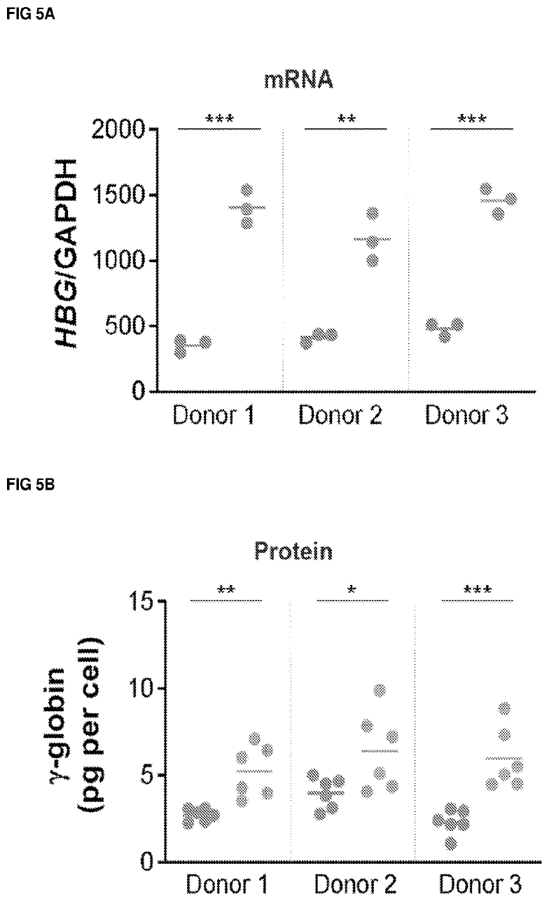

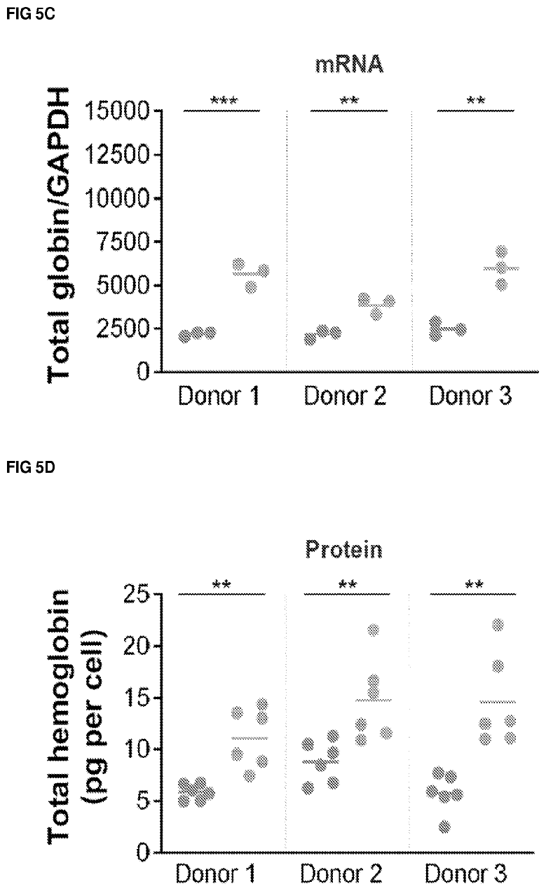

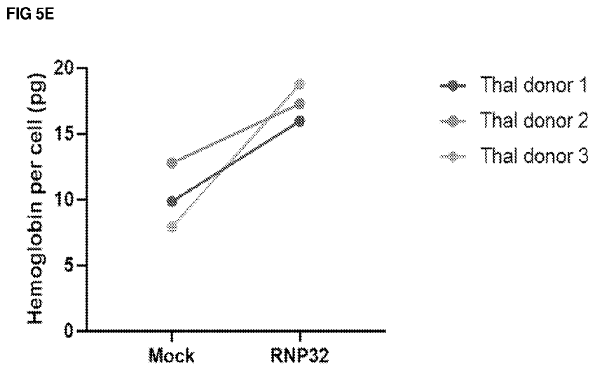

Changes in γ-globin and total globin production, both at the mRNA and protein levels, were evaluated in erythroid cells differentiated from beta thalassemia CD34+ donor cells edited with RNP32 or unedited cells using reverse-transcription droplet digital polymerase chain reaction and reverse phase ultra-performance liquid chromatography (RP-UPLC). Total area under the curve for alpha, beta, and gamma globins was calculated against a standard curve to determine the hemoglobin content per cell. Results indicated that the improved erythropoiesis was accompanied by significantly increased γ-globin and total hemoglobin levels compared with unedited controls at both the mRNA and protein levels (FIGS. 5A-5E). These data strongly support that editing of the HBG1 and HBG2 promoter CCAAT box using RNP32 can reverse the dyserythropoiesis associated with beta thalassemia and increase the hemoglobin production.

In summary, the data herein support the use of RNP32 in an autologous cell therapy for beta-thalassemia. Erythroid cells differentiated from beta thalassemia CD34+ donor cells edited with RNP32 exhibited significantly improved erythroid maturation and decreased erythroid death, therefore reversing the maturation blockade associated with TDT mutations. Erythroid cells differentiated from beta thalassemia CD34+ donor cells edited with RNP32 had significantly increased γ-globin production and total hemoglobin content per cell. Treatment with RNP32 can help to address the underlying disease mechanism of TDT and demonstrates improved erythropoiesis and increased hemoglobin content in its erythroid progeny. As edited mPB CD34+ cells retain their ability to engraft and result in robust HbF induction long-term, these data support that RNP32 can be used as a one-time efficacious autologous cell therapy for individuals with TDT to reverse dyserythropoiesis and ameliorate anemia.

### Example 2: Treatment of β-Hemoglobinopathy Using Edited Hematopoietic Stem Cells

The methods and genome editing systems disclosed herein may be used for the treatment of a β-hemoglobinopathy, such as sickle cell disease or beta-thalassemia, in a patient in need thereof. For example, genome editing may be performed on cells derived from the patient in an autologous procedure. Correction of the patient's cells ex-vivo and reintroduction of the cells into the patient may result in increased HbF expression and treatment of the β-hemoglobinopathy.

For example, HSCs may be extracted from the bone marrow of a patient with a 3-hemoglobinopathy using techniques that are well-known to skilled artisans. The HSCs may be modified using methods disclosed herein for genome editing. For example, an RNP comprised of a guide RNA (gRNA) that targets one or more regions in the HBG gene complexed with an RNA-guided nuclease may be used to edit the HSCs. In certain embodiments, the RNA-guided nuclease may be a Cpf1 protein. In certain embodiments, the Cpf1 protein may be a modified Cpf1 protein. In certain embodiments, the modified Cpf1 protein may be encoded by a sequence set forth in SEQ ID NOs:1000, 1001, 1008-1018, 1032, 1035-39, 1094-1097, 1107-09 (Cpf1 polypeptide sequences) or SEQ ID NOs:1019-1021, 1110-17 (Cpf1 polynucleotide sequences). For example, the modified Cpf1 protein may be encoded by the sequence set forth in SEQ ID NO:1097. In certain embodiments, the gRNA may be a modified or unmodified gRNA. In certain embodiments, the gRNA may comprise a sequence set forth in Tables 7, 8, 11, or 12. For example, in certain embodiments, the gRNA may comprise the sequence set forth in SEQ ID NO:1051. In certain embodiments, the RNP complex may comprise an RNP complex set forth in Table 10. For example, the RNP complex may include a gRNA comprising the sequence set forth in SEQ ID NO: 1051 and a modified Cpf1 protein encoded by the sequence set forth in SEQ ID NO: 1097 (RNP32, Table 10). In certain embodiments, modified HSCs have an increase in the frequency or level of an indel in the human HBG1 gene, HBG2 gene, or both, relative to unmodified HSCs. In certain embodiments, the modified HSCs can differentiate into erythroid cells that express an increased level of HbF. A population of the modified HSCs may be selected for reintroduction into the patient via transfusion or other methods known to skilled artisans. The population of modified HSCs for reintroduction may be selected based on, for example, increased HbF expression of the erythroid progeny of the modified HSCs or increased indel frequency of the modified HSCs. In some embodiments, any form of ablation prior to reintroduction of the cells may be used to enhance engraftment of the modified HSCs. In other embodiments, peripheral blood stem cells (PBSCs) can be extracted from a patient with a β-hemoglobinopathy using techniques that are well-known to skilled artisans (e.g., apheresis or leukapheresis) and stem cells can be removed from the PBSCs. The genome editing methods described above can be performed on the stem cells and the modified stem cells can be reintroduced into the patient as described above.

TABLE 6
Subdomains of the HBG genomic region

| Genomic | | |
|---|---|---|
| Coordinate | | Name of |
| of HbG* | Nucleotides | Region |
| Chr 11 | TCCTAAAGCTTGGAACACTTTCCCTTCCTTAAGAACC | Region 1: |
| (NC_000011.10): | ATCCTTGCTACTCAGCTGCAATCAATCCAGCCCCCAG | Downstream of |
| 5,247,883- | GTCTTCACTGAACCTTTTCCCATCTCTTCCAAAACATC | HBG1 |
| 5,248,186 | TGTTTCTGAGAAGTCCTGTCCTATAGAGGTCTTTCTTC | |
| | CCACCGGATTTCTCCTACACCATTTACTCCCACTTGCA | |
| | GAACTCCCGTGTACAAGTGTCTTTACTGCTTTTATTTG | |
| | CTCATCAAAATGCACATCTCATATAAAAATAAATGAG | |
| | GAGCATGCACACACCACAAACACAAACAGGCATGCA | |
| | GAAAT (SEQ ID NO: 1118) | |
| Chr 11 | ATAAAGATGAACCCATAGTGAGCTGAGAGCTCCAGC | Region 2: |
| (NC_000011.10): | CTGGCCTCCAGATAACTACACACCAAGCTTCCACCCA | HBG1 Intron |
| 5,248,509- | GAATCAAGCCTATGTTAACTTCCCTCAAAGCCTGAGA | 2-A |
| 5,249,173 | TTTTGCCTTCCCATTAAATGCAGGTAGTTGTTCCCCTT | |
| | CAAGCACTAGTCACTGGCCATAATTTAAATCTTGCTA | |
| | TCTTCTTGCCACCATGAACCCTGTATGTTGTAGGCTG | |
| | AAGACGTTAAAAGAAACACACGCTGACACACACACA | |
| | CACACGCGCGCGCGCACACACACACACACACACAGA | |
| | GCTGACTTTCAAAATCTACTCCAGCCCAAATGTTTCA | |
| | ATTGTTCCTCACCCCTGGACATACTTTGCCCCCATCTG | |
| | GAATTAAAGGATATAAGTTTGTAATGAAGCATTAGCA | |
| | GCATTTTATATGTGTCCAGCTGATATAGGAATAGCCT | |
| | TAGCAATGTATGTTTGGCCACCAAAGTTCCCCACTTT | |
| | GACTGAGCCAATATATGCCTTCTGCCTGCATCTTTTTA | |
| | ACGACCATACTTGTCCTGCCTCCAGATAGATGTTTTA | |
| | AAACAACAAAAATGAGGGAAAGATGAAAGTTCTTTC | |
| | TACTGGAATCTAATAAAGAAAAGTCATTTTCCTCATT | |
| | TCCACCTCTCTTTTCTCAAAGTCAAAATTGTCCATCT | |
| | (SEQ ID NO: 1119) | |
| Chr 11 | CCCTAAAACATTACCACTGGGTCTCAGCCCAGTTAGT | Region 3: |
| (NC_000011.10) | CCTCTGCAGTTTCTTCACCCCCAACCCCAGTATCTTCA | HBG1 Intron |
| 5,249,198- | AACAGCTCACACCCTGCTGTGCTCAGATCAATACTCC | 2-B |
| 5,249,362 | GTTGTCTAAGTTGCCTCGAGACTAAAGGCAACAGGG | |
| | CTGAAACATCTCCTGGA (SEQ ID NO: 1120) | |
| Chr 11 | CTGTGAGATTGACAAGAACAGTTTGACAGTCAGAAG | Region 4: |
| (NC_000011.10) | GTGCCACAAATCCTGAGAAGCGACCTGGACTTTTGCC | HBG1 Intron 1 |
| 5,249,591- | AGGCACAGGGTCCTTCCTTCCCTCCCTTGTCCTGGTC | |
| 5,249,712 | ACCAGAGCCTAC (SEQ ID NO: 1121) | |
| Chr 11 | GCCGCCGGCCCCTGGCCTCACTGG | Region 5: |
| (NC_000011.10): | (SEQ ID NO: 1122) | HBG1 −60 nt |
| 5,249,904- | | region from |
| 5,249,927 | | Transcription |
| | | Start Site (TSS) |
| Chr 11 | CCTTGTCAAGGCTATTGGTCAAGGCAAGGCTGG (SEQ | Region 6: |
| (NC_000011.10) | ID NO: 1123) | HBG1 −110 nt |
| 5,249,955- | | region from |
| 5,249,987 | | TSS |
| Chr 11 | TGAGATAGTGTGGGGAAGGGGCCCCCAAGAGGATAC | Region 7: |
| (NC_000011.10): | (SEQ ID NO: 1124) | HBG1 −200 nt |
| 5,250,040- | | region from |
| 5,250,075 | | TSS |
| Chr 11 | TATAGCCTTTGCCTTGTTCCGATTCAGTCATTCCAGTT | Region 8: |
| (NC_000011.10) | TT T (SEQ ID NO: 1125) | HBG1 −250 nt |
| 5,250,089- | | region from |
| 5,250,129 | | TSS |
| Chr 11 | TCTTCCCTTTAGCTAGTTTCCTTCTCCCATCATAGAGG | Region 9: |
| (NC_000011.10): | ATACCAGGACTTCTTTTGTCAGCCGTTTTTTACCTTCT | HBG1 −333 nt |
| 5,250,141- | TGTCTCTAGCTCCAGTGAGGCCTGTAGTTTAAAGCTA | region from |
| 5,250,254 | A (SEQ ID NO: 1126) | TSS |
| Chr 11 | CCACAGTTTCAGCGCAGTAATAGATTAGTGTTACATA | Region 10: |
| (NC_000011.10) | ATATAAGACCTAATGCTTACCTCAATATCTACTTATC | HBG1 −650 nt |
| 5,250,464- | CGTACCTATTTG (SEQ ID NO: 1127) | region from |
| 5,250,549 | | TSS |
| Chr 11 | TATTCAGGTATGTATGTATACACCAGATGATGTGTAT | Region 11: |
| (NC_000011.10): | TTACCACTGGATAAGTGTGTGTGCTGGCTGATGACCC | HBG1 −800 nt |
| 5,250,594- | AGGGTTTTGGCGTAGCTCTTCTATGCTCAGTAAAGAT | |
| 5,250,735 | GATGGTAGAATGTTCTTTGGCAGGTACTGTG (SEQ ID | region from |
| | NO: 1128) | TSS |
| Chr 11 | CAATAAAGATGAACCCATAGTGAGCTGAGAGCTCCA | Region 12: |
| (NC_000011.10): | GCCTGGCCTCCAGATAACTACACACCAAGCTTCCACC | HBG2 Intron |
| 5,253,425- | CAGAATCAAGCCTATGTTAACTTCCCTCAAAGCCTGA | 2-A |
| 5,254,121 | GATTTTGCTTTCCCATTAAATGCAGGTAGTTGTTCTTC | |
| | TTGCAGCACTAGTCACTGGCCATAATTTAAATCTTGT | |
| | TATCTTCTTGCCACCATGAACCCTGTATGCTGTAGGC | |
| | TGAAAACGTTAAAAGAAACACACGCTCTCACACACA | |
| | CACAAACACACGCGCGCACACACACACACACACACA | |
| | CAGAGCTGACTTTCAAAATCTACTCCAGCCCAAATGT | |
| | TTCAATTGTTCCTCACCCCTGGACATACTTTGCCCCCA | |
| | TCTGGAATTAAAGGATATAAGTTTGTAATGAAGCATT | |
| | AGCAGCATTTTATATGTGTCCAGCTGATATAGGAATA | |
| | GCCTTAGCAATGTATGTTTGGCCACCAAAGTTCCCCA | |
| | CTTTGACTGAGCCAATATATGCCTTCTGCCTGCATCTT | |
| | TTTAATGACCATACTTGTCCTGCCTCCAGATAGATGT | |
| | TTTAAAACGAATAACAAAAATAGGGGAAAGGTGAAA | |
| | GTTCTTTCTACCGAAATCTAATAAAGAAAAGTCATTT | |
| | TCCTCATTTCCACCTCTCTTTTCTCAAAGTCAAAGTTG | |
| | TCCATCTAGATTTTCAGAGGCACTCCTTAGG (SEQ ID | |
| | NO: 1129) | |
| Chr 11 | CCCTAAAACATTGCCACTGGGTCTCAGCCCAGTTAGT | Region 13: |
| (NC_000011.10): | CCTCTGCAGTTTCTTCACTCCCAACCCCAGTATCTTCA | HBG2 Intron |
| 5,254,122- | AACAGCTCACACCCTGCTGTGCTCAGATCAATACTCA | 2-B |
| 5,254,306 | GTTGTCTAAGTTGCCTCGAGACTAAAGGCAACAGTGC | |
| | TGAAACATCTCCTGGACTCACCTTGAAGTTCTCAGG | |
| | (SEQ ID NO: 1130) | |
| Chr 11 | AGCCTGTGAGATTGACAAGAACAGTTTGACAGTCAG | Region 14: |
| (NC_000011.10): | AAGGTGCCACAAATCCTGAGAAGCGACCTGGACTTTT | HBG2 Intron 1 |
| 5,254,511- | GCCAGGCACAGGGTCCTTCCTTCCCTCCCTTGTCCTG | |
| 5,254,648 | GTCACCAGAGCCTACCTTCCCAGGGTT (SEQ ID | |
| | NO: 1131) | |
| Chr 11 | CCGCCGGCCCCTGGCCTCACTGGATACTCTAAGACTA | Region 15: |
| (NC_000011.10): | T (SEQ ID NO:1132) | HBG2 −60 nt |
| 5,254,829- | | region from |
| 5,254,866 | | TSS |
| Chr 11 | CCTTGTCAAGGCTATTGGTCAAGGCAAGGCT (SEQ ID | Region 16: |
| (NC_000011.10): | NO: 1133) | HBG2 −110 nt |
| 5,254,879- | | region from |
| 5,254,909 | | TSS |
| Chr 11 | CAGGGACCGTTTCAGACAGATATTTGCATTGAGATAG | Region 17: |
| (NC_000011.10): | TGTGGGGAAGGGGCCCCCAAGAGGATACTGCTGCTT | HBG2 −200 nt |
| 5,254,935 | AA (SEQ ID NO: 1134) | region from |
| 5,255,009 | | TSS |
| Chr 11 | TTGCCTTGTTCCGATTCAGTCATTCCAAT (SEQ ID | Region 18: |
| (NC_000011.10): | NO: 1135) | HBG2 −250 nt |
| 5,255,025- | | region from |
| 5,255,053 | | TSS |
| Chr 11 | TTTAGCTAGTTTTCTTCTCCCACCATAGAAGATACCA | Region 19: |
| (NC_000011.10): | GGACTTCTTTTGTCAGCCGTTTTTCACCTTCTTGTCTG | HBG2 −330 nt |
| 5,255,076- | TAGCTCCAGTGAGGCCTGTAGTTTAAAGT (SEQ ID | region from |
| 5,255,179 | NO: 1136) | TSS |
| Chr 11 | GGACACGTCTTAGTCTCATTTAGTAAGCATTGGTTTC | Region 20: |
| (NC_000011.10): | C (SEQ ID NO: 1137) | HBG2 −500 nt |
| 5,255,255- | | region from |
| 5,255,292 | | TSS |
| Chr 11 | TTTTTTATATTCAGGTATGTATGTAGGCACCCGATGAT | Region 21: |
| (NC_000011.10): | GTGTATTTATCACTGGATAAGTGTATGTGCTGGCTGA | HBG2 −800 nt |
| 5,255,518- | TGACCCAGGGTTTTGGTGTAGCTCTTCTATGCTCGGT | region from |
| 5,255,641 | AAAGATGATGGT (SEQ ID NO: 1138) | TSS |

*NCBI Reference Sequence NC_000011, the coordinates are reported using the One-based coordinate system, “Homo sapiens chromosome 11, GRCh38.p12 Primary Assembly,” (Version NC_000011.10).

TABLE 7
Cpf1 guide RNAs

| gRNA | | | | | | |
|---|---|---|---|---|---|---|
| targeting | gRNA Targeting | gRNA Targeting | Genomic | Genomic | | |
| domain | domain sequence | domain | coordinates | coordinates | % | |
| sequence ID* | (RNA) | sequence (DNA) | at HbG1 ** | at HbG2 ** | Editing | Strand |
| AsCpf1 HBG1 | AGACAGAUAU | AGACAGATAT | Chr11:5250024: | Chr11:5254948: | 5.43 | + |
| Promoter-1 | UUGCAUUGAG | TTGCATTGAG | 5250043 | 5254967 | | |
| | (SEQ ID | (SEQ ID | | | | |
| | NO: 1139) | NO: 1140) | | | | |
| AsCpf1 HBG1 | CAUUGAGAUA | CATTGAGATA | Chr11:5250037: | Chr11:5254961: | 8.30 | + |
| Promoter-2 | GUGUGGGGAA | GTGTGGGGAA | 5250056 | 5254980 | | |
| | (SEQ ID | (SEQ ID | | | | |
| | NO: 1141) | NO: 1142) | | | | |
| AsCpf1 HBG1 | UAGCCUUUGC | TAGCCTTTGC | Chr11:5250091: | Chr11:5255019: | 0.23 | + |
| Promoter-3 | CUUGUUCCGA | CTTGTTCCGA | 5250110 | 5255038 | | |
| | (SEQ ID | (SEQ ID | | | | |
| | NO: 1143) | NO: 1144) | | | | |
| AsCpf1 HBG1 | CCUUGUUCCG | CCTTGTTCCG | Chr11:5250100: | Chr11:5255028: | 1.15 | + |
| Promoter-4 | AUUCAGUCAU | ATTCAGTCAT | 5250119 | 5255047 | | |
| | (SEQ ID | (SEQ ID | | | | |
| | NO: 1145) | NO: 1146) | | | | |
| AsCpf1 HBG1 | UCUAAUUUAU | TCTAATTTATT | Chr11:5250131: | Chr11:5255059: | 0.16 | + |
| Promoter-5 | UCUUCCCUUU | CTTCCCTTT | 5250150 | 5255078 | | |
| | (SEQ ID | (SEQ ID | | | | |
| | NO: 1147) | NO: 1148) | | | | |
| AsCpf1 HBG1 | CUUCUCCCAU | CTTCTCCCATC | Chr11:5250161: | | 12.73 | + |
| Promoter-6 | CAUAGAGGAU | ATAGAGGAT | 5250180 | | | |
| | (SEQ ID | (SEQ ID | | | | |
| | NO: 1149) | NO: 1150) | | | | |
| AsCpf1 HBG2 | UUCUCCCACC | TTCTCCCACC | | Chr11:5255090: | 8.11 | + |
| Promoter-7 | AUAGAAGAUA | ATAGAAGATA | | 5255109 | | |
| | (SEQ ID | (SEQ ID | | | | |
| | NO: 1151) | NO: 1152) | | | | |
| AsCpf1 HBG1 | CCACUGGAUA | CCACTGGATA | Chr11:5250634: | | 13.33 | + |
| Promoter-8 | AGUGUGUGUG | AGTGTGTGTG | 5250653 | | | |
| | (SEQ ID | (SEQ ID | | | | |
| | NO: 1153) | NO: 1154) | | | | |
| AsCpf1 HBG1 | GCGUAGCUCU | GCGTAGCTCT | Chr11:5250677: | | 13.48 | + |
| Promoter-9 | UCUAUGCUCA | TCTATGCTCA | 5250696 | | | |
| | (SEQ ID | (SEQ ID | | | | |
| | NO: 1155) | NO: 1156) | | | | |
| AsCpf1 HBG1 | CUGAGCAUAG | CTGAGCATAG | Chr11:5250678: | | 10.73 | − |
| Promoter-10 | AAGAGCUACG | AAGAGCTACG | 5250697 | | | |
| | (SEQ ID | (SEQ ID | | | | |
| | NO: 1157) | NO: 1158) | | | | |
| AsCpf1 HBG2 | UCACUGGAUA | TCACTGGATA | | Chr11:5255565: | 0.43 | + |
| Promoter-11 | AGUGUAUGUG | AGTGTATGTG | | 5255584 | | |
| | (SEQ ID | (SEQ ID | | | | |
| | NO: 1159) | NO: 1160) | | | | |
| AsCpf1 HBG2 | GUGUAGCUCU | GTGTAGCTCT | | Chr11:5255608: | 5.78 | + |
| Promoter-12 | UCUAUGCUCG | TCTATGCTCG | | 5255627 | | |
| | (SEQ ID | (SEQ ID | | | | |
| | NO: 1161) | NO: 1162) | | | | |
| AsCpf1 HBG2 | CCGAGCAUAG | CCGAGCATAG | | Chr11:5255609: | 3.24 | − |
| Promoter-13 | AAGAGCUACA | AAGAGCTACA | | 5255628 | | |
| | (SEQ ID | (SEQ ID | | | | |
| | NO: 1163) | NO: 1164) | | | | |
| HBG1-1 | CCUUGUCAAG | CCTTGTCAAG | Chr11:5249955: | Chr11:5254879: | 17.96 | + |
| AsCpf1 | GCUAUUGGUC | GCTATTGGTC | 5249974 | 5254898 | | |
| | (SEQ ID | (SEQ ID | | | | |
| | NO: 1002) | NO: 1003) | | | | |
| AsCpf1 RR | GACAGAUAUU | GACAGATATT | Chr11:5250025: | Chr11:5254949: | 8.48 | + |
| HBG1 | UGCAUUGAGA | TGCATTGAGA | 5250044 | 5254968 | | |
| Promoter-1 | (SEQ ID | (SEQ ID | | | | |
| | NO: 1167) | NO: 1168) | | | | |
| AsCpf1 RR | ACACUAUCUC | ACACTATCTC | Chr11:5250031: | Chr11:5254955: | 0.09 | − |
| HBG1 | AAUGCAAAUA | AATGCAAATA | 5250050 | 5254974 | | |
| Promoter-2 | (SEQ ID | (SEQ ID | | | | |
| | NO: 1169) | NO: 1170) | | | | |
| AsCpf1 RR | CACACUAUCU | CACACTATCT | Chr11:5250032: | Chr11:5254956: | 2.10 | − |
| HBG1 | CAAUGCAAAU | CAATGCAAAT | 5250051 | 5254975 | | |
| Promoter-3 | (SEQ ID | (SEQ ID | | | | |
| | NO: 1171) | NO: 1172) | | | | |
| AsCpf1 RR | CCACACUAUC | CCACACTATC | Chr11:5250033: | Chr11:5254957: | 2.52 | − |
| HBG1 | UCAAUGCAAA | TCAATGCAAA | 5250052 | 5254976 | | |
| Promoter-4 | (SEQ ID | (SEQ ID | | | | |
| | NO: 1173) | NO: 1174) | | | | |
| AsCpf1 RR | UUCCCCACAC | TTCCCCACAC | Chr11:5250037: | Chr11:5254961: | 0.05 | − |
| HBG1 | UAUCUCAAUG | TATCTCAATG | 5250056 | 5254980 | | |
| Promoter-5 | (SEQ ID | (SEQ ID | | | | |
| | NO: 1175) | NO: 1176) | | | | |
| AsCpf1 RR | GAUUCAGUCA | GATTCAGTCA | Chr11:5250109: | | 0.77 | + |
| HBG1 | UUCCAGUUUU | TTCCAGTTTT | 5250128 | | | |
| Promoter-6 | (SEQ ID | (SEQ ID | | | | |
| | NO: 1177) | NO: 1178) | | | | |
| AsCpf1 RR | AUUCAGUCAU | ATTCAGTCAT | Chr11:5250110: | | 0.24 | + |
| HBG1 | UCCAGUUUUU | TCCAGTTTTT | 5250129 | | | |
| Promoter-7 | (SEQ ID | (SEQ ID | | | | |
| | NO: 1179) | NO: 1180) | | | | |
| AsCpf1 RR | GUCAUUCCAG | GTCATTCCAG | Chr11:5250115: | | 1.00 | + |
| HBG1 | UUUUUCUCUA | TTTTTCTCTA | 5250134 | | | |
| Promoter-8 | (SEQ ID | (SEQ ID | | | | |
| | NO: 1181) | NO: 1182) | | | | |
| AsCpf1 RR | AGUUUUUCUC | AGTTTTTCTCT | Chr11:5250123: | | 0.15 | + |
| HBG1 | UAAUUUAUUC | AATTTATTC | 5250142 | | | |
| Promoter-9 | (SEQ ID | (SEQ ID | | | | |
| | NO: 1183) | NO: 1184) | | | | |
| AsCpf1 RR | GUUUUUCUCU | GTTTTTCTCTA | Chr11:5250124: | | 0.15 | + |
| HBG1 | AAUUUAUUCU | ATTTATTCT | 5250143 | | | |
| Promoter-10 | (SEQ ID | (SEQ ID | | | | |
| | NO: 1185) | NO: 1186) | | | | |
| AsCpf1 RR | GAUUCAGUCA | CAAGAGGATA | | Chr11:5254989: | *: ** | + |
| HBG2 | UUCCAAUUUU | CTGCTGCTTA | | 5255008 | | |
| Promoter-11 | (SEQ ID | (SEQ ID | | | | |
| | NO: 1187) | NO: 1188) | | | | |
| AsCpf1 RR | AUUCAGUCAU | AAGAGGATAC | | Chr11:5254990: | *** | + |
| HBG2 | UCCAAUUUUU | TGCTGCTTAA | | 5255009 | | |
| Promoter-12 | (SEQ ID | (SEQ ID | | | | |
| | NO: 1189) | NO: 1190) | | | | |
| AsCpf1 RR | GUCAUUCCAA | GATTCAGTCA | | Chr11:5255037: | 0.25 | + |
| HBG2 | UUUUUCUCUA | TTCCAATTTT | | 5255056 | | |
| Promoter-13 | (SEQ ID | (SEQ ID | | | | |
| | NO: 1191) | NO: 1192) | | | | |
| AsCpf1 RR | AAUUUUUCUC | ATTCAGTCAT | | Chr11:5255038: | 0.13 | + |
| HBG2 | UAAUUUAUUC | TCCAATTTTT | | 5255057 | | |
| Promoter-14 | (SEQ ID | (SEQ ID | | | | |
| | NO: 1193) | NO: 1194) | | | | |
| AsCpf1 RR | AUUUUUCUCU | GTCATTCCAA | | Chr11:5255043: | 0.32 | + |
| HBG2 | AAUUUAUUCU | TTTTTCTCTA | | 5255062 | | |
| Promoter-15 | (SEQ ID | (SEQ ID | | | | |
| | NO: 1195) | NO: 1196) | | | | |
| AsCpf1 RR | UUCUCCCAUC | AATTTTTCTCT | | Chr11:5255051: | 0.14 | + |
| HBG2 | AUAGAGGAUA | AATTTATTC | | 5255070 | | |
| Promoter-16 | (SEQ ID | (SEQ ID | | | | |
| | NO: 1197) | NO: 1198) | | | | |
| AsCpf1 RR | AUCAUAGAGG | ATTTTTCTCTA | | Chr11:5255052: | 0.10 | + |
| HBG2 | AUACCAGGAC | ATTTATTCT | | 5255071 | | |
| Promoter-17 | (SEQ ID | (SEQ ID | | | | |
| | NO: 1199) | NO: 1200) | | | | |
| AsCpf1 RR | ACCAUAGAAG | TTCTCCCATC | Chr11:5250162: | | 1.40 | + |
| HBG1 | AUACCAGGAC | ATAGAGGATA | 5250181 | | | |
| Promoter-18 | (SEQ ID | (SEQ ID | | | | |
| | NO: 1201) | NO: 1202) | | | | |
| AsCpf1 RR | CAGUACCUGC | ATCATAGAGG | Chr11:5250169: | | 7.88 | + |
| HBG1 | CAAAGAACAU | ATACCAGGAC | 5250188 | | | |
| Promoter-19 | (SEQ ID | (SEQ ID | | | | |
| | NO:1203) | NO: 1204) | | | | |
| AsCpf1 RR | UAGUAUCUGG | ACCATAGAAG | | Chr11:5255097: | 13.03 | + |
| HBG2 | UAAAGAGCAU | ATACCAGGAC | | 5255116 | | |
| Promoter-20 | (SEQ ID | (SEQ ID | | | | |
| | NO: 1205) | NO: 1206) | | | | |
| AsCpf1 RR | UCAAUGCAAA | CAGTACCTGC | Chr11:5250714: | | 13.31 | |
| HBG1 | UAUCUGUCUG | CAAAGAACAT | 5250733 | | | |
| Promoter-21 | (SEQ ID | (SEQ ID | | | | |
| | NO: 1207) | NO: 1208) | | | | |
| AsCpf1 RR | CUCUUGGGGG | TAGTATCTGG | | Chr11:5255645: | 4.07 | − |
| HBG2 | CCCCUUCCCC | TAAAGAGCAT | | 5255664 | | |
| Promoter-22 | (SEQ ID | (SEQ ID | | | | |
| | NO: 1209) | NO: 1210) | | | | |
| AsCpf1 RVR | GAUUCAGUCA | TCAATGCAAA | Chr11:5250023: | Chr11:5254947: | 0.15 | − |
| HBG1 | UUCCAAUUUU | TATCTGTCTG | 5250042 | 5254966 | | |
| Promoter-1 | (SEQ ID | (SEQ ID | | | | |
| | NO: 1211) | NO: 1212) | | | | |
| AsCpf1 RVR | AUUCAGUCAU | CTCTTGGGGG | Chr11:5250051: | Chr11:5254975: | 1.09 | − |
| HBG1 | UCCAAUUUUU | CCCCTTCCCC | 5250070 | 5254994 | | |
| Promoter-2 | (SEQ ID | (SEQ ID | | | | |
| | NO: 1213) | NO: 1214) | | | | |
| AsCpf1 RVR | AAAAAAAUUA | AAAAAAATTA | Chr11:5250069: | | | |
| HBG1 | GCAGUAUCCU | GCAGTATCCT | 5250088 | | | |
| Promoter-3 | (SEQ ID | (SEQ ID | | | | |
| | NO: 1215) | NO: 1216) | | | | |
| AsCpf1 RVR | GCCUUUGCCU | GCCTTTGCCTT | Chr11:5250093: | Chr11:5255021: | 3.96 | + |
| HBG1 | UGUUCCGAUU | GTTCCGATT | 5250112 | 5255040 | | |
| Promoter-4 | (SEQ ID | (SEQ ID | | | | |
| | NO: 1217) | NO: 1218) | | | | |
| AsCpf1 RVR | AAAAAAAUUA | AAAAAAATTA | | Chr11:5254997: | *: | − |
| HBG2 | AGCAGCAGUA | AGCAGCAGTA | | 5255016 | | |
| Promoter-5 | (SEQ ID | (SEQ ID | | | | |
| | NO: 1219) | NO: 1220) | | | | |
| AsCpf1 RVR | CUCAGUAAAG | CTCAGTAAAG | Chr11:5250693: | | 5.32 | + |
| HBG1 | AUGAUGGUAG | ATGATGGTAG | 5250712 | | | |
| Promoter-6 | (SEQ ID | (SEQ ID | | | | |
| | NO: 1221) | NO: 1222) | | | | |
| AsCpf1 RVR | ACUGGAUAAG | ACTGGATAAG | | Chr11:5255567: | 9.78 | + |
| HBG2 | UGUAUGUGCU | TGTATGTGCT | | 5255586 | | |
| Promoter-7 | (SEQ ID | (SEQ ID | | | | |
| | NO: 1223) | NO: 1224) | | | | |
| AsCpf1 RVR | UGCUGGCUGA | TGCTGGCTGA | Chr11:5250652: | Chr11:5255583: | 0.24 | + |
| HBG2 | UGACCCAGGG | TGACCCAGGG | 5250671 | 5255602 | | |
| Promoter-8 | (SEQ ID | (SEQ ID | | | | |
| | NO: 1225) | NO: 1226) | | | | |
| AsCpf1 RVR | CUCGGUAAAG | CTCGGTAAAG | | Chr11:5255624: | 5.75 | + |
| HBG2 | AUGAUGGUAG | ATGATGGTAG | | 5255643 | | |
| Promoter-9 | (SEQ ID | (SEQ ID | | | | |
| | NO: 1227) | NO: 1228) | | | | |
| AsCpf1 RVR | UGGUAAAGAG | TGGTAAAGAG | | Chr11:5255638: | 8.55 | − |
| HBG2 | CAUUCUACCA | CATTCTACCA | | 5255657 | | |
| Promoter-10 | (SEQ ID | (SEQ ID | | | | |
| | NO: 1229) | NO: 1230) | | | | |

* the gRNA ID name provides the particular Cpf1 molecule used in the RNP complex
** NCBI Reference Sequence NC_000011, the coordinates are reported using the One-based coordinate system, “Homo sapiens chromosome 11, GRCh38.p12 Primary Assembly,” (Version NC_000011.10).
*** represents gRNAs that were not tested.

TABLE 8
Cpf1 guide RNAs

| | | | | Length of | | |
|---|---|---|---|---|---|---|
| gRNA | | | | crRNA + | Length of | gRNA |
| Sequence | | | | gRNA | gRNA | Targeting |
| SEQ ID | | | | targeting | targeting | Domain |
| NO. | gRNA Sequence | 5′ mod.** | 3′ mod. | domain | domain | (RNA) |
| 1022 | rUrArArUrUrUrCr | — | — | 40 | 20 | CCUUGUC |
| | UrArCrUrCrUrUrG | | | | | AAGGCUA |
| | rUrArGrArUrCrCr | | | | | UUGGUC |
| | UrUrGrUrCrArArG | | | | | (SEQ ID |
| | rGrCrUrArUrUrGr | | | | | NO: 1002) |
| | GrUrC | | | | | |
| 1023 | rUrArArUrUrUrCr | — | 1xPS2- | 41 | 21 | CCUUGUC |
| | UrArCrUrCrUrUrG | | OMe + | | | AAGGCUA |
| | rUrArGrArUrCrCr | | 1xOMe | | | UUGGUCA |
| | UrUrGrUrCrArArG | | | | | (SEQ ID |
| | rGrCrUrArUrUrGr | | | | | NO: 1254) |
| | GrUmC/PS2/mA | | | | | |
| 1041 | rUrArArUrUrUrCr | — | 1xPS2- | 40 | 20 | CCUUGUC |
| | UrArCrUrCrUrUrG | | OMe + | | | AAGGCUA |
| | rUrArGrArUrCrCr | | 1xOMe | | | UUGGUC |
| | UrUrGrUrCrArArG | | | | | (SEQ ID |
| | rGrCrUrArUrUrGr | | | | | NO: 1002) |
| | GmU/PS2/mC | | | | | |
| 1042 | rCrUrUrUrUrUrAr | +5 RNA | — | 45 | 20 | CCUUGUC |
| | ArUrUrUrCrUrArC | | | | | AAGGCUA |
| | rUrCrUrUrGrUrAr | | | | | UUGGUC |
| | GrArUrCrCrUrUrG | | | | | (SEQ ID |
| | rUrCrArArGrGrCr | | | | | NO: 1002) |
| | UrArUrUrGrGrUrC | | | | | |
| 1043 | rArArGrArCrCrUr | +10 RNA | — | 50 | 20 | CCUUGUC |
| | UrUrUrUrArArUr | | | | | AAGGCUA |
| | UrUrCrUrArCrUrC | | | | | UUGGUC |
| | rUrUrGrUrArGrAr | | | | | (SEQ ID |
| | UrCrCrUrUrGrUrC | | | | | NO: 1002) |
| | rArArGrGrCrUrAr | | | | | |
| | UrUrGrGrUrC | | | | | |
| 1044 | rArUrGrUrGrUrUr | +25 RNA | — | 65 | 20 | CCUUGUC |
| | UrUrUrGrUrCrArA | | | | | AAGGCUA |
| | rArArGrArCrCrUr | | | | | UUGGUC |
| | UrUrUrUrArArUr | | | | | (SEQ ID |
| | UrUrCrUrArCrUrC | | | | | NO: 1002) |
| | rUrUrGrUrArGrAr | | | | | |
| | UrCrCrUrUrGrUrC | | | | | |
| | rArArGrGrCrUrAr | | | | | |
| | UrUrGrGrUrC | | | | | |
| 1045 | rArGrGrCrCrArGr | +60 RNA | — | 100 | 20 | CCUUGUC |
| | CrUrUrGrCrCrGrG | | | | | AAGGCUA |
| | rUrUrUrUrUrUrAr | | | | | UUGGUC |
| | GrUrCrGrUrGrCrU | | | | | (SEQ ID |
| | rGrCrUrUrCrArUr | | | | | NO: 1002) |
| | GrUrGrUrUrUrUr | | | | | |
| | UrGrUrCrArArArA | | | | | |
| | rGrArCrCrUrUrUr | | | | | |
| | UrUrArArUrUrUrC | | | | | |
| | rUrArCrUrCrUrUr | | | | | |
| | GrUrArGrArUrCrC | | | | | |
| | rUrUrGrUrCrArAr | | | | | |
| | GrGrCrUrArUrUrG | | | | | |
| | rGrUrC | | | | | |
| 1046 | CTTTTrUrArArUr | +5 DNA | — | 45 | 20 | CCUUGUC |
| | UrUrCrUrArCrUrC | | | | | AAGGCUA |
| | rUrUrGrUrArGrAr | | | | | UUGGUC |
| | UrCrCrUrUrGrUrC | | | | | (SEQ ID |
| | rArArGrGrCrUrAr | | | | | NO: 1002) |
| | UrUrGrGrUrC | | | | | |
| 1047 | AAGACCTTTTrUr | +10 DNA | — | 50 | 20 | CCUUGUC |
| | ArArUrUrUrCrUrA | | | | | AAGGCUA |
| | rCrUrCrUrUrGrUr | | | | | UUGGUC |
| | ArGrArUrCrCrUrU | | | | | (SEQ ID |
| | rGrUrCrArArGrGr | | | | | NO: 1002) |
| | CrUrArUrUrGrGrU | | | | | |
| | rC | | | | | |
| 1048 | ATGTGTTTTTGT | +25 DNA | — | 65 | 20 | CCUUGUC |
| | CAAAAGACCTT | | | | | AAGGCUA |
| | TTrUrArArUrUrUr | | | | | UUGGUC |
| | CrUrArCrUrCrUrU | | | | | (SEQ ID |
| | rGrUrArGrArUrCr | | | | | NO: 1002) |
| | CrUrUrGrUrCrArA | | | | | |
| | rGrGrCrUrArUrUr | | | | | |
| | GrGrUrC | | | | | |
| 1049 | AGGCCAGCTTG | +60 DNA | — | 100 | 20 | CCUUGUC |
| | CCGGTTTTTTAG | | | | | AAGGCUA |
| | TCGTGCTGCTTC | | | | | UUGGUC |
| | ATGTGTTTTTGT | | | | | (SEQ ID |
| | CAAAAGACCTT | | | | | NO: 1002) |
| | TTrUrArArUrUrUr | | | | | |
| | CrUrArCrUrCrUrU | | | | | |
| | rGrUrArGrArUrCr | | | | | |
| | CrUrUrGrUrCrArA | | | | | |
| | rGrGrCrUrArUrUr | | | | | |
| | GrGrUrC | | | | | |
| 1050 | ATGTGTTTTTGT | +25 DNA | 1xPS2- | 66 | 21 | CCUUGUC |
| | CAAAAGACCTT | | OMe + | | | AAGGCUA |
| | TTrUrArArUrUrUr | | 1xOMe | | | UUGGUCA |
| | CrUrArCrUrCrUrU | | | | | (SEQ ID |
| | rGrUrArGrArUrCr | | | | | NO: 1254) |
| | CrUrUrGrUrCrArA | | | | | |
| | rGrGrCrUrArUrUr | | | | | |
| | GrGrUmC/PS2/mA | | | | | |
| 1051 | ATGTGTTTTTGT | +25 DNA | 1xPS- | 66 | 21 | CCUUGUC |
| | CAAAAGACCTT | | OMe | | | AAGGCUA |
| | TTrUrArArUrUrUr | | | | | UUGGUCA |
| | CrUrArCrUrCrUrU | | | | | (SEQ ID |
| | rGrUrArGrArUrCr | | | | | NO: 1254) |
| | CrUrUrGrUrCrArA | | | | | |
| | rGrGrCrUrArUrUr | | | | | |
| | GrGrUrC*mA | | | | | |
| 1052 | TTTTTGTCAAAA | +20 DNA | — | 60 | 20 | CCUUGUC |
| | GACCTTTTrUrAr | | | | | AAGGCUA |
| | ArUrUrUrCrUrArC | | | | | UUGGUC |
| | rUrCrUrUrGrUrAr | | | | | (SEQ ID |
| | GrArUrCrCrUrUrG | | | | | NO: 1002) |
| | rUrCrArArGrGrCr | | | | | |
| | UrArUrUrGrGrUrC | | | | | |
| 1053 | GCTTCATGTGTT | +30 DNA | — | 70 | 20 | CCUUGUC |
| | TTTGTCAAAAG | | | | | AAGGCUA |
| | ACCTTTTrUrArAr | | | | | UUGGUC |
| | UrUrUrCrUrArCrU | | | | | (SEQ ID |
| | rCrUrUrGrUrArGr | | | | | NO: 1002) |
| | ArUrCrCrUrUrGrU | | | | | |
| | rCrArArGrGrCrUr | | | | | |
| | ArUrUrGrGrUrC | | | | | |
| 1054 | GCCGGTTTTTTA | +50 DNA | — | 90 | 20 | CCUUGUC |
| | GTCGTGCTGCTT | | | | | AAGGCUA |
| | CATGTGTTTTTG | | | | | UUGGUC |
| | TCAAAAGACCT | | | | | (SEQ ID |
| | TTTrUrArArUrUr | | | | | NO: 1002) |
| | UrCrUrArCrUrCrU | | | | | |
| | rUrGrUrArGrArUr | | | | | |
| | CrCrUrUrGrUrCrA | | | | | |
| | rArGrGrCrUrArUr | | | | | |
| | UrGrGrUrC | | | | | |
| 1055 | TAGTCGTGCTGC | +40 DNA | — | 80 | 20 | CCUUGUC |
| | TTCATGTGTTTT | | | | | AAGGCUA |
| | TGTCAAAAGAC | | | | | UUGGUC |
| | CTTTTrUrArArUr | | | | | (SEQ ID |
| | UrUrCrUrArCrUrC | | | | | NO: 1002) |
| | rUrUrGrUrArGrAr | | | | | |
| | UrCrCrUrUrGrUrC | | | | | |
| | rArArGrGrCrUrAr | | | | | |
| | UrUrGrGrUrC | | | | | |
| 1056 | C*C*GAAGTTTT | +20 DNA | — | 60 | 20 | CCUUGUC |
| | CTTCGGTTTTrUr | + | | | | AAGGCUA |
| | ArArUrUrUrCrUrA | 2xPS | | | | UUGGUC |
| | rCrUrCrUrUrGrUr | | | | | (SEQ ID |
| | ArGrArUrCrCrUrU | | | | | NO: 1002) |
| | rGrUrCrArArGrGr | | | | | |
| | CrUrArUrUrGrGrU | | | | | |
| | rC | | | | | |
| 1057 | T*T*TTTCCGAA | +25 DNA | — | 65 | 20 | CCUUGUC |
| | GTTTTCTTCGGT | + | | | | AAGGCUA |
| | TTTrUrArArUrUr | 2xPS | | | | UUGGUC |
| | UrCrUrArCrUrCrU | | | | | (SEQ ID |
| | rUrGrUrArGrArUr | | | | | NO: 1002) |
| | CrCrUrUrGrUrCrA | | | | | |
| | rArGrGrCrUrArUr | | | | | |
| | UrGrGrUrC | | | | | |
| 1058 | A*A*CGCTTTTT | +30 DNA | — | 70 | 20 | CCUUGUC |
| | CCGAAGTTTTCT | + | | | | AAGGCUA |
| | TCGGTTTTrUrAr | 2xPS | | | | UUGGUC |
| | ArUrUrUrCrUrArC | | | | | (SEQ ID |
| | rUrCrUrUrGrUrAr | | | | | NO: 1002) |
| | GrArUrCrCrUrUrG | | | | | |
| | rUrCrArArGrGrCr | | | | | |
| | UrArUrUrGrGrUrC | | | | | |
| 1059 | G*C*GTTGTTTT | +41 DNA | — | 81 | 20 | CCUUGUC |
| | CAACGCTTTTTC | + | | | | AAGGCUA |
| | CGAAGTTTTCTT | 2xPS | | | | UUGGUC |
| | CGGTTTTrUrArAr | | | | | (SEQ ID |
| | UrUrUrCrUrArCrU | | | | | NO: 1002) |
| | rCrUrUrGrUrArGr | | | | | |
| | ArUrCrCrUrUrGrU | | | | | |
| | rCrArArGrGrCrUr | | | | | |
| | ArUrUrGrGrUrC | | | | | |
| 1060 | G*G*CTTCTTTT | +62 DNA | — | 102 | 20 | CCUUGUC |
| | GAAGCCTTTTTG | + | | | | AAGGCUA |
| | CGTTGTTTTCAA | 2xPS | | | | UUGGUC |
| | CGCTTTTTCCGA | | | | | (SEQ ID |
| | AGTTTTCTTCGG | | | | | NO: 1002) |
| | TTTTrUrArArUrUr | | | | | |
| | UrCrUrArCrUrCrU | | | | | |
| | rUrGrUrArGrArUr | | | | | |
| | CrCrUrUrGrUrCrA | | | | | |
| | rArGrGrCrUrArUr | | | | | |
| | UrGrGrUrC | | | | | |
| 1061 | mUrArArUrUrUrC | 1xOMe | — | 40 | 20 | CCUUGUC |
| | rUrArCrUrCrUrUr | | | | | AAGGCUA |
| | GrUrArGrArUrCrC | | | | | UUGGUC |
| | rUrUrGrUrCrArAr | | | | | (SEQ ID |
| | GrGrCrUrArUrUrG | | | | | NO: 1002) |
| | rGrUrC | | | | | |
| 1062 | mU*rArArUrUrUr | 1xPS-OMe | — | 40 | 20 | CCUUGUC |
| | CrUrArCrUrCrUrU | | | | | AAGGCUA |
| | rGrUrArGrArUrCr | | | | | UUGGUC |
| | CrUrUrGrUrCrArA | | | | | (SEQ ID |
| | rGrGrCrUrArUrUr | | | | | NO: 1002) |
| | GrGrUrC | | | | | |
| 1063 | mUmArArUrUrUr | 2xOMe | — | 40 | 20 | CCUUGUC |
| | CrUrArCrUrCrUrU | | | | | AAGGCUA |
| | rGrUrArGrArUrCr | | | | | UUGGUC |
| | CrUrUrGrUrCrArA | | | | | (SEQ ID |
| | rGrGrCrUrArUrUr | | | | | NO: 1002) |
| | GrGrUrC | | | | | |
| 1064 | mU*mA*rArUrUr | 2xPS-OMe | — | 40 | 20 | CCUUGUC |
| | UrCrUrArCrUrCrU | | | | | AAGGCUA |
| | rUrGrUrArGrArUr | | | | | UUGGUC |
| | CrCrUrUrGrUrCrA | | | | | (SEQ ID |
| | rArGrGrCrUrArUr | | | | | NO: 1002) |
| | UrGrGrUrC | | | | | |
| 1065 | mUmAmArUrUrUr | 3xOMe | — | 40 | 20 | CCUUGUC |
| | CrUrArCrUrCrUrU | | | | | AAGGCUA |
| | rGrUrArGrArUrCr | | | | | UUGGUC |
| | CrUrUrGrUrCrArA | | | | | (SEQ ID |
| | rGrGrCrUrArUrUr | | | | | NO: 1002) |
| | GrGrUrC | | | | | |
| 1066 | mU*mA*mA*rUr | 3xPS-OMe | — | 40 | 20 | CCUUGUC |
| | UrUrCrUrArCrUrC | | | | | AAGGCUA |
| | rUrUrGrUrArGrAr | | | | | UUGGUC |
| | UrCrCrUrUrGrUrC | | | | | (SEQ ID |
| | rArArGrGrCrUrAr | | | | | NO: 1002) |
| | UrUrGrGrUrC | | | | | |
| 1067 | A*T*GTGTTTTT | +25 DNA | — | 65 | 20 | CCUUGUC |
| | GTCAAAAGACC | + | | | | AAGGCUA |
| | TTTTrUrArArUrUr | 2xPS | | | | UUGGUC |
| | UrCrUrArCrUrCrU | | | | | (SEQ ID |
| | rUrGrUrArGrArUr | | | | | NO: 1002) |
| | CrCrUrUrGrUrCrA | | | | | |
| | rArGrGrCrUrArUr | | | | | |
| | UrGrGrUrC | | | | | |
| 1068 | A*T*GTGTTTTT | +25 DNA | 1xPS- | 66 | 21 | CCUUGUC |
| | GTCAAAAGACC | + | OMe | | | AAGGCUA |
| | TTTTrUrArArUrUr | 2xPS | | | | UUGGUCA |
| | UrCrUrArCrUrCrU | | | | | (SEQ ID |
| | rUrGrUrArGrArUr | | | | | NO: 1254) |
| | CrCrUrUrGrUrCrA | | | | | |
| | rArGrGrCrUrArUr | | | | | |
| | UrGrGrUrC*mA | | | | | |
| 1069 | rUrArArUrUrUrCr | — | 1xPS- | 41 | 21 | CCUUGUC |
| | UrArCrUrCrUrUrG | | OMe | | | AAGGCUA |
| | rUrArGrArUrCrCr | | | | | UUGGUCA |
| | UrUrGrUrCrArArG | | | | | (SEQ ID |
| | rGrCrUrArUrUrGr | | | | | NO: 1254) |
| | GrUrC*mA | | | | | |
| 1070 | rUrArArUrUrUrCr | — | 1xPS- | 42 | 21 | CCUUGUC |
| | UrArCrUrCrUrUrG | | OMe + | | | AAGGCUA |
| | rUrArGrArUrCrCr | | rU | | | UUGGUCA |
| | UrUrGrUrCrArArG | | | | | (SEQ ID |
| | rGrCrUrArUrUrGr | | | | | NO: 1254) |
| | GrUrCmA*rU | | | | | |
| 1071 | rUrArArUrUrUrCr | — | rU | 41 | 20 | CCUUGUC |
| | UrArCrUrCrUrUrG | | | | | AAGGCUA |
| | rUrArGrArUrCrCr | | | | | UUGGUC |
| | UrUrGrUrCrArArG | | | | | (SEQ ID |
| | rGrCrUrArUrUrGr | | | | | NO: 1002) |
| | GrUrCrU | | | | | |
| 1072 | rUrArArUrUrUrCr | — | rU | 42 | 21 | CCUUGUC |
| | UrArCrUrCrUrUrG | | | | | AAGGCUA |
| | rUrArGrArUrCrCr | | | | | UUGGUCA |
| | UrUrGrUrCrArArG | | | | | (SEQ ID |
| | rGrCrUrArUrUrGr | | | | | NO: 1254) |
| | GrUrCrArU | | | | | |
| 1073 | rUrArArUrUrUrCr | — | rU | 44 | 23 | CCUUGUC |
| | UrArCrUrCrUrUrG | | | | | AAGGCUA |
| | rUrArGrArUrCrCr | | | | | UUGGUCA |
| | UrUrGrUrCrArArG | | | | | AG (SEQ ID |
| | rGrCrUrArUrUrGr | | | | | NO: 1258) |
| | GrUrCrArArGrU | | | | | |
| 1074 | A*T*GTGTTTTT | +25 DNA | rU | 66 | 20 | CCUUGUC |
| | GTCAAAAGACC | + | | | | AAGGCUA |
| | TTTTrUrArArUrUr | 2xPS | | | | UUGGUC |
| | UrCrUrArCrUrCrU | | | | | (SEQ ID |
| | rUrGrUrArGrArUr | | | | | NO: 1002) |
| | CrCrUrUrGrUrCrA | | | | | |
| | rArGrGrCrUrArUr | | | | | |
| | UrGrGrUrCrU | | | | | |
| 1075 | A*T*GTGTTTTT | +25 DNA | rU | 69 | 23 | CCUUGUC |
| | GTCAAAAGACC | + | | | | AAGGCUA |
| | TTTTrUrArArUrUr | 2xPS | | | | UUGGUCA |
| | UrCrUrArCrUrCrU | | | | | AG (SEQ ID |
| | rUrGrUrArGrArUr | | | | | NO: 1258) |
| | CrCrUrUrGrUrCrA | | | | | |
| | rArGrGrCrUrArUr | | | | | |
| | UrGrGrUrCrArArG | | | | | |
| | rU | | | | | |
| 1076 | rUrArArUrUrUrCr | — | — | 41 | 21 | CCUUGUC |
| | UrArCrUrCrUrUrG | | | | | AAGGCUA |
| | rUrArGrArUrCrCr | | | | | UUGGUCA |
| | UrUrGrUrCrArArG | | | | | (SEQ ID |
| | rGrCrUrArUrUrGr | | | | | NO: 1254) |
| | GrUrCrA | | | | | |
| 1077 | rUrArArUrUrUrCr | — | — | 43 | 23 | CCUUGUC |
| | UrArCrUrCrUrUrG | | | | | AAGGCUA |
| | rUrArGrArUrCrCr | | | | | UUGGUCA |
| | UrUrGrUrCrArArG | | | | | AG (SEQ ID |
| | rGrCrUrArUrUrGr | | | | | NO: 1258) |
| | GrUrCrArArG | | | | | |
| 1078 | A*T*GTGTTTTT | +25 DNA | — | 68 | 23 | CCUUGUC |
| | GTCAAAAGACC | + | | | | AAGGCUA |
| | TTTTrUrArArUrUr | 2xPS | | | | UUGGUCA |
| | UrCrUrArCrUrCrU | | | | | AG (SEQ ID |
| | rUrGrUrArGrArUr | | | | | NO: 1258) |
| | CrCrUrUrGrUrCrA | | | | | |
| | rArGrGrCrUrArUr | | | | | |
| | UrGrGrUrCrArArG | | | | | |
| 1079 | rUrArArUrUrUrCr | — | 4xrU | 44 | 20 | CCUUGUC |
| | UrArCrUrCrUrUrG | | | | | AAGGCUA |
| | rUrArGrArUrCrCr | | | | | UUGGUC |
| | UrUrGrUrCrArArG | | | | | (SEQ ID |
| | rGrCrUrArUrUrGr | | | | | NO: 1002) |
| | GrUrCrUrUrUrU | | | | | |
| 1080 | rUrArArUrUrUrCr | — | 3xPS- | 44 | 20 | CCUUGUC |
| | UrArCrUrCrUrUrG | | OMe-U | | | AAGGCUA |
| | rUrArGrArUrCrCr | | + rU | | | UUGGUC |
| | UrUrGrUrCrArArG | | | | | (SEQ ID |
| | rGrCrUrArUrUrGr | | | | | NO: 1002) |
| | GrUrCmU*mU*m | | | | | |
| | U*rU | | | | | |
| 1081 | A*T*GTGTTTTT | +25 DNA | 4xrU | 69 | 20 | CCUUGUC |
| | GTCAAAAGACC | + | | | | AAGGCUA |
| | TTTTrUrArArUrUr | 2xPS | | | | UUGGUC |
| | UrCrUrArCrUrCrU | | | | | (SEQ ID |
| | rUrGrUrArGrArUr | | | | | NO: 1002) |
| | CrCrUrUrGrUrCrA | | | | | |
| | rArGrGrCrUrArUr | | | | | |
| | UrGrGrUrCrUrUrU | | | | | |
| | rU | | | | | |
| 1082 | AAAAAAAAAAA | +25 A | 1xPS- | 66 | 21 | CCUUGUC |
| | AAAAAAAAAAA | | OMe | | | AAGGCUA |
| | AAArUrArArUrUr | | | | | UUGGUCA |
| | UrCrUrArCrUrCrU | | | | | (SEQ ID |
| | rUrGrUrArGrArUr | | | | | NO: 1254) |
| | CrCrUrUrGrUrCrA | | | | | |
| | rArGrGrCrUrArUr | | | | | |
| | UrGrGrUrC*mA | | | | | |
| 1083 | TTTTTTTTTTTTT | +25 T | 1xPS- | 66 | 21 | CCUUGUC |
| | TTTTTTTTTTTTr | | OMe | | | AAGGCUA |
| | UrArArUrUrUrCrU | | | | | UUGGUCA |
| | rArCrUrCrUrUrGr | | | | | (SEQ ID |
| | UrArGrArUrCrCrU | | | | | NO: 1254) |
| | rUrGrUrCrArArGr | | | | | |
| | GrCrUrArUrUrGrG | | | | | |
| | rUrC*mA | | | | | |
| 1084 | C*C*GAAGTTTT | +20 DNA | 1xPS- | 61 | 21 | CCUUGUC |
| | CTTCGGTTTTrUr | + | OMe | | | AAGGCUA |
| | ArArUrUrUrCrUrA | 2xPS | | | | UUGGUCA |
| | rCrUrCrUrUrGrUr | | | | | (SEQ ID |
| | ArGrArUrCrCrUrU | | | | | NO: 1254) |
| | rGrUrCrArArGrGr | | | | | |
| | CrUrArUrUrGrGrU | | | | | |
| | rC*mA | | | | | |
| 1085 | rUrArArUrUrUrCr | — | 1xPS- | 41 | 21 | AGACAGA |
| | UrArCrUrCrUrUrG | | OMe | | | UAUUUGC |
| | rUrArGrArUrArGr | | | | | AUUGAGA |
| | ArCrArGrArUrArU | | | | | (SEQ ID |
| | rUrUrGrCrArUrUr | | | | | NO: 1260) |
| | GrArG*mA | | | | | |
| 1086 | ATGTGTTTTTGT | +25 DNA | 1xPS- | 66 | 21 | AGACAGA |
| | CAAAAGACCTT | | OMe | | | UAUUUGC |
| | TTrUrArArUrUrUr | | | | | AUUGAGA |
| | CrUrArCrUrCrUrU | | | | | (SEQ ID |
| | rGrUrArGrArUrAr | | | | | NO: 1260) |
| | GrArCrArGrArUrA | | | | | |
| | rUrUrUrGrCrArUr | | | | | |
| | UrGrArG*mA | | | | | |
| 1087 | C*C*GAAGTTTT | +20 DNA | 1xPS- | 61 | 21 | AGACAGA |
| | CTTCGGTTTTrUr | + | OMe | | | UAUUUGC |
| | ArArUrUrUrCrUrA | 2xPS | | | | AUUGAGA |
| | rCrUrCrUrUrGrUr | | | | | (SEQ ID |
| | ArGrArUrArGrArC | | | | | NO: 1260) |
| | rArGrArUrArUrUr | | | | | |
| | UrGrCrArUrUGrAr | | | | | |
| | G*mA | | | | | |
| 1088 | rUrArArUrUrUrCr | — | 1xPS- | 41 | 21 | CAUUGAG |
| | UrArCrUrCrUrUrG | | OMe | | | AUAGUGU |
| | rUrArGrArUrCrAr | | | | | GGGGAAG |
| | UrUrGrArGrArUr | | | | | (SEQ ID |
| | ArGrUrGrUrGrGr | | | | | NO: 1262) |
| | GrGrArA*mG | | | | | |
| 1089 | ATGTGTTTTTGT | +25 DNA | 1xPS- | 66 | 21 | CAUUGAG |
| | CAAAAGACCTT | | OMe | | | AUAGUGU |
| | TTrUrArArUrUrUr | | | | | GGGGAAG |
| | CrUrArCrUrCrUrU | | | | | (SEQ ID |
| | rGrUrArGrArUrCr | | | | | NO: 1262) |
| | ArUrUrGrArGrAr | | | | | |
| | UrArGrUrGrUrGr | | | | | |
| | GrGrGrArA*mG | | | | | |
| 1090 | C*C*GAAGTTTT | +20 DNA | 1xPS- | 61 | 21 | CAUUGAG |
| | CTTCGGTTTTrUr | + | OMe | | | AUAGUGU |
| | ArArUrUrUrCrUrA | 2xPS | | | | GGGGAAG |
| | rCrUrCrUrUrGrUr | | | | | (SEQ ID |
| | ArGrArUrCrArUrU | | | | | NO: 1262) |
| | rGrArGrArUrArGr | | | | | |
| | UrGrUrGrGrGrGr | | | | | |
| | ArA*mG | | | | | |
| 1091 | rUrArArUrUrUrCr | — | 1xPS- | 41 | 21 | CUUCUCC |
| | UrArCrUrCrUrUrG | | OMe | | | CAUCAUA |
| | rUrArGrArUrCrUr | | | | | GAGGAUA |
| | UrCrUrCrCrCrArU | | | | | (SEQ ID |
| | rCrArUrArGrArGr | | | | | NO: 1264) |
| | GrArU*mA | | | | | |
| 1092 | ATGTGTTTTTGT | +25 DNA | 1xPS- | 66 | 21 | CUUCUCC |
| | CAAAAGACCTT | | OMe | | | CAUCAUA |
| | TTrUrArArUrUrUr | | | | | GAGGAUA |
| | CrUrArCrUrCrUrU | | | | | (SEQ ID |
| | rGrUrArGrArUrCr | | | | | NO: 1264) |
| | UrUrCrUrCrCrCrA | | | | | |
| | rUrCrArUrArGrAr | | | | | |
| | GrGrArU*mA | | | | | |
| 1093 | C*C*GAAGTTTT | +20 DNA | 1xPS- | 61 | 21 | CUUCUCC |
| | CTTCGGTTTTrUr | + | OMe | | | CAUCAUA |
| | ArArUrUrUrCrUrA | 2xPS | | | | GAGGAUA |
| | rCrUrCrUrUrGrUr | | | | | (SEQ ID |
| | ArGrArUrCrUrUrC | | | | | NO: 1264) |
| | rUrCrCrCrArUrCr | | | | | |
| | ArUrArGrArGrGr | | | | | |
| | ArU*mA | | | | | |
| 1098 | A*T*GTGTTTTT | +25 DNA | 1xPS- | 67 | 21 | CCUUGUC |
| | GTCAAAAGACC | + | OMe + | | | AAGGCUA |
| | TTTTrUrArArUrUr | 2xPS | rU | | | UUGGUCA |
| | UrCrUrArCrUrCrU | | | | | (SEQ ID |
| | rUrGrUrArGrArUr | | | | | NO: 1254) |
| | CrCrUrUrGrUrCrA | | | | | |
| | rArGrGrCrUrArUr | | | | | |
| | UrGrGrUrCmA*rU | | | | | |
| 1099 | A*T*GTGTTTTT | +25 DNA | 2xPS- | 68 | 22 | CCUUGUC |
| | GTCAAAAGACC | + | OMe + | | | AAGGCUA |
| | TTTTrUrArArUrUr | 2xPS | rU | | | UUGGUCA |
| | UrCrUrArCrUrCrU | | | | | A (SEQ ID |
| | rUrGrUrArGrArUr | | | | | NO: 1256) |
| | CrCrUrUrGrUrCrA | | | | | |
| | rArGrGrCrUrArUr | | | | | |
| | UrGrGrUrCmA*m | | | | | |
| | A*rU | | | | | |
| 1100 | A*T*GTGTTTTT | +25 DNA | 1xPS- | 66 | 20 | CCUUGUC |
| | GTCAAAAGACC | + | OMe + | | | AAGGCUA |
| | TTTTrUrArArUrUr | 2xPS | rU | | | UUGGUC |
| | UrCrUrArCrUrCrU | | | | | (SEQ ID |
| | rUrGrUrArGrArUr | | | | | NO: 1002) |
| | CrCrUrUrGrUrCrA | | | | | |
| | rArGrGrCrUrArUr | | | | | |
| | UrGrGrUmC*rU | | | | | |
| 1101 | A*T*GTGTTTTT | +25 DNA | 1xPS- | 65 | 20 | CCUUGUC |
| | GTCAAAAGACC | + | OMe | | | AAGGCUA |
| | TTTTrUrArArUrUr | 2xPS | | | | UUGGUC |
| | UrCrUrArCrUrCrU | | | | | (SEQ ID |
| | rUrGrUrArGrArUr | | | | | NO: 1002) |
| | CrCrUrUrGrUrCrA | | | | | |
| | rArGrGrCrUrArUr | | | | | |
| | UrGrGrU*mC | | | | | |
| 1102 | A*T*GTGTTTTT | +25 DNA | 2xPS- | 67 | 22 | CCUUGUC |
| | GTCAAAAGACC | + | OMe | | | AAGGCUA |
| | TTTTrUrArArUrUr | 2xPS | | | | UUGGUCA |
| | UrCrUrArCrUrCrU | | | | | A (SEQ ID |
| | rUrGrUrArGrArUr | | | | | NO: 1256) |
| | CrCrUrUrGrUrCrA | | | | | |
| | rArGrGrCrUrArUr | | | | | |
| | UrGrGrUrC*mA* | | | | | |
| | mA | | | | | |
| 1103 | mA*mU*rGrUrGr | +25 RNA | 1xPS- | 66 | 21 | CCUUGUC |
| | UrUrUrUrUrGrUrC | + | OMe | | | AAGGCUA |
| | rArArArArGrArCr | 2xPS | | | | UUGGUCA |
| | CrUrUrUrUrUrArA | | | | | (SEQ ID |
| | rUrUrUrCrUrArCr | | | | | NO: 1254) |
| | UrCrUrUrGrUrArG | | | | | |
| | rArUrCrCrUrUrGr | | | | | |
| | UrCrArArGrGrCrU | | | | | |
| | rArUrUrGrGrUrC* | | | | | |
| | mA | | | | | |
| 1104 | mA*mA*rArArAr | PolyA | 1xPS- | 66 | 21 | CCUUGUC |
| | Ar ArArArArArAr | RNA + | OMe | | | AAGGCUA |
| | ArArArArArArAr | 2xPS | | | | UUGGUCA |
| | ArArArArArArUr | | | | | (SEQ ID |
| | ArArUrUrUrCrUrA | | | | | NO: 1254) |
| | rCrUrCrUrUrGrUr | | | | | |
| | ArGrArUrCrCrUrU | | | | | |
| | rGrUrCrArArGrGr | | | | | |
| | CrUrArUrUrGrGrU | | | | | |
| | rC*mA | | | | | |
| 1105 | mU*mU*rUrUrUr | PolyU | 1xPS- | 66 | 21 | CCUUGUC |
| | UrUrUrUrUrUrUr | RNA + | OMe | | | AAGGCUA |
| | UrUrUrUrUrUrUr | 2×PS | | | | UUGGUCA |
| | UrUrUrUrUrUrUr | | | | | (SEQ ID |
| | ArArUrUrUrCrUrA | | | | | NO: 1254) |
| | rCrUrCrUrUrGrUr | | | | | |
| | ArGrArUrCrCrUrU | | | | | |
| | rGrUrCrArArGrGr | | | | | |
| | CrUrArUrUrGrGrU | | | | | |
| | rC*mA | | | | | |

All bases are in upper case
Lowercase “r” represents RNA, 2′-hydroxy; bases not modified by an “r” are DNA
All bases are linked via standard phosphodiester bonds except as noted:
“*” represents phosphorothioate modification
“PS” represents phosphorothioate modification
“PS2” represents phosphorodithioate modification
“OMe” represents a 2′-o-methyl modification
“m” represents a 2′-o-methyl modification
** Table 13 provides a listing of the sequences of the gRNA 5′ extensions

TABLE 9
Cpf1 Protein Variants

| | Cpf1 Variant Amino | Cpf1 Variant Nucleotide |
|---|---|---|
| Cpf1 Variant ID | Acid SEQ ID NO* | SEQ ID NO* |
| His-AsCpf1-sNLS- | SEQ ID NO: 1032 | SEQ ID NO: 1110 |
| sNLS H800A | | |
| Cpf1-1 | SEQ ID NO: 1094 | SEQ ID NO: 1111 |
| Cpf1-2 | SEQ ID NO: 1095 | SEQ ID NO: 1112 |
| Cpf1-3 | SEQ ID NO: 1096 | SEQ ID NO: 1113 |
| Cpf1-4 | SEQ ID NO: 1097 | SEQ ID NO: 1114 |
| Cpf1-5 | SEQ ID NO: 1107 | SEQ ID NO: 1115 |
| Cpf1-6 | SEQ ID NO: 1108 | SEQ ID NO: 1116 |
| Cpf1-7 | SEQ ID NO: 1109 | SEQ ID NO: 1117 |

*See FIG. 6

TABLE 10
RNP complexes

| RNP Name | gRNA SEQ ID NO* | Cpf1 Amino Acid SEQ ID NO** |
|---|---|---|
| RNP64 | SEQ ID NO: 1022 | SEQ ID NO: 1109 |
| RNP1 | SEQ ID NO: 1051 | SEQ ID NO: 1095 |
| RNP2 | SEQ ID NO: 1098 | SEQ ID NO: 1094 |
| RNP3 | SEQ ID NO: 1099 | SEQ ID NO: 1094 |
| RNP4 | SEQ ID NO: 1100 | SEQ ID NO: 1094 |
| RNP5 | SEQ ID NO: 1101 | SEQ ID NO: 1094 |
| RNP6 | SEQ ID NO: 1068 | SEQ ID NO: 1094 |
| RNP7 | SEQ ID NO: 1102 | SEQ ID NO: 1094 |
| RNP8 | SEQ ID NO: 1081 | SEQ ID NO: 1094 |
| RNP9 | SEQ ID NO: 1082 | SEQ ID NO: 1094 |
| RNP10 | SEQ ID NO: 1083 | SEQ ID NO: 1094 |
| RNP11 | SEQ ID NO: 1069 | SEQ ID NO: 1094 |
| RNP12 | SEQ ID NO: 1084 | SEQ ID NO: 1094 |
| RNP16 | SEQ ID NO: 1085 | SEQ ID NO: 1094 |
| RNP19 | SEQ ID NO: 1088 | SEQ ID NO: 1094 |
| RNP20 | SEQ ID NO: 1089 | SEQ ID NO: 1094 |
| RNP21 | SEQ ID NO: 1090 | SEQ ID NO: 1094 |
| RNP22 | SEQ ID NO: 1091 | SEQ ID NO: 1094 |
| RNP23 | SEQ ID NO: 1048 | SEQ ID NO: 1097 |
| RNP24 | SEQ ID NO: 1093 | SEQ ID NO: 1094 |
| RNP26 | SEQ ID NO: 1051 | SEQ ID NO: 1096 |
| RNP27 | SEQ ID NO: 1051 | SEQ ID NO: 1107 |
| RNP28 | SEQ ID NO: 1051 | SEQ ID NO: 1108 |
| RNP29 | SEQ ID NO: 1103 | SEQ ID NO: 1094 |
| RNP30 | SEQ ID NO: 1104 | SEQ ID NO: 1094 |
| RNP31 | SEQ ID NO: 1105 | SEQ ID NO: 1094 |
| RNP32 | SEQ ID NO: 1051 | SEQ ID NO: 1097 |
| RNP33 | SEQ ID NO: 1022 | SEQ ID NO: 1032 |
| RNP34 | SEQ ID NO: 1041 | SEQ ID NO: 1032 |
| RNP37 | SEQ ID NO: 1042 | SEQ ID NO: 1032 |
| RNP38 | SEQ ID NO: 1043 | SEQ ID NO: 1032 |
| RNP39 | SEQ ID NO: 1044 | SEQ ID NO: 1032 |
| RNP40 | SEQ ID NO: 1045 | SEQ ID NO: 1032 |
| RNP41 | SEQ ID NO: 1046 | SEQ ID NO: 1032 |
| RNP42 | SEQ ID NO: 1047 | SEQ ID NO: 1032 |
| RNP43 | SEQ ID NO: 1048 | SEQ ID NO: 1032 |
| RNP44 | SEQ ID NO: 1049 | SEQ ID NO: 1032 |
| RNP45 | SEQ ID NO: 1022 | SEQ ID NO: 1094 |
| RNP46 | SEQ ID NO: 1046 | SEQ ID NO: 1094 |
| RNP47 | SEQ ID NO: 1047 | SEQ ID NO: 1094 |
| RNP48 | SEQ ID NO: 1052 | SEQ ID NO: 1094 |
| RNP49 | SEQ ID NO: 1048 | SEQ ID NO: 1094 |
| RNP50 | SEQ ID NO: 1053 | SEQ ID NO: 1094 |
| RNP51 | SEQ ID NO: 1054 | SEQ ID NO: 1094 |
| RNP52 | SEQ ID NO: 1055 | SEQ ID NO: 1094 |
| RNP53 | SEQ ID NO: 1056 | SEQ ID NO: 1094 |
| RNP54 | SEQ ID NO: 1057 | SEQ ID NO: 1094 |
| RNP55 | SEQ ID NO: 1058 | SEQ ID NO: 1094 |
| RNP56 | SEQ ID NO: 1059 | SEQ ID NO: 1094 |
| RNP57 | SEQ ID NO: 1060 | SEQ ID NO: 1094 |
| RNP58 | SEQ ID NO: 1051 | SEQ ID NO: 1094 |
| RNP59 | SEQ ID NO: 1067 | SEQ ID NO: 1094 |
| RNP60 | SEQ ID NO: 1068 | SEQ ID NO: 1094 |
| RNP61 | SEQ ID NO: 1041 | SEQ ID NO: 1095 |
| RNP62 | SEQ ID NO: 1041 | SEQ ID NO: 1094 |
| RNP63 | SEQ ID NO: 1022 | SEQ ID NO: 1095 |

*See Table 8
**See Table 9

TABLE 11
Cpf1 HBG1 targeting domains and expected cleavage sites

| | gRNA | gRNA | Targeting | Expected | | |
|---|---|---|---|---|---|---|
| | Targeting | Targeting | Domain | cleavage site | | Targeting |
| | Domain | Domain | coordinates | coordinates at | | Domain |
| gRNA ID | (RNA) | (DNA) | at HBG1 * | HBG1^{#} | Strand | Length |
| HBG1-1 | CCUUGU | CCTTGTCA | Chr11:5249954- | Chr11:5249973, | + | 20 |
| | CAAGGC | AGGCTATT | 5249974 | Chr11:5249977 | | |
| | UAUUGG | GGTC (SEQ | | | | |
| | UC (SEQ | ID NO: 1003) | | | | |
| | ID | | | | | |
| | NO: 1002) | | | | | |
| HBG1-1 | CCUUGU | CCTTGTCA | Chr11:5249954- | Chr11:5249973, | + | 21 |
| (21mer) | CAAGGC | AGGCTATT | Chr11:5249975 | Chr11:5249977 | | |
| | UAUUGG | GGTCA (SEQ | | | | |
| | UCA (SEQ | ID NO: 1255) | | | | |
| | ID | | | | | |
| | NO: 1254) | | | | | |
| HBG1-1 | CCUUGU | CCTTGTCA | Chr11:5249954- | Chr11:5249973, | + | 22 |
| (22mer) | CAAGGC | AGGCTATT | Chr11:5249976 | Chr11:5249977 | | |
| | UAUUGG | GGTCAA | | | | |
| | UCAA | (SEQ ID | | | | |
| | (SEQ ID | NO: 1257) | | | | |
| | NO: 1256) | | | | | |
| HBG1-1 | CCUUGU | CCTTGTCA | Chr11:5249954- | Chr11:5249973, | + | 23 |
| (23mer) | CAAGGC | AGGCTATT | Chr11:5249976 | Chr11:5249977 | | |
| | UAUUGG | GGTCAAG | | | | |
| | UCAAG | (SEQ ID | | | | |
| | (SEQ ID | NO: 1259) | | | | |
| | NO: 1258) | | | | | |
| AsCpf1 | AGACAG | AGACAGAT | Chr11:5250023- | Chr11:5250042, | + | 20 |
| HBG1 | AUAUUU | ATTTGCATT | Chr11:5250043 | Chr11:5250046 | | |
| | GCAUUGAG | GAG (SEQ ID | | | | |
| | (SEQ ID | NO: 1140) | | | | |
| | NO: 1139) | | | | | |
| Promoter-1 | AGACAG | AGACAGAT | Chr11:5250023- | Chr11:5250042, | | |
| AsCpf1 | AUAUUU | ATTTGCATT | Chr11:5250044 | Chr11:5250046 | + | 21 |
| HBG1 | GCAUUG | GAGA (SEQ | | | | |
| | AGA (SEQ | ID NO: 1261) | | | | |
| | ID | | | | | |
| | NO: 1260) | | | | | |
| Promoter-1 | CAUUGA | CATTGAGA | Chr11:5250036- | Chr11:5250055, | + | 20 |
| (21mer) | GAUAGU | TAGTGTGG | Chr11:5250056 | Chr11:5250059 | | |
| AsCpf1 | GUGGGG | GGAA (SEQ | | | | |
| HBG1 | AA (SEQ | ID NO: 1142) | | | | |
| Promoter-2 | ID | | | | | |
| | NO: 1141) | | | | | |
| AsCpf1 | CAUUGA | CATTGAGA | Chr11:5250036- | Chr11:5250055, | + | 21 |
| HBG1 | GAUAGU | TAGTGTGG | Chr11:5250057 | Chr11:5250059 | | |
| Promoter-2 | GUGGGG | GGAAG | | | | |
| (21mer) | AAG (SEQ | (SEQ ID | | | | |
| | ID | NO: 1263) | | | | |
| | NO: 1262) | | | | | |
| AsCpf1 | CUUCUC | CTTCTCCCA | Chr11:5250160- | Chr11:5250179, | + | 20 |
| HBG1 | CCAUCA | TCATAGAG | Chr11:5250180 | Chr11:5250183 | | |
| | UAGAGG | GAT (SEQ ID | | | | |
| | AU (SEQ | NO: 1150) | | | | |
| | ID | | | | | |
| | NO: 1149) | | | | | |
| Promoter-6 | CUUCUC | CTTCTCCCA | Chr11:5250160- | Chr11:5250179, | | |
| AsCpf1 | CCAUCA | TCATAGAG | Chr11:5250181 | Chr11:5250183 | + | 21 |
| HBG1 | UAGAGG | GATA (SEQ | | | | |
| Promoter-6 | AUA (SEQ | ID NO: 1265) | | | | |
| (21mer) | ID | | | | | |
| | NO: 1264) | | | | | |

*NCBI Reference Sequence NC_000011, the coordinates are reported using the One-based coordinate system, “Homo sapiens chromosome 11, GRCh38.p12 Primary Assembly,” (Version NC_000011.10).
^{#}Expected cleavage sites based on Zetsche et al, 2015, coordinates are reported using zero-based coordinates.

TABLE 12
Cpf1 HBG2 targeting domains and expected cleavage sites

| | gRNA | gRNA | Targeting | Expected | | |
|---|---|---|---|---|---|---|
| | Targeting | Targeting | Domain | cleavage site | | Targeting |
| | Domain | Domain | coordinates at | coordinates at | | Domain |
| gRNA ID | (RNA) | (DNA) | HBG2 * | HBG2^{#} | Strand | Length |
| HBG1-1 | CCUUGUCA | CCTTGTCAA | Chr11:5254878 | Chr11:5254897, | + | 20 |
| | AGGCUAUU | GGCTATTGG | Chr11:5254898 | Chr11:5254901 | | |
| | GGUC | TC | | | | |
| | (SEQ ID | (SEQ ID | | | | |
| | NO: 1002) | NO: 1003) | | | | |
| HBG1-1 | CCUUGUCA | CCTTGTCAA | Chr11:5254878 | Chr11:5254897, | + | 21 |
| 21 mer | AGGCUAUU | GGCTATTGG | Chr11:5254899 | Chr11:5254901 | | |
| | GGUCA | TCA | | | | |
| | (SEQ ID | (SEQ ID | | | | |
| | NO: 1254) | NO: 1255) | | | | |
| HBG1-1 | CCUUGUCA | CCTTGTCAA | Chr11:5254878 | Chr11:5254897, | + | 22 |
| 22mer | AGGCUAUU | GGCTATTGG | Chr11:5254900 | Chr11:5254901 | | |
| | GGUCAA | TCAA | | | | |
| | (SEQ ID | (SEQ ID | | | | |
| | NO: 1256) | NO: 1257) | | | | |
| HBG1-1 | CCUUGUCA | CCTTGTCAA | Chr11:5254878 | Chr11:5254897, | + | 23 |
| 23mer | AGGCUAUU | GGCTATTGG | Chr11:5254901 | Chr11:5254901 | | |
| | GGUCAAG | TCAAG (SEQ | | | | |
| | SEQ ID | ID NO: 1259) | | | | |
| | NO: 1258) | | | | | |
| AsCpf1 | AGACAGAU | AGACAGATA | Chr11:5254947 | Chr11:5254966 | + | 20 |
| HBG1 | AUUUGCAU | TTTGCATTG | Chr11:5254967 | Chr11:5254970 | | |
| Promoter-1 | UGAG (SEQ | AG (SEQ ID | | | | |
| | ID NO: 1139) | NO: 1140) | | | | |
| AsCpf1 | AGACAGAU | AGACAGATA | Chr11:5254947 | Chr11:5254966 | + | 21 |
| HBG1 | AUUUGCAU | TTTGCATTG | Chr11:5254968 | Chr11:5254970 | | |
| Promoter-1 | UGAGA | AGA (SEQ ID | | | | |
| (21mer) | (SEQ ID | NO: 1261) | | | | |
| | NO: 1260) | | | | | |
| AsCpf1 | CAUUGAGA | CATTGAGAT | Chr11:5254960 | Chr11:5254979 | + | 20 |
| HBG1 | UAGUGUGG | AGTGTGGGG | Chr11:5254980 | Chr11:5254983 | | |
| Promoter-2 | GGAA (SEQ | AA | | | | |
| | ID NO: 1141) | (SEQ ID | | | | |
| | | NO: 1142) | | | | |
| AsCpf1 | CAUUGAGA | CATTGAGAT | Chr11:5254960 | Chr11:5254979 | + | 21 |
| HBG1 | UAGUGUGG | AGTGTGGGG | Chr11:5254981 | Chr11:5254983 | | |
| Promoter-2 | GGAAG | AAG (SEQ ID | | | | |
| (21mer) | (SEQ ID | NO: 1263) | | | | |
| | NO: 1262) | | | | | |

*NCBI Reference Sequence NC_000011, the coordinates are reported using the One-based coordinate system, “Homo sapiens chromosome 11, GRCh38.p12 Primary Assembly,” (Version NC_000011.10).
^{#}Expected cleavage sites based on (Zetsche et al, 2015), coordinates are reported using zero-based coordinates.

TABLE 13
gRNA 5′ Extensions

| 5′ extension | | |
|---|---|---|
| Sequence ID | | 5′ |
| No: | 5′ extension sequence | modification |
| 1231 | rCrUrUrUrU | +5 RNA |
| 1232 | rArArGrArCrCrUrUrUrU | +10 RNA |
| 1233 | rArUrGrUrGrUrUrUrUrUrGrUrCrArArArArGrAr | +25 RNA |
| | CrCrUrUrUrU | |
| 1234 | rArGrGrCrCrArGrCrUrUrGrCrCrGrGrUrUrUrUr | +60 RNA |
| | UrUrArGrUrCrGrUrGrCrUrGrCrUrUrCrArUrGrU | |
| | rGrUrUrUrUrUrGrUrCrArArArArGrArCrCrUrUr | |
| | UrU | |
| 1235 | CTTTT | +5 DNA |
| 1236 | AAGACCTTTT | +10 DNA |
| 1237 | ATGTGTTTTTGTCAAAAGACCTTTT | +25 DNA |
| 1238 | AGGCCAGCTTGCCGGTTTTTTAGTCGTGCTGCTTCATGT | +60 DNA |
| | GTTTTTGTCAAAAGACCTTTT | |
| 1239 | TTTTTGTCAAAAGACCTTTT | +20 DNA |
| 1240 | GCTTCATGTGTTTTTGTCAAAAGACCTTTT | +30 DNA |
| 1241 | GCCGGTTTTTTAGTCGTGCTGCTTCATGTGTTTTTGTCA | +50 DNA |
| | AAAGACCTTTT | |
| 1242 | TAGTCGTGCTGCTTCATGTGTTTTTGTCAAAAGACCTTT | +40 DNA |
| | T | |
| 1243 | C*C*GAAGTTTTCTTCGGTTTT | +20 DNA + 2xPS |
| 1244 | T*T*TTTCCGAAGTTTTCTTCGGTTTT | +25 DNA + 2xPS |
| 1245 | A*A*CGCTTTTTCCGAAGTTTTCTTCGGTTTT | +30 DNA + 2xPS |
| 1246 | G*C*GTTGTTTTCAACGCTTTTTCCGAAGTTTTCTTCGG | +41 DNA + 2xPS |
| | TTTT | |
| 1247 | G*G*CTTCTTTTGAAGCCTTTTTGCGTTGTTTTCAACGC | +62 DNA + 2xPS |
| | TTTTTCCGAAGTTTTCTTCGGTTTT | |
| 1248 | A*T*GTGTTTTTGTCAAAAGACCTTTT | +25 DNA + 2xPS |
| 1249 | AAAAAAAAAAAAAAAAAAAAAAAAA | +25 A |
| 1250 | TTTTTTTTTTTTTTTTTTTTTTTTT | +25 T |
| 1251 | mA*mU*rGrUrGrUrUrUrUrUrGrUrCrArArArArGr | +25 RNA + 2xPS |
| | ArCrCrUrUrUrU | |
| 1252 | mA*mA*rArArArArArArArArArArArArArArArAr | PolyA RNA + |
| | ArArArArArArA | 2xPS |
| 1253 | mU*mU*rUrUrUrUrUrUrUrUrUrUrUrUrUrUrUrUr | PolyU RNA + |
| | UrUrUrUrUrUrU | 2xPS |

All bases are in upper case
Lowercase “r” represents RNA, 2′-hydroxy; bases not modified by an “r” are DNA
All bases are linked via standard phosphodiester bonds except as noted:
“*” represents phosphorothioate modification
“PS” represents phosphorothioate modification

### SEQUENCES

Genome editing system components according to the present disclosure (including without limitation, RNA-guided nucleases, guide RNAs, donor template nucleic acids, nucleic acids encoding nucleases or guide RNAs, and portions or fragments of any of the foregoing), are exemplified by the nucleotide and amino acid sequences presented in the Sequence Listing. The sequences presented in the Sequence Listing are not intended to be limiting, but rather illustrative of certain principles of genome editing systems and their component parts, which, in combination with the instant disclosure, will inform those of skill in the art about additional implementations and modifications that are within the scope of this disclosure.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

### REFERENCES

Ahern et al., Br J Haematol 25(4):437-444 (1973)
Akinbami Hemoglobin 40:64-65 (2016)
Aliyu et al. Am J Hematol 83:63-70 (2008)
Anders et al. Nature 513(7519):569-573 (2014)
Angastiniotis & Modell Ann N Y Acad Sci 850:251-269 (1998)
Bae et al. Bioinformatics 30(10):1473-1475 (2014)
Barbosa et al. Braz J Med Bio Res 43(8):705-711 (2010)
Bauer et al. Nat. Med. 25(5):776-783 (2019)
Bothmer et al. CRISPR J 3(3):177-187 (2020)
Bouva Hematologica 91(1):129-132 (2006)
Briner et al. Mol Cell 56(2):333-339 (2014)
Brousseau Am J Hematol 85(1):77-78 (2010)
Caldecott Nat Rev Genet 9(8):619-631 (2008)
Canvers et al. Nature 527(12):192-197 (2015)
Chang et al. Mol Ther Methods Clin Dev 4:137-148 (2017)
Chassanidis Ann Hematol 88(6):549-555 (2009)
Chylinski et al. RNA Biol 10(5):726-737 (2013)
Cong et al. Science 399(6121):819-823 (2013)
Costa et al., Cad Saude Publica 18(5):1469-1471 (2002)
Davis & Maizels 2 Proc Natl Acad Sci USA 111(10):E924-932 (2014)
Fine et al. Sci Rep. 5:10777 (2015)
Frit et al. DNA Repair (Amst) 17:81-97 (2014)
Fu et al. Nat Biotechnol 32:279-284 (2014)
Gao et al. Nat Biotechnol.: 35(8):789-792 (2017)
Giarratana et al. Nat Biotechnol. 23(1):69-74 (2005)
Giarratana et al. Blood:118, 5071-5079 (2011)
Giannoukos et al. BMC Genomics 19(1):212 (2018)
Guilinger et al. Nat Biotechnol 32:577-582 (2014)
Heigwer et al. Nat Methods 11(2):122-123 (2014)
Hsu et al. Nat Biotechnol 31(9):827-832 (2013)
Iyama & Wilson DNA Repair (Amst) 12(8):620-636 (2013)
Jiang et al. Nat Biotechnol 31(3):233-239 (2013)
Jinek et al. Science 337(6096):816-821 (2012)
Jinek et al. Science 343(6176):1247997 (2014)
Kleinstiver et al. Nature 523(7561):481-485 (2015a)
Kleinstiver et al. Nat Biotechnol 33(12):1293-1298 (2015b)
Kleinstiver et al. Nature 529(7587):490-495 (2016)
Kleinstiver et al. Nat Biotechnol 37(3):276-282 (2019)
Komor et al. Nature 533(7603):420-424 (2016)
Kosicki et al. Nat Biotechnol 36(8): 765-771 (2018)
Lee et al. Nano Lett 12(12):6322-6327 (2012)
Lewis “Medical-Surgical Nursing: Assessment and Management of Clinical Problems” (2014)
Li Cell Res 18(1):85-98 (2008)
Makarova et al. Nat Rev Microbiol 9(6):467-477 (2011)
Mali et al. Science 339(6121):823-826 (2013)
Mantovani et al. Nucleic Acids Res 16(16):7783-7797 (1988)
Masala Methods Enzymol. 231:21-44 (1994)
Marteijn et al. Nat Rev Mol Cell Biol 15(7):465-481 (2014)
Martyn et al., Biochim Biophys Acta 1860(5):525-536 (2017)
Métais et al. Blood Adv. 3(21):3379-92 (2019)
Nishimasu et al. Cell 156(5):935-949 (2014)
Nishimasu et al. Cell 162:1113-1126 (2015)
Notta et al. Science 333(6039):218-21 (2011)
Pausch et al. Science 369(6501):333-337 (2020)
Ran & Hsu Cell 154(6):1380-1389 (2013)
Richardson et al. Nat Biotechnol 34:339-344 (2016)
Swarts et al. May 22:e1481. doi: 10.1002/wrna.1481. Epub ahead of print. PMID: 29790280 (2018)
Shmakov et al. Molecular Cell 60(3):385-397 (2015)
Sternberg et al. Nature 507(7490):62-67 (2014)
Strohkendl et al. Mol. Cell 71:816-824 (2018)
Superti-Furga et al. EMBO J 7(10):3099-3107 (1988)
Thein Hum Mol Genet 18(R2):R216-223 (2009)
Thorpe et al. Br J Haematol. 87(1):125-132 (1994)
Tsai et al. Nat Biotechnol 34(5): 483 (2016)
Waber et al. Blood 67(2):551-554 (1986)
Wang et al. Cell 153(4):910-918 (2013)
Weber et al. Sci Adv. 6(7):eaay9392 (2020)
Wu et al. Nat. Med. 25(5): 776-83 (2019)
Xiao et al. Bioinformatics 30(8):1180-1182 (2014)
Xu et al. Genes Dev 24(8):783-798 (2010)
Yamano et al. Cell 165(4): 949-962 (2016)
Zetsche et al. Nat Biotechnol 33(2):139-42 (2015)

1. A method of alleviating one or more symptoms of beta-thalassemia (β-Thal) in a subject in need thereof, the method comprising:
a) isolating a population of CD34+ or hematopoietic stem cells from the subject;
b) modifying the population of isolated cells ex vivo by delivering an RNP complex to the population of isolated cells, thereby altering a promoter of an HBG gene in one or more isolated cells in the population, the RNP complex comprising:
a Cpf1 and
a gRNA comprising:
a 5′ end and a 3′ end,
an RNA or DNA extension at the 5′ end,
a modification, e.g., a phosphorothioate linkage and/or a 2′-O-methyl modification at the 5′ or 3′ end, and
a targeting domain that is complementary to a target site in the promoter of the HBG gene, and
c) administering the modified population of isolated cells to the subject, thereby alleviating one or more symptoms of β-Thal in the subject.

2. The method of claim 1, wherein the DNA extension comprises a sequence selected from the group consisting of SEQ ID NOs:1235-1250.

3. The method of claim 1 or 2, wherein the targeting domain comprises, or consists of, a sequence set forth in Tables 7, 8, 11, and 12.

4. The method of claims 1-3, wherein the target site comprises nucleotides located between Chr 11 (NC_000011.10) 5,249,904-5,249,927 (Table 6, Region 6); Chr 11 (NC_000011.10) 5,254,879-5,254,909 (Table 6, Region 16); or a combination thereof.

5. The method of claims 1-4, wherein the Cpf1 comprises one or more modifications selected from the group consisting of one or more mutations in a wild-type Cpf1 amino acid sequence, one or more mutations in a wild-type Cpf1 nucleic acid sequence, one or more nuclear localization signals (NLS), one or more purification tags, and a combination thereof.

6. The method of claims 1-5, wherein the Cpf1 comprises or consists of a sequence selected from the group consisting of SEQ ID NOs: 1000, 1001, 1008-1018, 1032, 1035-39, 1094-1097, and 1107-09.

7. The method of claims 1-5, wherein the Cpf1 comprises or consists of a sequence selected from the group consisting of SEQ ID NOs:1019-1021 and 1110-17.

8. The method of claims 1-7, wherein the RNP complex is delivered to the cell using electroporation.

9. A method of inducing expression of hemoglobin (Hb) in a population of CD34+ or hematopoietic stem cells from a subject with beta-thalassemia (β-Thal), the method comprising:
delivering an RNP complex comprising a guide RNA (gRNA) and a Cpf1 to a population of unmodified CD34+ or hematopoietic stem cells from a subject with β-Thal to generate a population of modified CD34+ or hematopoietic stem cells comprising indels, the gRNA comprising a gRNA targeting domain,
wherein each modified CD34+ or hematopoietic stem cell comprises an indel in an HBG gene promoter, and
wherein the population of modified CD34+ or hematopoietic stem cells comprises higher Hb levels than the population of unmodified CD34+ or hematopoietic stem cells.

10. The method of claim 9, wherein the gRNA comprises a DNA extension comprising a sequence selected from the group consisting of SEQ ID NOs:1235-1250.

11. The method of claim 9 or 10, wherein the gRNA targeting domain comprises, or consists of, a sequence set forth in Tables 7, 8, 11, and 12.

12. The method of claims 9-11, wherein the gRNA comprises a targeting domain that is complementary to a target site in the promoter of an HBG gene, wherein the target site comprises nucleotides located between Chr 11 (NC_000011.10) 5,249,904-5,249,927 (Table 6, Region 6); Chr 11 (NC_000011.10) 5,254,879-5,254,909 (Table 6, Region 16); or a combination thereof.

13. The method of claims 9-12, wherein the RNP complex comprises a Cpf1 comprising one or more modifications selected from the group consisting of one or more mutations in a wild-type Cpf1 amino acid sequence, one or more mutations in a wild-type Cpf1 nucleic acid sequence, one or more nuclear localization signals (NLS), one or more purification tags, and a combination thereof.

14. The method of claims 9-13, wherein the Cpf1 comprises or consists of a sequence selected from the group consisting of SEQ ID NOs: 1000, 1001, 1008-1018, 1032, 1035-39, 1094-1097, and 1107-09.

15. The method of claims 9-14, wherein the Cpf1 comprises or consists of a sequence selected from the group consisting of SEQ ID NOs:1019-1021 and 1110-17.

16. The method of claims 9-15, wherein the RNP complex is delivered to the cell using electroporation.


SEQUENCE LISTING (1276 sequences)

SEQ ID NO: 1 - protein, 1345 aa, Streptococcus mutans
  REGION 10..21: note = misc_feature - N-terminal RuvC-like domain
  REGION 759..766: note = misc_feature - RuvC-like domain
  REGION 837..863: note = misc_feature - HNH-like domain
  REGION 982..989: note = misc_feature - RuvC-like domain
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 2 - protein, 1368 aa, Streptococcus pyogenes [sequence omitted]

SEQ ID NO: 3 - DNA, 4107 bp, Streptococcus pyogenes
  source 1..4107: mol_type = other DNA
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 4 - protein, 1388 aa, Streptococcus thermophilus
  REGION 10..21: note = misc_feature - N-terminal RuvC-like domain
  REGION 760..767: note = misc_feature - RuvC-like domain
  REGION 844..870: note = misc_feature - HNH-like domain
  REGION 989..996: note = misc_feature - RuvC-like domain
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 5 - protein, 1334 aa, Listeria innocua
  REGION 10..21: note = misc_feature - N-terminal RuvC-like domain
  REGION 762..769: note = misc_feature - RuvC-like domain
  REGION 840..866: note = misc_feature - HNH-like domain
  REGION 985..992: note = misc_feature - RuvC-like domain
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 6 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 7 - DNA, 3159 bp, Staphylococcus aureus
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 8 - DNA, 3159 bp, Staphylococcus aureus
  source 1..3159: mol_type = other DNA
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 9 - DNA, 3159 bp, Staphylococcus aureus
  source 1..3159: mol_type = other DNA
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 10 - DNA, 3159 bp, Staphylococcus aureus
  source 1..3159: mol_type = other DNA
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 11 - DNA, 3159 bp, Staphylococcus aureus
  source 1..3159: mol_type = other DNA
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 12 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 13 - DNA, 3249 bp, Neisseria meningitidis
  misc_feature 1..3249: note = Exemplary codon optimized Cas9
  source 1..3249: mol_type = other DNA
atggccgccttcaagcccaaccccatcaactacatcctgggcctggacatcggcatcgccagcgtgggctgggccatggtggagatcgacgaggacgagaaccccatctgcctgatcgacctgggtgtgcgcgtgttcgagcgcgctgaggtgcccaagactggtgacagtctggctatggctcgccggcttgctcgctctgttcggcgccttactcgccggcgcgctcaccgccttctgcgcgctcgccgcctgctgaagcgcgagggtgtgctgcaggctgccgacttcgacgagaacggcctgatcaagagcctgcccaacactccttggcagctgcgcgctgccgctctggaccgcaagctgactcctctggagtggagcgccgtgctgctgcacctgatcaagcaccgcggctacctgagccagcgcaagaacgagggcgagaccgccgacaaggagctgggtgctctgctgaagggcgtggccgacaacgcccacgccctgcagactggtgacttccgcactcctgctgagctggccctgaacaagttcgagaaggagagcggccacatccgcaaccagcgcggcgactacagccacaccttcagccgcaaggacctgcaggccgagctgatcctgctgttcgagaagcagaaggagttcggcaacccccacgtgagcggcggcctgaaggagggcatcgagaccctgctgatgacccagcgccccgccctgagcggcgacgccgtgcagaagatgctgggccactgcaccttcgagccagccgagcccaaggccgccaagaacacctacaccgccgagcgcttcatctggctgaccaagctgaacaacctgcgcatcctggagcagggcagcgagcgccccctgaccgacaccgagcgcgccaccctgatggacgagccctaccgcaagagcaagctgacctacgcccaggcccgcaagctgctgggtctggaggacaccgccttcttcaagggcctgcgctacggcaaggacaacgccgaggccagcaccctgatggagatgaaggcctaccacgccatcagccgcgccctggagaaggagggcctgaaggacaagaagagtcctctgaacctgagccccgagctgcaggacgagatcggcaccgccttcagcctgttcaagaccgacgaggacatcaccggccgcctgaaggaccgcatccagcccgagatcctggaggccctgctgaagcacatcagcttcgacaagttcgtgcagatcagcctgaaggccctgcgccgcatcgtgcccctgatggagcagggcaagcgctacgacgaggcctgcgccgagatctacggcgaccactacggcaagaagaacaccgaggagaagatctacctgcctcctatccccgccgacgagatccgcaaccccgtggtgctgcgcgccctgagccaggcccgcaaggtgatcaacggcgtggtgcgccgctacggcagccccgcccgcatccacatcgagaccgcccgcgaggtgggcaagagcttcaaggaccgcaaggagatcgagaagcgccaggaggagaaccgcaaggaccgcgagaaggccgccgccaagttccgcgagtacttccccaacttcgtgggcgagcccaagagcaaggacatcctgaagctgcgcctgtacgagcagcagcacggcaagtgcctgtacagcggcaaggagatcaacctgggccgcctgaacgagaagggctacgtggagatcgaccacgccctgcccttcagccgcacctgggacgacagcttcaacaacaaggtgctggtgctgggcagcgagaaccagaacaagggcaaccagaccccctacgagtacttcaacggcaaggacaacagccgcgagtggcaggagttcaaggcccgcgtggagaccagccgcttcccccgcagcaagaagcagcgcatcctgctgcagaagttcgacgaggacggcttcaaggagcgcaacctgaacgacacccgctacgtgaaccgcttcctgtgccagttcgtggccgaccgcatgcgcctgaccggcaagggcaagaagcgcgtgttcgccagcaacggccagatcaccaacctgctgcgcggcttctggggcctgcgcaaggtgcgcgccgagaacgaccgccaccacgccctggacgccgtggtggtggcctgcagcaccgtggccatgcagcagaagatcacccgcttcgtgcgctacaaggagatgaacgccttcgacggtaaaaccatcgacaaggagaccggcgaggtgctgcaccagaagacccacttcccccagccctgggagttcttcgcccaggaggtgatgatccgcgtgttcggcaagcccgacggcaagcccgagttcgaggaggccgacacccccgagaagctgcgcaccctgctggccgagaagctgagcagccgccctgaggccgtgcacgagtacgtgactcctctgttcgtgagccgcgcccccaaccgcaagatgagcggtcagggtcacatggagaccgtgaagagcgccaagcgcctggacgagggcgtgagcgtgctgcgcgtgcccctgacccagctgaagctgaaggacctggagaagatggtgaaccgcgagcgcgagcccaagctgtacgaggccctgaaggcccgcctggaggcccacaaggacgaccccgccaaggccttcgccgagcccttctacaagtacgacaaggccggcaaccgcacccagcaggtgaaggccgtgcgcgtggagcaggtgcagaagaccggcgtgtgggtgcgcaaccacaacggcatcgccgacaacgccaccatggtgcgcgtggacgtgttcgagaagggcgacaagtactacctggtgcccatctacagctggcaggtggccaagggcatcctgcccgaccgcgccgtggtgcagggcaaggacgaggaggactggcagctgatcgacgacagcttcaacttcaagttcagcctgcaccccaacgacctggtggaggtgatcaccaagaaggcccgcatgttcggctacttcgccagctgccaccgcggcaccggcaacatcaacatccgcatccacgacctggaccacaagatcggcaagaacggcatcctggagggcatcggcgtgaagaccgccctgagcttccagaagtaccagatcgacgagctgggcaaggagatccgcccctgccgcctgaagaagcgccctcctgtgcgctaa

SEQ ID NO: 14 - protein, 859 aa, synthetic construct
  SITE 4: note = misc_feature - Xaa can be any naturally occurring amino acid
  REGION 8..18: note = misc_feature - N-terminal RuvC-like domain
  SITE 21: note = misc_feature - Xaa can be any naturally occurring amino acid
  SITE 23: note = misc_feature - Xaa can be any naturally occurring amino acid
  SITE 24: note = misc_feature - Xaa can be any naturally occurring amino acid
  SITE 26: note = misc_feature - Xaa can be any naturally occurring amino acid
  REGION 29..31: note = misc_feature - Each Xaa can independently be any naturally occurring amino acid
  SITE 33: note = misc_feature - Xaa can be any naturally occurring amino acid
  SITE 36: note = misc_feature - Xaa can be any naturally occurring amino acid
  SITE 45: note = misc_feature - Xaa can be any naturally occurring amino acid
  SITE 54: note = misc_feature - Xaa can be any naturally occurring amino acid
  SITE 63: note = misc_feature - Xaa can be any naturally occurring amino acid
  SITE 71: note = misc_feature - Xaa can be any naturally occurring amino acid
  SITE 75: note = misc_feature - Xaa can be any naturally occurring amino acid
  SITE 76: note = misc_feature - Xaa can be any naturally occurring amino acid
  REGION 78..80: note = misc_feature - Each Xaa can independently be any naturally occurring amino acid
  SITE 82: note = misc_feature - Xaa can be any naturally occurring amino acid
  SITE 84: note = misc_feature - Xaa can be any naturally occurring amino acid
  SITE 85: note = misc_feature - Xaa can be any naturally occurring amino acid
  SITE 89: note = misc_feature - Xaa can be any naturally occurring amino acid
  SITE 90: note = misc_feature - Xaa can be any naturally occurring amino acid
  SITE 98: note = misc_feature - Xaa can be any naturally occurring amino acid
  SITE 100: note = misc_feature - Xaa can be any naturally occurring amino acid
  SITE 106: note = misc_feature - Xaa can be any naturally occurring amino acid
  SITE 113: note = misc_feature - Xaa can be any naturally occurring amino acid
  SITE 116: note = misc_feature - Xaa can be any naturally occurring amino acid
  SITE 125: note = misc_feature - Xaa can be any naturally occurring amino acid
  SITE 126: note = misc_feature - Xaa can be any naturally occurring amino acid
  REGION 128..133: note = misc_feature - Each Xaa can independently be any naturally occurring amino acid
  SITE 135: note = misc_feature - Xaa can be any naturally occurring amino acid
  SITE 137: note = misc_feature - Xaa can be any naturally occurring amino acid
  REGION 139..147: note = misc_feature - Each Xaa can independently be any naturally occurring amino acid
  REGION 153..155: note = misc_feature - Each Xaa can independently be any naturally occurring amino acid
  SITE 157: note = misc_feature - Xaa can be any naturally occurring amino acid
  SITE 159: note = misc_feature - Xaa can be any naturally occurring amino acid
  SITE 161: note = misc_feature - Xaa can be any naturally occurring amino acid
  SITE 163: note = misc_feature - Xaa can be any naturally occurring amino acid
  REGION 166..168: note = misc_feature - Each Xaa can independently be any naturally occurring amino acid
  SITE 170: note = misc_feature - Xaa can be any naturally occurring amino acid
  SITE 171: note = misc_feature - Xaa can be any naturally occurring amino acid
  REGION 173..175: note = misc_feature - Each Xaa can independently be any naturally occurring amino acid
  SITE 177: note = misc_feature - Xaa can be any naturally occurring amino acid
  SITE 183: note = misc_feature - Xaa can be any naturally occurring amino acid
  REGION 185..187: note = misc_feature - Each Xaa can independently be any naturally occurring amino acid
  SITE 189: note = misc_feature - Xaa can be any naturally occurring amino acid
  REGION 192..195: note = misc_feature - Each Xaa can independently be any naturally occurring amino acid
  SITE 198: note = misc_feature - Xaa can be any naturally occurring amino acid
  SITE 199: note = misc_feature - Xaa can be any naturally occurring amino acid
  SITE 202: note = misc_feature - Xaa can be any naturally occurring amino acid
  SITE 206: note = misc_feature - Xaa can be any naturally occurring amino acid
  SITE 207: note = misc_feature - Xaa can be any naturally occurring amino acid
  SITE 210: note = misc_feature - Xaa can be any naturally occurring amino acid
  SITE 212: note = misc_feature - Xaa can be any naturally occurring amino acid
  SITE 213: note = misc_feature - Xaa can be any naturally occurring amino acid
  SITE 219: note = misc_feature - Xaa can be any naturally occurring amino acid
  SITE 220: note = misc_feature - Xaa can be any naturally occurring amino acid
  SITE 222: note = misc_feature - Xaa can be any naturally occurring amino acid
  SITE 224: note = misc_feature - Xaa can be any naturally occurring amino acid
  SITE 226: note = misc_feature - Xaa can be any naturally occurring amino acid
  SITE 236: note = misc_feature - Xaa can be any naturally occurring amino acid
  SITE 240: note = misc_feature - Xaa can be any naturally occurring amino acid
  SITE 241: note = misc_feature - Xaa can be any naturally occurring amino acid
  REGION 244..246: note = misc_feature - Each Xaa can independently be any naturally occurring amino acid
  SITE 248: note = misc_feature - Xaa can be any naturally occurring amino acid
  SITE 249: note = misc_feature - Xaa can be any naturally occurring amino acid
  SITE 250: note = misc_feature - Xaa can be any naturally occurring amino acid
  REGION 252..254: note = misc_feature - Each Xaa can independently be any naturally occurring amino acid
  SITE 256: note = misc_feature - Xaa can be any naturally occurring amino acid
  SITE 257: note = misc_feature - Xaa can be any naturally occurring amino acid
  SITE 268: note = misc_feature - Xaa can be any naturally occurring amino acid
  SITE 271: note = misc_feature - Xaa can be any naturally occurring amino acid
  SITE 273: note = misc_feature - Xaa can be any naturally occurring amino acid
  SITE 277: note = misc_feature - Xaa can be any naturally occurring amino acid
  SITE 280: note = misc_feature - Xaa can be any naturally occurring amino acid
  SITE 281: note = misc_feature - Xaa can be any naturally occurring amino acid
  SITE 283: note = misc_feature - Xaa can be any naturally occurring amino acid
  SITE 289: note = misc_feature - Xaa can be any naturally occurring amino acid
  SITE 290: note = misc_feature - Xaa can be any naturally occurring amino acid
  REGION 292..294: note = misc_feature - Each Xaa can independently be any naturally occurring amino acid
  SITE 301: note = misc_feature - Xaa can be any naturally occurring amino acid
  SITE 308: note = misc_feature - Xaa can be any naturally occurring amino acid
  REGION 319..322: note = misc_feature - Each Xaa can independently be any naturally occurring amino acid
  SITE 328: note = misc_feature - Xaa can be any naturally occurring amino acid
  SITE 329: note = misc_feature - Xaa can be any naturally occurring amino acid
  REGION 335..337: note = misc_feature - Each Xaa can independently be any naturally occurring amino acid
  SITE 346: note = misc_feature - Xaa can be any naturally occurring amino acid
  SITE 347: note = misc_feature - Xaa can be any naturally occurring amino acid
  REGION 356..361: note = misc_feature - Each Xaa can independently be any naturally occurring amino acid
  SITE 363: note = misc_feature - Xaa can be any naturally occurring amino acid
  REGION 370..373: note = misc_feature - Each Xaa can independently be any naturally occurring amino acid
  SITE 375: note = misc_feature - Xaa can be any naturally occurring amino acid
  SITE 376: note = misc_feature - Xaa can be any naturally occurring amino acid
  SITE 379: note = misc_feature - Xaa can be any naturally occurring amino acid
  REGION 386..390: note = misc_feature - Each Xaa can independently be any naturally occurring amino acid
  SITE 393: note = misc_feature - Xaa can be any naturally occurring amino acid
  SITE 395: note = misc_feature - Xaa can be any naturally occurring amino acid
  SITE 396: note = misc_feature - Xaa can be any naturally occurring amino acid
  SITE 398: note = misc_feature - Xaa can be any naturally occurring amino acid
  SITE 400: note = misc_feature - Xaa can be any naturally occurring amino acid
  SITE 403: note = misc_feature - Xaa can be any naturally occurring amino acid
  SITE 407: note = misc_feature - Xaa can be any naturally occurring amino acid
  SITE 410: note = misc_feature - Xaa can be any naturally occurring amino acid
  SITE 411: note = misc_feature - Xaa can be any naturally occurring amino acid
  REGION 413..416: note = misc_feature - Each Xaa can independently be any naturally occurring amino acid
  SITE 418: note = misc_feature - Xaa can be any naturally occurring amino acid
  SITE 422: note = misc_feature - Xaa can be any naturally occurring amino acid
  SITE 428: note = misc_feature - Xaa can be any naturally occurring amino acid
  SITE 431: note = misc_feature - Xaa can be any naturally occurring amino acid
  SITE 433: note = misc_feature - Xaa can be any naturally occurring amino acid
  REGION 437..439: note = misc_feature - Each Xaa can independently be any naturally occurring amino acid
  SITE 445: note = misc_feature - Xaa can be any naturally occurring amino acid
  SITE 451: note = misc_feature - Xaa can be any naturally occurring amino acid
  SITE 456: note = misc_feature - Xaa can be any naturally occurring amino acid
  SITE 459: note = misc_feature - Xaa can be any naturally occurring amino acid
  REGION 465..469: note = misc_feature - Each Xaa can independently be any naturally occurring amino acid
  SITE 481: note = misc_feature - Xaa can be any naturally occurring amino acid
  SITE 482: note = misc_feature - Xaa can be any naturally occurring amino acid
  SITE 484: note = misc_feature - Xaa can be any naturally occurring amino acid
  SITE 490: note = misc_feature - Xaa can be any naturally occurring amino acid
  SITE 494: note = misc_feature - Xaa can be any naturally occurring amino acid
  REGION 495..502: note = misc_feature - RuvC-like domain
  SITE 497: note = misc_feature - Xaa can be any naturally occurring amino acid
  SITE 506: note = misc_feature - Xaa can be any naturally occurring amino acid
  SITE 510: note = misc_feature - Xaa can be any naturally occurring amino acid
  SITE 513: note = misc_feature - Xaa can be any naturally occurring amino acid
  SITE 514: note = misc_feature - Xaa can be any naturally occurring amino acid
  SITE 517: note = misc_feature - Xaa can be any naturally occurring amino acid
  SITE 520: note = misc_feature - Xaa can be any naturally occurring amino acid
  SITE 525: note = misc_feature - Xaa can be any naturally occurring amino acid
  SITE 526: note = misc_feature - Xaa can be any naturally occurring amino acid
  SITE 529: note = misc_feature - Xaa can be any naturally occurring amino acid
  SITE 531: note = misc_feature - Xaa can be any naturally occurring amino acid
  SITE 532: note = misc_feature - Xaa can be any naturally occurring amino acid
  SITE 534: note = misc_feature - Xaa can be any naturally occurring amino acid
  SITE 542: note = misc_feature - Xaa can be any naturally occurring amino acid
  SITE 546: note = misc_feature - Xaa can be any naturally occurring amino acid
  SITE 547: note = misc_feature - Xaa can be any naturally occurring amino acid
  SITE 553: note = misc_feature - Xaa can be any naturally occurring amino acid
  REGION 555..575: note = misc_feature - HNH-like domain
  SITE 556: note = misc_feature - Xaa can be any naturally occurring amino acid
  SITE 560: note = misc_feature - Xaa can be any naturally occurring amino acid
  SITE 563: note = misc_feature - Xaa can be any naturally occurring amino acid
  SITE 565: note = misc_feature - Xaa can be any naturally occurring amino acid
  SITE 567: note = misc_feature - Xaa can be any naturally occurring amino acid
  SITE 579: note = misc_feature - Xaa can be any naturally occurring amino acid
  SITE 582: note = misc_feature - Xaa can be any naturally occurring amino acid
  SITE 583: note = misc_feature - Xaa can be any naturally occurring amino acid
  SITE 585: note = misc_feature - Xaa can be any naturally occurring amino acid
  SITE 588: note = misc_feature - Xaa can be any naturally occurring amino acid
  SITE 590: note = misc_feature - Xaa can be any naturally occurring amino acid
  SITE 592: note = misc_feature - Xaa can be any naturally occurring amino acid
  REGION 594..596: note = misc_feature - Each Xaa can independently be any naturally occurring amino acid
  SITE 610: note = misc_feature - Xaa can be any naturally occurring amino acid
  SITE 616: note = misc_feature - Xaa can be any naturally occurring amino acid
  SITE 628: note = misc_feature - Xaa can be any naturally occurring amino acid
  SITE 631: note = misc_feature - Xaa can be any naturally occurring amino acid
  SITE 633: note = misc_feature - Xaa can be any naturally occurring amino acid
  SITE 634: note = misc_feature - Xaa can be any naturally occurring amino acid
  SITE 636: note = misc_feature - Xaa can be any naturally occurring amino acid
  REGION 638..641: note = misc_feature - Each Xaa can independently be any naturally occurring amino acid
  REGION 643..645: note = misc_feature - Each Xaa can independently be any naturally occurring amino acid
  SITE 653: note = misc_feature - Xaa can be any naturally occurring amino acid
  SITE 657: note = misc_feature - Xaa can be any naturally occurring amino acid
  SITE 659: note = misc_feature - Xaa can be any naturally occurring amino acid
  SITE 660: note = misc_feature - Xaa can be any naturally occurring amino acid
  SITE 665: note = misc_feature - Xaa can be any naturally occurring amino acid
  SITE 666: note = misc_feature - Xaa can be any naturally occurring amino acid
  SITE 668: note = misc_feature - Xaa can be any naturally occurring amino acid
  SITE 669: note = misc_feature - Xaa can be any naturally occurring amino acid
  REGION 670..677: note = misc_feature - RuvC-like domain
  SITE 680: note = misc_feature - Xaa can be any naturally occurring amino acid
  SITE 681: note = misc_feature - Xaa can be any naturally occurring amino acid
  SITE 683: note = misc_feature - Xaa can be any naturally occurring amino acid
  SITE 686: note = misc_feature - Xaa can be any naturally occurring amino acid
  SITE 696: note = misc_feature - Xaa can be any naturally occurring amino acid
  SITE 697: note = misc_feature - Xaa can be any naturally occurring amino acid
  SITE 704: note = misc_feature - Xaa can be any naturally occurring amino acid
  SITE 708: note = misc_feature - Xaa can be any naturally occurring amino acid
  SITE 710: note = misc_feature - Xaa can be any naturally occurring amino acid
  SITE 711: note = misc_feature - Xaa can be any naturally occurring amino acid
  SITE 714: note = misc_feature - Xaa can be any naturally occurring amino acid
  REGION 717..720: note = misc_feature - Each Xaa can independently be any naturally occurring amino acid
  SITE 722: note = misc_feature - Xaa can be any naturally occurring amino acid
  SITE 725: note = misc_feature - Xaa can be any naturally occurring amino acid
  SITE 727: note = misc_feature - Xaa can be any naturally occurring amino acid
  REGION 733..735: note = misc_feature - Each Xaa can independently be any naturally occurring amino acid
  SITE 740: note = misc_feature - Xaa can be any naturally occurring amino acid
  SITE 742: note = misc_feature - Xaa can be any naturally occurring amino acid
  REGION 749..754: note = misc_feature - Each Xaa can independently be any naturally occurring amino acid
  REGION 758..761: note = misc_feature - Each Xaa can independently be any naturally occurring amino acid
  REGION 763..768: note = misc_feature - Each Xaa can independently be any naturally occurring amino acid
  SITE 771: note = misc_feature - Xaa can be any naturally occurring amino acid
  REGION 774..777: note = misc_feature - Each Xaa can independently be any naturally occurring amino acid
  SITE 782: note = misc_feature - Xaa can be any naturally occurring amino acid
  REGION 784..786: note = misc_feature - Each Xaa can independently be any naturally occurring amino acid
  SITE 788: note = misc_feature - Xaa can be any naturally occurring amino acid
  SITE 790: note = misc_feature - Xaa can be any naturally occurring amino acid
  SITE 795: note = misc_feature - Xaa can be any naturally occurring amino acid
  SITE 799: note = misc_feature - Xaa can be any naturally occurring amino acid
  SITE 801: note = misc_feature - Xaa can be any naturally occurring amino acid
  SITE 802: note = misc_feature - Xaa can be any naturally occurring amino acid
  SITE 804: note = misc_feature - Xaa can be any naturally occurring amino acid
  REGION 806..813: note = misc_feature - Each Xaa can independently be any naturally occurring amino acid
  REGION 815..818: note = misc_feature - Each Xaa can independently be any naturally occurring amino acid
  SITE 820: note = misc_feature - Xaa can be any naturally occurring amino acid
  REGION 823..827: note = misc_feature - Each Xaa can independently be any naturally occurring amino acid
  SITE 829: note = misc_feature - Xaa can be any naturally occurring amino acid
  SITE 830: note = misc_feature - Xaa can be any naturally occurring amino acid
  SITE 832: note = misc_feature - Xaa can be any naturally occurring amino acid
  REGION 835..837: note = misc_feature - Each Xaa can independently be any naturally occurring amino acid
  REGION 842..844: note = misc_feature - Each Xaa can independently be any naturally occurring amino acid
  SITE 846: note = misc_feature - Xaa can be any naturally occurring amino acid
  SITE 848: note = misc_feature - Xaa can be any naturally occurring amino acid
  SITE 851: note = misc_feature - Xaa can be any naturally occurring amino acid
  SITE 857: note = misc_feature - Xaa can be any naturally occurring amino acid
  SITE 857: note = misc_feature - Xaa can be any naturally occurring amino acid
MKYXIGLDIGTNSVGWAVTDXYXXKXKGXXXIXKNXGLFDGTARXRTARRRRRXNRIYLQIFXEMDFFRLXSFVXXKXXXPXFXXEYHXXPTIYHLRXLXKDLRLXYLALAHXIKXRGNFLIEGXXNXXXXXXYXFXIXXXXXXXXXPEKGFXXXLXGXFXFXLEXXXKXXYXXXLXLLIGDXYXXXFXAKXXXXLSXXVTXALSXXMIXRXXHDLLKXXYXEXFXKGYAGYIDGXQFYXXKLXXXGXXXKXXXEXXLRKQRTFDNGXIPXQXHLEXAIXXQXYPFLNXXIXXXTFRIPYXVGPLAGXSFAWRKIPWNXXXXDSAFIXXMTDLPXXXVLPKHSLYXXVYNELTKVXXXXXXKXIFKRKVXXXXGXXFNXSTYHDLXXXXXLDXNXXEXIXLTXFEDXMIXXLXXXXKXLRRXYTGWGXLSXLXGIRXXXSTILDXLDNRNXMQLIXDLXFKIKQXXXXXGSPAIKKGILQXXKXVDELVXMGPXIVXEMARENQTXGNSXRKXXKEXGSXILKEXXNLXNXXLXLYYLQNGXDMYXXLDILSXYDXDHIXPQXFXDXSIDNVLSNRKDXVPXXVXKKXWXLXLXXXRKFDLTKAERGGLXDKAFIXRQLVETRQITKXVAXLXXNXDXXXXVXXXTLKSLVSXFRKXFXXLYKVXXNXXHHAHDAYLNVXXLXYPXLEEFVYGDYXXKATKFYXNIMXFXXGEXWKXXXXVXMQXNXVKKEQXXXPKNSXLXKDKYGGXXXXXXKGKXXXXIXXXXXXFLXGYXXXXLPKYXLXXXGXRXLASEXKGNXLXXLXAXXXXXXXXEXXXXFXANXXXXXLXXGXAFXXXIRRYXXXTXIXQSXTGLYEXRL

SEQ ID NO: 15 - protein, 8 aa, synthetic construct
  REGION 1..8: note = RuvC-like domain
  VARIANT 2: note = Xaa is Val or His
  VARIANT 3: note = Xaa is Ile, Leu, or Val
  VARIANT 5: note = Xaa is Met or Thr
IXXEXARE

SEQ ID NO: 16 - protein, 8 aa, synthetic construct
  REGION 1..8: note = RuvC-like domain
  VARIANT 3: note = Xaa is Ile, Leu, or Val
IVXEMARE

SEQ ID NO: 17 - protein, 8 aa, synthetic construct
  REGION 1..8: note = RuvC-like domain
  VARIANT 4: note = Xaa is His or Leu
  VARIANT 7: note = Xaa is Arg or Val
  VARIANT 8: note = Xaa is Glu or Val
HHAXDAXX

SEQ ID NO: 18 - protein, 8 aa, synthetic construct
  REGION 1..8: note = RuvC-like domain
HHAHDAYL

SEQ ID NO: 19 - protein, 30 aa, synthetic construct
  REGION 1..30: note = N-terminal RuvC-like domain
  VARIANT 2: note = Xaa is Lys or Pro
  VARIANT 4: note = Xaa is Val, Leu, Ile, or Phe
  VARIANT 5: note = Xaa is Gly, Ala, or Ser
  VARIANT 6: note = Xaa is Leu, Ile, Val, or Phe
  MOD_RES 7..26: note = N-terminal RuvC-like domain, each Xaa can be any amino acid orabsent, region may encompass 5-20 residues
  VARIANT 29: note = Xaa is Asp, Glu, Asn, or Gln
KXYXXXXXXXXXXXXXXXXXXXXXXXTDXY

SEQ ID NO: 20 - skipped

SEQ ID NO: 21 - protein, 12 aa, synthetic construct
  REGION 1..12: note = N-terminal RuvC-like domain
  VARIANT 2: note = Xaa is Ile, Val, Met, Leu, or Thr
  VARIANT 4: note = Xaa is Thr, Ile, Val, Ser, Asn, Tyr, Glu, or Leu
  VARIANT 5: note = Xaa is Asn, Ser, Gly, Ala, Asp, Thr, Arg, Met, or Phe
  VARIANT 7: note = Xaa is Val, Ile, Leu, Cys, Thr, or Phe
  VARIANT 9: note = Xaa is Trp, Phe, Val, Tyr, Ser, or Leu
  VARIANT 10: note = Xaa is Ala, Ser, Cys, Val, or Gly
  VARIANT 11: note = Xaa is Val, Ile, Leu, Ala, Met, or His
  VARIANT 12: note = Any amino acid or absent
DXGXXSXGXXXX

SEQ ID NO: 22 - protein, 12 aa, synthetic construct
  REGION 1..12: note = N-terminal RuvC-like domain
  VARIANT 4: note = Xaa is Thr, Ile, Val, Ser, Asn, Tyr, Glu, or Leu
  VARIANT 5: note = Xaa is Asn, Ser, Gly, Ala, Asp, Thr, Arg, Met, or Phe
  VARIANT 11: note = Xaa is Val, Ile, Leu, Ala, Met, or His
  VARIANT 12: note = Any amino acid or absent
DIGXXSVGWAXX

SEQ ID NO: 23 - protein, 12 aa, synthetic construct
  REGION 1..12: note = N-terminal RuvC-like domain
  MOD_RES 12: note = Any non-polar alkyl amino acid or a hydroxyl amino acid
DIGTNSVGWAVX

SEQ ID NO: 24 - protein, 73 aa, synthetic construct
  REGION 1..73: note = HNH-like domain
  VARIANT 8: note = Xaa is Lys or Arg
  VARIANT 12: note = Xaa is Val or Thr
  VARIANT 13: note = Xaa is Gly or Asp
  VARIANT 14: note = Xaa is Glu, Gln, or Asp
  VARIANT 15: note = Xaa is Glu or Asp
  VARIANT 19: note = Xaa is Asp, Asn, or His
  VARIANT 20: note = Xaa is Tyr, Arg, or Asn
  VARIANT 23: note = Xaa is Gln, Asp, or Asn
  MOD_RES 25..64: note = HNH-like domain, each Xaa can be any amino acid or absent, regionmay encompass 15-40 residues
  VARIANT 67: note = Xaa is Gly or Glu
  VARIANT 69: note = Xaa is Ser or Gly
  VARIANT 71: note = Xaa is Asp or Asn
LYYLQNGXDMYXXXXLDIXXLSXYXXXXXXXXXXXXXXXXXXXXXXXXXXXXXXXXXXXXXXXXNRXKXDXVP

SEQ ID NO: 25 - protein, 27 aa, synthetic construct
  REGION 1..27: note = HNH-like domain
  VARIANT 1: note = Xaa is Asp, Glu, Gln, or Asn
  VARIANT 2: note = Xaa is Leu, Ile, Arg, Gln, Val, Met, or Lys
  VARIANT 3: note = Xaa is Asp or Glu
  VARIANT 5: note = Xaa is Ile, Val, Thr, Ala, or Leu
  VARIANT 6: note = Xaa is Val, Tyr, Ile, Leu, Phe, or Trp
  VARIANT 8: note = Xaa is Gln, His, Arg, Lys, Tyr, Ile, Leu, Phe, or Trp
  VARIANT 9: note = Xaa is Ser, Ala, Asp, Thr, or Lys
  VARIANT 10: note = Xaa is Phe, Leu, Val, Lys, Tyr, Met, Ile, Arg, Ala, Glu, Asp, orGln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, orSer
  VARIANT 13: note = Xaa is Asp, Ser, Asn, Arg, Leu, or Thr
  VARIANT 14: note = Xaa is Asp, Asn, or Ser
  VARIANT 15: note = Xaa is Ser, Ala, Thr, Gly, or Arg
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met,Gly, Tyr, or Val
  VARIANT 19: note = Xaa is Lys, Leu, Arg, Met, Thr, or Phe
  VARIANT 20: note = Xaa is Val, Leu, Ile, Ala, or Thr
  VARIANT 21: note = Xaa is Leu, Ile, Val, or Ala
  VARIANT 22: note = Xaa is Thr, Val, Cys, Glu, Ser, or Ala
  VARIANT 23: note = Xaa is Arg, Phe, Thr, Trp, Glu, Leu, Asn, Cys, Lys, Val, Ser,Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln,Arg, or Tyr
  VARIANT 26: note = Xaa is Lys, Val, Ala, Glu, Tyr, Ile, Cys, Leu, Ser, Thr, Gly,Lys, Met, Asp, or Phe
XXXHXXPXXXXXXXXXXNXXXXXXXXN

SEQ ID NO: 26 - protein, 27 aa, synthetic construct
  REGION 1..27: note = HNH-like domain
  VARIANT 1: note = Xaa is Asp or Glu
  VARIANT 2: note = Xaa is Leu, Ile, Arg, Gln, Val, Met, or Lys
  VARIANT 3: note = Xaa is Asp or Glu
  VARIANT 5: note = Xaa is Ile, Val, Thr, Ala, or Leu
  VARIANT 6: note = Xaa is Val, Tyr, Ile, Leu, Phe, or Trp
  VARIANT 8: note = Xaa is Gln, His, Arg, Lys, Tyr, Ile, Leu, Phe, or Trp
  VARIANT 10: note = Xaa is Phe, Leu, Val, Lys, Tyr, Met, Ile, Arg, Ala, Glu, Asp, orGln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, orSer
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met,Gly, Tyr, or Val
  VARIANT 22: note = Xaa is Thr, Val, Cys, Glu, Ser, or Ala
  VARIANT 23: note = Xaa is Arg, Phe, Thr, Trp, Glu, Leu, Asn, Cys, Lys, Val, Ser,Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln,Arg, or Tyr
  VARIANT 26: note = Xaa is Lys, Val, Ala, Glu, Tyr, Ile, Cys, Leu, Ser, Thr, Gly,Lys, Met, Asp, or Phe
XXXHXXPXSXXXDDSXXNKVLXXXXXN

SEQ ID NO: 27 - protein, 27 aa, synthetic construct
  REGION 1..27: note = HNH-like domain
  VARIANT 1: note = Xaa is Asp or Glu
  VARIANT 3: note = Xaa is Asp or Glu
  VARIANT 8: note = Xaa is Gln, His, Arg, Lys, Tyr, Ile, Leu, or Trp
  VARIANT 10: note = Xaa is Phe, Leu, Val, Lys, Tyr, Met, Ile, Arg, Ala, Glu, Asp, orGln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, orSer
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met,Gly, Tyr, or Val
  VARIANT 23: note = Xaa is Arg, Phe, Thr, Trp, Glu, Leu, Asn, Cys, Lys, Val, Ser,Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln,Arg, or Tyr
  VARIANT 26: note = Xaa is Lys, Val, Ala, Glu, Tyr, Ile, Cys, Leu, Ser, Thr, Gly,Lys, Met, Asp, or Phe
XVXHIVPXSXXXDDSXXNKVLTXXXXN

SEQ ID NO: 28 - protein, 27 aa, synthetic construct
  REGION 1..27: note = HNH-like domain
  VARIANT 2: note = Xaa is Ile or Val
  VARIANT 6: note = Xaa is Ile or Val
  VARIANT 9: note = Xaa is Ala or Ser
  VARIANT 11: note = Xaa is Ile or Leu
  VARIANT 12: note = Xaa is Lys or Thr
  VARIANT 14: note = Xaa is Asp or Asn
  VARIANT 19: note = Xaa is Arg, Lys, or Leu
  VARIANT 22: note = Xaa is Thr or Val
  VARIANT 23: note = Xaa is Ser or Arg
  VARIANT 25: note = Xaa is Lys, Asp, or Ala
  VARIANT 26: note = Xaa is Glu, Lys, Gly, or Asn
DXDHIXPQXFXXDXSIDNXVLXXSXXN

SEQ ID NO: 29 - RNA, 116 nt, synthetic construct
  misc_feature 1..116: note = gRNA
  misc_feature 1..20: note = a, c, u, g, unknown or other
  misc_feature 1..20: note = targeting region
  misc_feature 21..42: note = first complementarity domain
  misc_feature 43..46: note = linking domain
  misc_feature 47..70: note = second complementarity domain
  source 1..116: mol_type = other RNA
nnnnnnnnnnnnnnnnnnnngttttagagctatgctgttttggaaacaaaacagcatagcaagttaaaataaggctagtccgttatcaacttgaaaaagtggcaccgagtcggtgc

SEQ ID NO: 30 - RNA, 116 nt, synthetic construct
  misc_feature 1..116: note = gRNA
  misc_feature 1..20: note = a, c, u, g, unknown or other
  misc_feature 1..20: note = targeting region
  misc_feature 21..42: note = first complementarity domain
  misc_feature 43..46: note = linking domain
  misc_feature 47..70: note = second complementarity domain
  source 1..116: mol_type = other RNA
nnnnnnnnnnnnnnnnnnnngtattagagctatgctgtattggaaacaatacagcatagcaagttaatataaggctagtccgttatcaacttgaaaaagtggcaccgagtcggtgc

SEQ ID NO: 31 - RNA, 96 nt, synthetic construct
  misc_feature 1..96: note = gRNA
  misc_feature 1..20: note = a, c, u, g, unknown or other
  misc_feature 1..20: note = targeting domain
  misc_feature 21..32: note = first complementarity domain
  misc_feature 33..36: note = linking domain
  misc_feature 37..50: note = second complementarity domain
  misc_feature 51..62: note = proximal domain
  source 1..96: mol_type = other RNA
nnnnnnnnnnnnnnnnnnnngtttaagagctagaaatagcaagtttaaataaggctagtccgttatcaacttgaaaaagtggcaccgagtcggtgc

SEQ ID NO: 32 - RNA, 47 nt, synthetic construct
  misc_feature 1..47: note = gRNA proximal and tail domains derived from S. pyogenes
  source 1..47: mol_type = other RNA
aaggctagtccgttatcaacttgaaaaagtggcaccgagtcggtgct

SEQ ID NO: 33 - RNA, 49 nt, synthetic construct
  misc_feature 1..49: note = gRNA proximal and tail domains
  source 1..49: mol_type = other RNA
aaggctagtccgttatcaacttgaaaaagtggcaccgagtcggtggtgc

SEQ ID NO: 34 - RNA, 51 nt, synthetic construct
  misc_feature 1..51: note = gRNA proximal and tail domains
  source 1..51: mol_type = other RNA
aaggctagtccgttatcaacttgaaaaagtggcaccgagtcggtgcggatc

SEQ ID NO: 35 - RNA, 31 nt, synthetic construct
  misc_feature 1..31: note = gRNA proximal and tail domains
  source 1..31: mol_type = other RNA
aaggctagtccgttatcaacttgaaaaagtg

SEQ ID NO: 36 - RNA, 18 nt, synthetic construct
  misc_feature 1..18: note = gRNA proximal and tail domains
  source 1..18: mol_type = other RNA
aaggctagtccgttatca

SEQ ID NO: 37 - RNA, 12 nt, synthetic construct
  misc_feature 1..12: note = gRNA proximal and tail domains
  source 1..12: mol_type = other RNA
aaggctagtccg

SEQ ID NO: 38 - RNA, 102 nt, synthetic construct
  misc_feature 1..102: note = Unimolecular gRNA derived from S. aureus
  misc_feature 1..20: note = n is a, c, g, or u
  misc_feature 1..20: note = Targeting domain
  source 1..102: mol_type = other RNA
nnnnnnnnnnnnnnnnnnnngttttagtactctggaaacagaatctactaaaacaaggcaaaatgccgtgtttatctcgtcaacttgttggcgagatttttt

SEQ ID NO: 39 - RNA, 42 nt, synthetic construct
  misc_feature 1..42: note = Modular gRNA derived from S. pyogenes
  misc_feature 1..20: note = a, c, u, g, unknown or other
  misc_feature 1..20: note = Targeting domain
  misc_feature 21..42: note = First complementarity domain
  source 1..42: mol_type = other RNA
nnnnnnnnnnnnnnnnnnnngttttagagctatgctgttttg

SEQ ID NO: 40 - RNA, 85 nt, synthetic construct
  misc_feature 1..85: note = Modular gRNA derived from S. pyogenes
  misc_feature 1..9: note = 5' extension domain
  misc_feature 10..33: note = Second complementarity domain
  misc_feature 34..45: note = Proximal domain
  misc_feature 46..85: note = Tail domain
  source 1..85: mol_type = other RNA
ggaaccattcaaaacagcatagcaagttaaaataaggctagtccgttatcaacttgaaaaagtggcaccgagtcggtgctttttt

SEQ ID NO: 41 - RNA, 62 nt, synthetic construct
  misc_feature 1..62: note = Unimolecular gRNA derived from S. pyogenes
  misc_feature 1..20: note = a, c, u, g, unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  source 1..62: mol_type = other RNA
nnnnnnnnnnnnnnnnnnnngttttagagctagaaatagcaagttaaaataaggctagtccg

SEQ ID NO: 42 - RNA, 102 nt, synthetic construct
  misc_feature 1..102: note = Unimolecular gRNA derived from S. pyogenes
  misc_feature 1..20: note = a, c, u, g, unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  misc_feature 63..102: note = Tail domain
  source 1..102: mol_type = other RNA
nnnnnnnnnnnnnnnnnnnngttttagagctagaaatagcaagttaaaataaggctagtccgttatcaacttgaaaaagtggcaccgagtcggtgctttttt

SEQ ID NO: 43 - RNA, 75 nt, synthetic construct
  misc_feature 1..75: note = Unimolecular gRNA derived from S. pyogenes
  misc_feature 1..20: note = Targeting domain
  misc_feature 1..20: note = a, c, u, g, unknown or other
  misc_feature 21..36: note = First complementarity domain
  misc_feature 37..40: note = Linking domain
  misc_feature 41..58: note = Second complementarity domain
  misc_feature 59..70: note = Proximal domain
  misc_feature 71..75: note = Tail domain
  source 1..75: mol_type = other RNA
nnnnnnnnnnnnnnnnnnnngttttagagctatgctgaaaagcatagcaagttaaaataaggctagtccgttatc

SEQ ID NO: 44 - RNA, 87 nt, synthetic construct
  misc_feature 1..87: note = Unimolecular gRNA derived from S. pyogenes
  misc_feature 1..20: note = a, c, u, g, unknown or other
  misc_feature 1..20: note = Targeting domain
  misc_feature 21..32: note = First complementarity domain
  misc_feature 43..46: note = Linking domain
  misc_feature 57..70: note = Second complementarity domain
  misc_feature 71..82: note = Proximal domain
  misc_feature 83..87: note = Tail domain
  source 1..87: mol_type = other RNA
nnnnnnnnnnnnnnnnnnnngttttagagctatgctgttttggaaacaaaacagcatagcaagttaaaataaggctagtccgttatc

SEQ ID NO: 45 - RNA, 42 nt, synthetic construct
  misc_feature 1..42: note = Modular gRNA derived from S. thermophilus
  misc_feature 1..20: note = a, c, u, g, unknown or other
  misc_feature 1..20: note = Targeting domain
  misc_feature 21..42: note = First complementarity domain
  source 1..42: mol_type = other RNA
nnnnnnnnnnnnnnnnnnnngttttagagctgtgttgtttcg

SEQ ID NO: 46 - RNA, 78 nt, synthetic construct
  misc_feature 1..78: note = Modular gRNA derived from S. thermophilus
  misc_feature 1..3: note = 5' extension domain
  misc_feature 4..27: note = Second complementarity domain
  misc_feature 28..40: note = Proximal domain
  misc_feature 41..78: note = Tail domain
  source 1..78: mol_type = other RNA
gggcgaaacaacacagcgagttaaaataaggcttagtccgtactcaacttgaaaaggtggcaccgattcggtgttttt

SEQ ID NO: 47 - RNA, 85 nt, synthetic construct
  misc_feature 1..85: note = Modular gRNA derived from S. pyogenes
  source 1..85: mol_type = other RNA
gaaccattcaaaacagcatagcaagttaaaataaggctagtccgttatcaacttgaaaaagtggcaccgagtcggtgcttttttt

SEQ ID NO: 48 - RNA, 96 nt, synthetic construct
  misc_feature 1..96: note = gRNA from S. pyogenes
  misc_feature 1..20: note = n is a, c, g, or u
  misc_feature 1..20: note = Targeting domain
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 49 - RNA, 96 nt, synthetic construct
  misc_feature 1..96: note = gRNA
  misc_feature 1..20: note = n is a, c, g, or u
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  source 1..96: mol_type = other RNA
nnnnnnnnnnnnnnnnnnnngtattagagctagaaatagcaagttaatataaggctagtccgttatcaacttgaaaaagtggcaccgagtcggtgc

SEQ ID NO: 50 - RNA, 104 nt, synthetic construct
  misc_feature 1..104: note = gRNA
  misc_feature 1..20: note = n is a, c, g, or u
  misc_feature 1..20: note = Targeting domain
  misc_feature 21..36: note = First complementarity domain
  misc_feature 37..40: note = Linking domain
  misc_feature 41..58: note = Second complementarity domain
  source 1..104: mol_type = other RNA
nnnnnnnnnnnnnnnnnnnngttttagagctatgctgaaaagcatagcaagttaaaataaggctagtccgttatcaacttgaaaaagtggcaccgagtcggtgc

SEQ ID NO: 51 - RNA, 106 nt, synthetic construct
  misc_feature 1..106: note = gRNA
  misc_feature 1..20: note = n is a, c, g, or u
  misc_feature 1..20: note = Targeting domain
  misc_feature 21..37: note = First complementarity domain
  misc_feature 38..41: note = Linking domain
  misc_feature 42..60: note = Second complementarity domain
  source 1..106: mol_type = other RNA
nnnnnnnnnnnnnnnnnnnngttttagagctatgctggaaacagcatagcaagttaaaataaggctagtccgttatcaacttgaaaaagtggcaccgagtcggtgc

SEQ ID NO: 52 - protein, 12 aa, Peptoniphilus duerdenii
DIGTASVGWAVT

SEQ ID NO: 53 - protein, 12 aa, Treponema denticola
DVGTGSVGWAVT

SEQ ID NO: 54 - protein, 12 aa, Streptococcus mutans
DIGTNSVGWAVV

SEQ ID NO: 55 - protein, 12 aa, Streptococcus pyogenes
DIGTNSVGWAVI

SEQ ID NO: 56 - protein, 12 aa, Listeria innocua
DIGTNSVGWAVL

SEQ ID NO: 57 - protein, 12 aa, Flavobacterium branchiophilum
DLGTNSIGWAVV

SEQ ID NO: 58 - protein, 11 aa, Pedobacter glucosidilyticus
DLGTNSIGWAI

SEQ ID NO: 59 - protein, 12 aa, Bacteroides fragilis
DLGTNSIGWALV

SEQ ID NO: 60 - protein, 12 aa, Fusobacterium nucleatum
DIGTNSVGWCVT

SEQ ID NO: 61 - protein, 12 aa, Acidaminococcus sp.
DIGTNSVGYAVT

SEQ ID NO: 62 - protein, 12 aa, Coprococcus catus
DMGTGSLGWAVT

SEQ ID NO: 63 - protein, 12 aa, Oenococcus kitaharae
DIGTSSVGWAAI

SEQ ID NO: 64 - protein, 12 aa, Catenibacterium mitsuokai
DLGTGSVGWAVV

SEQ ID NO: 65 - protein, 12 aa, Mycoplasma gallisepticum
DLGVGSVGWAIV

SEQ ID NO: 66 - protein, 12 aa, Mycoplasma ovipneumoniae
DLGIASIGWAII

SEQ ID NO: 67 - protein, 12 aa, Mycoplasma canis
DLGIASVGWAIV

SEQ ID NO: 68 - protein, 12 aa, Mycoplasma synoviae
DLGVASVGWSIV

SEQ ID NO: 69 - protein, 12 aa, Eubacterium rectale
DIGIASVGWAIL

SEQ ID NO: 70 - protein, 12 aa, Enterococcus faecalis
DLGISSVGWSVI

SEQ ID NO: 71 - protein, 12 aa, Ilyobacter polytropus
DIGIASVGWSVI

SEQ ID NO: 72 - protein, 12 aa, Ruminococcus albus
DVGIGSIGWAVI

SEQ ID NO: 73 - protein, 12 aa, Elusimicrobium minutum
DLGVGSIGFAIV

SEQ ID NO: 74 - protein, 12 aa, Akkermansia muciniphila
DIGYASIGWAVI

SEQ ID NO: 75 - protein, 12 aa, Prevotella ruminicola
DTGTNSLGWAIV

SEQ ID NO: 76 - protein, 12 aa, synthetic construct
DLGTNSIGWCLL

SEQ ID NO: 77 - protein, 12 aa, Rhodospirillum rubrum
DIGTDSLGWAVF

SEQ ID NO: 78 - protein, 12 aa, Lactobacillus rhamnosus
DIGSNSIGFAVV

SEQ ID NO: 79 - protein, 12 aa, Sphaerochaeta globosa
DLGVGSIGVAVA

SEQ ID NO: 80 - protein, 12 aa, Rhodopseudomonas palustris
DLGIASCGWGVV

SEQ ID NO: 81 - protein, 12 aa, Mycoplasma mobile
DLGIASVGWCLT

SEQ ID NO: 82 - protein, 12 aa, Streptococcus thermophilus
DIGIGSVGVGIL

SEQ ID NO: 83 - protein, 12 aa, Staphylococcus lugdunensis
DIGITSVGYGLI

SEQ ID NO: 84 - protein, 12 aa, Eubacterium dolichum
DIGITSVGFGII

SEQ ID NO: 85 - protein, 12 aa, Lactobacillus coryniformis
DVGITSTGYAVL

SEQ ID NO: 86 - protein, 12 aa, Nitratifractor salsuginis
DLGITSFGYAIL

SEQ ID NO: 87 - protein, 12 aa, Bifidobacterium bifidum
DIGNASVGWSAF

SEQ ID NO: 88 - protein, 12 aa, Lactobacillus gasseri
DVGTNSCGWVAM

SEQ ID NO: 89 - protein, 12 aa, Acidothermus cellulolyticus
DVGERSIGLAAV

SEQ ID NO: 90 - protein, 12 aa, Bifidobacterium longum
DVGLNSVGLAAV

SEQ ID NO: 91 - protein, 12 aa, Bifidobacterium dentium
DVGLMSVGLAAI

SEQ ID NO: 92 - protein, 12 aa, Corynebacterium diphtheriae
DVGTFSVGLAAI

SEQ ID NO: 93 - protein, 12 aa, Staphylococcus pseudintermedius
DIGTGSVGYACM

SEQ ID NO: 94 - protein, 12 aa, Capnocytophaga ochracea
DLGTTSIGFAHI

SEQ ID NO: 95 - protein, 12 aa, Prevotella denticola
DLGTNSIGSSVR

SEQ ID NO: 96 - protein, 12 aa, Ralstonia solanacearum
DIGTNSIGWAVI

SEQ ID NO: 97 - protein, 12 aa, Pasteurella multocida
DLGIASVGWAVV

SEQ ID NO: 98 - protein, 12 aa, Comamonas granuli
DIGIASVGWAVL

SEQ ID NO: 99 - protein, 12 aa, Helicobacter mustelae
DIGIASIGWAVI

SEQ ID NO: 100 - protein, 12 aa, Agathobacter rectalis
DIGIASVGWAII

SEQ ID NO: 101 - protein, 12 aa, Clostridium cellulolyticum
DVGIASVGWAVI

SEQ ID NO: 102 - protein, 11 aa, Methylophilus sp.
DIGIASVGWAL

SEQ ID NO: 103 - protein, 12 aa, Neisseria meningitidis
DIGIASVGWAMV

SEQ ID NO: 104 - protein, 12 aa, Clostridium perfringens
DIGITSVGWAVI

SEQ ID NO: 105 - protein, 12 aa, Wolinella succinogenes
DLGISSLGWAIV

SEQ ID NO: 106 - protein, 12 aa, Azospirillum sp.
DLGTNSIGWGLL

SEQ ID NO: 107 - protein, 12 aa, Verminephrobacter eiseniae
DLGSTSLGWAIF

SEQ ID NO: 108 - protein, 12 aa, Campylobacter jejuni
DIGISSIGWAFS

SEQ ID NO: 109 - protein, 12 aa, Parvibaculum lavamentivorans
DIGTTSIGFSVI

SEQ ID NO: 110 - protein, 12 aa, Dinoroseobacter shibae
DIGTSSIGWWLY

SEQ ID NO: 111 - protein, 12 aa, Nitrobacter hamburgensis
DLGSNSLGWFVT

SEQ ID NO: 112 - protein, 12 aa, Bradyrhizobium sp.
DLGANSLGWFVV

SEQ ID NO: 113 - protein, 15 aa, Bacillus cereus
DIGLRIGITSCGWSI

SEQ ID NO: 114 - protein, 12 aa, Sutterella wadsworthensis
DMGAKYTGVFYA

SEQ ID NO: 115 - protein, 12 aa, Wolinella succinogenes
DLGGKNTGFFSF

SEQ ID NO: 116 - protein, 12 aa, Francisella tularensis
DLGVKNTGVFSA

SEQ ID NO: 117 - protein, 12 aa, Gamma proteobacterium
DLGAKFTGVALY

SEQ ID NO: 118 - protein, 12 aa, Legionella pneumophila
DLGGKFTGVCLS

SEQ ID NO: 119 - protein, 12 aa, Parasutterella excrementihominis
DLGGTYTGTFIT

SEQ ID NO: 120 - protein, 12 aa, Streptococcus thermophilus
DIGTNSVGWAVT

SEQ ID NO: 121 - protein, 12 aa, Eubacterium yurii
DVGTNSVGWAVT

SEQ ID NO: 122 - protein, 12 aa, Butyrivibrio hungatei
DMGTNSVGWAVT

SEQ ID NO: 123 - protein, 12 aa, Solobacterium moorei
DVGTSSVGWAVT

SEQ ID NO: 124 - protein, 27 aa, Treponema denticola
DIDHIYPQSKIKDDSISNRVLVCSSCN

SEQ ID NO: 125 - protein, 27 aa, Coprococcus catus
DIDHIYPQSKTMDDSLNNRVLVKKNYN

SEQ ID NO: 126 - protein, 27 aa, Peptoniphilus duerdenii
DQDHIYPKSKIYDDSLENRVLVKKNLN

SEQ ID NO: 127 - protein, 27 aa, Catenibacterium mitsuokai
QIDHIVPQSLVKDDSFDNRVLVVPSEN

SEQ ID NO: 128 - protein, 27 aa, Streptococcus mutans
DIDHIIPQAFIKDNSIDNRVLTSSKEN

SEQ ID NO: 129 - protein, 27 aa, Streptococcus thermophilus
DIDHIIPQAFLKDNSIDNKVLVSSASN

SEQ ID NO: 130 - protein, 27 aa, Oenococcus kitaharae
DIDHIIPQAYTKDNSLDNRVLVSNITN

SEQ ID NO: 131 - protein, 27 aa, Listeria innocua
DIDHIVPQSFITDNSIDNLVLTSSAGN

SEQ ID NO: 132 - protein, 27 aa, Streptococcus pyogenes
DVDHIVPQSFLKDDSIDNKVLTRSDKN

SEQ ID NO: 133 - protein, 27 aa, Acidaminococcus sp.
NIDHIYPQSMVKDDSLDNKVLVQSEIN

SEQ ID NO: 134 - protein, 27 aa, Lactobacillus rhamnosus
DIDHILPQSLIKDDSLDNRVLVNATIN

SEQ ID NO: 135 - protein, 27 aa, Lactobacillus gasseri
DIDHILPQSFIKDDSLENRVLVKKAVN

SEQ ID NO: 136 - protein, 27 aa, Staphylococcus pseudintermedius
EVDHIFPRSFIKDDSIDNKVLVIKKMN

SEQ ID NO: 137 - protein, 27 aa, Olsenella uli
EVDHIIPRSYIKDDSFENKVLVYREEN

SEQ ID NO: 138 - protein, 27 aa, Bifidobacterium bifidum
DIDHIIPQAVTQNDSIDNRVLVARAEN

SEQ ID NO: 139 - protein, 27 aa, Mycoplasma gallisepticum
EIDHIIPYSISFDDSSSNKLLVLAESN

SEQ ID NO: 140 - protein, 27 aa, Mycoplasma canis
EIDHIIPYSLCFDDSSANKVLVHKQSN

SEQ ID NO: 141 - protein, 27 aa, Ilyobacter polytropus
DIDHIIPYSRSMDDSYSNKVLVLSGEN

SEQ ID NO: 142 - protein, 27 aa, synthetic construct
DIDHIIPYSKSMDDSFNNKVLCLAEEN

SEQ ID NO: 143 - protein, 27 aa, Campylobacter jejuni
EIDHIYPYSRSFDDSYMNKVLVFTKQN

SEQ ID NO: 144 - protein, 27 aa, Clostridium cellulolyticum
QIDHIYPYSRSMDDSYMNKVLVLTDEN

SEQ ID NO: 145 - protein, 27 aa, Clostridium perfringens
EIDHIIPFSRSFDDSLSNKILVLGSEN

SEQ ID NO: 146 - protein, 27 aa, Neisseria meningitidis
EIDHALPFSRTWDDSFNNKVLVLGSEN

SEQ ID NO: 147 - protein, 27 aa, Pasteurella multocida
EIDHALPFSRTWDDSFNNKVLVLASEN

SEQ ID NO: 148 - protein, 27 aa, Enterococcus faecalis
EIDHIIPISISLDDSINNKVLVLSKAN

SEQ ID NO: 149 - protein, 27 aa, Eubacterium dolichum
EVDHIIPISISLDDSITNKVLVTHREN

SEQ ID NO: 150 - protein, 27 aa, Acidovorax ebreus
QVDHALPYSRSYDDSKNNKVLVLTHEN

SEQ ID NO: 151 - protein, 27 aa, Streptococcus thermophilus
EVDHILPLSITFDDSLANKVLVYATAN

SEQ ID NO: 152 - protein, 27 aa, Eubacterium rectale
EIDHIIPRSISFDDARSNKVLVYRSEN

SEQ ID NO: 153 - protein, 27 aa, Staphylococcus lugdunensis
EVDHIIPRSVSFDNSYHNKVLVKQSEN

SEQ ID NO: 154 - protein, 27 aa, Roseburia intestinalis
DIDHILPYSITFDDSFRNKVLVTSQEN

SEQ ID NO: 155 - protein, 27 aa, Wolinella succinogenes
EIDHILPRSRSADDSFANKVLCLARAN

SEQ ID NO: 156 - protein, 27 aa, synthetic construct
EIEHLLPFSLTLDDSMANKTVCFRQAN

SEQ ID NO: 157 - protein, 27 aa, Azospirillum sp.
DIDHILPFSVSLDDSAANKVVCLREAN

SEQ ID NO: 158 - protein, 27 aa, Bradyrhizobium sp.
DIDHLIPFSISWDDSAANKVVCMRYAN

SEQ ID NO: 159 - protein, 27 aa, Nitrobacter hamburgensis
DIDHILPVAMTLDDSPANKIICMRYAN

SEQ ID NO: 160 - protein, 27 aa, Dinoroseobacter shibae
DVDHILPYSRTLDDSFPNRTLCLREAN

SEQ ID NO: 161 - protein, 27 aa, Verminephrobacter eiseniae
EIEHILPFSRTLDDSLNNRTVAMRRAN

SEQ ID NO: 162 - protein, 27 aa, Lactobacillus coryniformis
EVDHIIPYSISWDDSYTNKVLTSAKCN

SEQ ID NO: 163 - protein, 27 aa, Rhodopseudomonas palustris
QVDHILPWSRFGDDSYLNKTLCTARSN

SEQ ID NO: 164 - protein, 27 aa, Ralstonia syzygii
QVDHILPFSKTLDDSFANKVLAQHDAN

SEQ ID NO: 165 - protein, 27 aa, Helicobacter mustelae
QIDHAFPLSRSLDDSQSNKVLCLTSSN

SEQ ID NO: 166 - protein, 27 aa, Mycoplasma mobile
DIDHIVPRSISFDDSFSNLVIVNKLDN

SEQ ID NO: 167 - protein, 27 aa, Mycoplasma ovipneumoniae
EIEHIIPYSMSYDNSQANKILTEKAEN

SEQ ID NO: 168 - protein, 27 aa, Mycoplasma synoviae
EIDHVIPYSKSADDSWFNKLLVKKSTN

SEQ ID NO: 169 - protein, 27 aa, Aminomonas paucivorans
EMDHILPYSRSLDNGWHNRVLVHGKDN

SEQ ID NO: 170 - protein, 27 aa, Ruminococcus albus
EVDHIVPYSLILDNTINNKALVYAEEN

SEQ ID NO: 171 - protein, 27 aa, Fibrobacter succinogenes
EIEHVIPQSLYFDDSFSNKVICEAEVN

SEQ ID NO: 172 - protein, 27 aa, Bacteroides fragilis
DIEHIIPQARLFDDSFSNKTLEARSVN

SEQ ID NO: 173 - protein, 27 aa, Capnocytophaga sputigena
EIEHIVPKARVFDDSFSNKTLTFHRIN

SEQ ID NO: 174 - protein, 28 aa, Finegoldia magna
DKDHIIPQSMKKDDSIINNLVLVNKNAN

SEQ ID NO: 175 - protein, 27 aa, Parvibaculum lavamentivorans
EVEHIWPRSRSFDNSPRNKTLCRKDVN

SEQ ID NO: 176 - protein, 27 aa, Bacillus cereus
IVNHIIPYNRSFDDTYHNRVLTLTETK

SEQ ID NO: 177 - protein, 27 aa, Prevotella micans
DMEHTIPKSISFDNSDQNLTLCESYYN

SEQ ID NO: 178 - protein, 27 aa, Prevotella ruminicola
DIEHTIPRSAGGDSTKMNLTLCSSRFN

SEQ ID NO: 179 - protein, 27 aa, Flavobacterium columnare
DIEHTIPRSISQDNSQMNKTLCSLKFN

SEQ ID NO: 180 - protein, 27 aa, Rhodospirillum rubrum
DIDHVIPLARGGRDSLDNMVLCQSDAN

SEQ ID NO: 181 - protein, 27 aa, Elusimicrobium minutum
DIEHLFPIAESEDNGRNNLVISHSACN

SEQ ID NO: 182 - protein, 27 aa, Sphaerochaeta globosa
DVDHIFPRDDTADNSYGNKVVAHRQCN

SEQ ID NO: 183 - protein, 27 aa, Nitratifractor salsuginis
DIEHIVPQSLGGLSTDYNTIVTLKSVN

SEQ ID NO: 184 - protein, 27 aa, Acidothermus cellulolyticus
ELDHIVPRTDGGSNRHENLAITCGACN

SEQ ID NO: 185 - protein, 28 aa, Bifidobacterium longum
EMDHIVPRKGVGSTNTRTNFAAVCAECN

SEQ ID NO: 186 - protein, 28 aa, Bifidobacterium dentium
EMDHIVPRKGVGSTNTRVNLAAACAACN

SEQ ID NO: 187 - protein, 28 aa, Corynebacterium diphtheriae
EMDHIVPRAGQGSTNTRENLVAVCHRCN

SEQ ID NO: 188 - protein, 33 aa, Sutterella wadsworthensis
EIDHILPRSLIKDARGIVFNAEPNLIYASSRGN

SEQ ID NO: 189 - protein, 33 aa, Gamma proteobacterium
EIDHIIPRSLTGRTKKTVFNSEANLIYCSSKGN

SEQ ID NO: 190 - protein, 33 aa, Parasutterella excrementihominis
EIDHIIPRSLTLKKSESIYNSEVNLIFVSAQGN

SEQ ID NO: 191 - protein, 33 aa, Legionella pneumophila
EIDHIYPRSLSKKHFGVIFNSEVNLIYCSSQGN

SEQ ID NO: 192 - protein, 33 aa, Wolinella succinogenes
EIDHILPRSHTLKIYGTVFNPEGNLIYVHQKCN

SEQ ID NO: 193 - protein, 30 aa, Francisella tularensis
ELDHIIPRSHKKYGTLNDEANLICVTRGDN

SEQ ID NO: 194 - protein, 27 aa, Akkermansia muciniphila
ELEHIVPHSFRQSNALSSLVLTWPGVN

SEQ ID NO: 195 - protein, 27 aa, Solobacterium moorei
DIDHIYPRSKIKDDSITNRVLVEKDIN

SEQ ID NO: 196 - protein, 28 aa, Veillonella atypica
YDIDHIYPRSLTKDDSFDNLVLCERTAN

SEQ ID NO: 197 - protein, 28 aa, Fusobacterium nucleatum
DIDHIYPRSKVIKDDSFDNLVLVLKNEN

SEQ ID NO: 198 - protein, 27 aa, Filifactor alocis
DRDHIYPQSKIKDDSIDNLVLVNKTYN

SEQ ID NO: 199-325 - skipped (127 entries)

SEQ ID NO: 326 - RNA, 17 nt, synthetic construct
  misc_feature 1..17: note = Targeting domain
  source 1..17: mol_type = other RNA
aaggctggccaacccat

SEQ ID NO: 327-358 - skipped (32 entries)

SEQ ID NO: 359 - RNA, 20 nt, synthetic construct
  misc_feature 1..20: note = Targeting domain
  source 1..20: mol_type = other RNA
tagtcttagagtatccagtg

SEQ ID NO: 360-901 - skipped (542 entries)

SEQ ID NO: 902 - DNA, 4758 bp, Homo sapiens
  misc_feature 2716..2719: note = HPFH deletion site (4 bp del -225 to -222)
  misc_feature 2748..2753: note = GATA1 binding motif
  misc_feature 2762..2767: note = GATA1 binding motif
  misc_feature 2791..2799: note = FKLF transcription factor binding motif
  misc_feature 2823..2830: note = CP1/Coup TFII binding motif
  misc_feature 2824..2836: note = HPFH deletion site (13 bp del -114 to -102)
  misc_feature 2991..2993: note = Start codon
  source 1..4758: mol_type = other DNA
tttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagcttctagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggagctcatgagagcagatccccatcatagaattattttcctcatcagaagcagagagattagccatttctcttccttctggtgaggacacagtgggaagtcagccacctgcaacccaggaagagagccctgaccaggaaccagcagaaaagtgagaaaaaatcctgttgttgaagtcacccagtctatgctattttgttatagcaccttgcactaagtaaggcagatgaagaaagagaaaaaaataagcttcggtgttcagtggattagaaaccatgtttatctcaggtttacaaatctccacttgtcctctgtgtttcagaataaaataccaactctactactctcatctgtaagatgcaaatagtaagcctgagcccttctgtctaactttgaattctattttttcttcaacgtactttaggcttgtaatgtgtttatatacagtgaaatgtcaagttctttctttatatttctttctttcttttttttcctcagcctcagagttttccacatgcccttcctactttcaggaacttctttctccaaacgtcttctgcctggctccatcaaatcataaaggacccacttcaaatgccatcactcactaccatttcacaattcgcactttctttctttgtcctttttttttttagtaaaacaagtttataaaaaattgaaggaataaatgaatggctacttcataggcagagtagacgcaagggctactggttgccgatttttattgttatttttcaatagtatgctaaacaaggggtagattatttatgctgcccatttttagaccataaaagataacttcctgatgttgccatggcatttttttccttttaattttatttcatttcattttaatttcgaaggtacatgtgcaggatgtgcaggcttgttacatgggtaaatgtgtgtctttctggccttttagccatctgtatcaatgagcagatataagctttacacaggatcatgaaggatgaaagaatttcaccaatattataataatttcaatcaacctgatagcttaggggataaactaatttgaagatacagcttgcctccgataagccagaattccagagcttctggcattataatctagcaaggttagagatcatggatcactttcagagaaaaacaaaaacaaactaaccaaaagcaaaacagaaccaaaaaaccaccataaatacttcctaccctgttaatggtccaatatgtcagaaacagcactgtgttagaaataaagctgtctaaagtacactaatattcgagttataatagtgtgtggactattagtcaataaaaacaacccttgcctctttagagttgttttccatgtacacgcacatcttatgtcttagagtaagattccctgagaagtgaacctagcatttatacaagataattaattctaatccacagtacctgccaaagaacattctaccatcatctttactgagcatagaagagctacgccaaaaccctgggtcatcagccagcacacacacttatccagtggtaaatacacatcatctggtgtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggctttatagaaattgttttcactgcactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaaggtgagtccaggagatgtttcagccctgttgcctttagtctcgaggcaacttagacaacggagtattgatctgagcacagcagggtgtgagctgtttgaagatactggggttgggggtgaagaaactgcagaggactaactgggctgagacccagtggtaatgttttagggcctaaggagtgcctctaaaaatctagatggacaattttgactttgagaaaagagaggtggaaatgaggaaaatgacttttctttattagattccagtagaaagaactttcatctttccctcatttttgttgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgcgtgtgtgtgtgtgtgtcagcgtgtgtttcttttaacgtcttcagcctacaacatacagggttcatggtggcaagaagatagcaagatttaaattatggccagtgactagtgcttgaaggggaacaactacctgcatttaatgggaaggcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgtaggagaaatccg

SEQ ID NO: 903 - DNA, 4773 bp, Homo sapiens
  misc_feature 1233..1238: note = GATA1 binding site
  misc_feature 2672..2677: note = GATA1 binding site
  misc_feature 2686..2691: note = GATA1 binding site
  misc_feature 2715..2723: note = FKLF transcription factor binding motif
  misc_feature 2747..2754: note = CP1/Coup TFII binding motif
  misc_feature 2748..2760: note = HPFH deletion site (13 bp del -114 to -102)
  misc_feature 2915..2917: note = Start codon
  source 1..4773: mol_type = other DNA
ttatgtcattaccagagttaaaattctataatggcttctcactccctaccactgaggacaagtttatgtccttaggtttatgcttccctgaaacaataccacctgctattctccactttacatatcaacggcactggttctttatctaactctctggcacagcaggagtttgttttcttctgcttcagagctttgaatttactatttcagcttctaaactttatttggcaatgccttcccatggcagattccttctgtcattttgcctctgttcgaatactttctccttaatttcattcttagttaataatatctgaaattattttgttgtttaacttaattattaattttatgtatgttctacctagattataatcttcagaggaaagttttattctctgacttatttaacttaaatgcccactactttaaaaattatgacatttatttaacagatatttgctgaacaaatgtttgaaaatacatgggaaagaatgcttgaaaacacttgaaattgcttgtgtaaagaaacagttttatcagttaggatttaatcaatgtcagaagcaatgatataggaaaaatcgaggaataagacagttatggataaggagaaatcaacaaactcttaaaagatattgcctcaaaagcataagaggaaataagggtttatacatgacttttagaacactgccttggtttttggataaatggggaagttgtttgaaaacaggagggatcctagatattccttagtctgaggaggagcaattaagattcacttgtttagaggctgggagtggtggctcacgcctgtaatcccagaattttgggaggccaaggcaggcagatcacctgaggtcaagagttcaagaccaacctggccaacatggtgaaatcccatctctacaaaaatacaaaaattagacaggcatgatggcaagtgcctgtaatcccagctacttgggaggctgaggaaggagaattgcttgaacctggaaggcaggagttgcagtgagccgagatcataccactgcactccagcctgggtgacagaacaagactctgtctcaaaaaaaaaaaagagagattcaaaagattcacttgtttaggccttagcgggcttagacaccagtctctgacacattcttaaaggtcaggctctacaaatggaacccaaccagactctcagatatggccaaagatctatacacacccatctcacagatcccctatcttaaagagaccctaatttgggttcacctcagtctctataatctgtaccagcataccaataaaaatctttctcacccatccttagattgagagaagtcacttattattatgtgagtaactggaagatactgataagttgacaaatctttttctttcctttcttattcaacttttattttaacttccaaagaacaagtgcaatatgtgcagctttgttgcgcaggtcaacatgtatctttctggtcttttagccgcctaacactttgagcagatataagccttacacaggattatgaagtctgaaaggattccaccaatattattataattcctatcaacctgataggttaggggaaggtagagctctcctccaataagccagatttccagagtttctgacgtcataatctaccaaggtcatggatcgagttcagagaaaaaacaaaagcaaaaccaaacctaccaaaaaataaaaatcccaaagaaaaaataaagaaaaaaacagcatgaatacttcctgccatgttaagtggccaatatgtcagaaacagcactgagttacagataaagatgtctaaactacagtgacatcccagctgtcacagtgtgtggactattagtcaataaaacagtccctgcctcttaagagttgttttccatgcaaatacatgtcttatgtcttagaataagattccctaagaagtgaacctagcatttatacaagataattaattctaatccatagtatctggtaaagagcattctaccatcatctttaccgagcatagaagagctacaccaaaaccctgggtcatcagccagcacatacacttatccagtgataaatacacatcatcgggtgcctacatacatacctgaatataaaaaaaatacttttgctgagatgaaacaggcgtgatttatttcaaataggtacggataagtagatattgaagtaaggattcagtcttatattatattacataacattaatctattcctgcactgaaactgttgctttataggatttttcactacactaatgagaacttaagagataatggcctaaaaccacagagagtatattcaaagataagtatagcacttcttatttggaaaccaatgcttactaaatgagactaagacgtgtcccatcaaaaatcctggacctatgcctaaaacacatttcacaatccctgaacttttcaaaaattggtacatgctttaactttaaactacaggcctcactggagctacagacaagaaggtgaaaaacggctgacaaaagaagtcctggtatcttctatggtgggagaagaaaactagctaaagggaagaataaattagagaaaaattggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacacatggttgtcttcagttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaa

SEQ ID NO: 904-916 - skipped (13 entries)

SEQ ID NO: 917 - DNA, 20 bp, synthetic construct
  misc_feature 1..20: note = targeting domain
  source 1..20: mol_type = other DNA
cttgtcaaggctattggtca

SEQ ID NO: 918-940 - skipped (23 entries)

SEQ ID NO: 941 - RNA, 20 nt, synthetic construct
  misc_feature 1..20: note = targeting domain
  source 1..20: mol_type = other RNA
ggcaaggctggccaacccat

SEQ ID NO: 942-943 - skipped (2 entries)

SEQ ID NO: 944 - DNA, 20 bp, synthetic construct
  misc_feature 1..20: note = targeting domain
  source 1..20: mol_type = other DNA
ggcaaggctggccaacccat

SEQ ID NO: 945-967 - skipped (23 entries)

SEQ ID NO: 968 - DNA, 115 bp, synthetic construct
  misc_feature 1..115: note = □DHS enhancer
  source 1..115: mol_type = other DNA
caactttgaagctagtctagtgcaagctaacagttgcttttatcacaggctccaggaagggtttggcctctgattagggtgggggcgtgggtggggtagaagaggactggcagac

SEQ ID NO: 969 - DNA, 801 bp, synthetic construct
  misc_feature 1..801: note = □DHS enhancer region including ˜500 bp upstream and ˜200 bpdownstream
  source 1..801: mol_type = other DNA
aggtaccttttgtgtgtatgtgctgattgagggcccattgagaatattttgacttttagggaagctccaaactctcaaaccacagggatcacaacacatacgtgtgtctgttatgacgttatatgtaagcatcacaacaggcagagaatgtctgcaccccaccctggaaaacagcctgactgtgccccatgggcaaaccagactagtttatagggggttctactctgaggtactgatggaccttgggtgctattcctgtgataaggaaggcagctagacaggacttgggagttatctgtagtgagatggctgaaaagcgatacagggctggctctatgccccaggtgtgcataagtaagagcagatagctgattccagtgcaaagtccatacaggtaataacataggccagaaaagagatatggcatctactcttagacataacacaccagggtcaatacaactttgaagctagtctagtgcaagctaacagttgcttttatcacaggctccaggaagggtttggcctctgattagggtgggggcgtgggtggggtagaagaggactggcagacctctccatcggtggccgtttgcccaggggggcctctttcggaaggctctcttggtgatggagaattggattttatttctcaatgggaatgaaataatttgtatgccatgccgtgtggactcccaaaattgtaaaggaggtgaagcttcccctgtctgcactctcccctcctcataattgtccatttttcatctgtcgggctgtccacccatccatcacatataggca

SEQ ID NO: 970-973 - skipped (4 entries)

SEQ ID NO: 974 - DNA, 180 bp, synthetic construct
  misc_feature 1..180: note = Ptx ssODN 18nt - positive strand (OLI16409)
  misc_feature 1..2: note = Linked by phosphorothioate (PS) bond
  misc_feature 179..180: note = Linked by phosphorothioate (PS) bond
  source 1..180: mol_type = other DNA
ggtgcttccttttattcttcatccctagccagccgccggcccctggcctcactggatactctaagactattggtcaagtttgccttgtcaaggctggccaacccatgggtggagtttagccagggaccgtttcagacagatatttgcattgagatagtgtggggaaggggcccccaagag

SEQ ID NO: 975 - DNA, 180 bp, synthetic construct
  misc_feature 1..180: note = Ptx ssODN 18nt - negative strand (OLI16410)
  misc_feature 1..2: note = Linked by phosphorothioate (PS) bond
  misc_feature 179..180: note = Linked by phosphorothioate (PS) bond
  source 1..180: mol_type = other DNA
ctcttgggggccccttccccacactatctcaatgcaaatatctgtctgaaacggtccctggctaaactccacccatgggttggccagccttgacaaggcaaacttgaccaatagtcttagagtatccagtgaggccaggggccggcggctggctagggatgaagaataaaaggaagcacc

SEQ ID NO: 976 - DNA, 180 bp, synthetic construct
  misc_feature 1..180: note = Ptx ssODN 11nt - negative strand (OLI16411)
  misc_feature 1..2: note = Linked by phosphorothioate (PS) bond
  misc_feature 179..180: note = Linked by phosphorothioate (PS) bond
  source 1..180: mol_type = other DNA
ggccccttccccacactatctcaatgcaaatatctgtctgaaacggtccctggctaaactccacccatgggttggccagccttgccttgagacaaggcaaacttgaccaatagtcttagagtatccagtgaggccaggggccggcggctggctagggatgaagaataaaaggaagcaccc

SEQ ID NO: 977 - DNA, 180 bp, synthetic construct
  misc_feature 1..180: note = Ptx ssODN 13nt - negative strand (OLI16412)
  misc_feature 1..2: note = Linked by phosphorothioate (PS) bond
  misc_feature 179..180: note = Linked by phosphorothioate (PS) bond
  source 1..180: mol_type = other DNA
ggccccttccccacactatctcaatgcaaatatctgtctgaaacggtccctggctaaactccacccatgggttggccagccttgccttgacaaggcaaacttgaccaatagtcttagagtatccagtgaggccaggggccggcggctggctagggatgaagaataaaaggaagcaccctt

SEQ ID NO: 978 - DNA, 180 bp, synthetic construct
  misc_feature 1..180: note = Ptx ssODN 11nt - positive strand (OLI16413)
  misc_feature 1..2: note = Linked by phosphorothioate (PS) bond
  misc_feature 179..180: note = Linked by phosphorothioate (PS) bond
  source 1..180: mol_type = other DNA
gggtgcttccttttattcttcatccctagccagccgccggcccctggcctcactggatactctaagactattggtcaagtttgccttgtctcaaggcaaggctggccaacccatgggtggagtttagccagggaccgtttcagacagatatttgcattgagatagtgtggggaaggggcc

SEQ ID NO: 979 - DNA, 180 bp, synthetic construct
  misc_feature 1..180: note = Ptx ssODN 13nt - positive strand (OLI16414)
  misc_feature 1..2: note = Linked by phosphorothioate (PS) bond
  misc_feature 179..180: note = Linked by phosphorothioate (PS) bond
  source 1..180: mol_type = other DNA
aagggtgcttccttttattcttcatccctagccagccgccggcccctggcctcactggatactctaagactattggtcaagtttgccttgtcaaggcaaggctggccaacccatgggtggagtttagccagggaccgtttcagacagatatttgcattgagatagtgtggggaaggggcc

SEQ ID NO: 980 - DNA, 180 bp, synthetic construct
  misc_feature 1..180: note = Ptx ssODN 117G>A - negative strand (OLI16415)
  misc_feature 1..2: note = Linked by phosphorothioate (PS) bond
  misc_feature 179..180: note = Linked by phosphorothioate (PS) bond
  source 1..180: mol_type = other DNA
ggccccttccccacactatctcaatgcaaatatctgtctgaaacggtccctggctaaactccacccatgggttggccagccttgccttaaccaatagccttgacaaggcaaacttgaccaatagtcttagagtatccagtgaggccaggggccggcggctggctagggatgaagaataaa

SEQ ID NO: 981 - DNA, 180 bp, synthetic construct
  misc_feature 1..180: note = Ptx ssODN 117G>A - positive strand (OLI16416)
  misc_feature 1..2: note = Linked by phosphorothioate (PS) bond
  misc_feature 179..180: note = Linked by phosphorothioate (PS) bond
  source 1..180: mol_type = other DNA
tttattcttcatccctagccagccgccggcccctggcctcactggatactctaagactattggtcaagtttgccttgtcaaggctattggttaaggcaaggctggccaacccatgggtggagtttagccagggaccgtttcagacagatatttgcattgagatagtgtggggaaggggcc

SEQ ID NO: 982 - DNA, 180 bp, synthetic construct
  misc_feature 1..180: note = Ptx ssODN 1nt - negative strand (OLI16417)
  misc_feature 1..2: note = Linked by phosphorothioate (PS) bond
  misc_feature 179..180: note = Linked by phosphorothioate (PS) bond
  source 1..180: mol_type = other DNA
ggccccttccccacactatctcaatgcaaatatctgtctgaaacggtccctggctaaactccacccatgggttggccagccttgccttgccaatagccttgacaaggcaaacttgaccaatagtcttagagtatccagtgaggccaggggccggcggctggctagggatgaagaataaaa

SEQ ID NO: 983 - DNA, 180 bp, synthetic construct
  misc_feature 1..180: note = Ptx ssODN 1nt - positive strand (OLI16418)
  misc_feature 1..2: note = Linked by phosphorothioate (PS) bond
  misc_feature 179..180: note = Linked by phosphorothioate (PS) bond
  source 1..180: mol_type = other DNA
ttttattcttcatccctagccagccgccggcccctggcctcactggatactctaagactattggtcaagtttgccttgtcaaggctattggcaaggcaaggctggccaacccatgggtggagtttagccagggaccgtttcagacagatatttgcattgagatagtgtggggaaggggcc

SEQ ID NO: 984 - DNA, 120 bp, synthetic construct
  misc_feature 1..120: note = Ptx ssODN 4nt 40/80 - negative strand (OLI16419)
  misc_feature 1..2: note = Linked by phosphorothioate (PS) bond
  misc_feature 119..120: note = Linked by phosphorothioate (PS) bond
  source 1..120: mol_type = other DNA
tggctaaactccacccatgggttggccagccttgccttgatagccttgacaaggcaaacttgaccaatagtcttagagtatccagtgaggccaggggccggcggctggctagggatgaag

SEQ ID NO: 985 - DNA, 100 bp, synthetic construct
  misc_feature 1..100: note = Ptx ssODN 4nt 30/70 - negative strand (OLI16420)
  misc_feature 1..2: note = Linked by phosphorothioate (PS) bond
  misc_feature 99..100: note = Linked by phosphorothioate (PS) bond
  source 1..100: mol_type = other DNA
ccacccatgggttggccagccttgccttgatagccttgacaaggcaaacttgaccaatagtcttagagtatccagtgaggccaggggccggcggctggct

SEQ ID NO: 986 - DNA, 100 bp, synthetic construct
  misc_feature 1..100: note = Ptx ssODN 4nt - negative strand (OLI16421)
  misc_feature 1..2: note = Linked by phosphorothioate (PS) bond
  misc_feature 99..100: note = Linked by phosphorothioate (PS) bond
  source 1..100: mol_type = other DNA
aaacggtccctggctaaactccacccatgggttggccagccttgccttgatagccttgacaaggcaaacttgaccaatagtcttagagtatccagtgagg

SEQ ID NO: 987 - DNA, 120 bp, synthetic construct
  misc_feature 1..120: note = Ptx ssODN 4nt - negative strand (OLI16422)
  misc_feature 1..2: note = Linked by phosphorothioate (PS) bond
  misc_feature 119..120: note = Linked by phosphorothioate (PS) bond
  source 1..120: mol_type = other DNA
tatctgtctgaaacggtccctggctaaactccacccatgggttggccagccttgccttgatagccttgacaaggcaaacttgaccaatagtcttagagtatccagtgaggccaggggccg

SEQ ID NO: 988 - DNA, 140 bp, synthetic construct
  misc_feature 1..140: note = Ptx ssODN 4nt - negative strand (OLI16423)
  misc_feature 1..2: note = Linked by phosphorothioate (PS) bond
  misc_feature 139..140: note = Linked by phosphorothioate (PS) bond
  source 1..140: mol_type = other DNA
tcaatgcaaatatctgtctgaaacggtccctggctaaactccacccatgggttggccagccttgccttgatagccttgacaaggcaaacttgaccaatagtcttagagtatccagtgaggccaggggccggcggctggct

SEQ ID NO: 989 - DNA, 180 bp, synthetic construct
  misc_feature 1..180: note = Ptx ssODN 4nt - negative strand (OLI16424)
  misc_feature 1..2: note = Linked by phosphorothioate (PS) bond
  misc_feature 179..180: note = Linked by phosphorothioate (PS) bond
  source 1..180: mol_type = other DNA
ggccccttccccacactatctcaatgcaaatatctgtctgaaacggtccctggctaaactccacccatgggttggccagccttgccttgatagccttgacaaggcaaacttgaccaatagtcttagagtatccagtgaggccaggggccggcggctggctagggatgaagaataaaagga

SEQ ID NO: 990 - DNA, 120 bp, synthetic construct
  misc_feature 1..120: note = Ptx ssODN 4nt 40/80 - positive strand (OLI16425)
  misc_feature 1..2: note = Linked by phosphorothioate (PS) bond
  misc_feature 119..120: note = Linked by phosphorothioate (PS) bond
  source 1..120: mol_type = other DNA
tactctaagactattggtcaagtttgccttgtcaaggctatcaaggcaaggctggccaacccatgggtggagtttagccagggaccgtttcagacagatatttgcattgagatagtgtgg

SEQ ID NO: 991 - DNA, 100 bp, synthetic construct
  misc_feature 1..100: note = Ptx ssODN 4nt 30/70 - positive strand (OLI16426)
  misc_feature 1..2: note = Linked by phosphorothioate (PS) bond
  misc_feature 99..100: note = Linked by phosphorothioate (PS) bond
  source 1..100: mol_type = other DNA
ctattggtcaagtttgccttgtcaaggctatcaaggcaaggctggccaacccatgggtggagtttagccagggaccgtttcagacagatatttgcattga

SEQ ID NO: 992 - DNA, 100 bp, synthetic construct
  misc_feature 1..100: note = Ptx ssODN 4nt - positive strand (OLI16427)
  misc_feature 1..2: note = Linked by phosphorothioate (PS) bond
  misc_feature 99..100: note = Linked by phosphorothioate (PS) bond
  source 1..100: mol_type = other DNA
cctcactggatactctaagactattggtcaagtttgccttgtcaaggctatcaaggcaaggctggccaacccatgggtggagtttagccagggaccgttt

SEQ ID NO: 993 - DNA, 120 bp, synthetic construct
  misc_feature 1..120: note = Ptx ssODN 4nt - positive strand (OLI16428)
  misc_feature 1..2: note = Linked by phosphorothioate (PS) bond
  misc_feature 119..120: note = Linked by phosphorothioate (PS) bond
  source 1..120: mol_type = other DNA
cggcccctggcctcactggatactctaagactattggtcaagtttgccttgtcaaggctatcaaggcaaggctggccaacccatgggtggagtttagccagggaccgtttcagacagata

SEQ ID NO: 994 - DNA, 140 bp, synthetic construct
  misc_feature 1..140: note = Ptx ssODN 4nt - positive strand (OLI16429)
  misc_feature 1..2: note = Linked by phosphorothioate (PS) bond
  misc_feature 139..140: note = Linked by phosphorothioate (PS) bond
  source 1..140: mol_type = other DNA
agccagccgccggcccctggcctcactggatactctaagactattggtcaagtttgccttgtcaaggctatcaaggcaaggctggccaacccatgggtggagtttagccagggaccgtttcagacagatatttgcattga

SEQ ID NO: 995 - DNA, 180 bp, synthetic construct
  misc_feature 1..180: note = Ptx ssODN 4nt - positive strand (OLI16430)
  misc_feature 1..2: note = Linked by phosphorothioate (PS) bond
  misc_feature 179..180: note = Linked by phosphorothioate (PS) bond
  source 1..180: mol_type = other DNA
tccttttattcttcatccctagccagccgccggcccctggcctcactggatactctaagactattggtcaagtttgccttgtcaaggctatcaaggcaaggctggccaacccatgggtggagtttagccagggaccgtttcagacagatatttgcattgagatagtgtggggaaggggcc

SEQ ID NO: 996-999 - skipped (4 entries)

SEQ ID NO: 1000 - protein, 1332 aa, synthetic construct
  REGION 1..1332: note = His-AsCpf1-nNLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKG

SEQ ID NO: 1001 - protein, 1333 aa, synthetic construct
  REGION 1..1333: note = His-AsCpf1-sNLS-sNLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 1002 - RNA, 20 nt, synthetic construct
  misc_feature 1..20: note = HBG1-1 gRNA (OLI13620) targeting domain
  source 1..20: mol_type = other RNA
ccttgtcaaggctattggtc

SEQ ID NO: 1003 - DNA, 20 bp, synthetic construct
  misc_feature 1..20: note = HBG1-1 gRNA (OLI13620) targeting domain
  source 1..20: mol_type = other DNA
ccttgtcaaggctattggtc

SEQ ID NO: 1004 - RNA, 24 nt, synthetic construct
  misc_feature 1..24: note = HBG1-1 gRNA (OLI13620) targeting domain plus PAM
  source 1..24: mol_type = other RNA
tttgccttgtcaaggctattggtc

SEQ ID NO: 1005 - DNA, 24 bp, synthetic construct
  misc_feature 1..24: note = HBG1-1 gRNA (OLI13620) targeting domain plus PAM
  source 1..24: mol_type = other DNA
tttgccttgtcaaggctattggtc

SEQ ID NO: 1006 - protein, 16 aa, synthetic construct
  REGION 1..16: note = Nucleoplasmin NLS
KRPAATKKAGQAKKKK

SEQ ID NO: 1007 - protein, 7 aa, synthetic construct
  REGION 1..7: note = Simian Virus 40 NLS
PKKKRKV

SEQ ID NO: 1008 - protein, 1324 aa, synthetic construct
  REGION 1..1324: note = His-AsCpf1-sNLS
  REGION 3..8: note = MISC_FEATURE - His tag
  REGION 1318..1324: note = MISC_FEATURE - SV40 NLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKV

SEQ ID NO: 1009 - protein, 1326 aa, synthetic construct
  REGION 1..1326: note = His-sNLS-AsCpf1
  REGION 3..8: note = MISC_FEATURE - His tag
  REGION 11..17: note = MISC_FEATURE - SV40 NLS
MKHHHHHHGSPKKKRKVGSMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 1010 - protein, 1333 aa, synthetic construct
  REGION 1..1333: note = His-sNLS-sNLS-AsCpf1
  REGION 3..8: note = MISC_FEATURE - His tag
  REGION 11..17: note = MISC_FEATURE - SV40 NLS
  REGION 20..26: note = MISC_FEATURE - SV40 NLS
MKHHHHHHGSPKKKRKVGSPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 1011 - protein, 1327 aa, synthetic construct
  REGION 1..1327: note = sNLS-sNLS-AsCpf1
  REGION 5..11: note = MISC_FEATURE - SV40 NLS
  REGION 14..20: note = MISC_FEATURE - SV40 NLS
MKGSPKKKRKVGSPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 1012 - protein, 1332 aa, synthetic construct
  REGION 1..1332: note = His-sNLS-AsCpf1-sNLS
  REGION 3..8: note = MISC_FEATURE - His tag
  REGION 10..16: note = MISC_FEATURE - SV40 NLS
  REGION 1325..1331: note = MISC_FEATURE - SV40 NLS
MKHHHHHHMPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGPKKKRKVG

SEQ ID NO: 1013 - protein, 1352 aa, synthetic construct
  REGION 1..1352: note = His-sNLS-sNLS-AsCpf1-sNLS-sNLS
  REGION 3..8: note = MISC_FEATURE - His tag
  REGION 11..17: note = MISC_FEATURE - SV40 NLS
  REGION 20..26: note = MISC_FEATURE - SV40 NLS
  REGION 1337..1343: note = MISC_FEATURE - SV40 NLS
  REGION 1346..1352: note = MISC_FEATURE - SV40 NLS
MGHHHHHHGSPKKKRKVGSPKKKRKVGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 1014 - protein, 1332 aa, synthetic construct
  REGION 1..1332: note = His-AsCpf1-nNLS Cys-less
  REGION 3..8: note = MISC_FEATURE - His tag
  REGION 1316..1331: note = MISC_FEATURE - Nucleoplasmin NLS
MKHHHHHHMTQFEGFTNLYQVSKTLRFELIPQGKTLKHIQEQGFIEEDKARNDHYKELKPIIDRIYKTYADQSLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENSHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFSKYKTLLRNENVLETAEALFNELNSIDLTHIFISHKKLETISSALSDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKSSTQLKAVTAHFQTHTTPILLSNNFIEPLEITKEIYDLNNPEKEPKKFQTAYAKKTGDQKGYREALSKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNSLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVSFDSRFQNPEWPMDADANGAYHIALKGQLLLNHLKESKDLKLQNGISNQDWLAYIQELRNKRPAATKKAGQAKKKKG

SEQ ID NO: 1015 - protein, 1332 aa, synthetic construct
  REGION 1..1332: note = His-AsCpf1-nNLS Cys-low
  REGION 3..8: note = MISC_FEATURE - His tag
  REGION 1316..1331: note = MISC_FEATURE - Nucleoplasmin NLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFSKYKTLLRNENVLETAEALFNELNSIDLTHIFISHKKLETISSALSDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALSKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKG

SEQ ID NO: 1016 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 1017 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 1018 - protein, 1206 aa, synthetic construct
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 1019 - DNA, 4059 bp, Acidaminococcus sp.
  source 1..4059: mol_type = other DNA
atgacccagttcgagggcttcaccaacctgtaccaggtgagcaagaccctgcggttcgagctgatcccccagggcaagaccctgaagcacatccaggagcagggcttcatcgaggaggacaaggcccggaacgaccactacaaggagctgaagcccatcatcgaccggatctacaagacctacgccgaccagtgcctgcagctggtgcagctggactgggagaacctgagcgccgccatcgacagctaccggaaggagaagaccgaggagacccggaacgccctgatcgaggagcaggccacctaccggaacgccatccacgactacttcatcggccggaccgacaacctgaccgacgccatcaacaagcggcacgccgagatctacaagggcctgttcaaggccgagctgttcaacggcaaggtgctgaagcagctgggcaccgtgaccaccaccgagcacgagaacgccctgctgcggagcttcgacaagttcaccacctacttcagcggcttctacgagaaccggaagaacgtgttcagcgccgaggacatcagcaccgccatcccccaccggatcgtgcaggacaacttccccaagttcaaggagaactgccacatcttcacccggctgatcaccgccgtgcccagcctgcgggagcacttcgagaacgtgaagaaggccatcggcatcttcgtgagcaccagcatcgaggaggtgttcagcttccccttctacaaccagctgctgacccagacccagatcgacctgtacaaccagctgctgggcggcatcagccgggaggccggcaccgagaagatcaagggcctgaacgaggtgctgaacctggccatccagaagaacgacgagaccgcccacatcatcgccagcctgccccaccggttcatccccctgttcaagcagatcctgagcgaccggaacaccctgagcttcatcctggaggagttcaagagcgacgaggaggtgatccagagcttctgcaagtacaagaccctgctgcggaacgagaacgtgctggagaccgccgaggccctgttcaacgagctgaacagcatcgacctgacccacatcttcatcagccacaagaagctggagaccatcagcagcgccctgtgcgaccactgggacaccctgcggaacgccctgtacgagcggcggatcagcgagctgaccggcaagatcaccaagagcgccaaggagaaggtgcagcggagcctgaagcacgaggacatcaacctgcaggagatcatcagcgccgccggcaaggagctgagcgaggccttcaagcagaagaccagcgagatcctgagccacgcccacgccgccctggaccagcccctgcccaccaccctgaagaagcaggaggagaaggagatcctgaaaagccagctggacagcctgctgggcctgtaccacctgctggactggttcgccgtggacgagagcaacgaggtggaccccgagttcagcgcccggctgaccggcatcaagctggagatggagcccagcctgagcttctacaacaaggcccggaactacgccaccaagaagccctacagcgtggagaagttcaagctgaacttccagatgcccaccctggccagcggctgggacgtgaacaaggagaagaacaacggcgccatcctgttcgtgaagaacggcctgtactacctgggcatcatgcccaagcagaagggccggtacaaggccctgagcttcgagcccaccgagaagaccagcgagggcttcgacaagatgtactacgactacttccccgacgccgccaagatgatccccaagtgcagcacccagctgaaggccgtgaccgcccacttccagacccacaccacccccatcctgctgagcaacaacttcatcgagcccctggagatcaccaaggagatctacgacctgaacaaccccgagaaggagcccaagaagttccagaccgcctacgccaagaagaccggcgaccagaagggctaccgggaggccctgtgcaagtggatcgacttcacccgggacttcctgagcaagtacaccaagaccaccagcatcgacctgagcagcctgcggcccagcagccagtacaaggacctgggcgagtactacgccgagctgaaccccctgctgtaccacatcagcttccagcggatcgccgagaaggagatcatggacgccgtggagaccggcaagctgtacctgttccagatctacaacaaggacttcgccaagggccaccacggcaagcccaacctgcacaccctgtactggaccggcctgttcagccccgagaacctggccaagaccagcatcaagctgaacggccaggccgagctgttctaccggcccaagagccggatgaagcggatggcccaccggctgggcgagaagatgctgaacaagaagctgaaggaccagaagacccccatccccgacaccctgtaccaggagctgtacgactacgtgaaccaccggctgagccacgacctgagcgacgaggcccgggccctgctgcccaacgtgatcaccaaggaggtgagccacgagatcatcaaggaccggcggttcaccagcgacaagttcttcttccacgtgcccatcaccctgaactaccaggccgccaacagccccagcaagttcaaccagcgggtgaacgcctacctgaaggagcaccccgagacccccatcatcggcatcgaccggggcgagcggaacctgatctacatcaccgtgatcgacagcaccggcaagatcctggagcagcggagcctgaacaccatccagcagttcgactaccagaagaagctggacaaccgggagaaggagcgggtggccgcccggcaggcctggagcgtggtgggcaccatcaaggacctgaagcagggctacctgagccaggtgatccacgagatcgtggacctgatgatccactaccaggccgtggtggtgctggagaacctgaacttcggcttcaagagcaagcggaccggcatcgccgagaaggccgtgtaccagcagttcgagaagatgctgatcgacaagctgaactgcctggtgctgaaggactaccccgccgagaaggtgggcggcgtgctgaacccctaccagctgaccgaccagttcaccagcttcgccaagatgggcacccagagcggcttcctgttctacgtgcccgccccctacaccagcaagatcgaccccctgaccggcttcgtggaccccttcgtgtggaagaccatcaagaaccacgagagccggaagcacttcctggagggcttcgacttcctgcactacgacgtgaagaccggcgacttcatcctgcacttcaagatgaaccggaacctgagcttccagcggggcctgcccggcttcatgcccgcctgggacatcgtgttcgagaagaacgagacccagttcgacgccaagggcacccccttcatcgccggcaagcggatcgtgcccgtgatcgagaaccaccggttcaccggccggtaccgggacctgtaccccgccaacgagctgatcgccctgctggaggagaagggcatcgtgttccgggacggcagcaacatcctgcccaagctgctggagaacgacgacagccacgccatcgacaccatggtggccctgatccggagcgtgctgcagatgcggaacagcaacgccgccaccggcgaggactacatcaacagccccgtgcgggacctgaacggcgtgtgcttcgacagccggttccagaaccccgagtggcccatggacgccgacgccaacggcgcctaccacatcgccctgaagggccagctgctgctgaaccacctgaaggagagcaaggacctgaagctgcagaacggcatcagcaaccaggactggctggcctacatccaggagctgcggaacaagcggcccgccgccaccaagaaggccggccaggccaagaagaagaagggcagctacccctacgacgtgcccgactacgcctacccctacgacgtgcccgactacgcctacccctacgacgtgcccgactacgcctga

SEQ ID NO: 1020 - DNA, 3822 bp, synthetic construct
  source 1..3822: mol_type = other DNA
atgagcaagctggagaagttcaccaactgctacagcctgagcaagaccctgcggttcaaggccatccccgtgggcaagacccaggagaacatcgacaacaagcggctgctggtggaggacgagaagcgggccgaggactacaagggcgtgaagaagctgctggaccggtactacctgagcttcatcaacgacgtgctgcacagcatcaagctgaagaacctgaacaactacatcagcctgttccggaagaagacccggaccgagaaggagaacaaggagctggagaacctggagatcaacctgcggaaggagatcgccaaggccttcaagggcaacgagggctacaagagcctgttcaagaaggacatcatcgagaccatcctgcccgagttcctggacgacaaggacgagatcgccctggtgaacagcttcaacggcttcaccaccgccttcaccggcttcttcgacaaccgggagaacatgttcagcgaggaggccaagagcaccagcatcgccttccggtgcatcaacgagaacctgacccggtacatcagcaacatggacatcttcgagaaggtggacgccatcttcgacaagcacgaggtgcaggagatcaaggagaagatcctgaacagcgactacgacgtggaggacttcttcgagggcgagttcttcaacttcgtgctgacccaggagggcatcgacgtgtacaacgccatcatcggcggcttcgtgaccgagagcggcgagaagatcaagggcctgaacgagtacatcaacctgtacaaccagaagaccaagcagaagctgcccaagttcaagcccctgtacaagcaggtgctgagcgaccgggagagcctgagcttctacggcgagggctacaccagcgacgaggaggtgctggaggtgttccggaacaccctgaacaagaacagcgagatcttcagcagcatcaagaagctggagaagctgttcaagaacttcgacgagtacagcagcgccggcatcttcgtgaagaacggccccgccatcagcaccatcagcaaggacatcttcggcgagtggaacgtgatccgggacaagtggaacgccgagtacgacgacatccacctgaagaagaaggccgtggtgaccgagaagtacgaggacgaccggcggaaaagcttcaagaagatcggcagcttcagcctggagcagctgcaggagtacgccgacgccgacctgagcgtggtggagaagctgaaggagatcatcatccagaaggtggacgagatctacaaggtgtacggcagcagcgagaagctgttcgacgccgacttcgtgctggagaaaagcctgaagaagaacgacgccgtggtggccatcatgaaggacctgctggacagcgtgaaaagcttcgagaactacatcaaggccttcttcggcgagggcaaggagaccaaccgggacgagagcttctacggcgacttcgtgctggcctacgacatcctgctgaaggtggaccacatctacgacgccatccggaactacgtgacccagaagccctacagcaaggacaagttcaagctgtacttccagaacccccagttcatgggcggctgggacaaggacaaggagaccgactaccgggccaccatcctgcggtacggcagcaagtactacctggccatcatggacaagaagtacgccaagtgcctgcagaagatcgacaaggacgacgtgaacggcaactacgagaagatcaactacaagctgctgcccggccccaacaagatgctgcccaaggtgttcttcagcaagaagtggatggcctactacaaccccagcgaggacatccagaagatctacaagaacggcaccttcaagaagggcgacatgttcaacctgaacgactgccacaagctgatcgacttcttcaaggacagcatcagccggtaccccaagtggagcaacgcctacgacttcaacttcagcgagaccgagaagtacaaggacatcgccggcttctaccgggaggtggaggagcagggctacaaggtgagcttcgagagcgccagcaagaaggaggtggacaagctggtggaggagggcaagctgtacatgttccagatctacaacaaggacttcagcgacaagagccacggcacccccaacctgcacaccatgtacttcaagctgctgttcgacgagaacaaccacggccagatccggctgagcggcggcgccgagctgttcatgcggcgggccagcctgaagaaggaggagctggtggtgcaccccgccaacagccccatcgccaacaagaaccccgacaaccccaagaagaccaccaccctgagctacgacgtgtacaaggacaagcggttcagcgaggaccagtacgagctgcacatccccatcgccatcaacaagtgccccaagaacatcttcaagatcaacaccgaggtgcgggtgctgctgaagcacgacgacaacccctacgtgatcggcatcgaccggggcgagcggaacctgctgtacatcgtggtggtggacggcaagggcaacatcgtggagcagtacagcctgaacgagatcatcaacaacttcaacggcatccggatcaagaccgactaccacagcctgctggacaagaaggagaaggagcggttcgaggcccggcagaactggaccagcatcgagaacatcaaggagctgaaggccggctacatcagccaggtggtgcacaagatctgcgagctggtggagaagtacgacgccgtgatcgccctggaggacctgaacagcggcttcaagaacagccgggtgaaggtggagaagcaggtgtaccagaagttcgagaagatgctgatcgacaagctgaactacatggtggacaagaaaagcaacccctgcgccaccggcggcgccctgaagggctaccagatcaccaacaagttcgagagcttcaagagcatgagcacccagaacggcttcatcttctacatccccgcctggctgaccagcaagatcgaccccagcaccggcttcgtgaacctgctgaagaccaagtacaccagcatcgccgacagcaagaagttcatcagcagcttcgaccggatcatgtacgtgcccgaggaggacctgttcgagttcgccctggactacaagaacttcagccggaccgacgccgactacatcaagaagtggaagctgtacagctacggcaaccggatccggatcttccggaaccccaagaagaacaacgtgttcgactgggaggaggtgtgcctgaccagcgcctacaaggagctgttcaacaagtacggcatcaactaccagcagggcgacatccgggccctgctgtgcgagcagagcgacaaggccttctacagcagcttcatggccctgatgagcctgatgctgcagatgcggaacagcatcaccggccggaccgacgtggacttcctgatcagccccgtgaagaacagcgacggcatcttctacgacagccggaactacgaggcccaggagaacgccatcctgcccaagaacgccgacgccaacggcgcctacaacatcgcccggaaggtgctgtgggccatcggccagttcaagaaggccgaggacgagaagctggacaaggtgaagatcgccatcagcaacaaggagtggctggagtacgcccagaccagcgtgaagcacaagcggcccgccgccaccaagaaggccggccaggccaagaagaagaagggcagctacccctacgacgtgcccgactacgcctacccctacgacgtgcccgactacgcctacccctacgacgtgcccgactacgcctga

SEQ ID NO: 1021 - DNA, 3756 bp, synthetic construct
  source 1..3756: mol_type = other DNA
atgtactacgagagcctgaccaagcagtaccccgtgagcaagaccatccggaacgagctgatccccatcggcaagaccctggacaacatccggcagaacaacatcctggagagcgacgtgaagcggaagcagaactacgagcacgtgaagggcatcctggacgagtaccacaagcagctgatcaacgaggccctggacaactgcaccctgcccagcctgaagatcgccgccgagatctacctgaagaaccagaaggaggtgagcgaccgggaggacttcaacaagacccaggacctgctgcggaaggaggtggtggagaagctgaaggcccacgagaacttcaccaagatcggcaagaaggacatcctggacctgctggagaagctgcccagcatcagcgaggacgactacaacgccctggagagcttccggaacttctacacctacttcaccagctacaacaaggtgcgggagaacctgtacagcgacaaggagaaaagcagcaccgtggcctaccggctgatcaacgagaacttccccaagttcctggacaacgtgaaaagctaccggttcgtgaagaccgccggcatcctggccgacggcctgggcgaggaggagcaggacagcctgttcatcgtggagaccttcaacaagaccctgacccaggacggcatcgacacctacaacagccaggtgggcaagatcaacagcagcatcaacctgtacaaccagaagaaccagaaggccaacggcttccggaagatccccaagatgaagatgctgtacaagcagatcctgagcgaccgggaggagagcttcatcgacgagttccagagcgacgaggtgctgatcgacaacgtggagagctacggcagcgtgctgatcgagagcctgaaaagcagcaaggtgagcgccttcttcgacgccctgcgggagagcaagggcaagaacgtgtacgtgaagaacgacctggccaagaccgccatgagcaacatcgtgttcgagaactggcggaccttcgacgacctgctgaaccaggagtacgacctggccaacgagaacaagaagaaggacgacaagtacttcgagaagcggcagaaggagctgaagaagaacaagagctacagcctggagcacctgtgcaacctgagcgaggacagctgcaacctgatcgagaactacatccaccagatcagcgacgacatcgagaacatcatcatcaacaacgagaccttcctgcggatcgtgatcaacgagcacgaccggagccggaagctggccaagaaccggaaggccgtgaaggccatcaaggacttcctggacagcatcaaggtgctggagcgggagctgaagctgatcaacagcagcggccaggagctggagaaggacctgatcgtgtacagcgcccacgaggagctgctggtggagctgaagcaggtggacagcctgtacaacatgacccggaactacctgaccaagaagcccttcagcaccgagaaggtgaagctgaacttcaaccggagcaccctgctgaacggctgggaccggaacaaggagaccgacaacctgggcgtgctgctgctgaaggacggcaagtactacctgggcatcatgaacaccagcgccaacaaggccttcgtgaacccccccgtggccaagaccgagaaggtgttcaagaaggtggactacaagctgctgcccgtgcccaaccagatgctgcccaaggtgttcttcgccaagagcaacatcgacttctacaaccccagcagcgagatctacagcaactacaagaagggcacccacaagaagggcaacatgttcagcctggaggactgccacaacctgatcgacttcttcaaggagagcatcagcaagcacgaggactggagcaagttcggcttcaagttcagcgacaccgccagctacaacgacatcagcgagttctaccgggaggtggagaagcagggctacaagctgacctacaccgacatcgacgagacctacatcaacgacctgatcgagcggaacgagctgtacctgttccagatctacaacaaggacttcagcatgtacagcaagggcaagctgaacctgcacaccctgtacttcatgatgctgttcgaccagcggaacatcgacgacgtggtgtacaagctgaacggcgaggccgaggtgttctaccggcccgccagcatcagcgaggacgagctgatcatccacaaggccggcgaggagatcaagaacaagaaccccaaccgggcccggaccaaggagaccagcaccttcagctacgacatcgtgaaggacaagcggtacagcaaggacaagttcaccctgcacatccccatcaccatgaacttcggcgtggacgaggtgaagcggttcaacgacgccgtgaacagcgccatccggatcgacgagaacgtgaacgtgatcggcatcgaccggggcgagcggaacctgctgtacgtggtggtgatcgacagcaagggcaacatcctggagcagatcagcctgaacagcatcatcaacaaggagtacgacatcgagaccgactaccacgccctgctggacgagcgggagggcggccgggacaaggcccggaaggactggaacaccgtggagaacatccgggacctgaaggccggctacctgagccaggtggtgaacgtggtggccaagctggtgctgaagtacaacgccatcatctgcctggaggacctgaacttcggcttcaagcggggccggcagaaggtggagaagcaggtgtaccagaagttcgagaagatgctgatcgacaagctgaactacctggtgatcgacaagagccgggagcagaccagccccaaggagctgggcggcgccctgaacgccctgcagctgaccagcaagttcaagagcttcaaggagctgggcaagcagagcggcgtgatctactacgtgcccgcctacctgaccagcaagatcgaccccaccaccggcttcgccaacctgttctacatgaagtgcgagaacgtggagaaaagcaagcggttcttcgacggcttcgacttcatccggttcaacgccctggagaacgtgttcgagttcggcttcgactaccggagcttcacccagcgggcctgcggcatcaacagcaagtggaccgtgtgcaccaacggcgagcggatcatcaagtaccggaaccccgacaagaacaacatgttcgacgagaaggtggtggtggtgaccgacgagatgaagaacctgttcgagcagtacaagatcccctacgaggacggccggaacgtgaaggacatgatcatcagcaacgaggaggccgagttctaccggcggctgtaccggctgctgcagcagaccctgcagatgcggaacagcaccagcgacggcacccgggactacatcatcagccccgtgaagaacaagcgggaggcctacttcaacagcgagctgagcgacggcagcgtgcccaaggacgccgacgccaacggcgcctacaacatcgcccggaagggcctgtgggtgctggagcagatccggcagaaaagcgagggcgagaagatcaacctggccatgaccaacgccgagtggctggagtacgcccagacccacctgctgaagcggcccgccgccaccaagaaggccggccaggccaagaagaagaagggcagctacccctacgacgtgcccgactacgcctacccctacgacgtgcccgactacgcctacccctacgacgtgcccgactacgcctga

SEQ ID NO: 1022 - RNA, 40 nt, synthetic construct
  misc_feature 1..40: note = HBG1-1 full-length gRNA
  source 1..40: mol_type = other RNA
taatttctactcttgtagatccttgtcaaggctattggtc

SEQ ID NO: 1023 - RNA, 41 nt, synthetic construct
  misc_feature 1..41: note = HBG1-1 modified full-length gRNA
  misc_feature 40: note = Modified to include 2'-O-methyl
  misc_feature 40..41: note = Linked by phosphorodithioate (PS2) bond
  misc_feature 41: note = Modified to include 2'-O-methyl
  source 1..41: mol_type = other RNA
taatttctactcttgtagatccttgtcaaggctattggtca

SEQ ID NO: 1024 - RNA, 95 nt, synthetic construct
  misc_feature 1..95: note = tSpA dead gRNA
  misc_feature 1: note = Modified to contain 2'-O-methyl
  misc_feature 1..2: note = Linked by phosphorothioate (PS) bond
  misc_feature 2..3: note = Linked by phosphorothioate (PS) bond
  misc_feature 2: note = Modified to contain 2'-O-methyl
  misc_feature 3..4: note = Linked by phosphorothioate (PS) bond
  misc_feature 3: note = Modified to contain 2'-O-methyl
  misc_feature 92..93: note = Linked by phosphorothioate (PS) bond
  misc_feature 92: note = Modified to contain 2'-O-methyl
  misc_feature 93..94: note = Linked by phosphorothioate (PS) bond
  misc_feature 93: note = Modified to contain 2'-O-methyl
  misc_feature 94..95: note = Linked by phosphorothioate (PS) bond
  misc_feature 94: note = Modified to contain 2'-O-methyl
  source 1..95: mol_type = other RNA
ggctggccaacccatgttttagagctagaaatagcaagttaaaataaggctagtccgttatcaacttgaaaaagtggcaccgagtcggtgctttt

SEQ ID NO: 1025 - RNA, 100 nt, synthetic construct
  misc_feature 1..100: note = SpA full-length gRNA
  misc_feature 1: note = Modified to contain 2'-O-methyl
  misc_feature 1..2: note = Linked by phosphorothioate (PS) bond
  misc_feature 2: note = Modified to contain 2'-O-methyl
  misc_feature 2..3: note = Linked by phosphorothioate (PS) bond
  misc_feature 3: note = Modified to contain 2'-O-methyl
  misc_feature 3..4: note = Linked by phosphorothioate (PS) bond
  misc_feature 97: note = Modified to contain 2'-O-methyl
  misc_feature 97..98: note = Linked by phosphorothioate (PS) bond
  misc_feature 98: note = Modified to contain 2'-O-methyl
  misc_feature 98..99: note = Linked by phosphorothioate (PS) bond
  misc_feature 99..100: note = Linked by phosphorothioate (PS) bond
  misc_feature 99: note = Modified to contain 2'-O-methyl
  source 1..100: mol_type = other RNA
ggcaaggctggccaacccatgttttagagctagaaatagcaagttaaaataaggctagtccgttatcaacttgaaaaagtggcaccgagtcggtgctttt

SEQ ID NO: 1026 - RNA, 100 nt, synthetic construct
  misc_feature 1..100: note = SpG full-length gRNA
  misc_feature 1: note = Modified to contain 2'-O-methyl
  misc_feature 1..2: note = Linked by phosphorothioate (PS) bond
  misc_feature 2: note = Modified to contain 2'-O-methyl
  misc_feature 2..3: note = Linked by phosphorothioate (PS) bond
  misc_feature 3: note = Modified to contain 2'-O-methyl
  misc_feature 3..4: note = Linked by phosphorothioate (PS) bond
  misc_feature 97: note = Modified to contain 2'-O-methyl
  misc_feature 97..98: note = Linked by phosphorothioate (PS) bond
  misc_feature 98: note = Modified to contain 2'-O-methyl
  misc_feature 98..99: note = Linked by phosphorothioate (PS) bond
  misc_feature 99..100: note = Linked by phosphorothioate (PS) bond
  misc_feature 99: note = Modified to contain 2'-O-methyl
  source 1..100: mol_type = other RNA
tagtcttagagtatccagtggttttagagctagaaatagcaagttaaaataaggctagtccgttatcaacttgaaaaagtggcaccgagtcggtgctttt

SEQ ID NO: 1027 - RNA, 95 nt, synthetic construct
  misc_feature 1..95: note = Sp182 dead gRNA
  misc_feature 1: note = Modified to contain 2'-O-methyl
  misc_feature 1..2: note = Linked by phosphorothioate (PS) bond
  misc_feature 2..3: note = Linked by phosphorothioate (PS) bond
  misc_feature 2: note = Modified to contain 2'-O-methyl
  misc_feature 3..4: note = Linked by phosphorothioate (PS) bond
  misc_feature 3: note = Modified to contain 2'-O-methyl
  misc_feature 92..93: note = Linked by phosphorothioate (PS) bond
  misc_feature 92: note = Modified to contain 2'-O-methyl
  misc_feature 93..94: note = Linked by phosphorothioate (PS) bond
  misc_feature 93: note = Modified to contain 2'-O-methyl
  misc_feature 94..95: note = Linked by phosphorothioate (PS) bond
  misc_feature 94: note = Modified to contain 2'-O-methyl
  source 1..95: mol_type = other RNA
ttagagtatccagtggttttagagctagaaatagcaagttaaaataaggctagtccgttatcaacttgaaaaagtggcaccgagtcggtgctttt

SEQ ID NO: 1028 - RNA, 15 nt, synthetic construct
  misc_feature 1..15: note = Sp182 dead gRNA targeting domain
  source 1..15: mol_type = other RNA
ttagagtatccagtg

SEQ ID NO: 1029 - DNA, 15 bp, synthetic construct
  misc_feature 1..15: note = Sp182 dgRNA targeting domain
  source 1..15: mol_type = other DNA
ttagagtatccagtg

SEQ ID NO: 1030 - DNA, 20 bp, synthetic construct
  misc_feature 1..20: note = SpG gRNA targeting domain
  source 1..20: mol_type = other DNA
tagtcttagagtatccagtg

SEQ ID NO: 1031 - DNA, 15 bp, synthetic construct
  misc_feature 1..15: note = tSpA dead gRNA targeting domain
  source 1..15: mol_type = other DNA
ggctggccaacccat

SEQ ID NO: 1032 - protein, 1334 aa, synthetic construct
  REGION 1..1334: note = His-AsCpf1-sNLS-sNLS H800A
  REGION 3..8: note = MISC_FEATURE - His tag
  REGION 1319..1325: note = MISC_FEATURE - sNLS
  REGION 1328..1334: note = MISC_FEATURE - sNLS
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 1033 - protein, 1384 aa, synthetic construct
  REGION 1..1384: note = SpCas9WT
MKHHHHHHMPKKKRKVMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1034 - protein, 1384 aa, synthetic construct
  REGION 1..1384: note = His-NLS-SpCas9D10A
MKHHHHHHMPKKKRKVMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 1035 - protein, 1333 aa, synthetic construct; identical to SEQ ID NO: 1001
  REGION 1..1333: note = AsCpf1 His-AsCpf1-NLS-NLS

SEQ ID NO: 1036 - protein, 1332 aa, synthetic construct
  REGION 1..1332: note = AsCpf1 S542R/K607R(AsCpf1 RR)
MKHHHHHHMPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNKEKNNGAILFVKNGLYYLGIMPKQKGRYKALSFEPTEKTSEGFDKMYYDYFPDAAKMIPRCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGPKKKRKVG

SEQ ID NO: 1037 - protein, 1332 aa, synthetic construct
  REGION 1..1332: note = AsCpf1 S542R/K548V/N552R (AsCpf1 RVR)
MKHHHHHHMPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGPKKKRKVG

SEQ ID NO: 1038 - protein, 1334 aa, synthetic construct
  REGION 1..1334: note = His-AsCpf1-2xNLS-S542R/K607R/H800A (AsCpf1 RR-H800A)
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNKEKNNGAILFVKNGLYYLGIMPKQKGRYKALSFEPTEKTSEGFDKMYYDYFPDAAKMIPRCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 1039 - protein, 1334 aa, synthetic construct
  REGION 1..1334: note = His-AsCpf1-2xNLS-S542R-K548V-N552R-H800A (Cpf1 RVR H800A)
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 1040 - DNA, 100 bp, synthetic construct
  misc_feature 1..100: note = Ptx ssODN 18nt - positive strand (OLI16431)
  misc_feature 1..2: note = Linked by phosphorothioate (PS) bond
  misc_feature 99..100: note = Linked by phosphorothioate (PS) bond
  source 1..100: mol_type = other DNA
ccctggcctcactggatactctaagactattggtcaagtttgccttgtcaaggctggccaacccatgggtggagtttagccagggaccgtttcagacaga

SEQ ID NO: 1041 - RNA, 40 nt, synthetic construct; identical to SEQ ID NO: 1022
  misc_feature 1..40: note = gRNA
  misc_feature 39: note = Modified to contain 2'-O-methyl
  misc_feature 39..40: note = Linked by phosphorodithioate (PS2) bond
  misc_feature 40: note = Modified to contain 2'-O-methyl
  source 1..40: mol_type = other RNA

SEQ ID NO: 1042 - RNA, 45 nt, synthetic construct
  misc_feature 1..45: note = gRNA
  source 1..45: mol_type = other RNA
ctttttaatttctactcttgtagatccttgtcaaggctattggtc

SEQ ID NO: 1043 - RNA, 50 nt, synthetic construct
  misc_feature 1..50: note = gRNA
  source 1..50: mol_type = other RNA
aagacctttttaatttctactcttgtagatccttgtcaaggctattggtc

SEQ ID NO: 1044 - RNA, 65 nt, synthetic construct
  misc_feature 1..65: note = gRNA
  source 1..65: mol_type = other RNA
atgtgtttttgtcaaaagacctttttaatttctactcttgtagatccttgtcaaggctattggtc

SEQ ID NO: 1045 - RNA, 100 nt, synthetic construct
  misc_feature 1..100: note = gRNA
  source 1..100: mol_type = other RNA
aggccagcttgccggttttttagtcgtgctgcttcatgtgtttttgtcaaaagacctttttaatttctactcttgtagatccttgtcaaggctattggtc

SEQ ID NO: 1046 - DNA, 45 bp, synthetic construct
  misc_feature 1..5: note = DNA nucleotides
  misc_feature 6..45: note = RNA nucleotides
  source 1..45: mol_type = other DNA
ctttttaatttctactcttgtagatccttgtcaaggctattggtc

SEQ ID NO: 1047 - DNA, 50 bp, synthetic construct
  misc_feature 1..50: note = gRNA
  misc_feature 1..10: note = DNA nucleotides
  misc_feature 11..50: note = RNA nucleotides
  source 1..50: mol_type = other DNA
aagacctttttaatttctactcttgtagatccttgtcaaggctattggtc

SEQ ID NO: 1048 - DNA, 65 bp, synthetic construct
  misc_feature 1..65: note = gRNA
  misc_feature 1..25: note = DNA nucleotides
  misc_feature 26..65: note = RNA nucleotides
  source 1..65: mol_type = other DNA
atgtgtttttgtcaaaagaccttttyaatttctactcttgtagatccttgtcaaggctattggtc

SEQ ID NO: 1049 - DNA, 100 bp, synthetic construct
  misc_feature 1..100: note = gRNA
  misc_feature 1..60: note = DNA nucleotides
  misc_feature 61..100: note = RNA nucleotides
  source 1..100: mol_type = other DNA
aggccagcttgccggttttttagtcgtgctgcttcatgtgtttttgtcaaaagacctttttaatttctactcttgtagatccttgtcaaggctattggtc

SEQ ID NO: 1050 - DNA, 66 bp, synthetic construct
  misc_feature 1..66: note = gRNA
  misc_feature 1..25: note = DNA nucleotides
  misc_feature 26..66: note = RNA nucleotides
  misc_feature 65: note = Modified to contain 2'-O-methyl
  misc_feature 65..66: note = Linked by phosphorodithioate (PS2) bond
  misc_feature 66: note = Modified to contain 2'-O-methyl
  source 1..66: mol_type = other DNA
atgtgtttttgtcaaaagacctttttaatttctactcttgtagatccttgtcaaggctattggtca

SEQ ID NO: 1051 - DNA, 66 bp, synthetic construct; identical to SEQ ID NO: 1050
  misc_feature 1..66: note = gRNA
  misc_feature 1..25: note = DNA nucleotides
  misc_feature 26..66: note = RNA nucleotides
  misc_feature 65..66: note = Linked by phosphorothioate (PS) bond
  misc_feature 66: note = Modified to contain 2'-O-methyl
  source 1..66: mol_type = other DNA

SEQ ID NO: 1052 - DNA, 60 bp, synthetic construct
  misc_feature 1..60: note = gRNA
  misc_feature 1..20: note = DNA nucleotides
  misc_feature 21..60: note = RNA nucleotides
  source 1..60: mol_type = other DNA
tttttgtcaaaagacctttttaatttctactcttgtagatccttgtcaaggctattggtc

SEQ ID NO: 1053 - DNA, 70 bp, synthetic construct
  misc_feature 1..70: note = gRNA
  misc_feature 1..30: note = DNA nucleotides
  misc_feature 31..70: note = RNA nucleotides
  source 1..70: mol_type = other DNA
gcttcatgtgtttttgtcaaaagacctttttaatttctactcttgtagatccttgtcaaggctattggtc

SEQ ID NO: 1054 - DNA, 90 bp, synthetic construct
  misc_feature 1..90: note = gRNA
  misc_feature 1..50: note = DNA nucleotides
  misc_feature 51..90: note = RNA nucleotides
  source 1..90: mol_type = other DNA
gccggttttttagtcgtgctgcttcatgtgtttttgtcaaaagacctttttaatttctactcttgtagatccttgtcaaggctattggtc

SEQ ID NO: 1055 - DNA, 80 bp, synthetic construct
  misc_feature 1..80: note = gRNA
  misc_feature 1..40: note = DNA nucleotides
  misc_feature 41..80: note = RNA nucleotides
  source 1..80: mol_type = other DNA
tagtcgtgctgcttcatgtgtttttgtcaaaagacctttttaatttctactcttgtagatccttgtcaaggctattggtc

SEQ ID NO: 1056 - DNA, 60 bp, synthetic construct
  misc_feature 1..60: note = gRNA
  misc_feature 1..20: note = DNA nucleotides
  misc_feature 1..2: note = Linked by phosphorothioate (PS) bond
  misc_feature 2..3: note = Linked by phosphorothioate (PS) bond
  misc_feature 21..60: note = RNA nucleotides
  source 1..60: mol_type = other DNA
ccgaagttttcttcggtttttaatttctactcttgtagatccttgtcaaggctattggtc

SEQ ID NO: 1057 - DNA, 65 bp, synthetic construct
  misc_feature 1..65: note = gRNA
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..65: note = RNA nucleotides
  source 1..65: mol_type = other DNA
tttttccgaagttttcttcggtttttaatttctactcttgtagatccttgtcaaggctattggtc

SEQ ID NO: 1058 - DNA, 70 bp, synthetic construct
  misc_feature 1..70: note = gRNA
  misc_feature 1..30: note = DNA nucleotides
  misc_feature 1..2: note = Linked by phosphorothioate (PS) bond
  misc_feature 2..3: note = Linked by phosphorothioate (PS) bond
  misc_feature 31..70: note = RNA nucleotides
  source 1..70: mol_type = other DNA
aacgctttttccgaagttttcttcggtttttaatttctactcttgtagatccttgtcaaggctattggtc

SEQ ID NO: 1059 - DNA, 81 bp, synthetic construct
  misc_feature 1..81: note = gRNA
  misc_feature 1..41: note = DNA nucleotides
  misc_feature 1..2: note = Linked by phosphorothioate (PS) bond
  misc_feature 2..3: note = Linked by phosphorothioate (PS) bond
  misc_feature 42..81: note = RNA nucleotides
  source 1..81: mol_type = other DNA
gcgttgttttcaacgctttttccgaagttttcttcggtttttaatttctactcttgtagatccttgtcaaggctattggtc

SEQ ID NO: 1060 - DNA, 102 bp, synthetic construct
  misc_feature 1..102: note = gRNA
  misc_feature 1..62: note = DNA nucleotides
  misc_feature 1..2: note = Linked by phosphorothioate (PS) bond
  misc_feature 2..3: note = Linked by phosphorothioate (PS) bond
  misc_feature 63..102: note = RNA nucleotides
  source 1..102: mol_type = other DNA
ggcttcttttgaagcctttttgcgttgttttcaacgctttttccgaagttttcttcggtttttaatttctactcttgtagatccttgtcaaggctattggtc

SEQ ID NO: 1061 - RNA, 40 nt, synthetic construct; identical to SEQ ID NO: 1022
  misc_feature 1..40: note = gRNA
  misc_feature 1: note = Modified to contain 2'-O-methyl
  source 1..40: mol_type = other RNA

SEQ ID NO: 1062 - RNA, 40 nt, synthetic construct; identical to SEQ ID NO: 1022
  misc_feature 1..40: note = gRNA
  misc_feature 1: note = Modified to contain 2'-O-methyl
  misc_feature 1..2: note = Linked by phosphorothioate (PS) bond
  source 1..40: mol_type = other RNA

SEQ ID NO: 1063 - RNA, 40 nt, synthetic construct; identical to SEQ ID NO: 1022
  misc_feature 1..40: note = gRNA
  misc_feature 1: note = Modified to contain 2'-O-methyl
  misc_feature 2: note = Modified to contain 2'-O-methyl
  source 1..40: mol_type = other RNA

SEQ ID NO: 1064 - RNA, 40 nt, synthetic construct; identical to SEQ ID NO: 1022
  misc_feature 1..40: note = gRNA
  misc_feature 1: note = Modified to contain 2'-O-methyl
  misc_feature 1..2: note = Linked by phosphorothioate (PS) bond
  misc_feature 2: note = Modified to contain 2'-O-methyl
  source 1..40: mol_type = other RNA

SEQ ID NO: 1065 - RNA, 40 nt, synthetic construct; identical to SEQ ID NO: 1022
  misc_feature 1..40: note = gRNA
  misc_feature 1: note = Modified to contain 2'-O-methyl
  misc_feature 2: note = Modified to contain 2'-O-methyl
  misc_feature 3: note = Modified to contain 2'-O-methyl
  source 1..40: mol_type = other RNA

SEQ ID NO: 1066 - RNA, 40 nt, synthetic construct; identical to SEQ ID NO: 1022
  misc_feature 1..40: note = gRNA
  misc_feature 1: note = Modified to contain 2'-O-methyl
  misc_feature 1..2: note = Linked by phosphorothioate (PS) bond
  misc_feature 2..3: note = Linked by phosphorothioate (PS) bond
  misc_feature 2: note = Modified to contain 2'-O-methyl
  misc_feature 3..4: note = Linked by phosphorothioate (PS) bond
  misc_feature 3: note = Modified to contain 2'-O-methyl
  source 1..40: mol_type = other RNA

SEQ ID NO: 1067 - DNA, 65 bp, synthetic construct
  misc_feature 1..65: note = gRNA
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..65: note = RNA nucleotides
  source 1..65: mol_type = other DNA
atgtgtttttgtcaaaagacctttttaatttctactcttgtagatccttgtcaaggctattggtc

SEQ ID NO: 1068 - DNA, 66 bp, synthetic construct; identical to SEQ ID NO: 1050
  misc_feature 1..66: note = gRNA
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..66: note = RNA nucleotides
  misc_feature 65..66: note = Linked by phosphorothioate (PS) bond
  misc_feature 66: note = Modified to contain 2'-O-methyl
  source 1..66: mol_type = other DNA

SEQ ID NO: 1069 - RNA, 41 nt, synthetic construct; identical to SEQ ID NO: 1023
  misc_feature 1..41: note = gRNA
  misc_feature 40..41: note = Linked by phosphorothioate (PS) bond
  misc_feature 41: note = Modified to contain 2'-O-methyl
  source 1..41: mol_type = other RNA

SEQ ID NO: 1070 - RNA, 42 nt, synthetic construct
  misc_feature 1..42: note = gRNA
  misc_feature 41: note = Modified to contain 2'-O-methyl
  misc_feature 41..42: note = Linked by phosphorothioate (PS) bond
  source 1..42: mol_type = other RNA
taatttctactcttgtagatccttgtcaaggctattggtcat

SEQ ID NO: 1071 - RNA, 41 nt, synthetic construct
  misc_feature 1..41: note = gRNA
  source 1..41: mol_type = other RNA
taatttctactcttgtagatccttgtcaaggctattggtct

SEQ ID NO: 1072 - RNA, 42 nt, synthetic construct; identical to SEQ ID NO: 1070
  misc_feature 1..42: note = gRNA
  source 1..42: mol_type = other RNA

SEQ ID NO: 1073 - RNA, 44 nt, synthetic construct
  misc_feature 1..44: note = gRNA
  source 1..44: mol_type = other RNA
taatttctactcttgtagatccttgtcaaggctattggtcaagt

SEQ ID NO: 1074 - DNA, 66 bp, synthetic construct
  misc_feature 1..66: note = gRNA
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..66: note = RNA nucleotides
  source 1..66: mol_type = other DNA
atgtgtttttgtcaaaagacctttttaatttctactcttgtagatccttgtcaaggctattggtct

SEQ ID NO: 1075 - DNA, 69 bp, synthetic construct
  misc_feature 1..69: note = gRNA
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..69: note = RNA nucleotides
  source 1..69: mol_type = other DNA
atgtgtttttgtcaaaagacctttttaatttctactcttgtagatccttgtcaaggctattggtcaagt

SEQ ID NO: 1076 - RNA, 41 nt, synthetic construct; identical to SEQ ID NO: 1023
  misc_feature 1..41: note = gRNA
  source 1..41: mol_type = other RNA

SEQ ID NO: 1077 - RNA, 43 nt, synthetic construct
  misc_feature 1..43: note = gRNA
  source 1..43: mol_type = other RNA
taatttctactcttgtagatccttgtcaaggctattggtcaag

SEQ ID NO: 1078 - DNA, 68 bp, synthetic construct
  misc_feature 1..68: note = gRNA
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..68: note = RNA nucleotides
  source 1..68: mol_type = other DNA
atgtgtttttgtcaaaagacctttttaatttctactcttgtagatccttgtcaaggctattggtcaag

SEQ ID NO: 1079 - RNA, 44 nt, synthetic construct
  misc_feature 1..44: note = gRNA
  source 1..44: mol_type = other RNA
taatttctactcttgtagatccttgtcaaggctattggtctttt

SEQ ID NO: 1080 - RNA, 44 nt, synthetic construct; identical to SEQ ID NO: 1079
  misc_feature 1..44: note = gRNA
  misc_feature 41: note = Modified to contain 2'-O-methyl
  misc_feature 41..42: note = Linked by phosphorothioate (PS) bond
  misc_feature 42..43: note = Linked by phosphorothioate (PS) bond
  misc_feature 42: note = Modified to contain 2'-O-methyl
  misc_feature 43: note = Modified to contain 2'-O-methyl
  misc_feature 43..44: note = Linked by phosphorothioate (PS) bond
  source 1..44: mol_type = other RNA

SEQ ID NO: 1081 - DNA, 69 bp, synthetic construct
  misc_feature 1..69: note = gRNA
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..69: note = RNA nucleotides
  source 1..69: mol_type = other DNA
atgtgtttttgtcaaaagacctttttaatttctactcttgtagatccttgtcaaggctattggtctttt

SEQ ID NO: 1082 - DNA, 66 bp, synthetic construct
  misc_feature 1..66: note = gRNA
  misc_feature 1..25: note = DNA nucleotides
  misc_feature 26..66: note = RNA nucleotides
  misc_feature 65..66: note = Linked by phosphorothioate (PS) bond
  misc_feature 66: note = Modified to contain 2'-O-methyl
  source 1..66: mol_type = other DNA
aaaaaaaaaaaaaaaaaaaaaaaaataatttctactcttgtagatccttgtcaaggctattggtca

SEQ ID NO: 1083 - DNA, 66 bp, synthetic construct
  misc_feature 1..66: note = gRNA
  misc_feature 1..25: note = DNA nucleotides
  misc_feature 26..66: note = RNA nucleotides
  misc_feature 65..66: note = Linked by phosphorothioate (PS) bond
  misc_feature 66: note = Modified to contain 2'-O-methyl
  source 1..66: mol_type = other DNA
ttttttttttttttttttttttttttaatttctactcttgtagatccttgtcaaggctattggtca

SEQ ID NO: 1084 - DNA, 61 bp, synthetic construct
  misc_feature 1..61: note = gRNA
  misc_feature 1..20: note = DNA nucleotides
  misc_feature 1..2: note = Linked by phosphorothioate (PS) bond
  misc_feature 2..3: note = Linked by phosphorothioate (PS) bond
  misc_feature 21..61: note = RNA nucleotides
  misc_feature 60..61: note = Linked by phosphorothioate (PS) bond
  misc_feature 61: note = Modified to contain 2'-O-methyl
  source 1..61: mol_type = other DNA
ccgaagttttcttcggtttttaatttctactcttgtagatccttgtcaaggctattggtca

SEQ ID NO: 1085 - RNA, 41 nt, synthetic construct
  misc_feature 1..41: note = gRNA
  misc_feature 40..41: note = Linked by phosphorothioate (PS) bond
  misc_feature 41: note = Modified to contain 2'-O-methyl
  source 1..41: mol_type = other RNA
taatttctactcttgtagatagacagatatttgcattgaga

SEQ ID NO: 1086 - DNA, 66 bp, synthetic construct
  misc_feature 1..66: note = gRNA
  misc_feature 1..25: note = DNA nucleotides
  misc_feature 26..66: note = RNA nucleotides
  misc_feature 65..66: note = Linked by phosphorothioate (PS) bond
  misc_feature 66: note = Modified to contain 2'-O-methyl
  source 1..66: mol_type = other DNA
atgtgtttttgtcaaaagacctttttaatttctactcttgtagatagacagatatttgcattgaga

SEQ ID NO: 1087 - DNA, 61 bp, synthetic construct
  misc_feature 1..61: note = gRNA
  misc_feature 1..20: note = DNA nucleotides
  misc_feature 1..2: note = Linked by phosphorothioate (PS) bond
  misc_feature 2..3: note = Linked by phosphorothioate (PS) bond
  misc_feature 21..61: note = RNA nucleotides
  misc_feature 60..61: note = Linked by phosphorothioate (PS) bond
  misc_feature 61: note = Modified to contain 2'-O-methyl
  source 1..61: mol_type = other DNA
ccgaagttttcttcggtttttaatttctactcttgtagatagacagatatttgcattgaga

SEQ ID NO: 1088 - RNA, 41 nt, synthetic construct
  misc_feature 1..41: note = gRNA
  misc_feature 40..41: note = Linked by phosphorothioate (PS) bond
  misc_feature 41: note = Modified to contain 2'-O-methyl
  source 1..41: mol_type = other RNA
taatttctactcttgtagatcattgagatagtgtggggaag

SEQ ID NO: 1089 - DNA, 66 bp, synthetic construct
  misc_feature 1..66: note = gRNA
  misc_feature 1..25: note = DNA nucleotides
  misc_feature 26..66: note = RNA nucleotides
  misc_feature 65..66: note = Linked by phosphorothioate (PS) bond
  misc_feature 66: note = Modified to contain 2'-O-methyl
  source 1..66: mol_type = other DNA
atgtgtttttgtcaaaagacctttttaatttctactcttgtagatcattgagatagtgtggggaag

SEQ ID NO: 1090 - DNA, 61 bp, synthetic construct
  misc_feature 1..61: note = gRNA
  misc_feature 1..20: note = DNA nucleotides
  misc_feature 1..2: note = Linked by phosphorothioate (PS) bond
  misc_feature 2..3: note = Linked by phosphorothioate (PS) bond
  misc_feature 21..61: note = RNA nucleotides
  misc_feature 60..61: note = Linked by phosphorothioate (PS) bond
  misc_feature 61: note = Modified to contain 2'-O-methyl
  source 1..61: mol_type = other DNA
ccgaagttttcttcggtttttaatttctactcttgtagatcattgagatagtgtggggaag

SEQ ID NO: 1091 - RNA, 41 nt, synthetic construct
  misc_feature 1..41: note = gRNA
  misc_feature 40..41: note = Linked by phosphorothioate (PS) bond
  misc_feature 41: note = Modified to contain 2'-O-methyl
  source 1..41: mol_type = other RNA
taatttctactcttgtagatcttctcccatcatagaggata

SEQ ID NO: 1092 - DNA, 66 bp, synthetic construct
  misc_feature 1..66: note = gRNA
  misc_feature 1..25: note = DNA nucleotides
  misc_feature 26..66: note = RNA nucleotides
  misc_feature 65..66: note = Linked by phosphorothioate (PS) bond
  misc_feature 66: note = Modified to contain 2'-O-methyl
  source 1..66: mol_type = other DNA
atgtgtttttgtcaaaagacctttttaatttctactcttgtagatcttctcccatcatagaggata

SEQ ID NO: 1093 - DNA, 61 bp, synthetic construct
  misc_feature 1..61: note = gRNA
  misc_feature 1..20: note = DNA nucleotides
  misc_feature 1..2: note = Linked by phosphorothioate (PS) bond
  misc_feature 2..3: note = Linked by phosphorothioate (PS) bond
  misc_feature 21..61: note = RNA nucleotides
  misc_feature 60..61: note = Linked by phosphorothioate (PS) bond
  misc_feature 61: note = Modified to contain 2'-O-methyl
  source 1..61: mol_type = other DNA
ccgaagttttcttcggtttttaatttctactcttgtagatcttctcccatcatagaggata

SEQ ID NO: 1094 - protein, 1363 aa, synthetic construct
  REGION 1..1363: note = Cpf1
  REGION 1310..1331: note = MISC_FEATURE - NLS
  REGION 1358..1363: note = MISC_FEATURE - His tag
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1095 - protein, 1363 aa, synthetic construct
  REGION 1..1363: note = Cpf1
  REGION 1310..1331: note = MISC_FEATURE - NLS
  REGION 1358..1363: note = MISC_FEATURE - His tag
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1096 - protein, 1363 aa, synthetic construct
  REGION 1..1363: note = Cpf1
  REGION 1310..1331: note = MISC_FEATURE - NLS
  REGION 1358..1363: note = MISC_FEATURE - His tag
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1097 - protein, 1331 aa, synthetic construct
  REGION 1..1331: note = Cpf1
  REGION 1310..1331: note = MISC_FEATURE - NLS
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1098 - DNA, 67 bp, synthetic construct
  misc_feature 1..67: note = gRNA
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..67: note = RNA nucleotides
  misc_feature 66..67: note = Linked by phosphorothioate (PS) bond
  misc_feature 66: note = Modified to contain 2'-O-methyl
  source 1..67: mol_type = other DNA
atgtgtttttgtcaaaagacctttttaatttctactcttgtagatccttgtcaaggctattggtcat

SEQ ID NO: 1099 - DNA, 68 bp, synthetic construct
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..68: note = RNA nucleotides
  misc_feature 66..67: note = Linked by phosphorothioate (PS) bond
  misc_feature 66: note = Modified to contain 2'-O-methyl
  misc_feature 67: note = Modified to contain 2'-O-methyl
  misc_feature 67..68: note = Linked by phosphorothioate (PS) bond
  source 1..68: mol_type = other DNA
atgtgtttttgtcaaaagacctttttaatttctactcttgtagatccttgtcaaggctattggtcaat

SEQ ID NO: 1100 - DNA, 66 bp, synthetic construct; identical to SEQ ID NO: 1074
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..66: note = RNA nucleotides
  misc_feature 65: note = Modified to contain 2'-O-methyl
  misc_feature 65..66: note = Linked by phosphorothioate (PS) bond
  source 1..66: mol_type = other DNA

SEQ ID NO: 1101 - DNA, 65 bp, synthetic construct; identical to SEQ ID NO: 1067
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..65: note = RNA nucleotides
  misc_feature 64..65: note = Linked by phosphorothioate (PS) bond
  misc_feature 65: note = Modified to contain 2'-O-methyl
  source 1..65: mol_type = other DNA

SEQ ID NO: 1102 - DNA, 67 bp, synthetic construct
  misc_feature 1..25: note = DNA nucleotides
  misc_feature 1..2: note = Linked by phosphorothioate (PS) bond
  misc_feature 2..3: note = Linked by phosphorothioate (PS) bond
  misc_feature 26..67: note = RNA nucleotides
  misc_feature 65..66: note = Linked by phosphorothioate (PS) bond
  misc_feature 66..67: note = Linked by phosphorothioate (PS) bond
  misc_feature 66: note = Modified to contain 2'-O-methyl
  misc_feature 67: note = Modified to contain 2'-O-methyl
  source 1..67: mol_type = other DNA
atgtgtttttgtcaaaagacctttttaatttctactcttgtagatccttgtcaaggctattggtcaa

SEQ ID NO: 1103 - RNA, 66 nt, synthetic construct
  misc_feature 1..66: note = gRNA
  misc_feature 1..2: note = Linked by phosphorothioate (PS) bond
  misc_feature 1: note = Modified to contain 2'-O-methyl
  misc_feature 2: note = Modified to contain 2'-O-methyl
  misc_feature 2..3: note = Linked by phosphorothioate (PS) bond
  misc_feature 65..66: note = Linked by phosphorothioate (PS) bond
  misc_feature 66: note = Modified to contain 2'-O-methyl
  source 1..66: mol_type = other RNA
atgtgtttttgtcaaaagacctttttaatttctactcttgtagatccttgtcaaggctattggtca

SEQ ID NO: 1104 - RNA, 66 nt, synthetic construct
  misc_feature 1..66: note = gRNA
  misc_feature 1..2: note = Linked by phosphorothioate (PS) bond
  misc_feature 1: note = Modified to contain 2'-O-methyl
  misc_feature 2: note = Modified to contain 2'-O-methyl
  misc_feature 2..3: note = Linked by phosphorothioate (PS) bond
  misc_feature 65..66: note = Linked by phosphorothioate (PS) bond
  misc_feature 66: note = Modified to contain 2'-O-methyl
  source 1..66: mol_type = other RNA
aaaaaaaaaaaaaaaaaaaaaaaaataatttctactcttgtagatccttgtcaaggctattggtca

SEQ ID NO: 1105 - RNA, 66 nt, synthetic construct
  misc_feature 1..66: note = gRNA
  misc_feature 1..2: note = Linked by phosphorothioate (PS) bond
  misc_feature 1: note = Modified to contain 2'-O-methyl
  misc_feature 2: note = Modified to contain 2'-O-methyl
  misc_feature 2..3: note = Linked by phosphorothioate (PS) bond
  misc_feature 65..66: note = Linked by phosphorothioate (PS) bond
  misc_feature 66: note = Modified to contain 2'-O-methyl
  source 1..66: mol_type = other RNA
ttttttttttttttttttttttttttaatttctactcttgtagatccttgtcaaggctattggtca

SEQ ID NO: 1106 - DNA, 42 bp, synthetic construct
  misc_feature 1..2: note = Linked by phosphorothioate (PS) bond
  misc_feature 2..3: note = Linked by phosphorothioate (PS) bond
  source 1..42: mol_type = other DNA
taatttctactcttgtagatccttgtcaggctattggtcaag

SEQ ID NO: 1107 - protein, 1331 aa, synthetic construct
  REGION 1..1331: note = Cpf1
  REGION 1310..1331: note = MISC_FEATURE - NLS
STQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 1108 - protein, 1363 aa, synthetic construct
  REGION 1..1363: note = Cpf1
  REGION 1310..1331: note = MISC_FEATURE - NLS
  REGION 1358..1363: note = MISC_FEATURE - His tag
STQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 1109 - protein, 1361 aa, synthetic construct
  REGION 1..1361: note = Cpf1
  REGION 1342..1348: note = MISC_FEATURE - NLS
  REGION 1356..1361: note = MISC_FEATURE - His tag
MGRDPGKPIPNPLLGLDSTAPKKKRKVGIHGVPAATQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVKLAAALEHHHHHH

SEQ ID NO: 1110 - DNA, 4002 bp, synthetic construct
  misc_feature 1..4002: note = His-AsCpf1-sNLS-sNLS H800A
  misc_feature 7..24: note = His tag
  misc_feature 3955..3975: note = sNLS
  misc_feature 3982..4002: note = sNLS
  source 1..4002: mol_type = other DNA
atgggccatcatcatcaccatcacggtagcacccagtttgaaggctttaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacgtacgcagatcagtgtctgcagctggttcagctggattgggaaaatctgagcgcagcaattgatagttatcgcaaagagaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacacagattgatctgtataaccagctgttaggtggtattagccgtgaagcaggcaccgaaaaaatcaaaggtctgaatgaagtgctgaatctggccattcagaaaaatgatgaaaccgcacatattattgcaagcctgccgcatcgttttattccgctgtttaagcagattctgagcgatcgtaataccctgagctttattctggaagaattcaaatccgatgaagaggtgattcagagcttctgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgagccatgcacatgcagcactggatcagccgctgccgaccacactgaaaaaacaagaagaaaaagagatcctgaaaagccagctggatagcctgctgggtctgtatcatctgctggactggtttgcagttgatgaaagcaatgaagttgatccggaatttagcgcacgtctgaccggcattaagctggaaatggaaccgagcctgagtttttacaataaagcccgtaattatgccaccaaaaaaccgtatagcgtggaaaaattcaagctgaacttccagatgccgacactggcaagcggttgggatgttaataaagaaaagaacaatggtgccatcctgttcgtgaaaaatggcctgtattatctgggtatcatgccgaaacagaaaggtcgttataaagcactgagctttgaaccgacagaaaaaaccagtgaaggcttcgataaaatgtactatgattactttccggacgcagccaaaatgattccgaaatgcagcacccagctgaaagcagttaccgcacattttcagacccataccacaccgattctgctgagcaataactttattgaaccgctggaaatcaccaaagagatctacgatctgaataacccggaaaaagagccgaaaaagtttcagaccgcctatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaataatggatgcagttgaaaccggtaagctgtacctgttccagatttataacaaagactttgccaaaggccatcatggtaaaccgaatctgcataccctgtattggaccggtctgtttagccctgaaaatctggcaaaaaccagcattaaactgaatggtcaggcggaactgttttatcgtccgaaaagccgtatgaaacgtatggcagcacgtctgggtgaaaaaatgctgaataaaaagctgaaggatcagaaaaccccgattccggatacactgtatcaagagctgtatgattatgtgaatcatcgtctgagtcatgatctgagtgatgaagcacgtgccctgctgccgaatgttattaccaaagaagttagccacgagatcattaaagatcgtcgttttaccagcgacaagttttttttccatgttccgattacgctgaattatcaggcagcaaatagcccgagcaaatttaaccagcgtgtgaatgcatatctgaaagaacatccggaaacaccgattattggtattgatcgtggtgaacgcaacctgatttatatcaccgttattgatagcaccggcaagatcctggaacagcgtagcctgaataccattcagcagtttgattatcagaaaaagctggacaaccgtgaaaaagaacgtgttgcagcccgtcaggcatggtcagttgttggtacaattaaagacctgaaacagggttatctgagccaggttattcatgaaattgtggatctgatgattcattaccaggcagttgttgtgctggaaaacctgaattttggctttaaaagcaaacgtaccggcattgcggaaaaagcagtttatcagcagttcgaaaagatgctgatcgataaactgaactgtctggtgctgaaagattatccggctgaaaaagttggcggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggttttgatttcctgcattacgacgttaagaccggtgattttattctgcactttaagatgaatcggaacctgtcatttcagcgtggtctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgttagagaatgatgatagccatgccattgataccatggttgccctgattcgtagcgttctgcagatgcgtaatagcaatgcagcaaccggtgaagattacattaatagtccggttcgtgatctgaatggtgtttgttttgatagccgttttcagaatccggaatggccgatggatgcggatgcaaatggtgcatatcatattgcactgaaaggacagctgctgctgaaccacctgaaagaaagtaaagatttgaagctgcagaatggcattagcaatcaggattggctggcatatatccaagaactgcgtaacggttctcctaagaaaaagcgcaaagttggcagtccgaagaagaaacgtaaggtt

SEQ ID NO: 1111 - DNA, 4089 bp, synthetic construct
  misc_feature 1..4089: note = Cpf1
  misc_feature 3928..3993: note = NLS
  misc_feature 4072..4089: note = His tag
  source 1..4089: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagttggtggtagcggtggttcaggtggtagtggcggtagtggtggctcagggggttctggtggctctggtggtagcctcgagcaccaccaccaccaccac

SEQ ID NO: 1112 - DNA, 4089 bp, synthetic construct
  misc_feature 1..4089: note = Cpf1
  misc_feature 3928..3993: note = NLS
  misc_feature 4072..4089: note = His tag
  source 1..4089: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagatgccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcttttttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagttggtggtagcggtggttcaggtggtagtggcggtagtggtggctcagggggttctggtggctctggtggtagcctcgagcaccaccaccaccaccac

SEQ ID NO: 1113 - DNA, 4089 bp, synthetic construct
  misc_feature 1..4089: note = Cpf1
  misc_feature 3928..3993: note = NLS
  misc_feature 4072..4089: note = His tag
  source 1..4089: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcagctcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagttggtggtagcggtggttcaggtggtagtggcggtagtggtggctcagggggttctggtggctctggtggtagcctcgagcaccaccaccaccaccac

SEQ ID NO: 1114 - DNA, 3993 bp, synthetic construct
  misc_feature 1..3993: note = Cpf1
  misc_feature 3928..3993: note = NLS
  source 1..3993: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcagctcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagtt

SEQ ID NO: 1115 - DNA, 3993 bp, synthetic construct
  misc_feature 1..3993: note = Cpf1
  misc_feature 3928..3993: note = NLS
  source 1..3993: mol_type = other DNA
agcacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagtt

SEQ ID NO: 1116 - DNA, 4089 bp, synthetic construct
  misc_feature 1..4089: note = Cpf1
  misc_feature 3928..3993: note = NLS
  misc_feature 4072..4089: note = His tag
  source 1..4089: mol_type = other DNA
agcacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagttggtggtagcggtggttcaggtggtagtggcggtagtggtggctcagggggttctggtggctctggtggtagcctcgagcaccaccaccaccaccac

SEQ ID NO: 1117 - DNA, 4083 bp, synthetic construct
  misc_feature 1..4083: note = Cpf1
  misc_feature 4024..4044: note = NLS
  misc_feature 4066..4083: note = His tag
  source 1..4083: mol_type = other DNA
atgggtcgggatccaggtaaaccgattccgaatccgctgctgggtctggatagcaccgcaccgaaaaaaaaacgtaaagttggtattcatggtgttccggcagcaacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagatgccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcttttttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaaccctaaaaaaaaacgcaaagtgaagcttgcggccgcactcgagcaccaccaccaccaccac

SEQ ID NO: 1118 - DNA, 304 bp, Homo sapiens
  source 1..304: mol_type = other DNA
tcctaaagcttggaacactttcccttccttaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcatcaaaatgcacatctcatataaaaataaatgaggagcatgcacacaccacaaacacaaacaggcatgcagaaat

SEQ ID NO: 1119 - DNA, 665 bp, Homo sapiens
  source 1..665: mol_type = other DNA
ataaagatgaacccatagtgagctgagagctccagcctggcctccagataactacacaccaagcttccacccagaatcaagcctatgttaacttccctcaaagcctgagattttgccttcccattaaatgcaggtagttgttccccttcaagcactagtcactggccataatttaaatcttgctatcttcttgccaccatgaaccctgtatgttgtaggctgaagacgttaaaagaaacacacgctgacacacacacacacacgcgcgcgcgcacacacacacacacacacagagctgactttcaaaatctactccagcccaaatgtttcaattgttcctcacccctggacatactttgcccccatctggaattaaaggatataagtttgtaatgaagcattagcagcattttatatgtgtccagctgatataggaatagccttagcaatgtatgtttggccaccaaagttccccactttgactgagccaatatatgccttctgcctgcatctttttaacgaccatacttgtcctgcctccagatagatgttttaaaacaacaaaaatgagggaaagatgaaagttctttctactggaatctaataaagaaaagtcattttcctcatttccacctctcttttctcaaagtcaaaattgtccatct

SEQ ID NO: 1120 - DNA, 165 bp, Homo sapiens
  source 1..165: mol_type = other DNA
ccctaaaacattaccactgggtctcagcccagttagtcctctgcagtttcttcacccccaaccccagtatcttcaaacagctcacaccctgctgtgctcagatcaatactccgttgtctaagttgcctcgagactaaaggcaacagggctgaaacatctcctgga

SEQ ID NO: 1121 - DNA, 122 bp, Homo sapiens
  source 1..122: mol_type = other DNA
ctgtgagattgacaagaacagtttgacagtcagaaggtgccacaaatcctgagaagcgacctggacttttgccaggcacagggtccttccttccctcccttgtcctggtcaccagagcctac

SEQ ID NO: 1122 - DNA, 24 bp, Homo sapiens
  source 1..24: mol_type = other DNA
gccgccggcccctggcctcactgg

SEQ ID NO: 1123 - DNA, 33 bp, Homo sapiens
  source 1..33: mol_type = other DNA
ccttgtcaaggctattggtcaaggcaaggctgg

SEQ ID NO: 1124 - DNA, 36 bp, Homo sapiens
  source 1..36: mol_type = other DNA
tgagatagtgtggggaaggggcccccaagaggatac

SEQ ID NO: 1125 - DNA, 41 bp, Homo sapiens
  source 1..41: mol_type = other DNA
tatagcctttgccttgttccgattcagtcattccagttttt

SEQ ID NO: 1126 - DNA, 114 bp, Homo sapiens
  source 1..114: mol_type = other DNA
tcttccctttagctagtttccttctcccatcatagaggataccaggacttcttttgtcagccgttttttaccttcttgtctctagctccagtgaggcctgtagtttaaagctaa

SEQ ID NO: 1127 - DNA, 86 bp, Homo sapiens
  source 1..86: mol_type = other DNA
ccacagtttcagcgcagtaatagattagtgttacataatataagacctaatgcttacctcaatatctacttatccgtacctatttg

SEQ ID NO: 1128 - DNA, 142 bp, Homo sapiens
  source 1..142: mol_type = other DNA
tattcaggtatgtatgtatacaccagatgatgtgtatttaccactggataagtgtgtgtgctggctgatgacccagggttttggcgtagctcttctatgctcagtaaagatgatggtagaatgttctttggcaggtactgtg

SEQ ID NO: 1129 - DNA, 697 bp, Homo sapiens
  source 1..697: mol_type = other DNA
caataaagatgaacccatagtgagctgagagctccagcctggcctccagataactacacaccaagcttccacccagaatcaagcctatgttaacttccctcaaagcctgagattttgctttcccattaaatgcaggtagttgttcttcttgcagcactagtcactggccataatttaaatcttgttatcttcttgccaccatgaaccctgtatgctgtaggctgaaaacgttaaaagaaacacacgctctcacacacacacaaacacacgcgcgcacacacacacacacacacacagagctgactttcaaaatctactccagcccaaatgtttcaattgttcctcacccctggacatactttgcccccatctggaattaaaggatataagtttgtaatgaagcattagcagcattttatatgtgtccagctgatataggaatagccttagcaatgtatgtttggccaccaaagttccccactttgactgagccaatatatgccttctgcctgcatctttttaatgaccatacttgtcctgcctccagatagatgttttaaaacgaataacaaaaataggggaaaggtgaaagttctttctaccgaaatctaataaagaaaagtcattttcctcatttccacctctcttttctcaaagtcaaagttgtccatctagattttcagaggcactccttagg

SEQ ID NO: 1130 - DNA, 185 bp, Homo sapiens
  source 1..185: mol_type = other DNA
ccctaaaacattgccactgggtctcagcccagttagtcctctgcagtttcttcactcccaaccccagtatcttcaaacagctcacaccctgctgtgctcagatcaatactcagttgtctaagttgcctcgagactaaaggcaacagtgctgaaacatctcctggactcaccttgaagttctcagg

SEQ ID NO: 1131 - DNA, 137 bp, Homo sapiens
  source 1..137: mol_type = other DNA
agcctgtgagattgacaagaacagtttgacagtcagaaggtgccacaaatcctgagaagcgacctggacttttgccaggcacagggtccttccttccctcccttgtcctggtcaccagagcctaccttcccagggtt

SEQ ID NO: 1132 - DNA, 38 bp, Homo sapiens
  source 1..38: mol_type = other DNA
ccgccggcccctggcctcactggatactctaagactat

SEQ ID NO: 1133 - DNA, 31 bp, Homo sapiens
  source 1..31: mol_type = other DNA
ccttgtcaaggctattggtcaaggcaaggct

SEQ ID NO: 1134 - DNA, 75 bp, Homo sapiens
  source 1..75: mol_type = other DNA
cagggaccgtttcagacagatatttgcattgagatagtgtggggaaggggcccccaagaggatactgctgcttaa

SEQ ID NO: 1135 - DNA, 29 bp, Homo sapiens
  source 1..29: mol_type = other DNA
ttgccttgttccgattcagtcattccaat

SEQ ID NO: 1136 - DNA, 104 bp, Homo sapiens
  source 1..104: mol_type = other DNA
tttagctagttttcttctcccaccatagaagataccaggacttcttttgtcagccgtttttcaccttcttgtctgtagctccagtgaggcctgtagtttaaagt

SEQ ID NO: 1137 - DNA, 38 bp, Homo sapiens
  source 1..38: mol_type = other DNA
ggacacgtcttagtctcatttagtaagcattggtttcc

SEQ ID NO: 1138 - DNA, 124 bp, Homo sapiens
  source 1..124: mol_type = other DNA
ttttttatattcaggtatgtatgtaggcacccgatgatgtgtatttatcactggataagtgtatgtgctggctgatgacccagggttttggtgtagctcttctatgctcggtaaagatgatggt

SEQ ID NO: 1139 - RNA, 20 nt, synthetic construct
  misc_feature 1..20: note = gRNA targeting domain - AsCpf1 HBG1 Promoter-1
  source 1..20: mol_type = other RNA
agacagatatttgcattgag

SEQ ID NO: 1140 - DNA, 20 bp, synthetic construct
  misc_feature 1..20: note = gRNA targeting domain - AsCpf1 HBG1 Promoter-1
  source 1..20: mol_type = other DNA
agacagatatttgcattgag

SEQ ID NO: 1141 - RNA, 20 nt, synthetic construct
  misc_feature 1..20: note = gRNA targeting domain - AsCpf1 HBG1 Promoter-2
  source 1..20: mol_type = other RNA
cattgagatagtgtggggaa

SEQ ID NO: 1142 - DNA, 20 bp, synthetic construct
  misc_feature 1..20: note = gRNA targeting domain - AsCpf1 HBG1 Promoter-2
  source 1..20: mol_type = other DNA
cattgagatagtgtggggaa

SEQ ID NO: 1143 - RNA, 20 nt, synthetic construct
  misc_feature 1..20: note = gRNA targeting domain - AsCpf1 HBG1 Promoter-3
  source 1..20: mol_type = other RNA
tagcctttgccttgttccga

SEQ ID NO: 1144 - DNA, 20 bp, synthetic construct
  misc_feature 1..20: note = gRNA targeting domain - AsCpf1 HBG1 Promoter-3
  source 1..20: mol_type = other DNA
tagcctttgccttgttccga

SEQ ID NO: 1145 - RNA, 20 nt, synthetic construct
  misc_feature 1..20: note = gRNA targeting domain - AsCpf1 HBG1 Promoter-4
  source 1..20: mol_type = other RNA
ccttgttccgattcagtcat

SEQ ID NO: 1146 - DNA, 20 bp, synthetic construct
  misc_feature 1..20: note = gRNA targeting domain - AsCpf1 HBG1 Promoter-4
  source 1..20: mol_type = other DNA
ccttgttccgattcagtcat

SEQ ID NO: 1147 - RNA, 20 nt, synthetic construct
  misc_feature 1..20: note = gRNA targeting domain - AsCpf1 HBG1 Promoter-5
  source 1..20: mol_type = other RNA
tctaatttattcttcccttt

SEQ ID NO: 1148 - DNA, 20 bp, synthetic construct
  misc_feature 1..20: note = gRNA targeting domain - AsCpf1 HBG1 Promoter-5
  source 1..20: mol_type = other DNA
tctaatttattcttcccttt

SEQ ID NO: 1149 - RNA, 20 nt, synthetic construct
  misc_feature 1..20: note = gRNA targeting domain - AsCpf1 HBG1 Promoter-6
  source 1..20: mol_type = other RNA
cttctcccatcatagaggat

SEQ ID NO: 1150 - DNA, 20 bp, synthetic construct
  misc_feature 1..20: note = gRNA targeting domain - AsCpf1 HBG1 Promoter-6
  source 1..20: mol_type = other DNA
cttctcccatcatagaggat

SEQ ID NO: 1151 - RNA, 20 nt, synthetic construct
  misc_feature 1..20: note = gRNA targeting domain - AsCpf1 HBG2 Promoter-7
  source 1..20: mol_type = other RNA
ttctcccaccatagaagata

SEQ ID NO: 1152 - DNA, 20 bp, synthetic construct
  misc_feature 1..20: note = gRNA targeting domain - AsCpf1 HBG2 Promoter-7
  source 1..20: mol_type = other DNA
ttctcccaccatagaagata

SEQ ID NO: 1153 - RNA, 20 nt, synthetic construct
  misc_feature 1..20: note = gRNA targeting domain - AsCpf1 HBG1 Promoter-8
  source 1..20: mol_type = other RNA
ccactggataagtgtgtgtg

SEQ ID NO: 1154 - DNA, 20 bp, synthetic construct
  misc_feature 1..20: note = gRNA targeting domain - AsCpf1 HBG1 Promoter-8
  source 1..20: mol_type = other DNA
ccactggataagtgtgtgtg

SEQ ID NO: 1155 - RNA, 20 nt, synthetic construct
  misc_feature 1..20: note = gRNA targeting domain - AsCpf1 HBG1 Promoter-9
  source 1..20: mol_type = other RNA
gcgtagctcttctatgctca

SEQ ID NO: 1156 - DNA, 20 bp, synthetic construct
  misc_feature 1..20: note = gRNA targeting domain - AsCpf1 HBG1 Promoter-9
  source 1..20: mol_type = other DNA
gcgtagctcttctatgctca

SEQ ID NO: 1157 - RNA, 20 nt, synthetic construct
  misc_feature 1..20: note = gRNA targeting domain - AsCpf1 HBG1 Promoter-10
  source 1..20: mol_type = other RNA
ctgagcatagaagagctacg

SEQ ID NO: 1158 - DNA, 20 bp, synthetic construct
  misc_feature 1..20: note = gRNA targeting domain - AsCpf1 HBG1 Promoter-10
  source 1..20: mol_type = other DNA
ctgagcatagaagagctacg

SEQ ID NO: 1159 - RNA, 20 nt, synthetic construct
  misc_feature 1..20: note = gRNA targeting domain - AsCpf1 HBG2 Promoter-11
  source 1..20: mol_type = other RNA
tcactggataagtgtatgtg

SEQ ID NO: 1160 - DNA, 20 bp, synthetic construct
  misc_feature 1..20: note = gRNA targeting domain - AsCpf1 HBG2 Promoter-11
  source 1..20: mol_type = other DNA
tcactggataagtgtatgtg

SEQ ID NO: 1161 - RNA, 20 nt, synthetic construct
  misc_feature 1..20: note = gRNA targeting domain - AsCpf1 HBG2 Promoter-12
  source 1..20: mol_type = other RNA
gtgtagctcttctatgctcg

SEQ ID NO: 1162 - DNA, 20 bp, synthetic construct
  misc_feature 1..20: note = gRNA targeting domain - AsCpf1 HBG2 Promoter-12
  source 1..20: mol_type = other DNA
gtgtagctcttctatgctcg

SEQ ID NO: 1163 - RNA, 20 nt, synthetic construct
  misc_feature 1..20: note = gRNA targeting domain - AsCpf1 HBG2 Promoter-13
  source 1..20: mol_type = other RNA
ccgagcatagaagagctaca

SEQ ID NO: 1164 - DNA, 20 bp, synthetic construct
  misc_feature 1..20: note = gRNA targeting domain - AsCpf1 HBG2 Promoter-13
  source 1..20: mol_type = other DNA
ccgagcatagaagagctaca

SEQ ID NO: 1165-1166 - skipped (2 entries)

SEQ ID NO: 1167 - RNA, 20 nt, synthetic construct
  misc_feature 1..20: note = gRNA targeting domain - AsCpf1 RR HBG1 Promoter-1
  source 1..20: mol_type = other RNA
gacagatatttgcattgaga

SEQ ID NO: 1168 - DNA, 20 bp, synthetic construct
  misc_feature 1..20: note = gRNA targeting domain - AsCpf1 RR HBG1 Promoter-1
  source 1..20: mol_type = other DNA
gacagatatttgcattgaga

SEQ ID NO: 1169 - RNA, 20 nt, synthetic construct
  misc_feature 1..20: note = gRNA targeting domain - AsCpf1 RR HBG1 Promoter-2
  source 1..20: mol_type = other RNA
acactatctcaatgcaaata

SEQ ID NO: 1170 - DNA, 20 bp, synthetic construct
  misc_feature 1..20: note = gRNA targeting domain - AsCpf1 RR HBG1 Promoter-2
  source 1..20: mol_type = other DNA
acactatctcaatgcaaata

SEQ ID NO: 1171 - RNA, 20 nt, synthetic construct
  misc_feature 1..20: note = gRNA targeting domain - AsCpf1 RR HBG1 Promoter-3
  source 1..20: mol_type = other RNA
cacactatctcaatgcaaat

SEQ ID NO: 1172 - DNA, 20 bp, synthetic construct
  misc_feature 1..20: note = gRNA targeting domain - AsCpf1 RR HBG1 Promoter-3
  source 1..20: mol_type = other DNA
cacactatctcaatgcaaat

SEQ ID NO: 1173 - RNA, 20 nt, synthetic construct
  misc_feature 1..20: note = gRNA targeting domain - AsCpf1 RR HBG1 Promoter-4
  source 1..20: mol_type = other RNA
ccacactatctcaatgcaaa

SEQ ID NO: 1174 - DNA, 20 bp, synthetic construct
  misc_feature 1..20: note = gRNA targeting domain - AsCpf1 RR HBG1 Promoter-4
  source 1..20: mol_type = other DNA
ccacactatctcaatgcaaa

SEQ ID NO: 1175 - RNA, 20 nt, synthetic construct
  misc_feature 1..20: note = gRNA targeting domain - AsCpf1 RR HBG1 Promoter-5
  source 1..20: mol_type = other RNA
ttccccacactatctcaatg

SEQ ID NO: 1176 - DNA, 20 bp, synthetic construct
  misc_feature 1..20: note = gRNA targeting domain - AsCpf1 RR HBG1 Promoter-5
  source 1..20: mol_type = other DNA
ttccccacactatctcaatg

SEQ ID NO: 1177 - RNA, 20 nt, synthetic construct
  misc_feature 1..20: note = gRNA targeting domain - AsCpf1 RR HBG1 Promoter-6
  source 1..20: mol_type = other RNA
gattcagtcattccagtttt

SEQ ID NO: 1178 - DNA, 20 bp, synthetic construct
  misc_feature 1..20: note = gRNA targeting domain - AsCpf1 RR HBG1 Promoter-6
  source 1..20: mol_type = other DNA
gattcagtcattccagtttt

SEQ ID NO: 1179 - RNA, 20 nt, synthetic construct
  misc_feature 1..20: note = gRNA targeting domain - AsCpf1 RR HBG1 Promoter-7
  source 1..20: mol_type = other RNA
attcagtcattccagttttt

SEQ ID NO: 1180 - DNA, 20 bp, synthetic construct
  misc_feature 1..20: note = gRNA targeting domain - AsCpf1 RR HBG1 Promoter-7
  source 1..20: mol_type = other DNA
attcagtcattccagttttt

SEQ ID NO: 1181 - RNA, 20 nt, synthetic construct
  misc_feature 1..20: note = gRNA targeting domain - AsCpf1 RR HBG1 Promoter-8
  source 1..20: mol_type = other RNA
gtcattccagtttttctcta

SEQ ID NO: 1182 - DNA, 20 bp, synthetic construct
  misc_feature 1..20: note = gRNA targeting domain - AsCpf1 RR HBG1 Promoter-8
  source 1..20: mol_type = other DNA
gtcattccagtttttctcta

SEQ ID NO: 1183 - RNA, 20 nt, synthetic construct
  misc_feature 1..20: note = gRNA targeting domain - AsCpf1 RR HBG1 Promoter-9
  source 1..20: mol_type = other RNA
agtttttctctaatttattc

SEQ ID NO: 1184 - DNA, 20 bp, synthetic construct
  misc_feature 1..20: note = gRNA targeting domain - AsCpf1 RR HBG1 Promoter-9
  source 1..20: mol_type = other DNA
agtttttctctaatttattc

SEQ ID NO: 1185 - RNA, 20 nt, synthetic construct
  misc_feature 1..20: note = gRNA targeting domain - AsCpf1 RR HBG1 Promoter-10
  source 1..20: mol_type = other RNA
gtttttctctaatttattct

SEQ ID NO: 1186 - DNA, 20 bp, synthetic construct
  misc_feature 1..20: note = gRNA targeting domain - AsCpf1 RR HBG1 Promoter-10
  source 1..20: mol_type = other DNA
gtttttctctaatttattct

SEQ ID NO: 1187 - RNA, 20 nt, synthetic construct
  misc_feature 1..20: note = gRNA targeting domain - AsCpf1 RR HBG2 Promoter-11
  source 1..20: mol_type = other RNA
gattcagtcattccaatttt

SEQ ID NO: 1188 - DNA, 20 bp, synthetic construct
  misc_feature 1..20: note = gRNA targeting domain - AsCpf1 RR HBG2 Promoter-11
  source 1..20: mol_type = other DNA
caagaggatactgctgctta

SEQ ID NO: 1189 - RNA, 20 nt, synthetic construct
  misc_feature 1..20: note = gRNA targeting domain - AsCpf1 RR HBG2 Promoter-12
  source 1..20: mol_type = other RNA
attcagtcattccaattttt

SEQ ID NO: 1190 - DNA, 20 bp, synthetic construct
  misc_feature 1..20: note = gRNA targeting domain - AsCpf1 RR HBG2 Promoter-12
  source 1..20: mol_type = other DNA
aagaggatactgctgcttaa

SEQ ID NO: 1191 - RNA, 20 nt, synthetic construct
  misc_feature 1..20: note = gRNA targeting domain - AsCpf1 RR HBG2 Promoter-13
  source 1..20: mol_type = other RNA
gtcattccaatttttctcta

SEQ ID NO: 1192 - DNA, 20 bp, synthetic construct
  misc_feature 1..20: note = gRNA targeting domain - AsCpf1 RR HBG2 Promoter-13
  source 1..20: mol_type = other DNA
gattcagtcattccaatttt

SEQ ID NO: 1193 - RNA, 20 nt, synthetic construct
  misc_feature 1..20: note = gRNA targeting domain - AsCpf1 RR HBG2 Promoter-14
  source 1..20: mol_type = other RNA
aatttttctctaatttattc

SEQ ID NO: 1194 - DNA, 20 bp, synthetic construct
  misc_feature 1..20: note = gRNA targeting domain - AsCpf1 RR HBG2 Promoter-14
  source 1..20: mol_type = other DNA
attcagtcattccaattttt

SEQ ID NO: 1195 - RNA, 20 nt, synthetic construct
  misc_feature 1..20: note = gRNA targeting domain - AsCpf1 RR HBG2 Promoter-15
  source 1..20: mol_type = other RNA
atttttctctaatttattct

SEQ ID NO: 1196 - DNA, 20 bp, synthetic construct
  misc_feature 1..20: note = gRNA targeting domain - AsCpf1 RR HBG2 Promoter-15
  source 1..20: mol_type = other DNA
gtcattccaatttttctcta

SEQ ID NO: 1197 - RNA, 20 nt, synthetic construct
  misc_feature 1..20: note = gRNA targeting domain - AsCpf1 RR HBG2 Promoter-16
  source 1..20: mol_type = other RNA
ttctcccatcatagaggata

SEQ ID NO: 1198 - DNA, 20 bp, synthetic construct
  misc_feature 1..20: note = gRNA targeting domain - AsCpf1 RR HBG2 Promoter-16
  source 1..20: mol_type = other DNA
aatttttctctaatttattc

SEQ ID NO: 1199 - RNA, 20 nt, synthetic construct
  misc_feature 1..20: note = gRNA targeting domain - AsCpf1 RR HBG2 Promoter-17
  source 1..20: mol_type = other RNA
atcatagaggataccaggac

SEQ ID NO: 1200 - DNA, 20 bp, synthetic construct
  misc_feature 1..20: note = gRNA targeting domain - AsCpf1 RR HBG2 Promoter-17
  source 1..20: mol_type = other DNA
atttttctctaatttattct

SEQ ID NO: 1201 - RNA, 20 nt, synthetic construct
  misc_feature 1..20: note = gRNA targeting domain - AsCpf1 RR HBG1 Promoter-18
  source 1..20: mol_type = other RNA
accatagaagataccaggac

SEQ ID NO: 1202 - DNA, 20 bp, synthetic construct
  misc_feature 1..20: note = gRNA targeting domain - AsCpf1 RR HBG1 Promoter-18
  source 1..20: mol_type = other DNA
ttctcccatcatagaggata

SEQ ID NO: 1203 - RNA, 20 nt, synthetic construct
  misc_feature 1..20: note = gRNA targeting domain - AsCpf1 RR HBG1 Promoter-19
  source 1..20: mol_type = other RNA
cagtacctgccaaagaacat

SEQ ID NO: 1204 - DNA, 20 bp, synthetic construct
  misc_feature 1..20: note = gRNA targeting domain - AsCpf1 RR HBG1 Promoter-19
  source 1..20: mol_type = other DNA
atcatagaggataccaggac

SEQ ID NO: 1205 - RNA, 20 nt, synthetic construct
  misc_feature 1..20: note = gRNA targeting domain - AsCpf1 RR HBG2 Promoter-20
  source 1..20: mol_type = other RNA
tagtatctggtaaagagcat

SEQ ID NO: 1206 - DNA, 20 bp, synthetic construct
  misc_feature 1..20: note = gRNA targeting domain - AsCpf1 RR HBG2 Promoter-20
  source 1..20: mol_type = other DNA
accatagaagataccaggac

SEQ ID NO: 1207 - RNA, 20 nt, synthetic construct
  misc_feature 1..20: note = gRNA targeting domain - AsCpf1 RR HBG1 Promoter-21
  source 1..20: mol_type = other RNA
tcaatgcaaatatctgtctg

SEQ ID NO: 1208 - DNA, 20 bp, synthetic construct
  misc_feature 1..20: note = gRNA targeting domain - AsCpf1 RR HBG1 Promoter-21
  source 1..20: mol_type = other DNA
cagtacctgccaaagaacat

SEQ ID NO: 1209 - RNA, 20 nt, synthetic construct
  misc_feature 1..20: note = gRNA targeting domain - AsCpf1 RR HBG2 Promoter-22
  source 1..20: mol_type = other RNA
ctcttgggggccccttcccc

SEQ ID NO: 1210 - DNA, 20 bp, synthetic construct
  misc_feature 1..20: note = gRNA targeting domain - AsCpf1 RR HBG2 Promoter-22
  source 1..20: mol_type = other DNA
tagtatctggtaaagagcat

SEQ ID NO: 1211 - RNA, 20 nt, synthetic construct
  misc_feature 1..20: note = gRNA targeting domain - AsCpf1 RVR HBG1 Promoter-1
  source 1..20: mol_type = other RNA
gattcagtcattccaatttt

SEQ ID NO: 1212 - DNA, 20 bp, synthetic construct
  misc_feature 1..20: note = gRNA targeting domain - AsCpf1 RVR HBG1 Promoter-1
  source 1..20: mol_type = other DNA
tcaatgcaaatatctgtctg

SEQ ID NO: 1213 - RNA, 20 nt, synthetic construct
  misc_feature 1..20: note = gRNA targeting domain - AsCpf1 RVR HBG1 Promoter-2
  source 1..20: mol_type = other RNA
attcagtcattccaattttt

SEQ ID NO: 1214 - DNA, 20 bp, synthetic construct
  misc_feature 1..20: note = gRNA targeting domain - AsCpf1 RVR HBG1 Promoter-2
  source 1..20: mol_type = other DNA
ctcttgggggccccttcccc

SEQ ID NO: 1215 - RNA, 20 nt, synthetic construct
  misc_feature 1..20: note = gRNA targeting domain - AsCpf1 RVR HBG1 Promoter-3
  source 1..20: mol_type = other RNA
aaaaaaattagcagtatcct

SEQ ID NO: 1216 - DNA, 20 bp, synthetic construct
  misc_feature 1..20: note = gRNA targeting domain - AsCpf1 RVR HBG1 Promoter-3
  source 1..20: mol_type = other DNA
aaaaaaattagcagtatcct

SEQ ID NO: 1217 - RNA, 20 nt, synthetic construct
  misc_feature 1..20: note = gRNA targeting domain - AsCpf1 RVR HBG1 Promoter-4
  source 1..20: mol_type = other RNA
gcctttgccttgttccgatt

SEQ ID NO: 1218 - DNA, 20 bp, synthetic construct
  misc_feature 1..20: note = gRNA targeting domain - AsCpf1 RVR HBG1 Promoter-4
  source 1..20: mol_type = other DNA
gcctttgccttgttccgatt

SEQ ID NO: 1219 - RNA, 20 nt, synthetic construct
  misc_feature 1..20: note = gRNA targeting domain - AsCpf1 RVR HBG2 Promoter-5
  source 1..20: mol_type = other RNA
aaaaaaattaagcagcagta

SEQ ID NO: 1220 - DNA, 20 bp, synthetic construct
  misc_feature 1..20: note = gRNA targeting domain - AsCpf1 RVR HBG2 Promoter-5
  source 1..20: mol_type = other DNA
aaaaaaattaagcagcagta

SEQ ID NO: 1221 - RNA, 20 nt, synthetic construct
  misc_feature 1..20: note = gRNA targeting domain - AsCpf1 RVR HBG1 Promoter-6
  source 1..20: mol_type = other RNA
ctcagtaaagatgatggtag

SEQ ID NO: 1222 - DNA, 20 bp, synthetic construct
  misc_feature 1..20: note = gRNA targeting domain - AsCpf1 RVR HBG1 Promoter-6
  source 1..20: mol_type = other DNA
ctcagtaaagatgatggtag

SEQ ID NO: 1223 - RNA, 20 nt, synthetic construct
  misc_feature 1..20: note = gRNA targeting domain - AsCpf1 RVR HBG2 Promoter-7
  source 1..20: mol_type = other RNA
actggataagtgtatgtgct

SEQ ID NO: 1224 - DNA, 20 bp, synthetic construct
  misc_feature 1..20: note = gRNA targeting domain - AsCpf1 RVR HBG2 Promoter-7
  source 1..20: mol_type = other DNA
actggataagtgtatgtgct

SEQ ID NO: 1225 - RNA, 20 nt, synthetic construct
  misc_feature 1..20: note = gRNA targeting domain - AsCpf1 RVR HBG2 Promoter-8
  source 1..20: mol_type = other RNA
tgctggctgatgacccaggg

SEQ ID NO: 1226 - DNA, 20 bp, synthetic construct
  misc_feature 1..20: note = gRNA targeting domain - AsCpf1 RVR HBG2 Promoter-8
  source 1..20: mol_type = other DNA
tgctggctgatgacccaggg

SEQ ID NO: 1227 - RNA, 20 nt, synthetic construct
  misc_feature 1..20: note = gRNA targeting domain - AsCpf1 RVR HBG2 Promoter-9
  source 1..20: mol_type = other RNA
ctcggtaaagatgatggtag

SEQ ID NO: 1228 - DNA, 20 bp, synthetic construct
  misc_feature 1..20: note = gRNA targeting domain - AsCpf1 RVR HBG2 Promoter-9
  source 1..20: mol_type = other DNA
ctcggtaaagatgatggtag

SEQ ID NO: 1229 - RNA, 20 nt, synthetic construct
  misc_feature 1..20: note = gRNA targeting domain - AsCpf1 RVR HBG2 Promoter-10
  source 1..20: mol_type = other RNA
tggtaaagagcattctacca

SEQ ID NO: 1230 - DNA, 20 bp, synthetic construct
  misc_feature 1..20: note = gRNA targeting domain - AsCpf1 RVR HBG2 Promoter-10
  source 1..20: mol_type = other DNA
tggtaaagagcattctacca

SEQ ID NO: 1231 - skipped

SEQ ID NO: 1232 - RNA, 10 nt, synthetic construct
  misc_feature 1..10: note = RNA gRNA 5' extension
  source 1..10: mol_type = other RNA
aagacctttt

SEQ ID NO: 1233 - RNA, 25 nt, synthetic construct
  misc_feature 1..25: note = ≅RNA gRNA 5' extension
  source 1..25: mol_type = other RNA
atgtgtttttgtcaaaagacctttt

SEQ ID NO: 1234 - RNA, 60 nt, synthetic construct
  misc_feature 1..60: note = αRNA gRNA 5' extension
  source 1..60: mol_type = other RNA
aggccagcttgccggttttttagtcgtgctgcttcatgtgtttttgtcaaaagacctttt

SEQ ID NO: 1235 - skipped

SEQ ID NO: 1236 - DNA, 10 bp, synthetic construct
  misc_feature 1..10: note = DNA gRNA 5' extension
  source 1..10: mol_type = other DNA
aagacctttt

SEQ ID NO: 1237 - DNA, 25 bp, synthetic construct
  misc_feature 1..25: note = ≅DNA gRNA 5' extension
  source 1..25: mol_type = other DNA
atgtgtttttgtcaaaagacctttt

SEQ ID NO: 1238 - DNA, 60 bp, synthetic construct
  misc_feature 1..60: note = αDNA gRNA 5' extension
  source 1..60: mol_type = other DNA
aggccagcttgccggttttttagtcgtgctgcttcatgtgtttttgtcaaaagacctttt

SEQ ID NO: 1239 - DNA, 20 bp, synthetic construct
  misc_feature 1..20: note = °DNA gRNA 5' extension
  source 1..20: mol_type = other DNA
tttttgtcaaaagacctttt

SEQ ID NO: 1240 - DNA, 30 bp, synthetic construct
  misc_feature 1..30: note = +DNA gRNA 5' extension
  source 1..30: mol_type = other DNA
gcttcatgtgtttttgtcaaaagacctttt

SEQ ID NO: 1241 - DNA, 50 bp, synthetic construct
  misc_feature 1..50: note = ═DNA gRNA 5' extension
  source 1..50: mol_type = other DNA
gccggttttttagtcgtgctgcttcatgtgtttttgtcaaaagacctttt

SEQ ID NO: 1242 - DNA, 40 bp, synthetic construct
  misc_feature 1..40: note = ′DNA gRNA 5' extension
  source 1..40: mol_type = other DNA
tagtcgtgctgcttcatgtgtttttgtcaaaagacctttt

SEQ ID NO: 1243 - DNA, 20 bp, synthetic construct
  misc_feature 1..20: note = °DNA/2xPS gRNA 5' extension
  misc_feature 1..2: note = Linked by phosphorothioate (PS) bond
  misc_feature 2..3: note = Linked by phosphorothioate (PS) bond
  source 1..20: mol_type = other DNA
ccgaagttttcttcggtttt

SEQ ID NO: 1244 - DNA, 25 bp, synthetic construct
  misc_feature 1..25: note = ≅DNA/2xPS gRNA 5' extension
  misc_feature 1..2: note = Linked by phosphorothioate (PS) bond
  misc_feature 2..3: note = Linked by phosphorothioate (PS) bond
  source 1..25: mol_type = other DNA
tttttccgaagttttcttcggtttt

SEQ ID NO: 1245 - DNA, 30 bp, synthetic construct
  misc_feature 1..30: note = +DNA/2xPS gRNA 5' extension
  misc_feature 1..2: note = Linked by phosphorothioate (PS) bond
  misc_feature 2..3: note = Linked by phosphorothioate (PS) bond
  source 1..30: mol_type = other DNA
aacgctttttccgaagttttcttcggtttt

SEQ ID NO: 1246 - DNA, 41 bp, synthetic construct
  misc_feature 1..41: note = ″DNA/2xPS gRNA 5' extension
  misc_feature 1..2: note = Linked by phosphorothioate (PS) bond
  misc_feature 2..3: note = Linked by phosphorothioate (PS) bond
  source 1..41: mol_type = other DNA
gcgttgttttcaacgctttttccgaagttttcttcggtttt

SEQ ID NO: 1247 - DNA, 62 bp, synthetic construct
  misc_feature 1..62: note = βDNA/2xPS gRNA 5' extension
  misc_feature 1..2: note = Linked by phosphorothioate (PS) bond
  misc_feature 2..3: note = Linked by phosphorothioate (PS) bond
  source 1..62: mol_type = other DNA
ggcttcttttgaagcctttttgcgttgttttcaacgctttttccgaagttttcttcggtttt

SEQ ID NO: 1248 - DNA, 25 bp, synthetic construct
  misc_feature 1..25: note = ≅DNA/2xPS gRNA 5' extension
  misc_feature 1..2: note = Linked by phosphorothioate (PS) bond
  misc_feature 2..3: note = Linked by phosphorothioate (PS) bond
  source 1..25: mol_type = other DNA
atgtgtttttgtcaaaagacctttt

SEQ ID NO: 1249 - DNA, 25 bp, synthetic construct
  misc_feature 1..25: note = ≅A gRNA 5' extension
  source 1..25: mol_type = other DNA
aaaaaaaaaaaaaaaaaaaaaaaaa

SEQ ID NO: 1250 - DNA, 25 bp, synthetic construct
  misc_feature 1..25: note = ≅T gRNA 5' extension
  source 1..25: mol_type = other DNA
ttttttttttttttttttttttttt

SEQ ID NO: 1251 - RNA, 25 nt, synthetic construct
  misc_feature 1..25: note = ≅RNA/2xPS gRNA 5' extension
  misc_feature 1..2: note = Linked by phosphorothioate (PS) bond
  misc_feature 1: note = Modified to contain 2'-O-methyl
  misc_feature 2: note = Modified to contain 2'-O-methyl
  misc_feature 2..3: note = Linked by phosphorothioate (PS) bond
  source 1..25: mol_type = other RNA
atgtgtttttgtcaaaagacctttt

SEQ ID NO: 1252 - RNA, 25 nt, synthetic construct
  misc_feature 1..25: note = ≅polyA RNA/2xPS gRNA 5' extension
  misc_feature 1..2: note = Linked by phosphorothioate (PS) bond
  misc_feature 1: note = Modified to contain 2'-O-methyl
  misc_feature 2: note = Modified to contain 2'-O-methyl
  misc_feature 2..3: note = Linked by phosphorothioate (PS) bond
  source 1..25: mol_type = other RNA
aaaaaaaaaaaaaaaaaaaaaaaaa

SEQ ID NO: 1253 - RNA, 25 nt, synthetic construct
  misc_feature 1..25: note = ≅polyU RNA/2xPS gRNA 5' extension
  misc_feature 1..2: note = Linked by phosphorothioate (PS) bond
  misc_feature 1: note = Modified to contain 2'-O-methyl
  misc_feature 2: note = Modified to contain 2'-O-methyl
  misc_feature 2..3: note = Linked by phosphorothioate (PS) bond
  source 1..25: mol_type = other RNA
ttttttttttttttttttttttttt

SEQ ID NO: 1254 - RNA, 21 nt, synthetic construct
  misc_feature 1..21: note = HBG1-1 21mer targeting domain
  source 1..21: mol_type = other RNA
ccttgtcaaggctattggtca

SEQ ID NO: 1255 - DNA, 21 bp, synthetic construct
  misc_feature 1..21: note = HBG1-1 21mer targeting domain
  source 1..21: mol_type = other DNA
ccttgtcaaggctattggtca

SEQ ID NO: 1256 - RNA, 22 nt, synthetic construct
  misc_feature 1..22: note = HBG1-1 22mer targeting domain
  source 1..22: mol_type = other RNA
ccttgtcaaggctattggtcaa

SEQ ID NO: 1257 - DNA, 22 bp, synthetic construct
  misc_feature 1..22: note = HBG1-1 22mer targeting domain
  source 1..22: mol_type = other DNA
ccttgtcaaggctattggtcaa

SEQ ID NO: 1258 - RNA, 23 nt, synthetic construct
  misc_feature 1..23: note = HBG1-1 23mer targeting domain
  source 1..23: mol_type = other RNA
ccttgtcaaggctattggtcaag

SEQ ID NO: 1259 - DNA, 23 bp, synthetic construct
  misc_feature 1..23: note = HBG1-1 23mer targeting domain
  source 1..23: mol_type = other DNA
ccttgtcaaggctattggtcaag

SEQ ID NO: 1260 - RNA, 21 nt, synthetic construct
  misc_feature 1..21: note = AsCpf1 HBG1 promoter-1 21mer targeting domain
  source 1..21: mol_type = other RNA
agacagatatttgcattgaga

SEQ ID NO: 1261 - DNA, 21 bp, synthetic construct
  misc_feature 1..21: note = AsCpf1 HBG1 promoter-1 21mer targeting domain
  source 1..21: mol_type = other DNA
agacagatatttgcattgaga

SEQ ID NO: 1262 - RNA, 21 nt, synthetic construct
  misc_feature 1..21: note = AsCpf1 HBG1 promoter-2 21mer targeting domain
  source 1..21: mol_type = other RNA
cattgagatagtgtggggaag

SEQ ID NO: 1263 - DNA, 21 bp, synthetic construct
  misc_feature 1..21: note = AsCpf1 HBG1 promoter-2 21mer targeting domain
  source 1..21: mol_type = other DNA
cattgagatagtgtggggaag

SEQ ID NO: 1264 - RNA, 21 nt, synthetic construct
  misc_feature 1..21: note = AsCpf1 HBG1 promoter-6 21mer targeting domain
  source 1..21: mol_type = other RNA
cttctcccatcatagaggata

SEQ ID NO: 1265 - DNA, 21 bp, synthetic construct
  misc_feature 1..21: note = AsCpf1 HBG1 promoter-6 21mer targeting domain
  source 1..21: mol_type = other DNA
cttctcccatcatagaggata

SEQ ID NO: 1266 - DNA, 20 bp, synthetic construct
  misc_feature 1..20: note = Forward primer
  source 1..20: mol_type = other DNA
catggcgtctggactaggag

SEQ ID NO: 1267 - DNA, 24 bp, synthetic construct
  misc_feature 1..24: note = Reverse primer
  source 1..24: mol_type = other DNA
aaacacatttcacaatccctgaac

SEQ ID NO: 1268 - DNA, 53 bp, synthetic construct
  misc_feature 1..53: note = OLI13753 forward primer
  source 1..53: mol_type = other DNA
acactctttccctacacgacgctcttccgatctcatggcgtctggactaggag

SEQ ID NO: 1269 - DNA, 58 bp, synthetic construct
  misc_feature 1..58: note = OLI13752 reverse primer
  source 1..58: mol_type = other DNA
gtgactggagttcagacgtgtgctcttccgatctaaacacatttcacaatccctgaac

SEQ ID NO: 1270 - DNA, 499 bp, synthetic construct
  misc_feature 1..499: note = AMP3322
  source 1..499: mol_type = other DNA
catggcgtctggactaggagcttattgataacctcagacgttccagaagcgagtgtgtggaactgctgaagggtgcttccttttattcttcatccctagccagccgccggcccctggcctcactggatactctaagactattggtcaagtttgccttgtcaaggctattggtcaaggcaaggctggccaacccatgggtggagtttagccagggaccgtttcagacagatatttgcattgagatagtgtggggaaggggcccccaagaggatactgctaattttttttatagcctttgccttgttccgattcagtcattccagtttttctctaatttattcttccctttagctagtttccttctcccatcatagaggataccaggacttcttttgtcagccgttttttaccttcttgtctctagctccagtgaggcctgtagtttaaagctaaagcatgtaccaatttttgaaaagttcagggattgtgaaatgtgttt

SEQ ID NO: 1271 - DNA, 25 bp, synthetic construct
  misc_feature 1..25: note = AD-P-6 ddPCR Assay6
  source 1..25: mol_type = other DNA
tccctgatgaacatagatgccaaca

SEQ ID NO: 1272 - DNA, 21 bp, synthetic construct
  misc_feature 1..21: note = AD33 ddPCT Assay6
  source 1..21: mol_type = other DNA
actgggaaagaacataaccaa

SEQ ID NO: 1273 - DNA, 22 bp, synthetic construct
  misc_feature 1..22: note = AD34 ddPCT Assay6
  source 1..22: mol_type = other DNA
gatatgctgtcggatgctatta

SEQ ID NO: 1274 - DNA, 26 bp, synthetic construct
  misc_feature 1..26: note = AD-P-9 ddPCT Assay9
  source 1..26: mol_type = other DNA
agaatgttctttggcaggtactgtgg

SEQ ID NO: 1275 - DNA, 20 bp, synthetic construct
  misc_feature 1..20: note = AD95 ddPCT Assay9
  source 1..20: mol_type = other DNA
cgtagctcttctatgctcag

SEQ ID NO: 1276 - DNA, 18 bp, synthetic construct
  misc_feature 1..18: note = AD96 ddPCT Assay9
  source 1..18: mol_type = other DNA
ccatgtacacgcacatct